SEQUENCE LISTING (26 sequences)

SEQ ID NO: 1 - protein, 11 aa, synthetic construct
RASKSISKYLA

SEQ ID NO: 2 - protein, 7 aa, synthetic construct
SGSTLQS

SEQ ID NO: 3 - protein, 10 aa, synthetic construct
CQQHNEYTLT

SEQ ID NO: 4 - protein, 5 aa, synthetic construct
DYAWN

SEQ ID NO: 5 - protein, 17 aa, synthetic construct
ISYTLTTGYNPSLKSRI

SEQ ID NO: 6 - protein, 13 aa, synthetic construct
RSGWLLPYWYFDV

SEQ ID NO: 7 - protein, 108 aa, synthetic construct
DVQITQSPSYLAASPGETITINCRASKSISKYLAWYQEKPGKTNELLIYSGSTLQSGIPSRFRGSGSGTDFTLTISSLEPEDFAMYYCQQHNEYTLTFGGGTKLELKR

SEQ ID NO: 8 - protein, 121 aa, synthetic construct
DVQLQESGPGLVKPSQSLSLTCTVSGYSITSDYAWNWIRQFPGNKLEWMGYISYTLTTGYNPSLKSRISITRDSSKNQFFLQLNSVTTEDTATYYCARSGWLLPYWYFDVWGAGTTVTVSS

SEQ ID NO: 9 - protein, 10 aa, synthetic construct
RATSSVVYMH

SEQ ID NO: 10 - protein, 7 aa, synthetic construct
ATSNLAS

SEQ ID NO: 11 - protein, 9 aa, synthetic construct
QQWNSNPYT

SEQ ID NO: 12 - protein, 5 aa, synthetic construct
NYWIE

SEQ ID NO: 13 - protein, 19 aa, synthetic construct
EILPGNSYTNYNEKFRGKA

SEQ ID NO: 14 - protein, 6 aa, synthetic construct
PLSGNY

SEQ ID NO: 15 - protein, 213 aa, synthetic construct
QIVLSQSPAILSASPGEKVTMTCRATSSVVYMHWYQQKPGSSPKPWIYATSNLASGVPTRFSGSGSGTSYSLTISRVEAEDAATYYCQQWNSNPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 444 aa, synthetic construct
QVQLQQSGAELMKPGASVKISCKAAGYTFSNYWIEWVKQRPGHGLEWIGEILPGNSYTNYNEKFRGKATITADTSSNTAYMQLSSLTSEDSAVYYCASPLSGNYVRFAYWGQGTLVTVSTAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 17 - protein, 140 aa, synthetic construct
MGWSYIILFLVATATDVHSQVQLQQPGAELVKPGASVKLSCKASGYTFTSYWMHWVKQRPGQGLEWIGEINPNNGRTNYNAKFKSKATLTVDTSSSTAYMQLTSLTSEDSAVYYCANYGNYQSYYALDYWGQGTSVSVSS

SEQ ID NO: 18 - protein, 127 aa, synthetic construct
MSVPTQVLGLLLLWLTGARCDIQMTQSPASLSASVGETVTITCRASENIHSYLEWYQQKQGKSPHLLVYNAKTLAEGVPSRFSGSGSGTQFSLKINSLQPEDFGSYYCQHHYGTPFTFGSGTKLEIK

SEQ ID NO: 19 - protein, 136 aa, synthetic construct
MGWSYIILFLVATATDVHSQVQLKESGPGLVAPSQSLSITCTVSGFSLTSYGVHWVRQPPGKGLEWLVVIWSDGSTKYNEKFKGQATLTADTSSSTAYMQLSSLTSEDSAVYFCARSGEGSSYYYWGQGTTLTVSS

SEQ ID NO: 20 - protein, 130 aa, synthetic construct
MSVPTQVLGLLLLWLTGARCDIVLTQSPASLAVSLGQRATISCRASKSVSSSGYSFLHWYQQKPGQPPKLLIYLASNLDSGVPARFSGSGSGTDFTLNIHPVEEEDATTYYCQHSWELPAFGGGTKLEIK

SEQ ID NO: 21 - protein, 137 aa, synthetic construct
MEWSGVFIFLLSVTADVHSQVQLQQSGAELVRPGTSVKISCKASGYTFINYWLGWVKQRPGHGLEWIGDIYPGGGHTKYNEKFKGKATLTADTSSSTAYMQLSSLTSEDSAVFFCARSGVGSSYYYWGQGTTLTVSS

SEQ ID NO: 22 - protein, 130 aa, synthetic construct
METDTLLLWVLLLWVPGSTGDIVLTQSPASLAVSLGQRATISCRASKSVSSSGYSFLHWYQQKPGQPPKLLIYLASNLDSGVPARFSGSGSGTDFTLNIHPVEEEDATTYYCQHSWELPTFGGGTKLEIK

SEQ ID NO: 23 - protein, 129 aa, synthetic construct
MEWSGVFIFLLSVTADVHSQVQLKESGPGLVAPSQSLSITCTVSGFSLTSYGVHWVRQPPGKGLEWLVVKYNEKFKGKATLTADTSSSTAYMQLSSLTSEDSAVFFCARSGVGSSYYYWGQGTTLTVSS

SEQ ID NO: 24 - protein, 132 aa, synthetic construct
METDTLLLWVLLLWVPGSTGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 25 - protein, 136 aa, synthetic construct
MEWSGVFIFLLSVTADVHSQVQLKESGPGLVAPSQSLSITCTVSGFSLTSYGVHWVRQPPGKGLEWLVVIWSDGSTKYNEKFKGKATLTADTSSSTAYMQLSSLTSEDSAVFFCARSGVGSSYYYWGQGTTLTVSS

SEQ ID NO: 26 - protein, 130 aa, synthetic construct
METDTLLLWVLLLWVPGSTGDIVLTQSPASLAVSLGQRATISCRASKSVSSSGYSFLHWYQQKPGQPPKLLIYLASNLDSGVPARFSGSGSGTDFTLNIHPVEEEDATTYYCQHSWELPTFGGGTRLEIK

(19) United States
(12) Patent Application Publication
(10) Pub. No.: US 2026/0347413 A1
(43) Pub. Date: Nov. 5, 2026

(54) HUMANIZED MONOCLONAL ANTIBODY AND VACCINES AGAINST MEASLES VIRUS

(71) Applicants: THE TRUSTEES OF COLUMBIA UNIVERSITY IN THE CITY OF NEW YORK; INTERNATIONAL CENTER FOR RESEARCH IN INFECTIOLOGY, Lyon (FR); La Jolla Institute for Immunology, La Jolla, CA (US)

(72) Inventors: Matteo POROTTO, New York, NY (US); Anne MOSCONA, New York, NY (US); Illya TRAKHT, Bronx, NY (US); Gavreel KALANTAROV, Fort Lee, NJ (US); Roberta Della MARCA, New York, NY (US); Gilian ZIPURSKY, New York, NY (US); Erica Ollmann SAPHIRE, La Jolla, CA (US); Dawid ZYLA, La Jolla, CA (US); Branka HORVAT, Lyon (FR); Cyrille MATHIEU, Lyon (FR)

(21) Appl. No.: 19/653,002

(22) Filed: Apr. 20, 2026

Related U.S. Application Data
(63) Continuation-in-part of application No. PCT/US2024/052261, filed on Oct. 21, 2024.
(60) Provisional application No. 63/592,147, filed on Oct. 20, 2023.
(60) Provisional application No. 63/592,145, filed on Oct. 20, 2023.
(60) Provisional application No. 63/663,690, filed on Jun. 24, 2024.

(51) Int. Cl. C07K 14/005; A61K 39/00; A61K 39/155; A61K 39/42; A61P 37/02; A61P 37/04; C07K 16/11; C12N 7/00

(52) U.S. Cl. CPC C07K 14/005; A61K 39/155; A61K 39/42; A61P 37/02; A61P 37/04; C07K 16/11; C12N 7/00; A61K 2039/525; A61K 2039/543; C07K 2317/24; C07K 2317/565; C07K 2317/76; C07K 2319/00; C12N 2760/18022; C12N 2760/18033

(57) ABSTRACT

A chimeric monoclonal antibody (mAb 77.1) neutralizes measles fusion protein and its cryo-EM structure in complex with MeV F in the prefusion conformation. Vaccine compositions using MeV F protein or its ectodomain are mutated to have stabilized pre-fusion conformation. Vaccine compositions may be varied, targeting other paramyxoviruses; they are mutated forms of paramyxovirus F protein with mutations at specific positions on the protein. Thermostable full-length MeV fusion proteins (FFLs) induce protective immunity and neutralizing antibodies in vivo. Vaccination with the FFL induces neutralization titer higher than with live vaccine, providing robust humoral response, likely protective also for individuals with impaired cellular immunity. The 8 specific mAbs, alone or combined with mAb 77, neutralize post fusion transition of MeV fusion protein, interrupting the F refolding process and preventing conformational changes required for membrane fusion and viral entry: mAbs G3, F3, E11, 9E1D3, H8, 10H2G3, D9, and Y10F.

### RELATED APPLICATIONS

This application is a continuation-in-part application of International Application No. PCT/US2024/052261, filed on Oct. 21, 2024, which claims the benefit of U.S. provisional patent application 63/592,145, filed Oct. 20, 2023, to The Trustees of Columbia University, titled “HUMANIZED MONOCLONAL ANTIBODY AGAINST MEASLES VIRUS,” U.S. provisional patent application 63/592,147, filed Oct. 20, 2023, to The Trustees of Columbia University, titled “VACCINES AGAINST MEASLES VIRUS,” and U.S. provisional patent application 63/663,690, filed Jun. 24, 2024, titled “DEVELOPMENT AND USE OF MONOCLONAL ANTIBODIES AGAINST F-PROTEIN OF MEASLES VIRUS,” the entirety of the disclosures of which are hereby incorporated by this reference.

### STATEMENT REGARDING FEDERALLY SPONSORED RESEARCH

This invention was made with government support under AI176833, AI159085, NS105699, and NS091263 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 25711 byte XML file named “219WO-PCT_seq_listing.xml” created on Apr. 20, 2026.

### TECHNICAL FIELD

The subject matter disclosed herein is generally directed to stabilized paramyxovirus F protein and antibodies against F protein.

### BACKGROUND

Measles virus (MeV) is a highly transmissible (R₀ of 12-18) respiratory, enveloped, single-stranded negative-sense RNA virus. MeV belongs to the Paramyxoviridae family and causes significant morbidity and mortality among young children worldwide, despite the availability of safe and effective MeV vaccines. Measles accounts for more childhood deaths than any other vaccine-preventable disease. MeV was presumed to be eliminated in the U.S. in 2000 (i.e. ≥12 months without transmission). However, reintroduction of the virus has resulted in a resurgence in recent years, causing repeated outbreaks. Outbreaks in the U.S. and other countries with high vaccination coverage are often attributed to unvaccinated individuals. However, a significant number of cases also occur in previously vaccinated people, since up to 5% of vaccine recipients do not develop adequate protective antibodies even after the recommended two-dose vaccination regimen, and because vaccine-elicited immunity likely wanes over time. In addition, the influence of anti-vaccination proponents in social media led to vaccine hesitancy among parents and pockets of unvaccinated children.

With no effective antiviral regimen currently available for MeV, insufficient vaccine coverage (which is defined as ≤95% vaccination rate by WHO) can lead to a rise in MeV disease among vulnerable populations. Indeed, MeV has already undergone a global resurgence; estimated global MeV deaths rose from 89,780 in 2016 to 207,500 in 2019. The situation may worsen since the SARS-CoV-2 pandemic reduced routine childhood vaccination coverage and may expose more vulnerable individuals to infection. There have been over 122,000 cases in the first half of 2023 alone. Without global eradication, a significant increase in the number of MeV cases can be unavoidable. Even mild cases of measles infection can result in immune “amnesia”, and loss of preexisting antibodies against other infections, leaving measles convalescent individuals more vulnerable to future diseases.

Of particular concern during this resurgence in MeV cases are the rising number of individuals with compromised immune systems, such as cancer patients, transplant recipients, persons with autoimmune disorders on immunosuppressive therapies, and human immunodeficiency virus (HIV)-infected individuals, in addition to pregnant women. In such immunocompromised people, vaccination with current vaccines containing live-attenuated virus is often risky and inefficient; antibody responses are impaired, and even pre-existing immunity wanes. Measles resurgence in other parts of the world, combined with increased vaccine hesitancy, increases the risk of future measles outbreaks and the risk to vulnerable patient populations. Notably, despite the foreseeable rise in measles cases and measles deaths, there are still no effective antivirals approved for human use. Neutralizing monoclonal antibodies offer lifesaving treatments against a number of viruses, but surprisingly, there are none yet available for prevention or treatment of measles virus. Even more surprisingly, there are not yet structures of any antibodies against the measles virus by which Applicants might interpret epitopes or activities that prevent or treat infection.

The current measles virus (MeV) vaccine, developed in the early 1960s, is a live-attenuated virus created by repeatedly passaging MeV in cell culture. Although this vaccine is safe and effective for most healthy people, the MMR vaccine is contraindicated in immunocompromised individuals because the live vaccine itself can cause fatal illness in those with severely weakened immune systems. These groups must instead rely on herd immunity for protection which is a risky proposition. Even about 5% of healthy people do not develop sufficient immune responses to the vaccine. A greater issue, which causes frequent surges in MeV cases in the United States, is insufficient vaccine coverage at home and abroad coupled with the extraordinary transmissibility of MeV (R0 of 12-18 people per each infected person, with infection by the respiratory route). Even among healthy individuals, immune responses to measles vaccine can fail or wane. Coupled with the extraordinary transmissibility of MeV (R° of 12-18 people per each infected person, with infection by the respiratory route), insufficient vaccine coverage presents a substantial hurdle to eradication and high likelihood that outbreaks will continue causing 10-20 million measles cases each year. In 2019, approximately 207,500 people died from MeV infection worldwide, and yet vaccination rates dropped to 81%. From 2021 to 2022 the death toll from measles increased by 43%. In 2019, 1,282 measles cases were reported in the U.S. alone (the highest number since 1992), with 128 hospitalizations and 61 cases with complications (e.g., pneumonia and encephalitis), including a vertical transmission (mother to fetus) at Columbia University Irving Medical Center. The SARS-CoV-2 pandemic reduced routine childhood vaccination coverage and may expose more vulnerable individuals to infection. Without global eradication, a significant increase in MeV cases may be unavoidable. Apart from acute measles with all its sequelae, MeV infection causes immune amnesia, resulting in loss of specific immune responses against unrelated infectious agents and thus increasing susceptibility to other infectious diseases.

The MeV envelope glycoproteins hemagglutinin (H, the receptor binding protein) and fusion (F) protein play critical roles in viral cell entry. Fis synthesized as a precursor (F₀). After its synthesis, F₀ is cleaved by furin within the virus-producing cell to yield the pre-fusion F complex, consisting of three C-terminal F₁ subunits associated covalently via disulfide bonds between three N-terminal F₂ subunits. Even after this cleavage, F is kinetically trapped in a metastable conformation and is primed for fusion activation upon engagement of H with a cellular receptor (either CD150 or nectin-4). After H engages the receptor, the pre-fusion F undergoes a conformational transition, starting with the refolding of N-terminal heptad repeat (HRN) exposing the fusion peptide (FP) into the target cells, and irreversibly folding into a post-fusion conformation by formation of 6-helix bundles between HRN and C-terminal heptad repeat (HRC) as it drives virus and host membrane fusion.

The H and F proteins (i.e., the fusion complex) are the main targets of neutralizing antibodies. The current vaccine elicits mostly antibodies against hemagglutinin (H): 80% of the human sera antibodies are specific for H, and only 20% for F, and the anti-F antibodies elicited by the current vaccine are poorly neutralizing. The primary technical challenge for vaccine design has been obtaining the F protein in its pre-fusion state, representing an ideal target to elicit protective immunity. Several strategies have been proposed for stabilizing the pre-fusion state of the F proteins, which involve the introduction of several residues that may also alter the natural viral epitopes.

A vaccine strategy based on recombinant subunit forms of the viral envelope fusion glycoprotein is needed in order to provide a safe vaccine for the growing number of people who cannot be vaccinated, including young children, pregnant women, and people with weakened immune systems.

Citation or identification of any document in this application is not an admission that such a document is available as prior art to the present invention.

### SUMMARY

In one aspect, the present invention provides for a thermostable full-length paramyxovirus fusion protein (F_{FL}), wherein the F_{FL} comprises at least one mutation that stabilizes the pre-fusion conformation, wherein the at least one mutation comprises at least one substitution mutation at a position corresponding to residue 170, residue 442, residue 455, residue 459, or residue 506 of measles virus (MeV) F protein. In certain embodiments, the F_{FL} further comprises at least one mutation that removes or inactivates the furin cleavage site corresponding to the furin cleavage site of MeV F protein. In certain embodiments, the F_{FL} comprises the following mutations corresponding to MeV F protein: E455G; or E170G/E455G; or E170G/E455G/G506E; or E170G/E455G/V459I/G506E; or E170G/Y442H/E455G/G506E; or E170G/Y442H/E455G/V459I/G506E. In certain embodiments, the paramyxovirus is measles virus (MeV).

In another aspect, the present invention provides for a paramyxovirus vaccine comprising the F_{FL} of any embodiment herein.

In another aspect, the present invention provides for a method of inducing protective immunity and neutralizing antibodies, the method comprising administration of a therapeutically effective dose of the paramyxovirus vaccine of any embodiment herein to a subject in need thereof.

In another aspect, the present invention provides for a paramyxovirus vaccine composition comprising paramyxovirus F protein or its ectodomain, wherein the paramyxovirus F protein or its ectodomain comprises at least one mutation that stabilizes the pre-fusion conformation, wherein the at least one mutation comprises at least one substitution mutation at a position corresponding to residue 170, residue 442, residue 455, residue 459, or residue 506 of measles virus (MeV) F protein. In certain embodiments, the at least one mutation corresponds to E170G, Y442H, E455G, V459I, or G506E in MeV F protein. In certain embodiments, the paramyxovirus F protein or its ectodomain comprises the following mutations corresponding to MeV F protein: E455G; or E170G/E455G; or E170G/E455G/G506E; or E170G/E455G/V459I/G506E; or E170G/Y442H/E455G/G506E; or E170G/Y442H/E455G/V459I/G506E. In certain embodiments, the paramyxovirus F protein or its ectodomain further comprises at least one mutation that removes or inactivates the furin cleavage site corresponding to the furin cleavage site of MeV F protein. In certain embodiments, the vaccine composition is a measles virus (MeV) vaccine composition.

In another aspect, the present invention provides for a method of vaccinating against a paramyxovirus comprising administering to the subject the paramyxovirus vaccine composition corresponding to any embodiment herein, wherein the paramyxovirus vaccine composition comprises the F protein or its ectodomain of the paramyxovirus vaccinating against.

In another aspect, the present invention provides for a method producing neutralizing antibodies against a paramyxovirus, the method comprising: administering to a subject the paramyxovirus vaccine composition of any embodiment herein; confirming the subject produces neutralizing antibodies from the paramyxovirus vaccine composition; isolating B cells from the subject; culturing the B cells to form hybridomas; and isolating the hybridoma that produces neutralizing antibodies against the paramyxovirus. In certain embodiments, the method further comprises isolating neutralizing antibodies against the paramyxovirus from the isolated the hybridoma that produces neutralizing antibodies against the paramyxovirus. In certain embodiments, the paramyxovirus is measles virus (MeV).

In another aspect, the present invention provides for an antibody against measles virus F protein, comprising: one or more light chain variable region complementarity determining regions (CDRs) selected from the group consisting of: a first CDR comprising the sequence RASKSISKYLA (SEQ ID NO: 1), a second CDR comprising the sequence SGSTLQS (SEQ ID NO: 2), and a third CDR comprising the sequence CQQHNEYTLT (SEQ ID NO: 3); or one or more heavy chain variable region CDRs selected from the group consisting of: a first CDR comprising the sequence DYAWN (SEQ ID NO: 4), a second CDR comprising the sequence ISYTLTTGYNPSLKSRI (SEQ ID NO: 5), and a third CDR comprising the sequence RSGWLLPYWYFDV (SEQ ID NO: 6). In certain embodiments, the one or more light chain variable region CDRs are grafted onto a human antibody. In certain embodiments, the one or more heavy chain variable region CDRs are grafted onto a human antibody. In certain embodiments, the light chain variable region comprises a first CDR comprising the sequence RASKSISKYLA (SEQ ID NO: 1), a second CDR comprising the sequence SGSTLQS (SEQ ID NO: 2), and a third CDR comprising the sequence CQQHNEYTLT (SEQ ID NO: 3). In certain embodiments, the heavy chain variable region comprises a first CDR comprising the sequence DYAWN (SEQ ID NO: 4), a second CDR comprising the sequence ISYTLTTGYNPSLKSRI (SEQ ID NO: 5), and a third CDR comprising the sequence RSGWLLPYWYFDV (SEQ ID NO: 6). In certain embodiments, the light chain variable region comprises a first CDR comprising the sequence RASKSISKYLA (SEQ ID NO: 1), a second CDR comprising the sequence SGSTLQS (SEQ ID NO: 2), and a third CDR comprising the sequence CQQHNEYTLT (SEQ ID NO: 3); and the heavy chain variable region comprises a first CDR comprising the sequence DYAWN (SEQ ID NO: 4), a second CDR comprising the sequence ISYTLTTGYNPSLKSRI (SEQ ID NO: 5), and a third CDR comprising the sequence RSGWLLPYWYFDV (SEQ ID NO: 6). In certain embodiments, the sequence of the light chain variable region is: DVQITQSPSYLAASPGETITINCRASKSISKYLAWYQEKPGKTNELLIYSGSTLOSGIPS RFRGSGSGTDFTLTISSLEPEDFAMYYCQQHNEYTLTFGGGTKLELKR (SEQ ID NO: 7). In certain embodiments, the sequence of the heavy chain variable region is: DVQLQESGPGLVKPSQSLSLTCTVSGYSITSDYAWNWIRQFPGNKLEWMGYISYTLT TGYNPSLKSRISITRDSSKNQFFLQLNSVTTEDTATYYCARSGWLLPYWYFDVWGAG TTVTVSS (SEQ ID NO: 8). In certain embodiments, the antibody is encoded by GS66077-1 pTRIOZ-hIgG1_HRV3C_77.1.

In another aspect, the present invention provides for an antibody against measles virus F protein, comprising: one or more light chain variable region complementarity determining regions (CDRs) selected from the group consisting of: a first CDR comprising the sequence RATSSVVYMH (SEQ ID NO: 9), a second CDR comprising the sequence ATSNLAS (SEQ ID NO: 10), and a third CDR comprising the sequence QQWNSNPYT (SEQ ID NO: 11); or one or more heavy chain variable region CDRs selected from the group consisting of: a first CDR comprising the sequence NYWIE (SEQ ID NO: 12), a second CDR comprising the sequence EILPGNSYTNYNEKFRGKA (SEQ ID NO: 13), and a third CDR comprising the sequence PLSGNY (SEQ ID NO: 14). In certain embodiments, the one or more light chain variable region CDRs are grafted onto a human antibody. In certain embodiments, the one or more heavy chain variable region CDRs are grafted onto a human antibody. In certain embodiments, the light chain variable region comprises a first CDR comprising the sequence RATSSVVYMH, a second CDR comprising the sequence ATSNLAS, and a third CDR comprising the sequence QQWNSNPYT. In certain embodiments, the heavy chain variable region comprises a first CDR comprising the sequence NYWIE, a second CDR comprising the sequence EILPGNSYTNYNEKFRGKA, and a third CDR comprising the sequence PLSGNY. In certain embodiments, the light chain variable region comprises a first CDR comprising the sequence RATSSVVYMH, a second CDR comprising the sequence ATSNLAS, and a third CDR comprising the sequence QQWNSNPYT; and the heavy chain variable region comprises a first CDR comprising the sequence NYWIE, a second CDR comprising the sequence EILPGNSYTNYNEKFRGKA, and a third CDR comprising the sequence PLSGNY. In certain embodiments, the sequence of the light chain sequence is: QIVLSQSPAILSASPGEKVTMTCRATSSVVYMHWYQQKPGSSPKPWIYATSNLASGV PTRFSGSGSGTSYSLTISRVEAEDAATYYCQQWNSNPYTFGGGTKLEIKRADAAPTV SIFPPSSEQLTSGGASVVCFLNNFYPKDINVKWKIDGSERQNGVLNSWTDQDSKDST YSMSSTLTLTKDEYERHNSYTCEATHKTSTSPIVKSFNRNEC (SEQ ID NO: 15). In certain embodiments, the sequence of the heavy chain sequence is:

(SEQ ID NO: 16)
QVQLQQSGAELMKPGASVKISCKAAGYTFSNYWIEWVKQRPGHGLEWIGE
ILPGNSYTNYNEKFRGKATITADTSSNTAYMQLSSLTSEDSAVYYCASPL
SGNYVRFAYWGQGTLVTVSTAKTTPPSVYPLAPGSAAQTNSMVTLGCLVK
GYFPEPVTVTWNSGSLSSGVHTFPAVLQSDLYTLSSSVTVPSSTWPSETV
TCNVAHPASSTKVDKKIVPRDCGCKPCICTVPEVSSVFIFPPKPKDVLTI
TLTPKVTCVVVDISKDDPEVQFSWFVDDVEVHTAQTQPREEQFNSTFRSV
SELPIMHQDWLNGKEFKCRVNSAAFPAPIEKTISKTKGRPKAPQVYTIPP
PKEQMAKDKVSLTCMITDFFPEDITVEWQWNGQPAENYKNTQPIMDTDGS
YFVYSKLNVQKSNWEAGNTFTCSVLHEGLHNHHTEKSLSHSPGK.

In another aspect, the present invention provides for a humanized antibody comprising one or more CDRs from a single antibody selected from the group consisting of: C6, 9E1D3, H8, D9, and 10H2G3.

In another aspect, the present invention provides for a method of neutralizing postfusion transition of measles virus (MeV) fusion protein in a subject, the method comprising administration of an effective amount of a first monoclonal antibody (mAb) selected from the group consisting of: G3, C6, F3, E11, 9E1D3, G4, H8, 10H2G3, D9, and Y10F, wherein the antibody is administered alone or in combination with mAb 77 to a subject in need thereof.

In certain embodiments, the administration allows initial activation and refolding of the MeV F ectodomain and interrupts the refolding process of the F to prevent conformational changes required for membrane fusion and viral entry. In certain embodiments, the first mAb, alone or in combination with mAb 77, forms a complex with F.

In another aspect, the present invention provides for a method of inhibiting measles virus spread through cells, the method comprising administering to the cells the humanized monoclonal antibody of any embodiment herein.

In another aspect, the present invention provides for a method of treating measles virus infection in a subject, the method comprising administering to the subject the humanized monoclonal antibody of any embodiment herein.

In certain embodiments, the humanized monoclonal antibody administered within 24 hours of exposure to measles virus.

In another aspect, the present invention provides for a method of prophylactically treating measles, the method comprising administering to the subject the humanized monoclonal antibody of any embodiment herein. In certain embodiments, the humanized monoclonal antibody is administered no more than 12 hours before anticipated exposure to measles virus.

In certain embodiments, the humanized monoclonal antibody is administered onto a mucosal surface. In certain embodiments, the humanized monoclonal antibody is administered intranasally.

The foregoing and other aspects, features, and advantages will be apparent from the DESCRIPTION and DRAWINGS, and from the CLAIMS if any are included.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will hereinafter be described in conjunction with the appended and/or included DRAWINGS, where like designations denote like elements, and:

FIG. 1A-1B-mAb 77.1 displays robust antiviral activity in vitro and in vivo. A) Antiviral potency of mAb 77.1 (violet) in a viral entry assay to measure inhibition of infection in Vero cells expressing SLAM/CD150. The activity was compared to the established MeV inhibitors, 3G (green) and HRC4 (blue). Values for IC₅₀ and Hill slope were estimated using logistic regression, and values were: 3G: IC₅₀=150.1±16.2 nM, Hills slope=0.37±0.01, HRC4: IC₅₀=1.17±0.27 nM, Hills slope=0.62±0.07, mAb 77.1: IC₅₀=0.034±0.005 nM, Hills slope=0.52±0.04. B) In vivo passive immunization experiment to assess in vivo efficacy of mAb 77.1. Cotton rats received intraperitoneal (IP) injection of 0.1 mg/kg or 1 mg/kg mAb 77.1 12 hours before challenge with MeV. Animals in the control group received PBS before challenge. Values correspond to MeV titers in lung tissue collected 4 days post-infection. Significance showed using the Mann-Whitney-Wilcoxon test. P-value annotation legend: ns (non-significant): p<=1.00, *: 0.01<p<=0.05, **: 0.001<p<=0.01, ***: 0.0001<p<=0.001.

FIG. 2A-2E-mAb 77.1 mirrors the action of the HRC4 inhibitor and stabilizes the extended state of F protein. A) Schematic representation of the red blood cell (RBC) assay conducted in HEK293T cells expressing a chimeric HN/H receptor with MeV F protein. Without fusion inhibitors, the HN/H receptor binds RBCs. The MeV H stalk region subsequently activates the F protein, facilitating its conformational refolding to a fusion intermediate that anchors fusion peptide in the RBC membrane, culminating in the fusion of RBC and H293T membranes. B) In the presence of zanamivir, a small-molecule inhibitor that competes with sialic acids present at RBCs on HN, reversible binding of RBCs to HN occurs. The addition of 3G locks the F protein in its pre-fusion conformation, resulting in only reversible binding of most RBCs to the HN/H. C) The subsequent fusion checkpoint is evaluated through the combined administration of zanamivir and ACK buffer. Zanamivir acts as described in panel B, whereas ACK buffer induces lysis of RBCs. At this checkpoint, fusion intermediates with anchored fusion peptides within RBC membranes resist zanamivir-induced dissociation but are lysed in the presence of ACK buffer. D) At the final fusion checkpoint, complete fusion of RBC and HEK293T membranes is achieved. Here, ACK buffer addition has no impact on the fused RBCs. E) A bar chart summarizing the results of a mechanistic assay comparing various known inhibitors. For each condition, cells were categorized based on their interaction with RBCs: reversibly bound (dark blue) where the RBCs were only adhered by chimeric H and could be easily removed by treatment with zanamivir; irreversibly bound (pink) where RBCs were initially attached by chimeric H, but subsequent F protein refolding anchored the fusion peptide in the RBC membrane, making the RBCs attachment zanamivir-insensitive and retaining RBC on the HEK293T cell surface; and fused (light blue) where RBCs underwent membrane fusion with HEK293T cells expressing the fusion complex.

FIG. 3A-3G—F_{ECTO} structure shows that the 77.1 Fab fragment engages F_{ECTO} at the base of the head domain, as elucidated by cryo-EM. A) Primary structure annotation of F_{ECTO}. Functional domains are delineated as color-coded rectangular regions, with the following key: SP for Signal Peptide, FP for Fusion Peptide, HRN for N-terminal Heptad Repeat, DIII for Domain III, DI for Domain I, DII for Domain II, and HRC for C-terminal Heptad Repeat. The presence of Twin Strep-Tag II is indicated by ‘2xST’. White hexagonal symbols denote glycosylation sites within the F₂ region. A black loop represents natural disulfide bonds. The furin cleavage site, which divides the precursor F0 into F2 and F1 subunits, is marked with black scissors icons. The locations of two stabilizing mutations are indicated by arrows. B) A ribbon representation of the 2.3 Å structure of F_{ECTO} in the presence of FIP. C) Domain description of F_{ECTO}, colored from N-terminus to C-terminus (red-purple), as in A). D) A ribbon representation of F_{ECTO} in complex with three 77.1 Fab fragments. The Fabs interact symmetrically with the lower section of the head domain in a cleft between domains I and II. The F_{ECTO} F1 chain is represented in green, the F₂ chain in gray, the 77.1 Fab heavy variable domain (VH) in violet, and the light variable domain (VL) in pale pink. The cryo-EM map delineated as a silhouette, also encompasses the masked-out region, including the constant light and heavy segments (CL and CH) of the Fab 77.1. E) CDR loops H1 and H2 establish hydrogen bonds with three distinct F_{ECTO} polypeptide chains: F2_{B} (light gray), F1_{B} (dark green), and F1_{A} (light green). The interacting residues on F_{ECTO} and Fab 77.1 are shown in the respective colors of their chains. F) The majority of the Fab 77.1 interactions are executed by the VH part of the antibody, with all three heavy chain complementarity-determining regions (CDRs H1, H2, and H3) engaging with the head domain, and only a single CDR L2 from the VL domain contributing to the interaction. Black rectangles show regions enlarged in E) and G). G) The CDR loops H-3 and L-2 only interact with a single F_{ECTO} chain, F₁. Like in panel E, the interacting residues are highlighted in the color of the respective chains.

FIG. 4A-4F—The post-fusion conformation of F_{ECTO} exhibits rearrangement of key hinge regions and domain repositioning in projection of heptad repeats and fusion machinery. A) The cryo-EM structure of F_{ECTO} in its post-fusion conformation reveals a transformation of the HRN and HRC, forming a canonical post-fusion 6-helix bundle. The F₁ chains are illustrated in shades of blue, and the F2 chains are in shades of pink. The cryo-EM map is displayed as a silhouette, with two notable glycosylation sites indicated and visualized only at the map level. B) Domain-based coloring of FECTO in post-fusion conformation as in FIG. 2B. C) A global alignment comparison between F_{ECTO} in pre-fusion (depicted in white with silhouette) and post-fusion (in domain color scheme) conformations is shown. Optimal global alignment per chain was attained by aligning F_{ECTO} on the domain I (highlighted in light green). Regions that undergo major conformational changes are surrounded in boxes and shown in panels D and E. D) Domain III undergoes the most dramatic shift, moving its center of mass over 11 Å and rotating by 52°. Most of the fold remains unchanged when local alignment is performed on the sequence region corresponding to the domain region, with the most notable changes seen in the HRN, which releases the fusion peptide and refolds it into an extended helix. The furin-containing loop is depicted as a dashed line, and yellow scissors symbolize the furin cleavage site. E) Domain II undergoes a twisting motion during the refolding step. However, only slight differences are observed in the fold after local alignment for the residue range corresponding to the domain. The most significant change is noted in the C-terminal part of the domain, comprised of the HRC, which alters direction almost 180° around residue G433. HRC in color indicates the post-fusion structure, while HRC in black denotes the pre-fusion structure. F) Mapping of residues comprising the mAb 77.1 epitope on both the pre- and post-fusion states of F_{ECTO} reveals the displacement of the epitope in the post-fusion state, thereby elucidating the absence of 77.1 binding to F_{ECTO} in its post-fusion conformation.

FIG. 5A-5E-Structural analysis of MeV F sequence conservation and viral evolution assay reveals consistent conservation across the protein and a lack of viral variants that are resistant to mAb 77.1. A) A conservation map of the MeV F full-length (FL) primary sequence was derived from over 830 F sequences sourced from the NCBI protein database. The sequences were aligned using Clustal Omega multiple sequence alignment (MSA), and the AL2CO algorithm was employed to calculate the conservation score. MeV F demonstrates uniform sequence conservation, with only two regions indicating lower conservation: the signal peptide and the cytoplasmic tail depicted with black lines on top. B) When mapping the AL2CO score onto the tertiary structure of F_{ECTO} in both its pre- and post-fusion states, no conservation hotspots are evident. In general, residues less exposed show higher conservation compared to solvent-exposed residues. C) Although most residues forming the 77.1 epitope demonstrate high sequence conservation (light gray), two residues, G37 and R437 (shown in shades of yellow), have been identified with natural variants. D) Results from the cell-based fusion inhibition assay elucidate the impact of G37R and R436K mutations on the binding affinity of 77.1. The R436K mutation, although frequent, exhibited minimal influence on antibody-mediated fusion inhibition. Conversely, the G37R mutation demonstrated a capability to evade inhibition by 77.1. E) Viral evolution assay results from control (mock), as well as passages in the presence of HRC4 and passages 4 and 5 in the presence of mAb 77.1, were evaluated for mutations in the F gene. Only mutations with an allele frequency greater than 5% and a minimum read depth of 20 were included in the analysis. Notably, passage 5 under mAb 77.1 conditions did not yield any mutations that met these filtering criteria.

FIG. 6A-6F-Elucidation of mAb 77.1 mechanism of action through NS EM and cryo-EM analyses. A) 2D class averages obtained from NS EM of the FECTO-Fab 77.1 complex subjected to a heating experiment at 50 °C for 5 minutes. Upon thermal activation, FECTO initiates its refolding process. The presence of Fab 77.1 restricts FECTO from following the canonical folding pathway. A stepwise separation sequence is below the NS EM 2D class averages, detailing the retention and subsequent FECTO separation. B) 2D cryo-EM class averages of Fab 77.1 stabilizing a truncated fragment of FECTO. The accompanying schematic is presented on the right for clarification. C) Approximately 7 Å resolution density map of the Fab 77.1-FECTO fragment superimposed on the trimeric FECTO-Fab 77.1 complex. A zoomed-in view of the corresponding density associated with only a stabilized portion of FECTO is shown on the right. The interaction between Fab 77.1 and FECTO maintains the previously characterized epitope in a stabilized form, inhibiting further refolding. Domain coloring follows the scheme presented in FIG. 3. D) Post-triggered antibody recognition in the presence or absence of mAb 77.1. In the presence of mAb 77.1, more post-trigger epitope of F is present on cells expressing F after incubation at 55 °C. E) Canonical model outlining the typical refolding pathway of F protein. F) Proposed mechanism of action for mAb 77.1, supported by NS EM 2D class averages, depicting the observed populations of FECTO-Fab 77.1 complexes throughout the experimental conditions.

FIG. 7—Detailed plot from FIG. 2 showing red blood cell assay after 48 h of transfection incubated at 37 °C for 60 min, including controls for inhibitors and mAb 77.1 concentrations.

FIG. 8A-8F—Purification assessment, NS EM of F_{ECTO}, binding affinity of FFab 77.1 to F_{ECTO}, complex formation, and assessment of F_{ECTO} stability using DSF, focusing on the effects of pH and salt concentration. A) The purity of F_{ECTO} and Fab 77.1 is verified using SDS-PAGE. F_{ECTO} exhibits a single band corresponding to approximately 54 kDa (F1+F2) in non-reducing conditions (non-red) and about 37 kDa in reducing conditions (red). Due to its high glycosylation, F₂ travels as a smear at around 20 kDa. B) Negative stain electron microscopy (NS EM) 2D classes of pre-fusion confirmation of F_{ECTO} after purification. C) Bio-Layer interferometry binding curves of F_{ECTO} and Fab 77.1 reveal high association (3.48·10⁵ M⁻¹s⁻¹) and slow dissociation kinetics (1.25·10⁻⁴s⁻¹) of Fab 77.1, with a calculated K_{D} of 3.61·10⁻¹⁰. D) The size exclusion profile of the F_{ECTO} and Fab 77.1 complex. The purple line signifies the 1:1 mixture of F_{ECTO}: Fab 77.1, with a fully formed complex peak at approximately 11.7 ml and a Fab 77.1 peak at 16.5 ml. The green line represents the F_{ECTO} elution profile. E) Screening of buffer pH to analyze its influence on F_{ECTO}'s apparent melting temperature (Tma). Five different pH values were examined, with the resulting F_{ECTO} Tma values measured and depicted in the corresponding graphs. The trend across the pH levels is shown in the last chart. F) Analysis of F_{ECTO} stability across a range of salt concentrations. The study explored six salt concentrations (60-1460 mM) and their impact on F_{ECTO} stability expressed as Tma. The trend demonstrating the relationship between salt concentration and F_{ECTO} stability is illustrated in the graph below.

FIG. 9A-9C—Representative micrographs for each dataset with particle picks from the final particle stack (left) and selected 2D classes (right). A) FECTO-Fab 77.1, B) FECTO in post-fusion conformation, C) FECTO-[FIP-HRC]₂-PEG₁₁.

FIG. 10A-10C—Final map quality, local resolution, FSC, and particle angular distribution for each recorded dataset. A) F_{ECTO}-Fab 77.1, B) F_{ECTO} in post-fusion conformation, C) F_{ECTO}-[FIP-HRC]₂-PEG₁₁.

FIG. 11A-11C—Cryo-EM data processing for each recorded dataset as described in the methods section. A) F_{ECTO}-Fab 77.1, B) F_{ECTO} in post-fusion conformation, C) F_{ECTO}-[FIP-HRC]₂-PEG₁₁.

FIG. 12A-12E—Structure analysis and comparison of F_{ECTO}. A) Application of local scaling to the F_{ECTO}-[FIP-HRC]₂-PEG₁₁ map using Occupy. On the left, the relative occupancy of the unsharpened map is shown, and on the right, an equalized map that enhances regions with low occupancy or resolution, illustrating glycans (N29, N61, and N67), the furin-containing loop, and the density from [FIP-HRC]₂-PEG₁₁. B) The equalized map is presented at both low (on the left) and high (on the right) contour levels. The locations of FIP and HRC are indicated with arrows. The density corresponding to FIP was clearly visible in the unequalized map, whereas the density associated with HRC only became noticeable at the high contour level in the equalized map. C) Differences between disulfide-stabilized F_{ECTO} (X-ray), and native F_{ECTO} (cryo-EM). The graph shows the Ca root mean square deviation (RMSD) between the X-ray structures (F_{ECTO} APO: 5YXW, F_{ECTO} FIP: 5YZD, F_{ECTO} AS-48: 5YZC), and the cryo-EM F_{ECTO}-[FIP-HRC]₂-PEG₁₁. Each model was globally aligned based on a single monomer (F1+F2), after which the RMSD was calculated between residues present in both structures. Regions with the highest RMSD include the loop 248-265 and the stalk region highlighted in D and E. D) The ribbon diagram presents the global alignment of the X-ray and cryo-EM models, as in C. The areas exhibiting high RMSD are indicated with arrows. E) Close-up view of the areas marked in D), with the starting and ending residues indicated. The color coding matches that in D).

FIG. 13A-13F—Quantitative analysis of expression, cell-surface staining and fusion inhibition of F protein variants by 77.1 antibody. A) Western blot analysis using anti-HRC antibodies to detect F protein variants after 24 h and 48 h in cell-surface biotinylation assays conducted at 32 °C and 37 °C. B) Bar graph representing the relative integrated band intensities derived from C). The data are normalized to the WT F protein band intensity at 24 h 37 °C. C) Cell surface staining of F protein variants after 48 h at 32 °C in the presence of 77.1 antibody, adjusted for average expression levels as determined from panel D. Dotted lines connect discrete data points for clarity. D) Fusion inhibition assay conducted at 32 °C comparing 77.1 antibody with various F protein variants. Data are normalized to the highest signal per variant. Solid lines represent nonlinear regression logistic curves fit to the data, whereas dotted lines merely serve to connect discrete data points for ease of interpretation. Fit values were obtained from global fits to all data points with shared IC₅₀ and Hill slope and were: WT: IC₅₀=1.2±0.3 nM, slope=0.73±0.11, R436K: IC₅₀=0.58±0.27 nM, slope=0.48±0.08, R436G: IC₅₀=65.7±41.7 nM, slope=0.12±0.02. E) Same as in panel D but presenting unnormalized data to elucidate the relative magnitude of fusion activity among different F protein variants. F) Computational analysis of the impact of point mutations on the electrostatic surface potential maps of F2MECTO. The color scale represents electrostatic potential in volts, with red indicating-10, white indicating 0, and blue indicating +10. Arrows pinpoint the locations of mutations under investigation.

FIG. 14A-14B—Allele frequency distribution of nonsynonymous and synonymous mutations in viral evolution assay. MeV was cultured in the presence of HRC4 (dimeric lipopeptide), mAb 77, or without any inhibitors for 4 to 5 passages. Nucleotide polymorphisms and corresponding amino acid changes relative to the wild-type strain are depicted. A) Comprehensive mutational profile for all samples. B) Refined mutational profile, filtered for allele frequencies ≥5% and sequencing depth ≥20. Points are color-coded to match the sequence depth, as indicated by the color bar at the bottom.

FIG. 15A-15B—Comparative analysis of the shape and dimensions between F_{ECTO} in its pre- and post-fusion conformations. A) The final models derived for both F_{ECTO} conformations are presented, with their respective dimensions indicated. B) Visual representation of the domains, elucidating the changes in domain conformations and overall shape transitioning from the pre- to post-fusion state. The domains are color-coded consistently across both models for ease of comparison.

FIG. 16A-16C—Structural and biophysical investigation of the effects of 77.1 on F_{ECTO}. A) Per residue RMSD analysis between F_{ECTO}-Fab 77.1 and [F_{ECTO}-FIP-HRC]₂-PEG₁₁ structure. Two RMSD statistics per residue were calculated: based on all atoms in the residue (blue) and only between Ca (orange). Black dots positioned on top of the lines indicate the locations of the residues involved in the 77.1 epitope. The dashed lines indicate the mean RMSD across the entire residue range. B) The influence of Fab 77.1 binding is demonstrated through structural mapping of the Ca RMSD between two pre-fusion F_{ECTO} structures in complex with either Fab 77.1 or [FIP-HRC]₂-PEG₁₁. The intensifying red gradient indicates an increase in Ca RMSD, with arrows pinpointing the antibody-binding epitope. C) Differential scanning fluorimetry (DSF) experiments were conducted with F_{ECTO} and complexes of F_{ECTO} with [FIP-HRC]₂-PEG₁₁ and Fab 77.1. The thermal stability of F_{ECTO}, stabilized by two point mutations, was with a Tₘ^{app} of 47.6 °C. The addition of [FIP-HRC]₂-PEG₁₁ enhanced the thermal stability to 56.7 °C, whereas the Fab 77.1 only slightly stabilized F_{ECTO}, raising the temperature to 51.6 °C.

FIG. 17A-17G-Analysis of Time-Resolved Heating Experiment with F_{ECTO}-Fab 77.1. A, B) 5-minute, 50 °C heat-treated controls for Fab 77.1 and F_{ECTO}2M. C) Unheated control of the F_{ECTO}-Fab 77.1 complex. D, E, F, G) Time-resolved thermal analysis of F_{ECTO}-Fab 77.1. The most representative or the sole class obtained during the 2D classification round are shown. All datasets were processed using identical parameters. Obtained states are color-coded: violet: Fab, green: F_{ECTO}/F_{ECTO}-Fab 77.1 in pre-fusion conformation, slate blue: F_{ECTO} in post-fusion conformation, orange: relaxed/open state of F_{ECTO}-Fab 77.1, red: Fab-stabilized F_{ECTO} fragment.

FIG. 18A-18D—Comparison of Paramyxovirus-specific, pre-fusion F targeted antibodies. Three main epitopes can be distinguished among all 12 characterized antibodies, including mAb 77.1. A) Apex binding antibodies (Fab PIA174: 6MJZ, Fab 12B2: 7KI4, Fab66: 6T3F, Fab 4H3: 7UOP (unpublished), Fab 3x1: 8DG8, Fab 2D3: 7UP9 (unpublished) recognize top part of domain III and F₂. B) Face binding antibodies (Fab 1A9: 7UPK (unpublished), Fab 5B3: 6TYS, Fab 1F5: 7KI6, Fab 1H8: 7UPA (unpublished)) mainly focus on domain I and lower part of domain III. C) Base binding antibodies (Fab 2B12: 7UPD (unpublished), Fab 1H1: 7UPB (unpublished), Fab 77.1: this study) bind primarily domain II, bottom of domain I and the linker region between domain II and HRC. Interestingly, Fab 1H1 also recognizes post-fusion conformation of Nipah F. D) Angle of approach of antibodies included in A-C. Axis was determined based on the long dimension of a whole Fab structure fitting into available Fab fragment. Axes are colored based on the antibody colors from A-C.

FIG. 19—Grafting 77.1 mAb. The 77.1 mAb, with variable domains (VL and VH) derived from 77.4 (mouse) was cloned in pTRIOZ-hIgG1 vector.

FIG. 20A-20B—Inhibition of viral spread. Effect of humanized monoclonal antibody (77.1 mAb) on the areas of plaques formed by the indicated recombinant MeV viruses with eGFP in infected Vero SLAM (A) or Vero cells (B). The mAb were added after the 120-min adsorption period. Representative images of Vero SLAM cells after 72 h post-infection were taken using a Cytation 5 Cell Imaging Multi-Mode Reader (BioTek) (bars, 200 μm). Areas of infection were measured using BioTek Gen5 software on images randomly acquired from one experiment in duplicate. Statistical analysis was performed using the Unpaired t test with Welch's correction (**, p<0.05; *** p<0.005; ****, p<0.0001).

FIGS. 21—77.1 mAb inhibits MeV infection in vivo. Cotton rats were infected intranasally with 10⁵ TCID₅₀/ml MeV B3-eGFP wild type. Animals received either the indicated amount of the mAb 77.1 or vehicle subcutaneously 12 hours prior infection. Animals were euthanized 5 days after infection. The results are presented as TCID₅₀/g of lung tissue. Statistical analysis was performed using the one-way ANOVA with Dunnett's multiple comparison test (** p=0.001, **** p<0.0001).

FIG. 22A-22C—Vaccination with full-length stabilized measles F is protective in vivo. A) 2D class averages derived from cryo-EM of the full-length E170G/E455G F construct embedded in either a lipid nanodisc (upper panel) or amphipol (lower panel). The high degree of flexibility in the membrane-proximal region impedes the reconstruction of the transmembrane segment. B) Cryo-EM maps of the F protein constructs, resolved at resolutions of 2.5 Å (within nanodisc) and 2.7 Å (within amphipol), demonstrating the high quality of obtained protein constructs. The lower contour map, depicted as a silhouette, shows the unresolved transmembrane (TM) region. C) Immunization with the stabilized, full-length Fin amphipol yields complete lung protection (red). Compared to this, immunization with purified MeV antigen (gradient purified, UV-inactivated MeV (Pueschel, K.; Tietz, A.; Carsillo, M.; Steward, M.; Niewiesk, S., Measles virus-specific CD4 T-cell activity does not correlate with protection against lung infection or viral clearance. Journal of virology 2007, 81 (16), 8571-8.1951373; and Reich, A.; Erlwein, O.; Niewiesk, S.; ter Meulen, V.; Liebert, U. G., CD4+ T cells control measles virus infection of the central nervous system. Immunology 1992, 76 (2), 185-91)) led to lower, ~tenfold reduction in viral load (blue). Statistical significance was determined using the Mann-Whitney-Wilcoxon test. P-value annotations: ns denotes p<=1, * denotes 0.01<p<=0.05, ** denotes 0.001<p<=0.01, and *** denotes 0.0001<p<=0.001.

FIG. 23A-23C—Ectodomain of MeV F bearing E170G and E455G is highly thermostable. A) Molecular representation of the 2.6 Å-resolution structure of the F ectodomain (residues 1-495) in complex with mAb77.1 Fab fragments overlayed on the lower contour cryo-EM map. The antibody interacts with domain II of F, stabilizing the lower section of the head domain through its simultaneous interaction with two F₁ and one F₂ chain. B) In vivo efficacy of mAb77.1 demonstrated through cotton rat passive immunization experiments. mAb77.1, administered prior to MeV infection at doses of 0.1 and 1 mg/kg, resulted in lower viral titer at both concentrations compared to the virus-only control group. Significance showed using the Mann-Whitney-Wilcoxon test. P-value annotation legend: ns: p<=1.00e+00, *: 1.00e-02<p<=5.00e-02, **: 1.00e-03<p<=1.00e-02, ***: 1.00e-04<p<=1.00e-03. C) Differential scanning fluorimetry shows the high thermostability of the F ectodomain and the influence exerted by the mAb77.1 Fab fragments and [FIP-HRC-PEG4]₂-chol fusion inhibitors (Bovier, F. T.; Rybkina, K.; Biswas, S.; Harder, O.; Marcink, T. C.; Niewiesk, S.; Moscona, A.; Alabi, C. A.; Porotto, M., Inhibition of Measles Viral Fusion Is Enhanced by Targeting Multiple Domains of the Fusion Protein. ACS Nano 2021).

FIG. 24—Vaccine-enhanced disease/lung histology. For induction of vaccine-enhanced disease, cotton rats (5 animals/group) were immunized with formalin-inactivated (FA) 10⁵ pfu MeV twice s.c. in a three-week interval and challenged with wildtype virus after 52 days. Lung pathology was scored on a scale from 0 (nothing) to 100 (severe).

FIG. 25A-25G—Structural Characterization of F Protein Constructs. A) Primary structure annotation of F_{FL}: Functional domains are delineated by color-coded rectangular regions with the following designations: SP (Signal Peptide), FP (Fusion Peptide), HRN (N-terminal Heptad Repeat), DIII (Domain III), DI (Domain I), DII (Domain II), HRC (C-terminal Heptad Repeat), TM (Transmembrane Region), and CT (Cytoplasmic Tail). White hexagons indicate glycosylation sites within the F2 region. Natural disulfide bonds and the furin cleavage site (which divides the precursor F0 into F2 and F1 subunits) are represented by a black loop and black scissors, respectively. B) SDS-PAGE analysis of purified F_{FL} constructs: Constructs of wild-type (wt), 2M, and 3M. The introduction of stabilizing mutations is shown to enhance protein yield. C) Structural representation of F_{FL}: ribbon diagrams depict F_{FL} in pre-fusion (left) and post-fusion (right) conformations, highlighting point mutations E170G and E455G (2M), 2M+Δfur (3M), and 3M+Y442H+V459I+G506E (6M). The cell membrane is illustrated as a gray rectangle, with unmodelled portions of F depicted schematically. The color scheme corresponds to that in A). D) Negative stain electron microscopy 2D classes depict the enhanced stability of the pre-fusion conformation across all F_{FL} constructs. While F wt was purified only in the post-fusion conformation, the 6M construct lacked detectable post-fusion conformation. Particles come from ~100 micrographs for each construct. E) Cryo-EM 2D classes of F_{FL} 2M visualized in nanodisc (upper) and amphipol (lower) environments. The transmembrane region is noted for lacking detailed structural information, possibly due to high flexibility or alignment procedure focusing on the head region. F) Cryo-EM reconstruction of F_{FL} constructs of F_{FL} 2M in nanodisc and amphipol and F_{FL} 3M in amphipol. High resolution is observed in the head region, but the transmembrane domain is consistently absent in the reconstructions. A low-resolution silhouette in black shows the Gaussian blurred map at 1.5 standard deviations, highlighting the membrane region. G) Model of F_{FL} 2M head region that contains F1 and F2 fragments (in green and gray, respectively), with three glycosylation sites per protomer emphasized in magenta. Each unique glycosylation site is annotated alongside a pie chart representing the glycosylation heterogeneity at that site, as determined by mass spectrometry.

FIG. 26A-26D—Immunization of cotton rats with stabilized F protein variants demonstrates protection against viral challenge and elevated antibody responses to pre-fusion epitopes. A) Cotton rat (CR) immunization with stabilized F proteins. Animals were immunized either with 2 doses of 5 μg of the indicated protein in alum at four-week intervals or with a single dose of 10⁴ TCID₅₀ of the Edmonston-Zagreb MeV vaccine. Viral titers are reported, with a detection limit of 2 log₁₀/g. The line within each box plot indicates the median viral titer. Significance was assessed by Mann-Whitney-Wilcoxon test, with p-value categorized as non-significant (ns): p≤1.00, *: 0.01<p<0.05, **: 0.001<p<0.01, ***: 0.0001<p≤0.001. B) Neutralizing antibody titers were obtained from CR sera four weeks after the last immunization. F_{FL} 3M and F_{FL} 6M induced complete protection against viral challenge and induced higher neutralizing antibody titers than immunization with live MeV vaccine. Individual animal titers are represented by points, with the mean depicted by bars and the 95% confidence interval indicated by error bars. C) Conformation-specific sera binding assay. Conformation-specific antibody binding to the F protein (using cells expressing wt F) was assessed using sera from immunized rats. The presence of antibodies targeting both pre- and post-triggered epitopes of the F protein is noted. Post-triggered epitopes were induced by incubation of cells expressing F at 55 °C for 30 minutes. Proteins with F_{FL} 2M which are more likely to shift to the post-fusion conformation, show a higher recognition of this conformation than those with F_{FL} 3M and 6M. The graphical representation uses bars for means and error bars for the 95% confidence interval, consistent with B). D) Neutralization activity directly correlates with F protein pre-fusion recognition. Data from B and C, excluding PBS and MeV controls, are represented in a correlation plot. Immunizations are shown in columns (1 left, 2 right) and epitope specificity as rows (pre-fusion top, post-fusion bottom). A linear regression is shown as a solid (R²>0.5) and dashed (R²<0.5) lines and a 95% confidence interval is shown in gray.

FIG. 27A-27D—Time-resolved stability of F variants on the cell surface exposed to 55 °C and 65 °C. A) Comparison between the wild-type (wt) strain F and the vaccine strain of MeV F. Lines represent exponential decay function. B) Stability increase resulting from the addition of point mutations identified in central nervous system (CNS) MeV strains. Solid lines indicate the exponential decay function fit, while dashed lines represent data connectivity. C) Accumulation of stabilizing point mutations enhances the stability of pre-trigger F on the cell surface exposed to 65 °C. All data were fitted using a single exponential decay function. Data were obtained from a single biological replicate consisting of three technical replicates. D) Stability of variants shown in panel C, presented as unfolding half-lives derived from the exponential decay fit. Error bars indicate the standard error of the half-lives.

FIG. 28—Reducing SDS-PAGE gel analysis of purified MeV F constructs. The reducing SDS-PAGE analysis of purified MeV F constructs demonstrates distinct band patterns for different constructs. The F_{ECTO}2M construct shows as two bands corresponding to F1 and F2. The F_{FL} 2M construct, similar to F_{ECTO} 2M (Science in press), shows a prominent F1 band at approximately 45 kDa, with the F2 band not well visible. In contrast, the F_{FL} 3M and F_{FL} 6M constructs display only the F0 band, indicating the absence of proteolytic cleavage.

FIG. 29—Biophysical Characterization of Investigated F Constructs via Nanotemper Prometheus Panta. Top Panel: The first derivative of the nano-differential scanning fluorimetry (nano-DSF) signal at 330 nm indicates the apparent melting temperatures of F_{ECTO} 2M, F_{FL} 2M, F_{FL} 3M, and F_{FL} 6M. The incorporation of stabilizing mutations results in increased melting temperatures of the purified F constructs. Bottom Panel: As determined from dynamic light scattering experiments, the cumulant radius reveals that the full-length (FL) constructs exhibit significantly less aggregation than the ectodomain. Additionally, constructs with abolished Furin cleavage sites show markedly lower aggregation following unfolding.

FIG. 30A-30E-Cryo-EM data processing flowchart. Selected classes are shown in green. A) F_{FL} 2M in nanodisc. B) F_{FL} 2M+[FIP-HRC]₂-PEG₁₁ in nanodisc. C) F_{FL} 2M+[FIP-HRC]₂-PEG₁₁ in amphipol. D) F_{FL} 3M in amphipol. E) F_{FL} 6M in amphipol.

FIG. 31A-31E-Cryo-EM statistics of recorded datasets. The first row corresponds to local resolution mapped on the final volume, the second shows the Fourier shell correlation, the third shows local resolution histogram derived from the data in row 1, the fourth row shows the angular distribution, and the fifth and sixth rows show representative micrograph and particle locations present in the final particle stack. A) F_{FL} 2M in nanodisc. B) F_{FL} 2M+[FIP-HRC]₂-PEG₁₁ in nanodisc. C) F_{FL} 2M+[FIP-HRC]₂-PEG₁₁ in amphipol. D) F_{FL} 3M in amphipol. E) F_{FL} 6M in amphipol.

FIG. 32A-32F-Cryo-EM Data on the MeV Transmembrane Region. A) Cryo-EM 2D classes of F_{FL} 2M in nanodisc. Particles were aligned on either the ectodomain (left) or nanodisc (right). Despite alignment on the nanodisc region, no internal structure (transmembrane region) was observed. The cartoon to the right of the 2D classes illustrates the effect of the alignment on the observed image. In the ectodomain-aligned particles, the nanodisc density is blurred and features are missing. In nanodisc-aligned particles, features inside the nanodisc (lipid bilayer) are visible, but the head domain is blurred and heterogeneously positioned. B) Maximum projection of the final volume (left), raw map (center), and amplified locally scaled map in Occupy (right) of F_{FL} 2M in nanodisc (top), F_{FL} 2M+[FIP-HRC]₂-PEG₁₁ in amphipol (center), and F_{FL} 3M in amphipol (bottom) show the absence of structural features within the transmembrane region. The amplified locally scaled map, however, reveals clear density for other flexible parts of the structure, such as glycans N29, N61, and N67, as well as partial density for the Δfur loop. Local-scale values are shown at the bottom and apply to all maps. C) Computational investigation of the C-terminal part of the F protein. For the F stalk region (residues 460-487), linker (residues 488-492), transmembrane region (TM, residues 495-515), and cytoplasmic tail (CT, residues 516-550), secondary structure was predicted using AlphaFold 2 (AF2) and the protein large language model ProtT5. Both models indicate that the linker region lacks defined secondary structure, similar to the CT. Secondary structure for ProtT5 is depicted as follows: •: helix, [symbol] : beta sheet, ▬: loop. D) Hypothetical hybrid model of F_{FL} incorporating the cryo-EM structure of F_{FL} 2M in amphipol (residues 24-485) and AF2 prediction (residues 487-528). The linker was modeled based on partial density present in the cryo-EM map, and the TM and CT regions were based on AF2 prediction. The conformation of the linker may result in the separation of the TM helices and increase the flexibility of the head region. Additionally, the predicted TM helix spans ~40 Å, matching the width of the lipid bilayer. E) Multiple sequence alignment (MSA) of representative members of paramyxoviruses. The stalk and TM regions are mapped based on the MeV F sequence. A similar pattern of residues is observed in the linker region across other family members. F) AF2 predictions of the stalk-linker-TM region based on the MSA of MeV F. Out of 19 species, more than half show a flexible linker depicted as a kink in the secondary structure. Only one representative structure is shown out of five predicted; some structures displayed mixed straight-bent predictions, but only the bent form is presented.

FIG. 33A-33B—Glycan shielding of F. A) Glycan envelope of F shown with conformer library of determined glycans. Two glycans, N61 (purple) and N67 (dark pink) create a dense barrier on the F apex. N29 (pink) is placed at the bottom of the protein. Possibly due to the placement and inaccessibility to downstream glycosylation enzyme, this glycan is high mannose. The minimal glycosylation leaves majority of the F face exposed to the solvent. B) Glycan shielding effect calculated based on A). The highest shielding effect is present close to the glycosylation site and apex of the protein shows higher shielding than the bottom.

FIG. 34A-34E—F_{FL} constructs immunization does not induce vaccine-enhanced respiratory disease in cotton rats. F protein constructs (5 μg), vehicle control (1 mL 1:1 PBS: Adjuphos), or measles Edmonston-Zagreb strain (10⁴ TCID₅₀) was administered subcutaneously to cotton rats. At day 28 post-primary immunization, F protein constructs or vehicle control were administered again. At day 56 post-primary immunization, cotton rats were infected with measles virus (wt B3). Cotton rats were euthanized at day 4 post-infection and the right lung lobes were prepared for histopathological evaluation. A-F) Representative regions of peribronchiolar inflammatory infiltrates for each treatment or control group. A) The vehicle control group exhibits minimal peribronchiolar infiltrates. B) Edmonston-Zagreb vaccinated animals exhibit more pronounced peribronchiolar inflammatory infiltrates, occasionally forming complete cuffs (*) around affected airways, with a higher proportion of lymphocytes and plasma cells. C-F) Animals vaccinated and boosted with F protein constructs demonstrate similar or lower degree of peribronchiolar infiltrates compared to Edmonston-Zagreb vaccinated animals (C=F_{FL} 2M; D=F_{FL} 3M; E=F_{FL} 6M). Substantial interstitial and perivascular inflammatory infiltrates are absent.

FIG. 35—Neutralization of Live Virus and Screen for hybridomas producing neutralizing antibodies. Hybridomas from a mouse vaccinated with purified and stabilized MeV F protein were screened for neutralization of Measles B3 virus. Vero SLAM cells (Vero cells stably expressing Measles receptor CD150) were treated with serial dilutions of supernatant from cultured hybridomas prior to infections with 1,000 PFU/well of MeV B3 with mCherry. Fluorescent plaques were counted two days following infection and percent inhibition of plaque formation was calculated by normalizing to the no treatment condition for each plate. Hybridoma supernatants that neutralized >80% (dotted line) at the top concentration were selected. Mouse hybridoma producing previously characterized mouse anti measles F antibody 77.4 was used as a positive control. In addition, a purified humanized version of 77.4, 77.1, was used as a technical control within each plate.

FIG. 36—Neutralization of Live Virus dose response. Antibodies from neutralizing hybridoma supernatant were purified using protein G conjugated magnetic beads and neutralization of live virus was tested using 10 nM antibody with 1:2 serial dilutions.

FIG. 37—Recognition of pre vs post triggered MeV F conformational state. Screening for conformation specific MeV F antibodies. HEK-293T cells transiently transfected with Measles B3 strain F were incubated at 4 degrees Celsius or 55 degrees Celsius for 30 minutes to maintain the pre-triggered state of F (4 °C) or induce the post triggered state of F (55 °C). Hybridoma supernatants were added to the cells at 4 °C and a fluorescent secondary anti-protein G antibody. Transition to the post-triggered state was confirmed by disappearance of previously characterized 77.4.

FIG. 38—Sequences of antibodies. (SEQ ID NOS: 17-26)

FIG. 39A-39B—Mechanism of Action. A) Red blood cell (RBC) fusion assay was conducted in HEK293T cells expressing a chimeric H/HN receptor with MeV F protein. (i) Without fusion inhibitors, the H/HN receptor binds RBCs, and the MeV H stalk region activates the F protein, facilitating its refolding to a fusion intermediate that anchors the fusion peptide in the RBC membrane, culminating in the fusion of RBC and H293T membranes. (ii) In the presence of Zanamivir, a small-molecule inhibitor that competitively binds the HN sialic acid receptor binding site, reversible binding of RBCs to HN is observed. Adding 3G locks the F protein in its pre-fusion conformation, resulting in reversible binding of most RBCs to the H/HN. (iii) The subsequent fusion checkpoint is evaluated through combined administration of Zanamivir and ACK buffer. ACK buffer induces lysis of RBCs. At this checkpoint, fusion intermediates with anchored fusion peptides within RBC membranes resist zanamivir-induced dissociation but are lysed in the presence of ACK buffer. (iv) At the final fusion checkpoint, complete fusion of RBC and HEK293T membranes is achieved. Here, ACK buffer addition has no impact on the fused RBCs. B) Bar chart summarizing the results of a mechanistic assay comparing known inhibitors and neutralizing hybridoma supernatant fluid. For each condition, cells were categorized based on their interaction with red blood cells (RBCs): reversibly bound (dark blue) in which RBCs were adhered only by chimeric H and could be removed by treatment with Zanamivir; irreversibly bound (pink) in which RBCs were initially attached by chimeric H, but subsequent F protein refolding anchored the fusion peptide in the RBC membrane, making RBC attachment insensitive to Zanamivir with RBC retained on the HEK293T cell surface; and fused (light blue) in which RBCs underwent membrane fusion with HEK293T cells expressing the fusion complex.

FIG. 40—Illustrates that current vaccines elicit mostly antibodies against hemagglutinin: 80% of human sera antibodies are against H, and only 20% against F.

FIG. 41—Plaque reduction assay. Vero SLAM cells were infected with 1000 PFU/well of MeV B3 WT in the presence of serial dilutions of monoclonal antibodies Y10F or 77.1. After 2 days, the plate was imaged, and total fluorescence was calculated by Cytation 5. The percentage of inhibition was calculated relative to no treatment.

FIG. 42A-42B—Structural characterization of mAbs Y10F and 77.1. A) Cryo-EM structure (2.6 Å) of the F_{ECTO} in complex with the Y10F Fab fragment. Y10F specifically recognizes and binds the apex region of the F protein, engaging asymmetrically with two F1 protomers and one F2 chain. B) Cryo-EM structure (3.1 Å) of a mAb77.1 and Y10F antibodies binding a single F_{ECTO}.

FIG. 43A-43C—77.1 and Y10F activity on MeV F stability. MeV B3 F expressing cells were incubated with varying concentrations of Y10F or 77.1 human monoclonal antibodies for 30′, afterward the cells were transferred to 55 °C for 0, 15, or 30 minutes and subsequently incubated with 10 nM of C6 (mouse mAb that recognized the MeV F in the post-triggered state) at 4 °C. Secondary antibodies (anti-mouse Alexa fluor 488 and anti-human Alexa Fluor 594). Cells were imaged on Cytation 5 Imager, and fluorescent cells were assessed. A) Illustrates MeV F arrangements from pre- to post-fusion state. B) Y10F maintains signal after incubation at 55 °C while 77.1 does not. C) C6 is a mouse mAb that recognizes MeV F protein in its post-triggered state. C6 signal post-incubation at 55 °C in the presence of Y10F is lower compared to 77.1 or mock treated.

FIG. 44A-44C—77.1 and Y10F block fusion via different mechanisms. A) Illustrates a RBC fusion assay. Cells expressing MeV and MeV H/HPIV3 HNChimeric receptor binding protein (to enable sialic acid binding) were allowed to bind to red bloods cells (RBCs) at 4 °C for 30 min. Unbound RBCs were washed, and cells were treated with monoclonal antibodies in A) 77.1, in B) Y10F, for 30 min at 4 °C. The cells were then brought to 37 °C for 60 min to enable F triggering and fusion with RBCs. RBCs either: 1) were released by loss of receptor engagement or viral neuraminidase activity during incubation at 37 °C; 2) remain bound by H/HN chimera but could be dislodged by treatment with small molecule zanamivir and were therefore reversibly bound; 3) remain bound by insertion of F's fusion peptide into RBC membrane but not fused with glycoprotein bearing cell (irreversibly bound); 4) fused with glycoprotein bearing cells. B) mAb77.1 blocks fusion at the irreversibly bound stage, indicating that it allows F activation and insertion into the target cells, but it blocks the subsequent refolding required for membrane merger. C) mAb Y10F blocks fusion at the reversibly bound stage, indicating that it prevents F activation.

FIG. 45—Sequences of Y10F kappa light chain and Y10F heavy chain (SEQ ID NOS: 15-16).

FIG. 46—Illustrates the measles virus (MeV).

FIG. 47—Illustrates the measles virus (MeV) entry.

FIG. 48—Illustrates MeV architecture and structure.

FIG. 49—Illustrates single particle cryogenic electron microscopy.

FIG. 50—Illustrates structural studies of MeV glycoproteins.

FIG. 51—Illustrates MeV F refolding.

FIG. 52—Illustrates MeV F refolding.

FIG. 53—Viral entry inhibition assay comparing mAb77 to previously described inhibitors.

FIG. 54—Shows the structure of F with Fab77.

FIG. 55—Shows the structure of F with Fab77.

FIG. 56—Shows the structure of F with Fab77.

FIG. 57—Shows the structure of F with Fab77.

FIG. 58—Red blood cell fusion assay using 3G, HRC4, and mAb77.1.

FIG. 59—Differential scanning fluorimetry of Fab77 and F.

FIG. 60—Temperature-resolved dynamic light scattering of F and Fab77.

FIG. 61—Negative stain electron microscopy (NS EM) images of F.

FIG. 62—Negative stain electron microscopy (NS EM) images of F and Fab77.

FIG. 63—Cryo-electron microscopy (cryo-EM) images of F and Fab77.

FIG. 64—Structural images showing that Fab stabilizes the epitope.

FIG. 65—Illustration showing the neutralization mechanism of a novel neutralizing antibody against MeV that halts the refolding before the membrane merging.

FIG. 66—Shows that the mAb77 epitope is highly conserved.

FIG. 67A-67D—Epitope Recognition and Neutralizing Activity of Anti-F Chimeric Monoclonal Antibodies. A) Recognition of cell-surface expressed F protein from the wild-type B3 and D8 strains (the only two currently circulating wt MeV strains), vaccine strain Moraten, destabilized mutant B3 L454W, and stabilized mutant B3 E455G. Cells expressing F variants were incubated for 24 hours and analyzed for binding to chimeric monoclonal antibodies (Y10F, 77.1, H8, and C6). Data are presented as mean±SEM of biologically independent experiments, each performed in technical triplicate. Source data are provided as a Source Data file. B) Antiviral potency of mAbs measured by plaque reduction neutralization tests (PRNT). Applicants assessed the antiviral potency of the four mAbs in a viral entry assay using two circulating wild-type strains (B3 and D8), a vaccine strain (Moraten), and a neuropathogenic variant (B3 L454W F). Vero-hCD150 cells were infected with approximately 250 plaque-forming units (PFU)/well of each virus in the presence of increasing concentrations of mAbs 77.1 (violet circles), H8 (blue squares), Y10F (red triangles), and C6 (green diamonds) as indicated on the x-axis. After 2 hours, the inoculum was removed and replaced with 1% methylcellulose. Infection was monitored for 72 hours, with images acquired every 24 hours using a Cytation 5 imager. Plaques were quantified at 48 hours post-infection. Data represent mean±SEM from three independent experiments. Source data are provided as a Source Data file. C) Surface plasmon resonance (SPR) sensorgrams collected on a Carterra LSA with immobilized antibodies and prefusion F_{ECTO} as the analyte. Association (0-350 s) and dissociation phases (350-1300 s) were globally fit to a 1:1 Langmuir model across a concentration series to obtain kₒₙ and k_{off}; equilibrium dissociation constants (K_{D}=k_{off}/kₒₙ) are reported for each antibody. Source data are provided as a Source Data file. D) 2D class averages obtained from negative-stain electron microscopy (NS-EM) of the Fabs-F_{ECTO} complexes. The last column shows a graphic overlay with labels showing the locations of F_{ECTO} and Fabs.

FIG. 68A-68J—Thermal stability, size, and conformational effects of F-mAb complexes. A) First-derivative nanoDSF traces for F_{ECTO}-Fab complexes. F_{ECTO}-Fab complexes (F_{ECTO}-Y10F, F_{ECTO}-H8, and F_{ECTO}-C6) were assembled 15 min before measurement, loaded into capillaries, and monitored for thermal unfolding by intrinsic fluorescence at 330 nm. Dashed, color-coded lines mark F_{ECTO} unfolding temperatures: F_{ECTO} alone, 47.2 °C; with Y10F, 55.5 °C; with H8, 44.3 °C; with C6, 47.9 °C. Unfolding of Fabs was observed in the range of 60-80° C. Source data are provided as a Source Data file. B) In parallel, dynamic light scattering (DLS) was recorded, and the cumulant radius was plotted as a function of temperature (°C). Source data are provided as a Source Data file. C) C6 binding to post-triggered F in the presence of H8 or Y10F, measured after incubation at 4° C. or following a 50° C. temperature shift. To assess the effect of H8 and Y10F on native full-length MeV F stability, cells expressing wild-type B3 F were incubated with 200 nM of either antibody. Exposure of the post-triggered F epitope was measured using C6 after 90 min at 4° C. or following a temperature shift (30 min at 4° C. then 30 min at 50 °C). The number of C6-positive cells, reflecting post-triggered F exposure, is plotted on the y-axis. Data represent the mean±SEM of n=3 (mock) or n=5 (antibody-treated) biologically independent experiments. The y-axis is on a logarithmic scale. Source data are provided as a Source Data file. D) H8-induced activation of the MeV F protein was assessed in the presence of Y10F. To evaluate the effect of Y10F on H8-mediated F triggering, HEK293T cells expressing either wild-type B3 F or the B3 E455G F mutant were pre-incubated with Y10F at the indicated concentrations (room temperature), and subsequently exposed to increasing concentrations of H8 (x-axis) at 37 °C. Post-triggered F exposure was detected after 90 min using the conformation-specific mAb C6. The number of C6-positive cells, reflecting the extent of post-triggered F exposure, is plotted on the y-axis (in logarithmic scale). Data points represent the mean±SD of technical triplicates from a single representative experiment. Source data are provided as a Source Data file. E) Size-exclusion chromatography analysis of purified MeV F ectodomain (F_{ECTO}). Chromatograms show F_{ECTO} immediately after thawing (0 h, light green), after 24 h at room temperature (dark green), and Fab H8 alone (blue). F_{ECTO} incubated for 24 h showed increased absorbance in the void volume (Vₒ), indicating aggregation and/or large conformational rearrangements (such as refolding to postfusion state). The integrated Vₒ region (7-10 mL) is shaded. Source data are provided as a Source Data file. F) SEC profiles of F_{ECTO} incubated with H8 Fab at an approximately 1:1 molar ratio (monomer: Fab) for increasing times (0.25-24 h). With longer incubation, the Vₒ peak progressively increased while the peak corresponding to the intact F_{ECTO}-H8 complex decreased, consistent with time-dependent triggering and structural rearrangement of F. Source data are provided as a Source Data file. G) Quantification of the integrated Vₒ signal from panel C plotted as a function of incubation time. Fitting to a single-exponential model yielded a half-life (t_{1/2}) of 0.98±0.58 h for H8-induced triggering. The residuals of the fit are shown in the inset. Source data are provided as a Source Data file. H) Representative two-dimensional class averages from single-particle NS EM across six time points from the same reaction. Class 1 corresponds to prefusion, unbound F_{ECTO}. Class 2 shows prefusion F_{ECTO} bound by a single H8 Fab. Class 3 represents a top view of F bound by three H8 Fabs. Class 4 shows the postfusion conformation bound by three H8 Fabs. Class 5 contains H8 Fab bound to a fragment of F_{ECTO}; due to its heterogeneity, this class was not included in occupancy analyses. Classes 3 and 4 are shown separately because some datasets contained class 3 without class 4, suggesting an alternative or intermediate conformational state. I) Time-resolved occupancies of the EM classes shown in panel E, determined from single-particle NS EM datasets. Class distributions were obtained using an identical processing workflow and a comparable number of micrographs at each time point. Empty cells indicate that a given class was not detected at the given time point. Source data are provided as a Source Data file. J) Stacked area plot showing the evolution of class occupancies over time. The x-axis is displayed on a pseudo-logarithmic scale (In(t+1)) to simultaneously visualize rapid initial conformational changes and long-term equilibrium.

FIG. 69A-69C—Cryo-EM analysis of F_{ECTO}-Y10F and dual F_{ECTO}-Y10F-77.1 complexes. A) Cryo-EM density and atomic model of the F_{ECTO} trimer with a single Y10F Fab bound asymmetrically at the apex. The model is shown as ribbons: Y10F heavy chain in light red, light chain in yellow; F1 protomers in shades of green and F2 protomers in shades of gray. Upper and lower panels show side and top views, respectively. Glycans near Y10F Fab are indicated with arrows. B) Epitope-paratope contacts between Y10F and F_{ECTO}. The top panel highlights heavy-chain interactions, and the bottom panel highlights light-chain interactions. Both chains engage an asymmetric epitope on F. Interacting residues are color-coded by chain, matching the coloring from A). C) Cryo-EM map and model of F_{ECTO} bound by Y10F and 77.1 Fabs. Due to a symmetry mismatch (C1 for Y10F and C3 for 77.1) and partial Fab occupancy, only one copy of 77.1 is resolved in the final locally refined reconstruction.

FIG. 70A-70D—Cryo-EM structures of H8 bound to the F_{ECTO} in pre- and postfusion states. A) Cryo-EM density and atomic model of prefusion F_{ECTO} symmetrically bound by three copies of Fab H8. Ribbon representation shows H8 heavy chains in light purple and light chains in light blue. Side (top) and top (bottom) views are shown. B) Close-up of the prefusion interface. H8 engages the outer face of domain II exclusively through its heavy chain. C) Cryo-EM density and atomic model of postfusion F_{ECTO} with three copies of Fab H8 bound. Side (top) and top (bottom) views are shown. D) Close-up of the postfusion interface. As in the prefusion state, H8 binds exclusively via its heavy chain and targets the same region on domain II.

FIG. 71A-71F—In Vitro, Ex Vivo, and In Vivo Efficacy of Anti-F Monoclonal Antibodies. A) Neutralizing activity of Y10F, H8, and 77.1 against recombinant MeV strain rMeV-KS-Venus (3) was assessed in B-LCL, SLAMF1 (CD150)-expressing Vero cells (Vero-hCD150), and Nectin-4-expressing Vero cells (Vero-hNectin-4). Serial dilutions of each mAb (100-0.05 nM) were incubated with MeV for 1 h prior to inoculation onto target cells. Infection was monitored for 7 days, and neutralization efficacy is expressed as IC₅₀ values (nM). uLoD=upper limit of detection. Source data are provided as a Source Data file. B) Activity of Y10F, H8, and 77.1 in a MeV dissemination assay. Infection percentages in B-LCLs were determined 48 h post co-culture of 150 rMeV-KS-Venus (3) infected B-LCLs with 6×10⁵ uninfected B-LCLs. Inhibition of MeV dissemination is shown as a dose-response curve; inhibition of dissemination was expressed as half-max inhibitory concentrations (IC₅₀). Individual data points representing technical triplicates are plotted. Source data are provided as a Source Data file. C) Airway organoids differentiated at air-liquid interface (AO at ALI) were prepared by removing mucus and mechanical disruption to expose Nectin-4. Y10F, H8, and 77.1 (1000, 100, and 10 nM) were incubated with rMeV-KS-Venus (3) for 1 h and applied to the apical compartment for 2 h. After removal of the virus-antibody mixture, ALI conditions were restored. Infection was visualized by fluorescence imaging at 3 days post-inoculation (infected areas shown in black). D) Semi-quantitative interpretation of infection in AO at ALI: “+++” indicates extensive infection, “++” moderate infection, “+” low-level infection, and “−” no detectable infection. The untreated (NT) sample showed the highest infection levels among all tested conditions. E-F) Antibody transfer experiment to assess in vivo protective efficacy of mAbs. Cotton rats (n=5 biologically independent animals per group) received intraperitoneal (IP) injection of 1 mg/kg of the indicated mAbs or IgG purified from human sera (Carimune, CSL Behring) 14 hours before (E) or 24 hours after (F) challenge with MeV. Animals in the control group received PBS before the challenge. Values correspond to MeV titers in lung tissue collected 4 days post-infection. Significance was determined using a two-sided Mann-Whitney-Wilcoxon test with Holm-Bonferroni correction for multiple comparisons against the PBS control group. Exact P-values for E): PBS vs. Y10F P=0.0119, PBS vs. 77.1 P=0.0097. Exact P-values for F): PBS vs. Y10F P=0.1161 (not shown), PBS vs. 77.1 P=0.0079. P-value annotation legend: ns (non-significant): p>0.05, *: 0.01<p≤0.05, **: 0.001<p≤0.01. Box plots represent the distribution of viral titers: the center line represents the median (50th percentile), the box limits indicate the 25th and 75th percentiles, and the whiskers extend to the minimum and maximum values within 1.5-times interquartile range (IQR) from the box limits. Individual data points are overlaid. LoD=limit of detection. Source data are provided as a Source Data file.

FIG. 72A-72C—Mechanism of Action of Anti-F Monoclonal Antibodies. A) Synergistic inhibition of cell-cell fusion by 77.1 and Y10F antibodies. B3 H and F co-expressing effector cells were co-cultured with hCD150-expressing target cells in the presence of 77.1 and Y10F antibodies at the indicated concentrations (x- and y-axes). Fusion was quantified after 18 hours using a β-galactosidase complementation assay, and synergy was assessed using the BLISS independence model. A moderate synergistic effect was observed, with a BLISS score of 13.22. The area of highest synergism is highlighted by a white rectangle. Source data are provided as a Source Data file. B) A bar chart summarizing the results of a mechanistic assay comparing various known inhibitors, including the newly described mAbs. For each condition, cells were categorized based on their interaction with red blood cells (RBCs): reversibly bound (dark blue) where the RBCs were only adhered by chimeric H and could be easily removed by treatment with zanamivir; irreversibly bound (pink) where RBCs were initially attached by chimeric H, but subsequent F protein refolding anchored the fusion peptide in the RBC membrane, making the RBCs attachment zanamivir-insensitive and retaining RBC on the HEK293T cell surface; and fused (light blue) where RBCs underwent membrane fusion with HEK293T cells expressing the fusion complex. The data are the results of three separate experiments (see FIG. 82). C) Mechanistic assay over a range of mAb concentrations (x-axis), in which both 77.1 and Y10F efficiently block fusion (light blue triangles). 77.1 traps the F in the extended state (red squares). Y10F prevents F activation and F insertion (dark blue circles). Data represent the mean±SEM of n=3 biologically independent experiments. Source data are provided as a Source Data file.

FIG. 73A-73B—Viral Evolution Under Selective Pressure of 77.1 and Y10F mAbs. A) Viral evolution assay results from control (mock), as well as passages in the presence of Y10F and 77.1, with four replicates per mAb, were evaluated for mutations in the F gene. Only mutations with an allele frequency greater than 10% and a minimum read depth of 20 were included in the analysis. Source data are provided as a Source Data file. B) Location of point mutations identified in A) mapped onto a single protomer of the prefusion F structure. Mutated residues are highlighted, including I498T, which is located in the membrane proximal region and shown schematically (in FIG. 83 the MeV F protein with domains color-coded).

FIGS. 74—C6 reactivity during stabilization of distinct fusion intermediates. HEK293T cells transiently expressing the MeV H_Y17H-HPIV3_T193A chimera and MeV F (T461I) were incubated overnight at 37 °C in the presence of 1 pM FIP-HRC4 (prefusion stabilizer) (Zyla, et al., 2024; Bovier, et al., 2021), 1 pM HRC4 (stabilizer of the extended intermediate after fusion peptide insertion but prior to full postfusion refolding) (Bovier, et al., 2021), or were mock treated. C6 mAb reactivity was assessed as described in FIG. 67. The data show that the C6 epitope is exposed prior to complete refolding into the postfusion conformation, confirming that C6 is a post-trigger-specific antibody. Data represent the mean±SE of three independent experiments.

FIG. 75A-75C—Dynamic light scattering and thermal stability analysis of F_{ECTO}-Fab complexes. A) Autocorrelation functions of freshly prepared F_{ECTO} alone (gray) or in complex with Y10F (orange), H8 (blue), or C6 (green), measured using a NanoTemper Prometheus Panta. B) Regularization-derived size distributions calculated from the autocorrelation functions in A. Note that F_{ECTO} alone and F_{ECTO}-C6 display a bimodal distribution with a population of larger species (~30 nm and ~17.5 nm, respectively), whereas Y10F and H8 complexes appear monodisperse despite originating from the same F_{ECTO} stock. C) Summary table of hydrodynamic radii and thermal parameters derived from nanoDSF/DLS experiments. Radius from regularization corresponds to the peak maxima observed in B. Melting temperature denotes the inflection point of the first derivative of the intrinsic fluorescence at 330 nm. Onset temperature represents the point where the cumulant radius or scattering signal deviates from the baseline, indicating the initiation of aggregation.

FIG. 76A-76H—(A-H). Cryo-EM processing pipeline and map statistics. A-B: Cryo-EM processing pipeline (A) and final map statistics (B) for F_{ECTO}-Y10F structure. C-D) Processing pipeline (C) and statistics (D) for F_{ECTO}-Y10F-77.1 structure. E-F) Processing pipeline (E) and statistics (F) for F_{ECTO}-H8 structure in prefusion conformation. G-H) Processing pipeline (G) and statistics (H) for F_{ECTO}-H8 structure in postfusion conformation.

FIG. 77—Antibody footprints of Y10F, H8, and 77.1 on the F_{ECTO} trimer in prefusion and postfusion conformations. Surface representations of the F protein ectodomain (rendered at 12 Å resolution) show the binding footprints of the antibodies characterized in this study. Y10F is shown with heavy and light chains in orange and yellow, respectively. H8 is shown with heavy and light chains in purple and blue. 77.1 is shown with heavy and light chains in pink and purple, respectively. The final panel displays the H8 epitope mapped onto the postfusion conformation of the F protein.

FIG. 78A-78D—In vivo stability and pharmacokinetics of passively transferred monoclonal antibodies in cotton rats. A) Sera collected at 14, 38, 62, 86, and 110 hours post-inoculation of 77.1, Y10F, H8, and human IgG were serially diluted and assessed for neutralizing activity. Two-fold serial dilutions of serum samples were prepared in Advanced MEM in duplicate in 96-well flat-bottom tissue culture plates, starting at a dilution of 1:10 (50 μl/well). MeV-GFP B3 (50 TCID₅₀ in 50 μl of Advanced MEM) was added to each well and incubated for 1 hour. Vero-huCD150 cells (5,000 cells/well in 100 μl MEM supplemented with 5% FBS) were then added, and plates were incubated at 37 °C with 5% CO₂ for 4 days. The endpoint neutralization titer was defined as the reciprocal of the highest serum dilution that resulted in 100% inhibition of measles virus-induced cytopathic effect. Only mAb 77.1 exhibited neutralizing activity, whereas Y10F, H8, and human IgG showed no detectable neutralization at the starting dilution (1:40). B) Serum titration curves. Sera collected at 14, 38, 62, 86, and 110 hours post-inoculation was serially diluted and tested for binding to F-expressing cells. Data were fitted using a 4-parameter logistic regression (with the minimum signal constrained to 0) to determine the EC₅₀ dilution factor. Distinct colors represent individual animals (n=3+3). C) Longitudinal stability of serum antibodies. The data from B are stratified by dilution factor (1:10 to 1:640) and plotted over time to visualize retention of antibody binding capacity during the 110-hour study period. D) Standard calibration curves. Purified monoclonal antibodies (Y10F, H8, and 77.1) were titrated against the same F-expressing cells to serve as a reference for quantification. The half-maximal binding concentration (indicated as EC₅₀) is displayed for each antibody.

FIG. 79—Allele frequency distribution of synonymous and nonsynonymous mutations in evolved viral populations. MeV was cultured in the presence of mAb 77.1, Y10F, or without any inhibitors for over 50 days as indicated in Materials and Methods. Nucleotide polymorphisms and corresponding amino acid changes relative to the wild-type strain are depicted. Refined mutational profile, filtered for allele frequencies >=10% and sequencing depth >=20. Points are color-coded to match the sequence depth, as indicated by the color bar at the bottom.

FIG. 80A-80F—Inhibitory activity of viral populations following viral evolution. Viral entry inhibition by either mAb 77.1 (purple circles) or mAb Y10F (orange circles) of the viral population after passages in the presence of Y10F (A-D) and 77.1 (E), compared to wt (F). 200 pfu/well were incubated in the presence of the indicated concentration of the mAbs (x-axis). After 3 hours, the medium was replaced with a medium containing 2% methylcellulose Avicel and 2× complete medium (1:1). Two days post-infection, the number of infected cells was counted using a Cytation 5 (BioTek) and analyzed with Gen5 3.11 software. The percentage of inhibition was calculated relative to the no-treatment control. Data represent the mean±SD of two independent experiments.

FIG. 81—Fusion-inhibition activity of anti-F monoclonal antibodies against F variants. Fusion inhibition by mAb 77.1 (purple circles), mAb H8 (blue circles), and mAb Y10F (orange circles) was assessed using F proteins bearing mutations identified during viral evolution (M46V, M94V, 198T, G253E, F375S, V428E, and I498T). In addition, F proteins carrying the G37R and R436D substitutions (previously shown to escape recognition by mAb 77.1 (Zyla, et al., 2024)) were included, along with the parental wild-type F (B3) and the Moraten F. As shown, Y10F exhibited limited fusion-inhibition activity overall, except against the G37R and R436D variants. As expected, 77.1 did not inhibit fusion mediated by G37R or R436D. H8 displayed potent and broad fusion-inhibition activity across all F variants tested. Percent inhibition was calculated relative to the no-treatment control. Data represent the mean +SE of three independent experiments.

FIG. 82—Detailed plot from FIG. 6C showing red blood cell assay incubation at 37 °C for 60 min. Inhibitors and controls are shown, as are their concentrations.

FIG. 83—Structure of the MeV F protein with domains color-coded. HRN: N-terminal heptad repeat. HRC: C-terminal heptad repeat. FP: Fusion peptide. MPR: Membrane proximal region. TM: Transmembrane region. CT: Cytoplasmic tail. The fusion peptide and transmembrane region in the postfusion structure are depicted schematically, as the detailed structure was not resolved.

The figures herein are for illustrative purposes only and are not necessarily drawn to scale.

### DETAILED DESCRIPTION OF THE EXAMPLE EMBODIMENTS

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The words “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

The term “optional” or “optionally” means that the subsequent described event, circumstance or substituent may or may not occur, and that the description includes instances where the event or circumstance occurs and instances where it does not.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily—but may be—all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to Zyla D S, Della Marca R, Niemeyer G, et al. A neutralizing antibody prevents postfusion transition of measles virus fusion protein. Science. 2024; 384 (6703): eadm8693.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide for mAb 77.4 and its humanized mAb (77.1), for which the structure and activities are uncovered here, and is one of the few neutralizing monoclonal antibodies (mAbs) yet described against the MeV fusion protein.

Measles virus (MeV) is a highly transmissible (R₀ of 12-18) respiratory, enveloped, single-stranded negative-sense RNA virus. MeV belongs to the Paramyxoviridae family and causes significant morbidity and mortality among young children worldwide, despite the availability of safe and effective MeV vaccines. Measles accounts for more childhood deaths than any other vaccine-preventable disease. MeV was presumed to be eliminated in the U.S. in 2000 (i.e. ≥12 months without transmission). However, reintroduction of the virus has resulted in a resurgence in recent years, causing repeated outbreaks. Outbreaks in the U.S. and other countries with high vaccination coverage are often attributed to unvaccinated individuals. However, a significant number of cases also occur in previously vaccinated people, since up to 5% of vaccine recipients do not develop adequate protective antibodies even after the recommended two-dose vaccination regimen, and because vaccine-elicited immunity likely wanes over time. In addition, the influence of anti-vaccination proponents in social media led to vaccine hesitancy among parents and pockets of unvaccinated children.

With no effective antiviral regimen currently available for MeV, insufficient vaccine coverage (which is defined as ≤95% vaccination rate by WHO) can lead to a rise in MeV disease among vulnerable populations. Indeed, MeV has already undergone a global resurgence; estimated global MeV deaths rose from 89,780 in 2016 to 207,500 in 2019. The situation may worsen since the SARS-CoV-2 pandemic reduced routine childhood vaccination coverage and may expose more vulnerable individuals to infection. There have been over 122,000 cases in the first half of 2023 alone. Without global eradication, a significant increase in the number of MeV cases can be unavoidable. Even mild cases of measles infection can result in immune “amnesia”, and loss of preexisting antibodies against other infections, leaving measles convalescent individuals more vulnerable to future diseases.

Of particular concern during this resurgence in MeV cases are the rising number of individuals with compromised immune systems, such as cancer patients, transplant recipients, persons with autoimmune disorders on immunosuppressive therapies, and human immunodeficiency virus (HIV)-infected individuals, in addition to pregnant women. In such immunocompromised people, vaccination with current vaccines containing live-attenuated virus is often risky and inefficient; antibody responses are impaired, and even pre-existing immunity wanes. Measles resurgence in other parts of the world, combined with increased vaccine hesitancy, increases the risk of future measles outbreaks and the risk to vulnerable patient populations (see CDC reports). Notably, despite the foreseeable rise in measles cases and measles deaths, there are still no effective antivirals approved for human use. Neutralizing monoclonal antibodies offer lifesaving treatments against a number of viruses, but surprisingly, there are none yet available for prevention or treatment of measles virus. Even more surprisingly, there are not yet structures of any antibodies against the measles virus by which Applicants might interpret epitopes or activities that prevent or treat infection. Mab 77.4 (Mathieu, et al., 2021), for which structure and activities are uncovered here, is one of the few neutralizing mAbs yet described against the MeV fusion protein.

MeV infects via coordinated action of the hemagglutinin (H, the receptor binding protein) and fusion (F) envelope glycoproteins, to mediate virus attachment and entry into target lymphocyte cells. Vaccination results in neutralizing antibodies against these two proteins, although earlier work suggests that more neutralizing antibodies are directed against hemagglutinin (H), and that anti-F antibodies elicited by the current vaccine are poorly neutralizing. Better understanding of the structures of F, conformational changes that drive fusion, and how antibodies may inhibit structural changes to neutralize MeV are needed.

F is synthesized as a precursor (F₀) that is cleaved in the producer cell to yield N-terminal F₂ and C-terminal F₁ subunits that remain linked by a disulfide bond. The pre-fusion F complex on the viral surface is a trimer comprising three F₁ and three F₂ subunits. This trimeric F structure is kinetically trapped in a metastable conformation, primed for fusion activation upon engagement of the H glycoprotein by the cell-surface receptor. After receptor engagement by H, the pre-fusion F undergoes a structural transition, extending and inserting the hydrophobic fusion peptide into the target cell. During entry, F refolds into the classical 6-helix bundle, post-fusion structure, which brings together the N-terminal heptad repeat (HRN) and the C-terminal heptad repeat (HRC), triggering subsequent fusion of viral and cell membranes. Before refolding to the low-energy, post-fusion state, however, the F protein adopts a series of intermediate conformations that require domain conformational rearrangement and refolding.

Targeting the intermediate state of F is a key antiviral approach. Applicants previously demonstrated that peptides derived from a consensus sequence of the MeV HRC domain can inhibit viral entry, fusion, and spread in vitro and can prevent infection in vivo. HRC-derived peptides (e.g. HRC, residues 450-485) mimic the interactions between the native HRC domains and the HRN domains of MeV F proteins, thus inhibiting the transition of Finto its post-fusion state and, consequently, viral-host membrane fusion. The MeV HRC-peptides have been shown to block fusion after F activation and before refolding, likely targeting an extended intermediate of the F protein. Applicants have recently modified the MeV HRC peptide, by adding a carbobenzoxy-d-Phe-1-Phe-Gly sequence at the N-terminal domain of the previously described HRC peptide. This new peptide (called FIP-HRC) is superior in potency to the original HRC, can interact with the F before its activation, and stabilize the pre-fusion state of the protein. Applicants have also recently described a mouse monoclonal antibody (mAb 77.4) that, like HRC-peptides, blocks MeV viral infection through fusion inhibition (Mathieu, et al., 2021). The structural basis for the activity of a humanized version of mAb 77.4, termed mAb 77.1, and FIP-HRC molecules is not yet known. Here, Applicants describe three cryo-EM structures: prefusion MeV F in complex with mAb 77.1, prefusion MeV F in complex with FIP-HRC fusion inhibiting peptide, and F alone in the post-fusion conformation.

The present study features the first structure of MeV F in complex with any antibody; the structure is in the prefusion conformation, and this is the only neutralizing antibody yet described for F. The mAb is potent in vitro and in vivo; suggesting that other anti-F antibodies yet to be uncovered can have complementary efficacy. This work also provides the first structure of MeV F in the post-fusion conformation, providing the first pre- and post-fusion sequence matched pair for the paramyxovirus family. These structures and biochemical characterization enable Applicants to model the conformational changes occurring during the transition from pre-fusion to post-fusion states. The findings indicate that the antibody anchors to a tripartite epitope specific to the pre-fusion form of MeV F and achieves its neutralizing effect by arresting F at an intermediate stage, blocking the protein from completing its final refolding step to achieve the post-fusion conformation and membrane fusion.

Embodiments disclosed herein also provide for a thermostable (e.g., stabilized) recombinant subunit vaccine that can fill this unmet need in developed countries and is so stable even at high temperatures that it needs no cold chain. This vaccine would eliminate the cold chain requirement of the current live attenuated vaccine and thereby enable distribution in developing nations and facilitate global measles eradication.

The MeV transmembrane glycoproteins hemagglutinin (H) and fusion protein (F) play critical roles in viral cell entry. The Fis kinetically trapped in a metastable conformation, primed for fusion activation upon engagement of H with a cellular receptor (either CD150 or nectin-4). After H engages the receptor, the pre-fusion F undergoes a conformational transition, irreversibly refolding into a post-fusion conformation as it drives fusion of virus and host membranes. The H and F proteins (i.e., the fusion complex) are the main targets of neutralizing antibodies. The technical challenge for vaccine design has been stabilizing F for vaccine use. Applicants have identified specific mutations in the measles F protein that stabilize the pre-fusion conformation. Using these substitutions, Applicants have purified both full-length and ectodomain forms of the pre-fusion state of the measles F protein, and Applicants found that they stably maintain the pre-fusion state at high temperature and induce a protective immune response in vivo. Importantly, these are natural mutations combined from selected strains.

Here, Applicants described MeV specific pre-fusion F stabilizing mutations occurring during viral evolution. MeV field isolates from the central nervous system (CNS) demonstrate that the MeV fusion complex- and specifically mutations in the MeV F protein—can facilitate viral spread in the CNS, even in the absence of canonical receptors. Acquired mutations in the F protein derived from the CNS of infected individuals demonstrate a balance between mutations that decrease thermostability and those that partially compensate for decreases in stability. In previous work, Applicants reported that introducing these compensatory mutations alone into the wild-type fusion protein increases its stability. Leveraging the findings, Applicants recently obtained the structure of a stabilized Fectodomain bearing two stabilizing mutations (E170G and E455G). The full-length (FL) structure of MeV F has not yet been resolved. To obtain a stable purified MeV F_{FL}, Applicants introduced initial mutations (E170G and E455G) and explored additional stabilizing substitutions. Applicants found that the stabilizing mutations are additive, and the combination of 5 mutations (E170G, Y442H, E455G, V459I, and G506E) produced the most stable F protein. In addition, Applicants have mutated the furin cleavage site to further enhance protein stability. When expressed on the surface of cells, the F bearing E170G, Y442H, E455G, V459I, and G506E maintains the pre-fusion state (assessed by recognition of pre-fusion antibody) even after exposure to 55 °C, 60 °C, and 65 °C for a prolonged period. Applicants hypothesized that these stabilized pre-fusion F_{FL} proteins will elicit in vivo protection. Applicants purified F_{FL} forms of the pre-fusion state of the MeV F protein bearing these stabilizing mutations, and Applicants have found that they indeed induce a protective immune response in vivo and elicit neutralizing antibodies.

### TERMINOLOGY AND DEFINITIONS

Paramyxoviridae (from Greek para-“by the side of” and myxa “mucus”) is a family of negative-strand RNA viruses in the order Mononegavirales. Vertebrates serve as natural hosts. Diseases associated with this family include measles, mumps, and respiratory tract infections. The family has four subfamilies, 17 genera, three of which are unassigned to a subfamily, and 78 species. Measles is caused by the measles virus of the genus Morbillivirus within the family Paramyxoviridae. Virions are enveloped and can be spherical or pleomorphic and capable of producing filamentous virions. The diameter is around 150 nm. Genomes are linear, around 15 kb in length. Fusion proteins and attachment proteins appear as spikes on the virion surface. Matrix proteins inside the envelope stabilize virus structure. The nucleocapsid core is composed of the genomic RNA, nucleocapsid proteins, phosphoproteins and polymerase proteins. The genome is nonsegmented, negative-sense RNA, 15-19 kilobases in length, and contains six to 10 genes. Viruses in the Paramyxoviridae family are also antigenically stable, meaning that the glycoproteins on the viruses are consistent between different strains of the same type.

The gene sequence is: Nucleocapsid-phosphoprotein-matrix-fusion-attachment-large (polymerase). The fusion protein F projects from the envelope surface as a trimer, and mediates cell entry by inducing fusion between the viral envelope and the cell membrane by class I fusion. The cell attachment proteins, H/HN/G, span the viral envelope and project from the surface as spikes. They bind to proteins on the surface of target cells to facilitate cell entry. Proteins are designated H (hemagglutinin) for morbilliviruses as they possess hemagglutination activity, observed as an ability to cause red blood cells to clump in laboratory tests. HN (Hemagglutinin-neuraminidase) attachment proteins occur in respiroviruses, rubulaviruses and avulaviruses. These possess both hemagglutination and neuraminidase activity, which cleaves sialic acid on the cell surface, preventing viral particles from reattaching to previously infected cells. Attachment proteins with neither hemagglutination nor neuraminidase activity are designated G (glycoprotein). These occur in henipaviruses.

The nucleotide and protein, polypeptide and peptide sequences corresponding to various paramyxovirus fusion protein genes have been previously disclosed, and may be found at computerized databases known to those of ordinary skill in the art. One such database is the National Center for Biotechnology Information's Genbank and GenPept databases located at the National Institutes of Health website.

As used herein “thermostable” refers to a protein that maintains its conformation at a temperature for greater than 1, 3, 5, 7, 9, 12, 30, or greater than 60 minutes, such as greater than 1, 2, 3, 4, 5, 6, 7, 8, 9, 10, or 30 days. The temperature that a protein is thermostable at can include 20, 25, 30, 35, 37, 40, 45, 50, 55, 60, 65, 70, or 75 °C.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

A “pharmaceutical composition” refers to a composition that usually contains an excipient, such as a pharmaceutically acceptable carrier that is conventional in the art and that is suitable for administration to cells or to a subject.

### Neutralizing Antibodies

In example embodiments, neutralizing antibodies are disclosed that recognize the F protein of a paramyxovirus (e.g., measles virus). In example embodiments, the antibodies recognize the F protein while it is in its pre-fusion state. In example embodiments, the neutralizing antibody recognize the F protein of MeV. In example embodiments, the antibodies include CDRs specific for binding the F protein. In example embodiments, the CDRs may be included in any antibody backbone disclosed herein. In example embodiments, the antibodies are humanized. In example embodiments, the antibodies are monoclonal antibodies.

In example embodiments, the antibodies comprise the following CDRs: one or more light chain variable region complementarity determining regions (CDRs) selected from a first CDR comprising the sequence RASKSISKYLA, a second CDR comprising the sequence SGSTLQS, and a third CDR comprising the sequence CQQHNEYTLT; or one or more heavy chain variable region CDRs selected from a first CDR comprising the sequence DYAWN, a second CDR comprising the sequence ISYTLTTGYNPSLKSRI, and a third CDR comprising the sequence RSGWLLPYWYFDV.

In example embodiments, the antibodies comprise the following CDRs: one or more light chain variable region complementarity determining regions (CDRs) selected from a first CDR comprising the sequence RATSSVVYMH, a second CDR comprising the sequence ATSNLAS, and a third CDR comprising the sequence QQWNSNPYT; or one or more heavy chain variable region CDRs selected from a first CDR comprising the sequence NYWIE, a second CDR comprising the sequence EILPGNSYTNYNEKFRGKA, and a third CDR comprising the sequence PLSGNY.

The term “antibody” is used interchangeably with the term “immunoglobulin” herein, and includes intact antibodies, fragments of antibodies, e.g., Fab, F(ab′)2 fragments, scFva, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic′ treatment of an intact or complete antibody or antibody chain. Fragments can also be obtained by recombinant means. Exemplary fragments include Fab, Fab′, F(ab′)2, Fabc, Fd, dAb, VHH and scF and/or Fv fragments. As used herein, a preparation of antibody protein “having less than about 50% of non-antibody protein (also referred to herein as a “contaminating protein”), or of chemical precursors, is considered to be “substantially free.” 40%, 30%, 20%, 10% and more preferably 5% (by dry weight) of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

In an embodiment, the antibody is a fragment or portion thereof. In an example embodiment, the antibody is an epitope binding protein or portion thereof. The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

In an embodiment, the antibody is an antibody fragment or portion. Examples of portions of antibodies encompassed by the present definition include: (i) the Fab fragment, having V_{L}, C_{L}, V_{H} and C_{H}1 domains; (ii) the Fab′ fragment, which’ is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii)′isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain oligopeptides, form a pair of “antigen binding regions” (Zapata et al., Protein Eng. 8(10): 1057-62 (1995); and U.S. Pat. No. 5,641,870).

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the disclosure, provided that the antibody or fragment binds specifically to a target molecule.

In an embodiment, the antibody is a single-chain antibody (scFvs). The term “single-chain variable fragment”, as used herein refers to a fusion protein containing the variable region(s) of the heavy (VH) and light (VL) of an immunoglobulin that are connected via a linker peptide. The linker peptide typically ranges from about 10 to about 25 amino acids. The linker can be flexible and can contain one or more glycine residues for flexibility. The linker can contain one or more serine or threonine residues to increase or modify solubility. The V_{H} and light (V_{L}) can be linked via the linker in any order. In an embodiment, N terminus of the V_{H} and is coupled, via a linker, C terminus of the (V_{L}). In an embodiment, C terminus of the V_{H} and is coupled, via a linker, N terminus of the (V_{L}). In an embodiment, the scFV is a bivalent or trivalent scFvs. In an embodiment bitrivalent or trivalent scFvs are bi or trispecific, meaning that they can target 2 or 3, respectively, different epitopes. See also e.g., Hollinger, Philipp; Prospero, T; Winter, G (July 1993). “Diabodies”: small bivalent and bispecific antibody fragments”. Proceedings of the National Academy of Sciences of the United States of America. 90 (14): 6444-8; incq, S; Bosman, F; Buyse, M A; Degrieck, R; Celis, L; De Boer, M; Van Doorsselaere, V; Sablon, E (2001). “Expression and purification of monospecific and bispecific recombinant antibody fragments derived from antibodies that block the CD80/CD86-CD28 costimulatory pathway”. Protein Expression and Purification. 22 (1): 11-24. doi:10.1006/prep.2001.1417; Le Gall, F.; Kipriyanov, S M; Moldenhauer, G; Little, M (1999). “Di-, tri- and tetrameric single chain Fv antibody fragments against human CD19: effect of valency on cell binding”. FEBS Letters. 453 (1): 164-168. doi:10.1016/S0014-5793(99)00713-9; Huston, J. S.; Levinson, D.; Mudgett-Hunter, M.; Tai, M. S.; Novotný, J.; Margolies, M. N.; Crea, R. (1988). “Protein engineering of antibody binding sites: recovery of specific activity in an anti-digoxin single-chain Fv analogue produced in Escherichia coli”. Proceedings of the National Academy of Sciences of the United States of America. 85 (16): 5879-5883; de Graaf et al., Methods Mol Biol. 2002; 178:379-87. doi:10.1385/1-59259-240-6:379; Zhou, H.X., J Mol Biol. 2003 May 23; 329(1):1-8. doi:10.1016/s0022-2836(03)00372-3; Bird and Walker. Trends Biotechnol. 1991 April;9(4):132-7. doi:10.1016/0167-7799(91)90044-I; Worn et al., J Mol Biol. 2001 Feb. 2; 305(5):989-1010. doi: 10.1006/jmbi.2000.4265.

As used herein, “heavy chain antibody,” “VHH” or “single-domain antibodies” (sdAbs) refers to an antibody which is composed only of two heavy chains and lacks the two light chains usually found in antibodies (see, e.g., Henry and Mackenzie, Antigen recognition by single-domain antibodies: structural latitudes and constraints. MAbs. 2018 August-September; 10(6): 815-826). VHH can refer to an antibody or VHH domain. Single-domain antibodies (sdAb) are also referred to as a “nanobody”, which is defined herein as an antibody fragment composed of a single monomeric variable antibody domain. As used herein “VHH” is used interchangeably with “nanobody.” The ~12-15 kDa variable domains of these antibodies (VHHs and VNARs) can be produced recombinantly and can recognize antigen in the absence of the remainder of the antibody heavy chain. In common antibodies, the antigen binding region consists of the variable domains of the heavy and light chains (VH and VL). Heavy-chain antibodies can bind antigens despite having only VH domains. In an embodiment, the heavy chain antibody is an antibody derived from cartilaginous fishes (immunoglobulin new antigen receptor (IgNAR)) or camelid ungulates. Non-limiting examples of camelids include dromedaries, camels, llamas and alpacas.

It is intended that the term “antibody” encompass any Ig class or any Ig subclass (e.g., the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric f-rm, and IgA antibodies exist in monomeric, dimeric or multimeric form.

The term “IgG subclass” refers to the four subclasses of immunoglobulin class IgG-IgG1, IgG2, IgG3, and IgG4 that have “been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 or 4 peptide loops) stabilized, for example, by β pleated sheet and/or intrachain disulfide bond. Domains are further referred to herein as “constant” or “variable”, based on the relative lack of sequence variation within the domains of various class members in the case of “constant” domain, or the significant variation within the domains of various class members in the case of a “variable” domain. Antibody or polypeptide “domains” are often referred to interchangeably in the antibody or polypeptide “regions”. The “constant” domains of an antibody light chain are referred to interchangeably as “light chain constant regions”, “light chain constant domains”, “CL” regions or “CL” domains.” The “constant” domains of an antibody heavy chain are referred to interchangeably as “heavy chain constant regions”, “heavy chain constant domains”, “CH” regions or “CH” domains.” The “variable” domains of an antibody light chain are referred to interchangeably as “light chain variable regions”, “light chain variable domains”, “VL” regions or “VL” domains. The “variable” domains of an antibody heavy chain are referred to interchangeably as “heavy chain constant regions”, “heavy chain constant domains”, “VH” regions or “VH” domains.

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present disclosure and antibodies of the disclosure bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant cross reactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly cross react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein. “Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

The antibodies as defined for the present disclosure include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Vaccine Compositions

In example embodiments, a paramyxovirus fusion protein is used as an immunogen in a vaccine against a paramyxovirus. In example embodiments, the fusion protein is the MeV fusion protein. In example embodiments, the immunogen includes the full-length fusion protein. In example embodiments, the fusion protein includes the ectodomain of the fusion protein. In example embodiments, the MeV fusion protein includes one or more stabilizing mutations. In example embodiments, the stabilizing mutations make the fusion protein more thermostable. In example embodiments, orthologous mutations can be made in the fusion protein of any other paramyxovirus to generate a stabilized immunogen. As used herein, “orthologous mutations” refers to mutations that occur in orthologous genes in different strains or species of paramyxoviruses. Orthologous mutations may not occur at the same residue number, however one skilled in the art occur can identify conserved residues that occur in the homologous position or domain of the orthologous gene. For example, one skilled in the art can perform an alignment of orthologous genes.

### In Vitro Peptide/Polypeptide Synthesis

Proteins or peptides may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. The nucleotide and protein, polypeptide and peptide sequences corresponding to various paramyxovirus fusion protein genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963). In certain embodiments, peptides are prepared by (1) parallel solid-phase synthesis on multi-channel instruments using uniform synthesis and cleavage conditions; (2) purification over a P-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different-patients.

Alternatively, a nucleic acid (e.g., a polynucleotide) encoding a peptide of the invention may be used to produce the peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. In one embodiment, in vitro translation is used to produce the peptide. Many exemplary systems exist that one skilled in the art could utilize (e.g., Retic Lysate IVT Kit, Life Technologies, Waitham, MA).

An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression, if necessary, the DNA may be linked to the appropriate transcriptional and translational regulatory control nucleotide sequences recognized by the desired host (e.g., bacteria, insect cells, or mammalian cells), although such controls are generally available in the expression vector.

Expression vectors comprising the polynucleotides encoding the fusion protein, as well as host cells containing the expression vectors, are also contemplated. The protein may be provided in the form of RNA or cDNA molecules encoding the desired protein. One or more proteins of the invention may be encoded by a single expression vector.

The term “polynucleotide encoding a polypeptide” encompasses a polynucleotide which includes only coding sequences for the polypeptide as well as a polynucleotide which includes additional coding and/or non-coding sequences. Polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the protein fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In embodiments, the polynucleotides can comprise the coding sequence for the protein fused in the same reading frame to a marker sequence that allows, for example, for purification of the encoded polypeptide. For example, the marker sequence can be a hexa-histidine tag supplied by a vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

The isolated fusion proteins described herein can be produced in vitro by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al, Proc. Nat'l. Acad. Sci. USA 81:5662-5066 (1984) and U.S. Pat. No. 4,588,585.

In example embodiments, oligonucleotides encoding a fusion protein can be designed based on the amino acid sequence of the desired polypeptide and selecting those codons that are favored in the host cell in which the recombinant polypeptide of interest is produced. In example embodiments, oligonucleotides encoding a fusion protein is inserted into an expression vector and optionally operatively linked to an expression control sequence appropriate for expression of the protein in a desired host.

Recombinant expression vectors may be used to amplify and express DNA encoding the fusion protein. Recombinant expression vectors are replicable DNA constructs which have synthetic or cDNA-derived DNA fragments encoding a fusion protein operatively linked to suitable transcriptional or translational regulator elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product. Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli.

Suitable host cells for expression of a fusion protein include prokaryotes, yeast, insect or higher eukaryotic cells under the control of appropriate promoters. Prokaryotes include gram-negative or gram-positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al, Cloning Vectors: A Laboratory Manual, Elsevier, 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), 293, HeLa, HEK293, and BHK cell lines. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the fusion protein to allow easy purification by passage over an appropriate affinity column. Isolated proteins can also be physically characterized using such techniques as proteolysis, nuclear magnetic resonance and x-ray crystallography.

### In Vivo Peptide/Polypeptide Synthesis

The present invention also contemplates the use of nucleic acid molecules as vehicles for delivering immunogens to a subject, in vivo, in the form of, e.g., DNA/RNA vaccines (see, e.g., WO2012/159643, and WO2012/159754, hereby incorporated by reference in their entirety).

In one embodiment fusion proteins may be administered to a patient in need thereof by use of an mRNA vaccine (see, e.g., Sahin, U, Kariko, K and Tureci, O (2014). mRNA-based therapeutics-developing a new class of drugs. Nat Rev Drug Discov 13:759-780; Weissman D, Karikó K. mRNA: Fulfilling the Promise of Gene Therapy. Mol Ther. 2015; 23(9):1416-1417. doi:10.1038/mt.2015.138; Kowalski P S, Rudra A, Miao L, Anderson D G. Delivering the Messenger: Advances in Technologies for Therapeutic mRNA Delivery. Mol Ther. 2019; 27(4):710-728. doi:10.1016/j.ymthe.2019.02.012; Magadum A, Kaur K, Zangi L. mRNA-Based Protein Replacement Therapy for the Heart. Mol Ther. 2019; 27(4):785-793. doi:10.1016/j.ymthe.2018.11.018; Reichmuth A M, Oberli M A, Jaklenec A, Langer R, Blankschtein D. mRNA vaccine delivery using lipid nanoparticles Ther Deliv. 2016; 7(5):319-334. doi:10.4155/tde-2016-0006; and Khalil A S, Yu X, Umhoefer J M, et al. Single-dose mRNA therapy via biomaterial-mediated sequestration of overexpressed proteins. Sci Adv. 2020; 6(27):eaba2422). In an exemplary embodiment, mRNA encoding for a fusion protein is delivered using lipid nanoparticles (see, e.g., Reichmuth, et al., 2016). In an exemplary embodiment, mRNA encoding for a fusion protein is delivered using biomaterial-mediated sequestration (see, e.g., Khalil, et al., 2020).

In one embodiment, a fusion protein may be administered to a patient by use of a plasmid. These are plasmids which usually consist of a strong viral promoter to drive the in vivo transcription and translation of the gene (or complementary DNA) of interest (Mor, et al., (1995). The Journal of Immunology 155 (4):2039-2046). Intron A may sometimes be included to improve mRNA stability and hence increase protein expression (Leitner et ai, (1997). The Journal of Immunology 159 (12): 6112-61 19). Plasmids also include a strong polyadenylation/transcriptional termination signal, such as bovine growth hormone or rabbit beta-globulin polyadenylation sequences (Alarcon et al., (1999). Adv. Parasitol. Advances in Parasitology 42:343-410; Robinson et al., (2000). Adv. Virus Res. Advances in Virus Research 55:1-74; Bohm et al, (1996). Journal of Immunological Methods 193 (i): 29-40.). Multicistronic vectors are sometimes constructed to express more than one immunogen, or to express an immunogen and an immunostimulatory protein (Lewis et al., (1999). Advances in Virus Research (Academic Press) 54:129-88).

Because the plasmid is the “vehicle” from which the immunogen is expressed, optimizing vector design for maximal protein expression is essential (Lewis et al., (1999), Advances in Vims Research (Academic Press) 54:129-88). One way of enhancing protein expression is by optimizing the codon usage of mRNAs for eukaryotic cells. Another consideration is the choice of promoter. Such promoters may be the SV40 promoter or Rous Sarcoma Vims (RSV).

Plasmids may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (IM) in skeletal muscle, or intradermaliy (ID), with DNA being delivered to the extracellular spaces. This can be assisted by electroporation by temporarily damaging muscle fibers with myotoxins such as bupivacaine; or by using hypertonic solutions of saline or sucrose (Alarcon J B, Waine G W, McManus D P. DNA vaccines: technology and application as anti-parasite and anti-microbial agents. Adv Parasitol. 1999; 42:343-410). Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999), Adv Parasitol. 1999; 42:343-410; Lewis et al., (1999), Advances in Virus Research (Academic Press) 54:12.9-88). Alternative delivery methods may include aerosol instillation of naked DNA on mucosal surfaces, such as the nasal and lung mucosa, (Lewis et al., (1999). Advances in Virus Research (Academic Press) 54:129-88) and topical administration of pDNA to the eye and vaginal mucosa (Lewis et al., (1999) Advances in Virus Research (Academic Press) 54:129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors.

In example embodiments, the fusion protein of the invention may be encoded and expressed in vivo using a viral based system (e.g., an adenovirus system, an adeno associated virus (AAV) vector, a poxvirus, or a lentivirus). In one embodiment, the immunogenic composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (1PCAVD 001). J Infect Dis. 2013 Jan. 15; 207(2):240-7). Plasmids that can be used for adeno associated virus, adenovirus, and lentivirus delivery have been described previously (see e.g., U.S. Pat. Nos. 6,955,808 and 6,943,019, a d U.S. patent application No. 20080254008).

In another embodiment a Poxvirus is used in the immunogenic composition. These include orthopoxvirus, avipox, vaccinia, MVA, NYVAC, canarypox, ALVAC, fowlpox, TROVAC, etc. (see e.g., Verardi et. al.., Hum Vaccin Immunother. 2012 July;8(7):961-70; and Moss, Vaccine. 2013: 31 (39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

### Vaccine or Immunogenic Composition Administration

Effective vaccine or immunogenic compositions advantageously include a strong adjuvant to initiate an immune response. An effective immune response advantageously includes a strong adjuvant to activate the immune system (Speiser and Romero, Molecularly defined vaccines for cancer immunotherapy, and protective T cell immunity Seminars in Immunol 22:144 (2010); and Zhao, T., Cai, Y., Jiang, Y. et al. Vaccine adjuvants: mechanisms and platforms. Sig Transduct Target Ther 8, 283 (2023)).

Prime/boost regimens refer to the successive administrations of a vaccine or immunogenic or immunological compositions. In certain embodiments, administration of the immunogenic composition is in a prime/boost dosing regimen, for example administration of the immunogenic composition at weeks 1, 2, 3 or 4 as a prime and administration of the immunogenic composition is at months 2, 3 or 4 as a boost. In another embodiment DNA encoding the fusion protein is used to prime followed by a protein boost. In another embodiment, protein is used to prime followed by boosting with a virus encoding the fusion protein (see, e.g., Hutchings C L, Birkett A J, Moore A C, Hill A V. Combination of protein and viral vaccines induces potent cellular and humoral immune responses and enhanced protection from murine malaria challenge. Infect Immun. 2007; 75(12):5819-5826). In another embodiment a virus encoding the fusion protein is used to prime and another virus is used to boost. In another embodiment protein is used to prime and DNA is used to boost.

The pharmaceutical compositions can be processed in accordance with conventional methods of pharmacy to produce medicinal agents for administration to patients in need thereof, including humans and other mammals. For example, the immunogenic composition can include a pharmaceutically acceptable carrier and/or excipient. In example embodiments, the pharmaceutically acceptable carrier comprises water. In example embodiments, the pharmaceutically acceptable carrier further comprises dextrose. In example embodiments, the pharmaceutically acceptable carrier further comprises dimethylsulfoxide. A carrier may take a wide variety of forms depending on the form of preparation desired for administration, e.g., ocular, oral, topical or parenteral, including gels, creams ointments, lotions and time released implantable preparations, among numerous others. Solutions or suspensions used for ocular, parenteral, intradermal, subcutaneous, or topical application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. Liposomal suspensions may also be pharmaceutically acceptable carriers. These may be prepared according to methods known to those skilled in the art. In example embodiments, the vaccine or immunogenic composition is administered intravenously or subcutaneously.

The invention provides for pharmaceutical compositions containing a fusion protein as described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopeia, European Pharmacopeia or other generally recognized pharmacopeia for use in mammals, and more particularly in humans. These compositions can be useful for treating and/or preventing viral infection and/or autoimmune disease.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic. Pharmaceutically acceptable carriers, excipients, or diluents include, but are not limited, to saline, buffered saline, dextrose, water, glycerol, ethanol, sterile isotonic aqueous buffer, and combinations thereof.

Further embodiments are illustrated in the following Examples which are given for illustrative purposes only and are not intended to limit the scope of the invention.

### EXAMPLES

### Example 1—Human Chimeric mAb 77.1

### Human Chimeric mAb 77.1 Potently Inhibits MeV Fusion and Replication.

There are currently no approved antiviral treatments for measles virus infection, although the provision of such treatments would provide important medical defense to those who cannot be vaccinated or in whom vaccines didn't take. Antibody is a crucial life-saving protective intervention for several other viruses. Applicants previously isolated and sequenced the F-targeted, neutralizing mouse antibody mAb 77.4. To facilitate future use as a human therapeutic, Applicants grafted the 77.4 variable regions into a human IgG1 framework to obtain a chimeric antibody termed mAb 77.1 As mAb 77.1 is thus far the only neutralizing antibody described against MeV F, Applicants wished to assess its in vivo efficacy against MeV exposure.

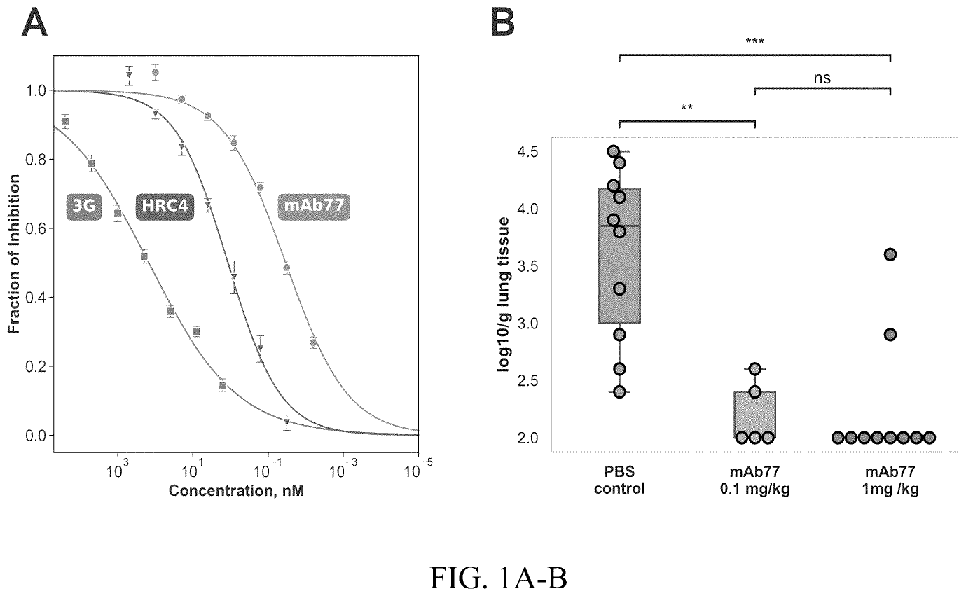

Applicants first evaluated the antiviral activity of mAb 77.1 using an in vitro viral entry assay in which cells, Vero-SLAM were treated with serial dilution of mAb 77.1 or previously described inhibitors, 3G and HRC4 and infected with MeV WT B3-GFP virus. Applicants found that mAb 77.1 inhibited fusion with a potency of 0.034 nM (5.1 ng/mL) (FIG. 1A). Notably, the activity of mAb 77.1 was substantially greater than that of two other known inhibitors evaluated side-by-side with the antibody. A fusion-inhibiting lipopeptide termed HRC4, derived from the C-terminal heptad repeat of F, blocked fusion with a potency of 1.17 nM while the small molecule 3G, which locks F in the pre-fusion conformation, had a potency of only 150.12 nM.

Applicants next assessed mAb 77.1 for in vivo efficacy against MeV exposure using the standard cotton rat model. Cotton rats were treated with 0.1 mg/kg (n=5) or 1 mg/kg (n=10) of mAb 77.1 intraperitoneally 12 hours before challenge with 10⁵ TCID₅₀ (i.e., 50% tissue culture infectious) dose of MeV wt B3-GFP. Antibody pre-treatment resulted in a significant reduction in viral replication as compared to mock-treated animals (FIG. 1B). The average titer in mock-treated animals was 3.6 log 10 TCID₅₀/g lung tissue, whereas three animals pretreated with 0.1 mg/kg body weight of monoclonal antibody were virus negative and two had reduced virus titers in lung tissue (2.4 and 2.6 log 10 TCID₅₀/g). Eight animals treated with 1 mg/kg mAb were virus negative and two had reduced virus titers (2.9 and 3.6 log 10 TCID₅₀/g). The activity of mAb 77.1 at even a low 0.1 mg/kg dose was striking. Therefore, Applicants next wished to determine its mechanism of action.

### 77.1 Prevents Conformational Changes Required for Fusion.

The F protein is primed to undergo substantial conformational change and refolding to drive the fusion of the viral and host membranes. This refolding is thought to be initiated by a signal from the stalk of the H protein transmitted after it engages the receptor. As a first step in F conformational rearrangement, the N-terminal heptad repeat (contained in Domain III of the F1 head region) refolds from three short helices and beta-harpin motifs into an approximately 100 Å-long helix. This assembled and now extended helix projects the fusion peptide, previously concealed within Domain III, toward and into the host membrane. This conformation of F is often referred to as the ‘pre-harpin intermediate’. The sequence of events leading to complete refolding remains elusive but involves substantial rearrangement and repositioning of the stalk region containing C-terminal Heptad Repeat (HRC). This movement enables HRC to collapse onto HRN into a 6-helix bundle that draws the viral and host membranes into proximity for fusion.

Previous studies demonstrated that mAb 77.1 inhibits fusion of viral and host cell membranes, but the step at which it functions remains unclear. The possibilities were that it could (I) stabilize the F prefusion conformation to prevent F from triggering, (II) stabilize the intermediate state and interrupt the refolding sequence to prevent viral and host membranes from joining, or (III) functionally impair F refolding by inducing protomer separation or misfolding.

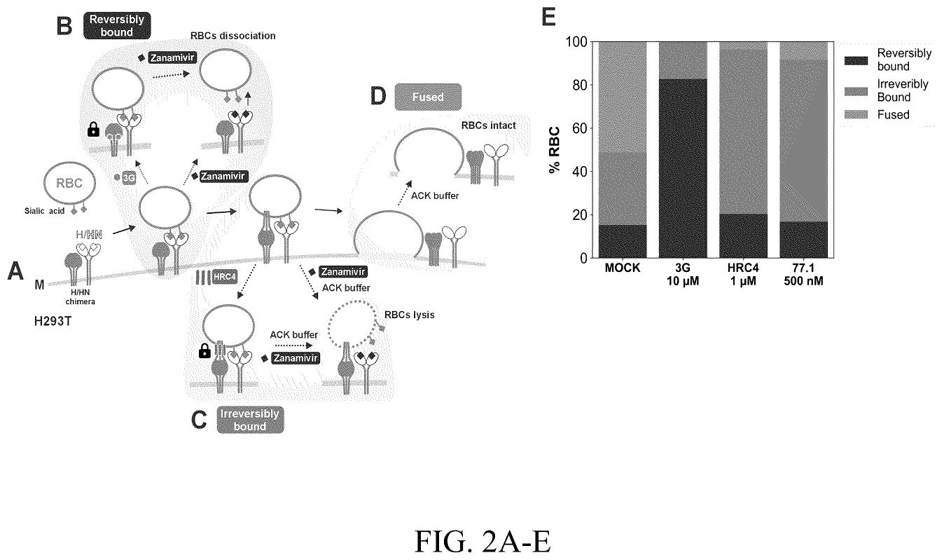

To elucidate the stage of the fusion process at which mAb 77.1 works, Applicants used an assay that measures fusion of red blood cells (RBC) with HEK293T cells bearing versions of H and F (FIG. 2A and B). In this assay, Applicants needed a version of H that would release its hold on receptor to discern anchoring by F from anchoring by H. Further, RBCs do not express the MeV receptors SLAM/CD150 or Nectin-4 and are instead coated with the sialic acid receptor naturally targeted by the hemagglutinin-neuraminidase surface protein (HN) of the related human paramyxovirus parainfluenza virus 3 (HPIV3). Moreover, the HN-sialic acid interaction can be interrupted by addition of the small molecule drug Zanamivir. Applicants thus employed a previously described chimeric receptor-binding protein which contains the MeV H stalk (the portion of H that interacts with MeV F) combined with the receptor-binding head domain of HPIV3 HN. The resulting chimeric protein H-HN enables both releasable attachment via sialic acid receptors and MeV H-driven triggering of F. Applicants also employed a point mutation T461I in F, which destabilizes the protein and allows easy activation for fusion. HEK293 Ts co-transfected with F_{T461I} and the H-HN chimera were incubated with RBCs for 30 mins at 4 °C to allow for receptor engagement but not fusion. The cells were washed to remove unbound RBCs and subsequently incubated with mAb 77.1 or diluent media. Two distinct MeV F viral entry inhibitors were included as additional controls. One inhibitor, N-(3-cyanophenyl)-2-phenylacetamide (compound 3G) binds to prefusion F and blocks H-mediated fusion activation. The other inhibitor is an HRC-derived peptide termed HRC4, which binds to HRN in the pre-hairpin intermediate and blocks the conformational transition to the 6-helix bundle.

After treatment with mAb, control inhibitor, or diluent media, the cells were transferred to 37 °C for 60 min to permit F activation (and fusion of the RBCs with the HEK293T in the mock control). RBCs were then stratified into three distinct groups: (1) those that were reversibly bound to the HEK293T cells; (2) those that remained anchored to HEK293 Ts but had not fused; and (3) those that had successfully fused with the HEK293 Ts. The first group was quantified by the amount of RBCs that were released from the HEK293 Ts (i.e., reversibly bound) via zanamivir; release by zanamivir indicates that the RBCs were anchored to the HEK293T target cells through H-HN interactions only and that no F-mediated fusion had commenced. The second group was quantified through osmotic lysis of RBCs; these were anchored to the HEK293 Ts through F (irreversibly bound) but had not yet fused. The third group of RBCs-those which had fused with the HEK293T cells-were quantified by lysis of the remaining cells using a detergent. In each case, the released hemoglobin in the lysate was measured via absorbance at 410 nm, with the amount of reversibly bound+irreversibly bound+fused RBCs set to 100%.

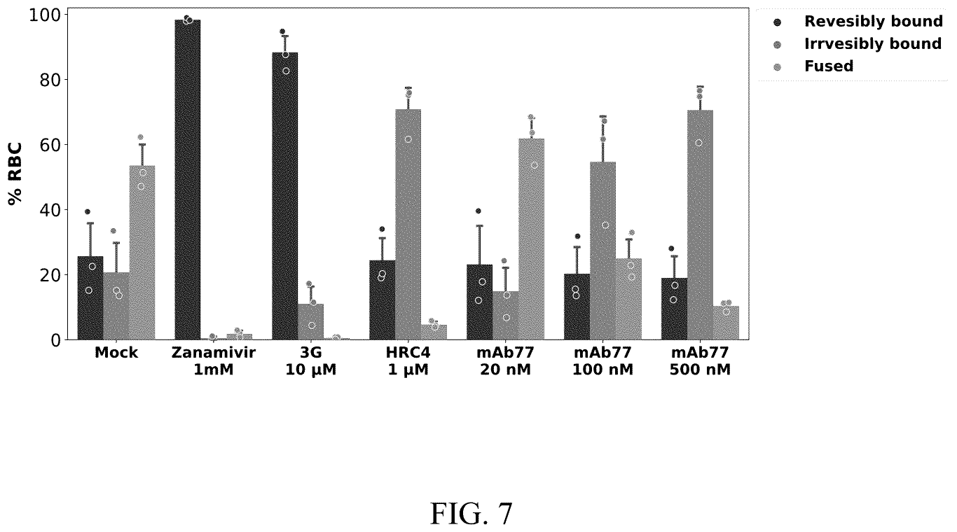

Applicants found that treatment with diluent media resulted in the fusion of approximately 50% of total RBCs with the HEK293T cells (FIG. 2A and B, FIG. 7). The remaining RBCs had either begun, but not completed fusion (irreversibly bound through F), or had not yet initiated fusion (reversibly bound through HN only). Treatment with the 3G inhibitor prevented nearly all F-mediated anchoring, indicating, as expected, that this inhibitor prevents F activation. Meanwhile, treatment with the HRC4 peptide inhibitor resulted in most RBCs being irreversibly bound, indicating F was activated, but could not complete fusion. Treatment with mAb 77.1 had the same effect as HRC4, where the majority of RBCs were irreversibly bound. This suggests mAb 77.1 does not prevent the activation of F nor initiation of fusion and formation of the pre-hairpin intermediate, but instead prevents the collapse of this intermediate into the 6-helix bundle required for complete fusion.

### Stabilization of MeV F in the Pre-Fusion State

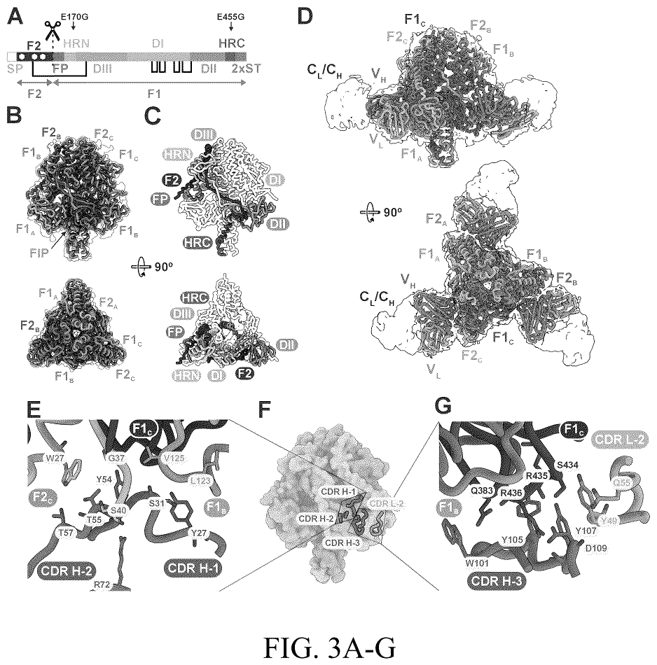

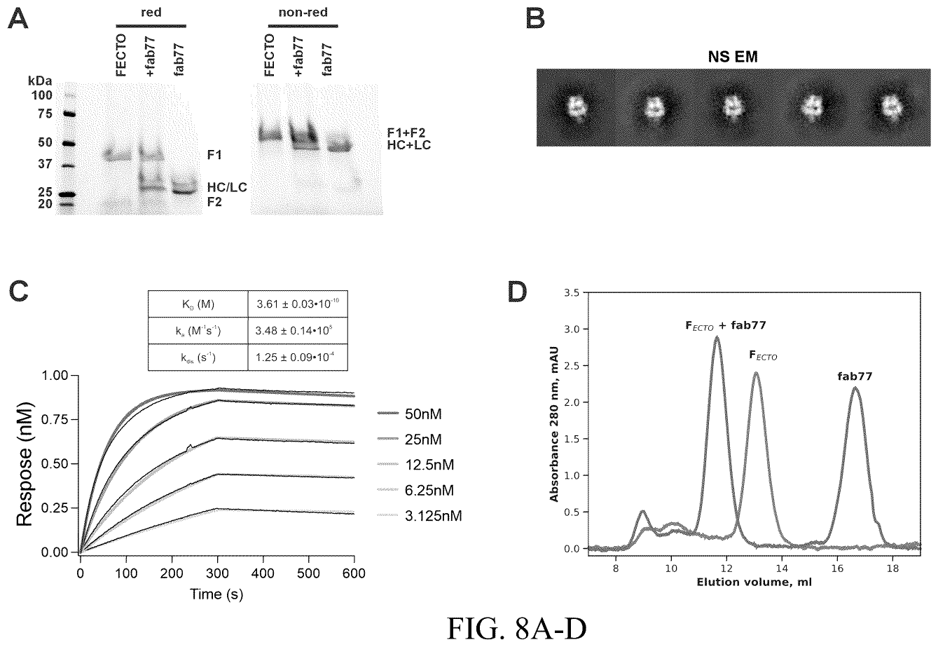

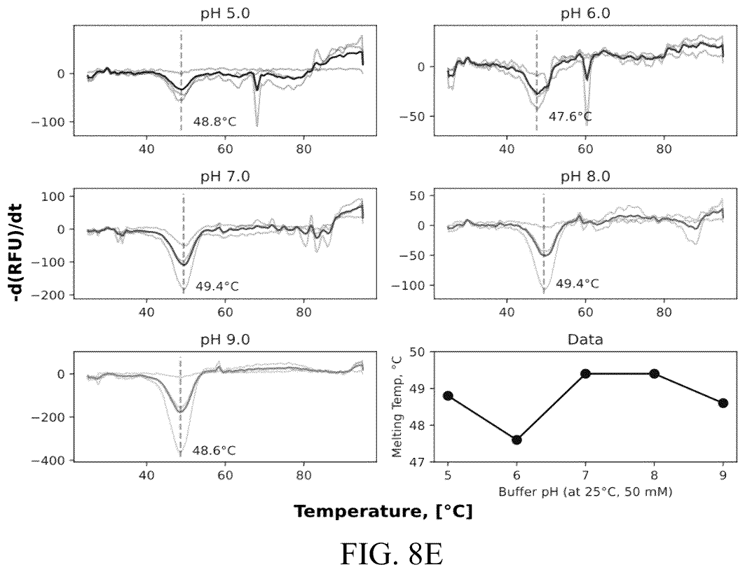

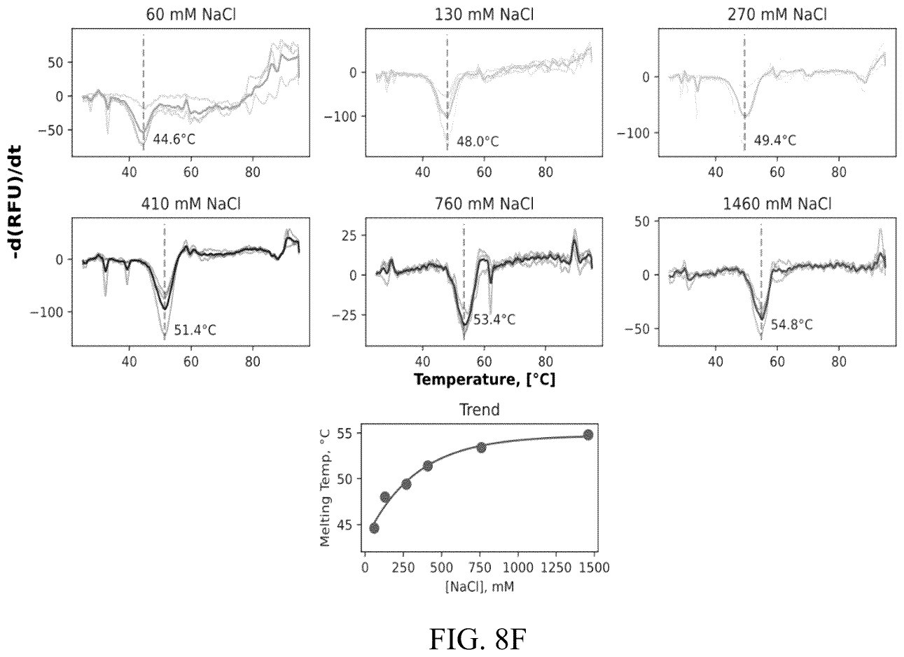

Expression of the ectodomain of wild-type F (F_{ECTO}, comprising amino acids 1-495) results in production of the post-fusion conformation only. Previous structural characterization of MeV F was achieved by engineering four cysteines in the stalk region which results in disulfide linkages between the three protomers. However, Applicants found that protein produced from this construct aggregated, even at lower concentrations. As such, Applicants took a different approach and instead generated a MeV F ectodomain that incorporates two naturally occurring point mutations, E170G and E455G (FIG. 3A). These mutations were identified in independent central-nervous system isolates of MeV and stabilize F in the pre-fusion state When paired, stabilization was further enhanced (data not shown). This protein, termed F2M_{ECTO}, was ~80% cleaved by furin and approximately 85% of the resulting particles were in the pre-fusion conformation (FIG. 8A, B).

Although F2M_{ECTO} can be purified in the pre-fusion state, the protein spontaneously springs to the post-fusion state over time. To enhance the stability of the pre-fusion conformation following purification, Applicants used differential scanning fluorimetry (DSF) to systematically explore the impact of pH and salt concentration on the apparent melting temperature (Tₘ^{app}) of F2M_{ECTO} (FIG. 8 C, D). Applicants found that while pH had little influence on the Tₘ^{app} of F2M_{ECTO}, higher salt concentration increased the Tₘ^{app}, from 44.6 °C at 60 mM NaCl to 54.8 °C at 1460 mM NaCl (FIG. 8 D). Hence, buffering conditions of 50 mM Hepes pH 8.0 and 800 mM NaCl were used in subsequent purification of F2M_{ECTO}. Notably, Applicants do not observe any F2M_{ECTO} in the post-fusion conformation even after more than 2 weeks of storage in this buffer (FIG. 8 B).

Applicants also measured the binding affinity of the 77.1 Fab fragment to purified F2M_{ECTO} using Bio-Layer Interferometry. In agreement with the potent activity of mAb 77.1 in the cell based assay, Fab 77.1 bound to F with a K_{D} of 360 pM (FIG. 8 E).

### Mab 77.1 Binds to Prefusion MeV F

The cell-based assays indicate that mAb 77.1 locks an intermediate state of F refolding after the fusion peptide has been released but before the collapse of the trimer into the 6-helix bundle required for fusion to complete. To elucidate the binding epitope of mAb 77.1 and further discern the mechanism of action, Applicants determined the structure of F2M_{ECTO} in complex with Fab 77.1. At the same time, Applicants solved the structure of F2M_{ECTO} in complex [FIP-HRC]₂-PEG₁₁ (a tandem peptide conjugate of two copies each of FIP and HRC, linked by a PEG₁₁ moiety. These structures were solved in order to 1) confirm that F2M_{ECTO} adopts the expected prefusion structure, and 2) identify any antibody-dependent conformational changes in F2M_{ECTO}. Note that this inhibitor contains both the tri-peptide FIP, which stabilizes prefusion F, and an HRC mimic, which binds to the pre-hairpin intermediate. Hence, this inhibitor could, in theory, be used to capture either state of F2M_{ECTO}, provided the correct state is available for binding.

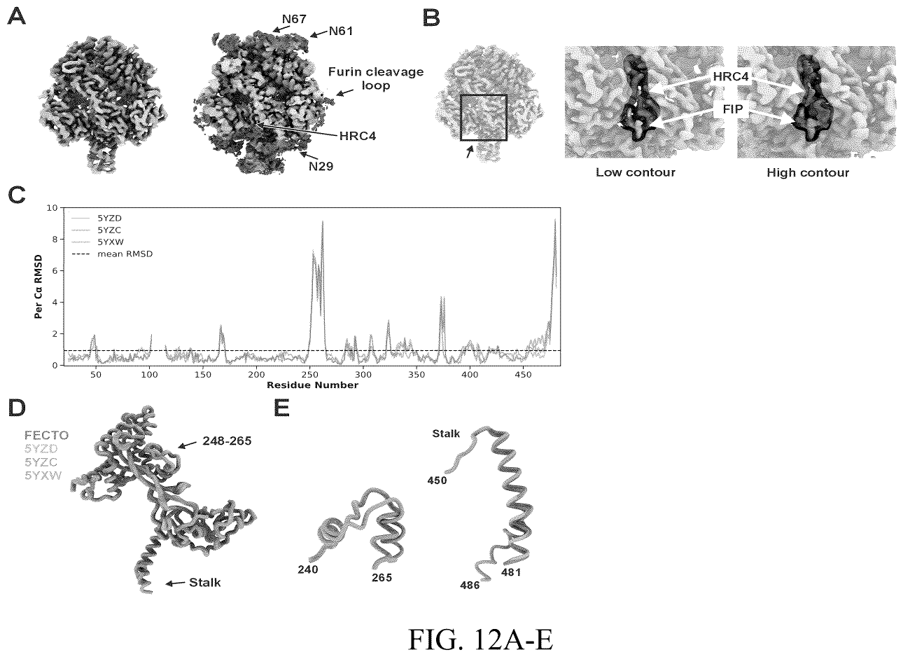

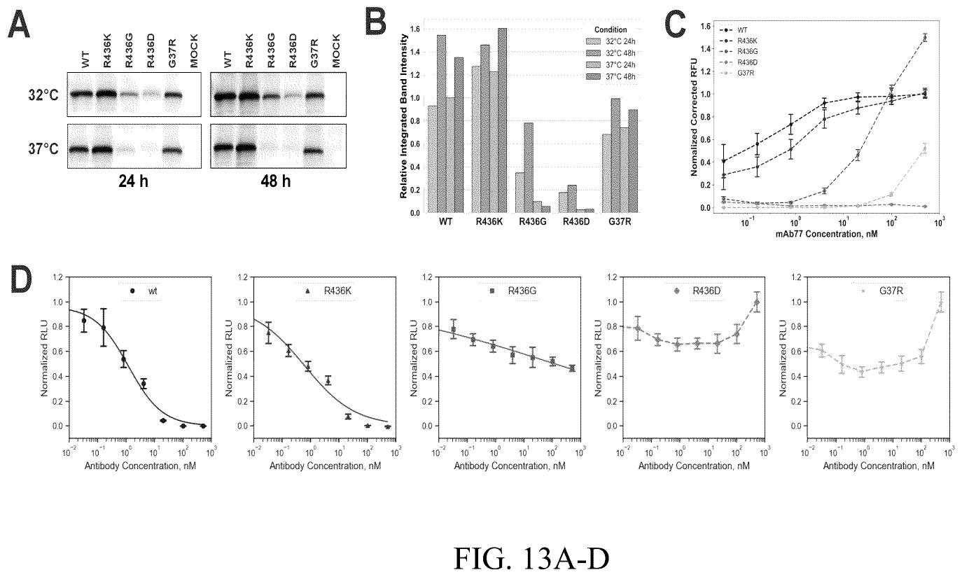

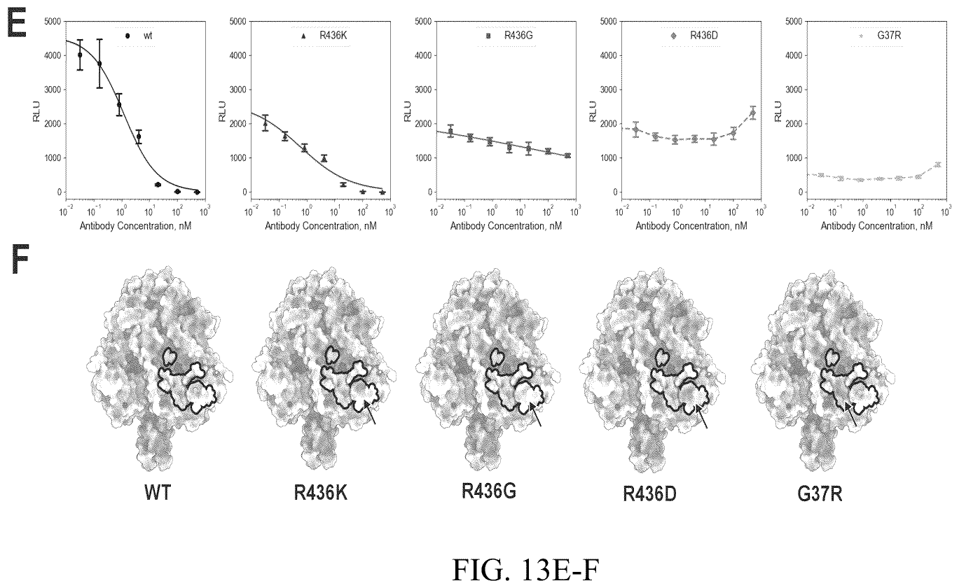

F2M_{ECTO} was incubated with either [FIP-HRC]₂-PEG₁₁ or Fab 77.1 and purified via size exclusion chromatography (SEC) (FIG. 8 F). The resulting complexes were analyzed by single-particle cryogenic electron microscopy (cryo-EM). Applicants obtained a high-resolution structure of the F2M_{ECTO}-[FIP-HRC]₂-PEG₁₁ complex at 2.3 Å (FIG. 3B). All F2M_{ECTO} particles adopt a prefusion state with clear density for FIP. However, Applicants did not see density for the HRC-derived mimic, likely due to the high flexibility of the peptide when tethered to FIP (FIG. 12A, B). Moreover, Applicants did not observe alternative conformations of F in 2D class averages suggesting that a fusion-intermediate state was not available for HRC to stably bind.

The overall structure of F2M_{ECTO} closely resembles previous cysteine-stabilized prefusion F configurations, where the head region is a composite structure made up of segments from both F1 and F2, which are linked by disulfide bonds. The F2 subunit twists around the trimer of hetero dimers and intercalates between the N-terminal heptad repeat (HRN), DI, and DIII of the F1 subunit. Meanwhile, the fusion peptide, located at the extreme N-terminus of F1, reaches out from one monomer to lay on top of the immunoglobulin-like domain II of a neighboring monomer. Together, these domains form a pyramidal shape. Finally, the C-terminal region of F1 (C-terminal heptad repeat, HRC) coils around domain II and extends downward to form the stalk of the trimer (FIG. 3 C). Applicants note, however, two differences in previous F structures as compared to those presented here. First, comparison of the previously published X-ray structures (Cys-stabilized, APO: 5YXW, FIP-bound: 5YZD and AS-48-bound: 5YZC) to these cryo-EM structures (no Cys stabilization, but two naturally occurring point mutations that confer stability, and bound to either Fab or inhibitory peptide), shows loop 248-265 in different conformations, with an RMSD of ~4 Å. This conformational variance is likely attributable to crystal contacts between loop 248-265 and F2 helix 75-86 from a symmetry mate (FIG. 12C, D, E). Second, Applicants observe differences in the conformation of the stalk regions between the structures. In the cryo-EM-based model, the alpha-helices of the HRC in the stalk region begin to super-coil around each other, starting at residue 473. Conversely, in the Cys-stabilized construct, the stalk region remains linear and does not exhibit supercoiling (FIG. 12C, D, E). Overall, the differences are minor and might be related to the construct stabilization (stalk conformation) or technique used for structure determination (loop 248-265) but the general protein structure remains the same.

Similar to the previous structure of MeV F in complex with a related peptide inhibitor, here Applicants find that FIP stabilizes F by occupying the hydrophobic pocket created by F1 residues from all 3 protomers including residues F1_{A} 311W, 370L, 451P, 452I, 453S, 459V and 466A, F1_{B} 463L, 464G, 4671, 471E and F1_{C} 352S and 353P. Together, these interactions lock MeV F in the prefusion state and prevent the refolding of heptad repeats.

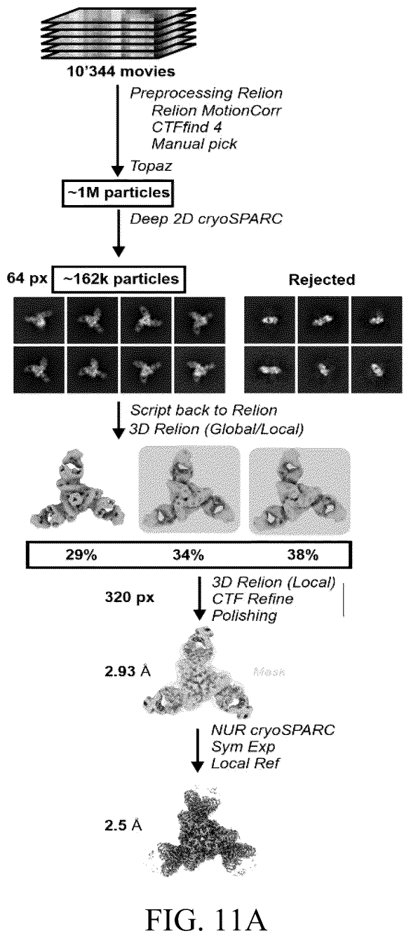

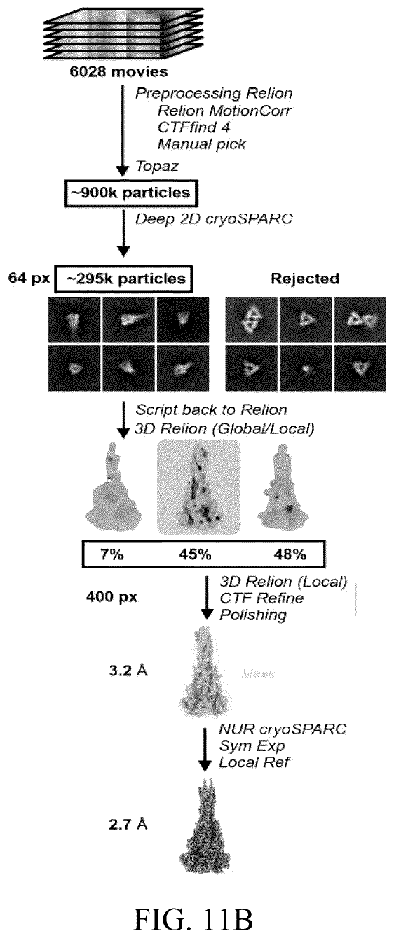

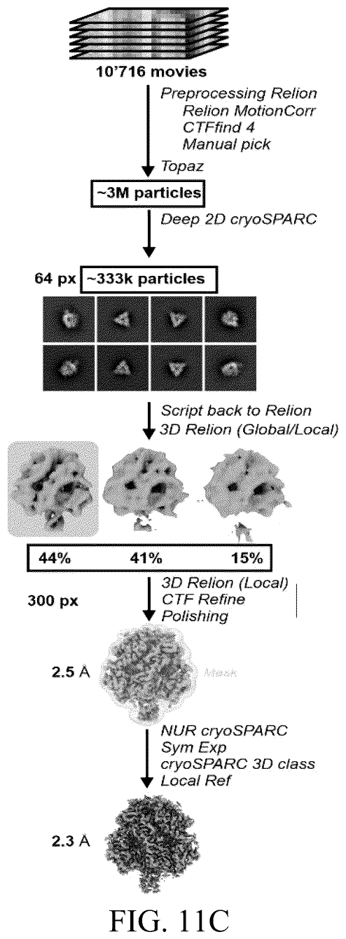

The final map of the F2M_{ECTO}-Fab 77.1 complex was resolved to 2.6 Å by masking the flexible constant regions of the Fab and conducting a final local reconstruction in C1 from the C3 symmetry-expanded particle set (FIG. 11A). Three copies of Fab 77.1 are bound to the prefusion F2M_{ECTO} trimer. The epitope is located at the base of the trimer and encompasses regions in F1 and F2 from one protomer and F1 from a neighboring protomer (FIG. 3 D, E, G). The heavy chain of Fab 77.1 contributes the bulk of the interaction, burying 889 Å² of surface area on F_{ECTO}, as compared to the 283 A2 buried by the light chain (Table 3). The predominant interactions between the antibody and F2M_{ECTO} occur via the heavy chain complementarity-determining region (CDR)-3, with additional contributions from CDR-H1, CDR-H2, and CDR-L2 (FIG. 3B, Table 2).

CDR-H3 occupies a shallow cleft between domains I and II of F1_{B}, forming six hydrogen bonds and one salt bridge. In particular, F1B residue R436 forms hydrogen bonds to the backbone oxygens of CDR-H3 residues Y105 and Y107 and a salt bridge with CDR-H3 D109 (FIG. 3 G). CDR-L2 residues Y49 and E55 also bind to this region, making a hydrogen bond with the backbone nitrogen of R435 and the hydroxyl group of S434 (Table 4). Meanwhile, CDR-H2 interacts with F2 through contacts with F2 residues W27, G37, and S40. Notably, CDR-H1 residues Y27 and S31 form hydrogen bonds with the backbone of residues L123 and V125, located in the fusion peptide of the neighboring protomer, F1c.

Together, this extensive network of 16 hydrogen bonds and one salt-bridge anchor domains I and II of F1 in the prefusion state.

### 77.1 Binding Interface is Well Preserved in MeV Variants.

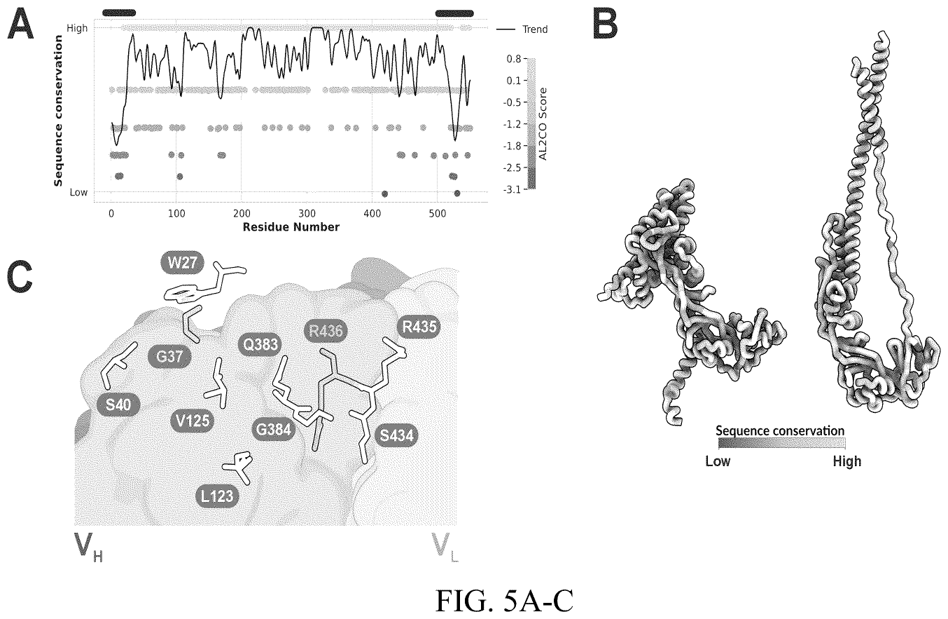

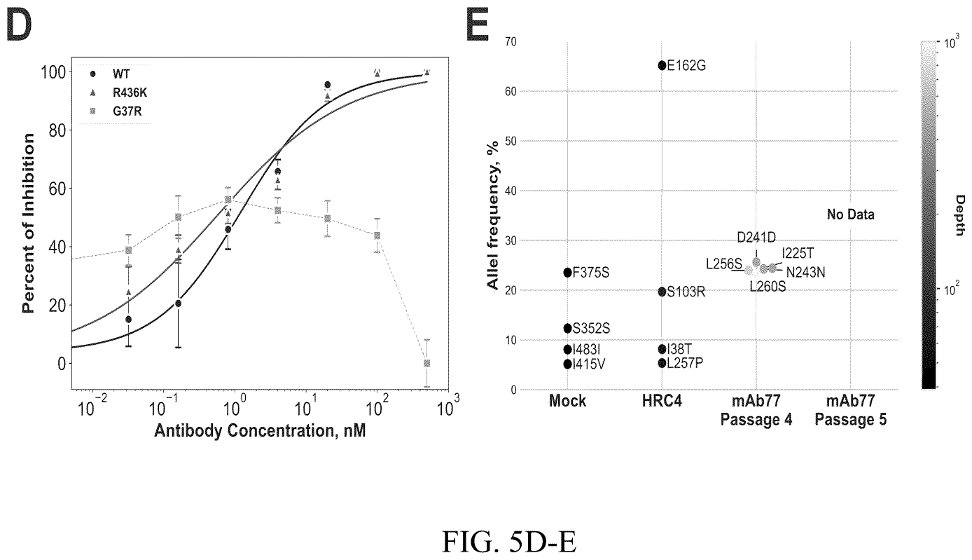

Although MeV has been generally considered a serologically monotypic virus, genetic characterization of wild-type viruses has identified eight clades (A-H), which have been further divided into 22 genotypes plus one additional proposed genotype. To evaluate whether natural genetic variation in MeV might impact mAb 77.1 binding, Applicants examined sequence conservation across the available MeV F protein sequences in the NCBI database. Of the 550 residues in MeV F, 319 are fully conserved, while 231 (42%) have single or multiple amino acid substitutions (FIG. 5A). The signal peptide and cytoplasmic tail have the greatest sequence variability, but substitutions are distributed throughout the tertiary structures of pre- and post-fusion MeV F (FIG. 5A, B). Within the mAb 77.1 epitope, however, only two residues, G37 and R436, vary across the range of emerged MeV sequences (FIG. 5C). One single F sequence (0.12%) had a G37R substitution which could sterically hinder the F-77.1 interface. The second mutation, however, R436K, occurs in more than ¼ of F sequences. Mutation of R436 to a lysine could impact association of mAb 77.1 with F, particularly the hydrogen bond network and salt bridge formed between R346 and mAb residue D109. To assess the impact of each of these substitutions, Applicants evaluated the ability of mAb 77.1 to block fusion of F variants bearing either the naturally occurring G37R or R436K or an artificially created charge reversal mutant of R436D or charge loss of R436G.

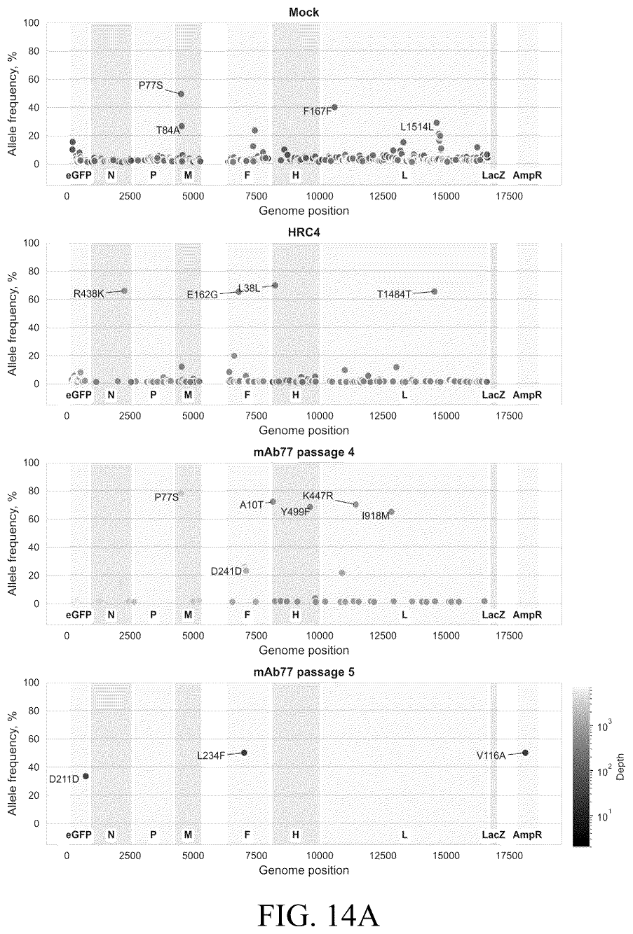

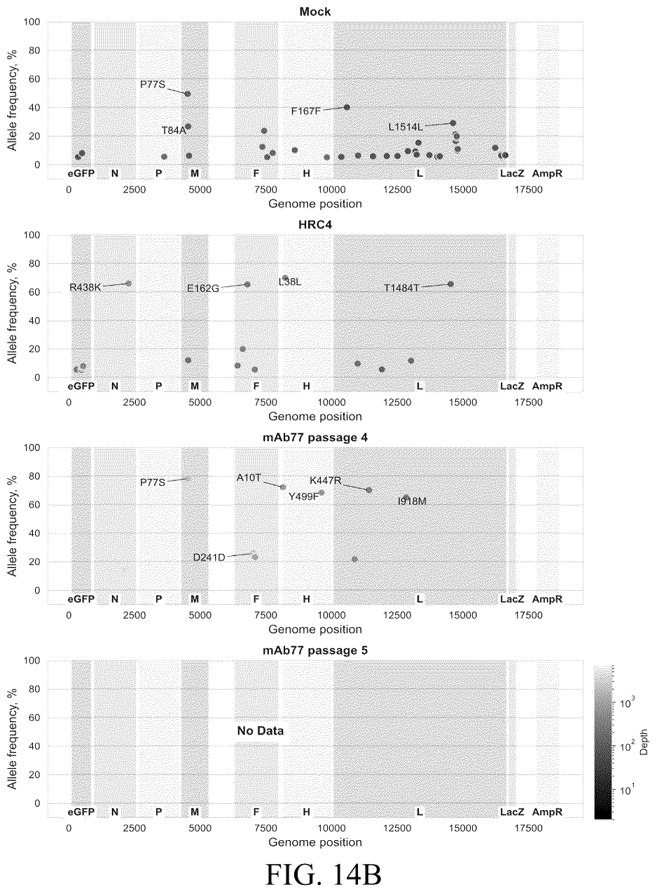

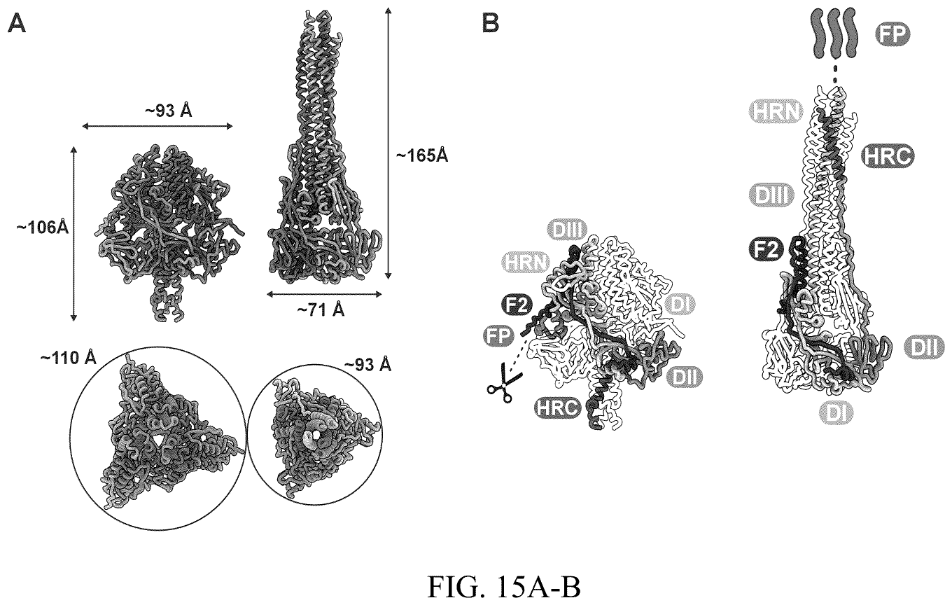

Applicants first evaluated the expression of each F mutant, as well as recognition by mAb 77.1. Both expression and mAb 77.1 binding to FR436K were comparable to that of F_{WT} (FIG. 14A, B, C). In contrast, while FG37R maintained wild-type expression levels, recognition by mAb 77.1 was substantially reduced (FIG. 14A, B, C). Lastly, the compromised expression of FR436G and FR436D at 37 °C was only partially recovered at 32 °C. Recognition of either mutant by mAb 77.1 was also reduced (R436G) or abrogated (R436D), likely due to the charge loss or reversal, respectively, at this site (FIG. 14A, B, C, F).

Notably, Applicants found that both fusion capacity and mAb-mediated fusion inhibition of FR436K paralleled that of F_{WT} (FIG. 5D, FIG. 14D, E). FG37R, on the other hand, had ~8-fold less fusion as compared to F_{WT} and escaped mAb-mediated inhibition (FIG. 5D, FIG. 14D, E). The lower fusogenicity of the FG37R variant likely explains its very low incidence in naturally 31 occurring MeV strains. As expected, FR436G and FR436D also escaped mAb-mediated fusion inhibition (FIG. 14 D, E).

This data suggests that mAb 77.1 is likely to maintain efficacy towards currently circulating measles virus strains. However, it could be susceptible to infrequent mutations, such as G37R, or other emergent mutations not yet described, particularly if used as a therapeutic for active MeV infection.

To understand the potential impact of mAb 77.1 on viral evolution, Applicants attempted to select escape mutations. MeV was incubated with increasing concentrations of mAb 77.1 for five viral replication cycles. Viral proliferation ceased after cycle five likely due to the accumulation of deleterious mutations. Nevertheless, the viral population from cycles four and five was harvested and deep-sequenced to identify potential genetic changes. Intriguingly, despite the extended exposure of the virus to mAb 77.1, the analysis failed to uncover any significant point mutations (allele frequency ≥5%, sequencing depth ≥20) (FIG. 5E, FIG. 15A, B). Together, the analysis of naturally occurring mutations and in vitro viral evolution suggests a high barrier to viral escape from mAb 77.1.

### F Post-Fusion Structure

The structure of F_{ECTO} in complex with Fab 77.1 unequivocally demonstrates this mAb recognizes the pre-fusion conformation of F and anchors the fusion peptide through interactions with CDR-H1. However, the RBC fusion assay (FIG. 2) suggests that mAb 77.1 prevents viral infection by arresting the fusion process after the fusion peptide has been released from the trimer and inserted into the host cell membrane.

To reconcile the divergence between the Fab 77.1 epitope and the cell biology results, Applicants determined the cryo-EM structure of F2M_{ECTO} in its post-fusion state, which was not yet available. In particular, direct structural comparison of the pre- and post-fusion states from the same protein sequence would provide a more comprehensive understanding of the conformational dynamics of F2M_{ECTO} and clarify how mAb 77.1 might selectively interact with the pre-fusion form or reveal conservation of the epitope in the post-fusion form.

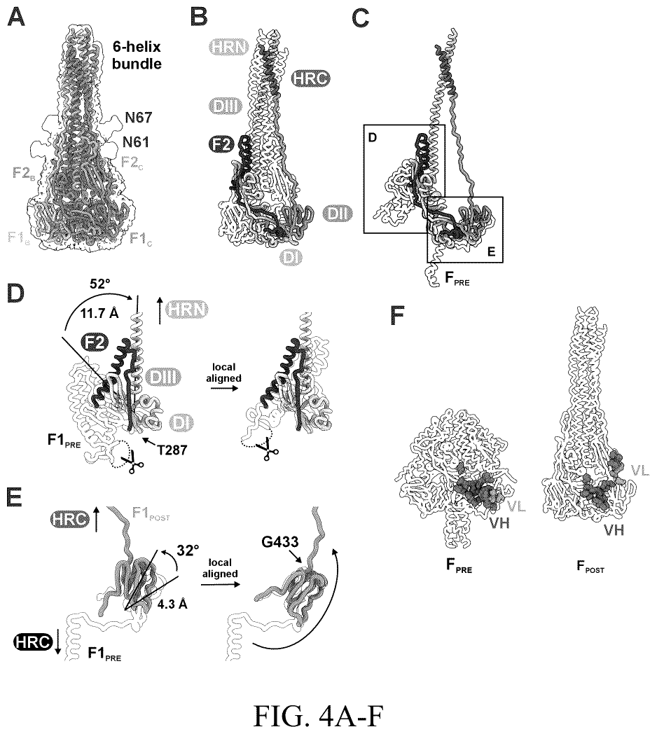

As previously described, while storage of F2M_{ECTO} in a high-salt buffer results in conformational stability, storage in a low-salt buffer results in spontaneous refolding to the post-fusion state. Hence, by incubation of the protein in low-salt buffer (i.e., 150 mM NaCl), Applicants were able to capture the 2.7 Å resolution cryo-EM structure of F2M_{ECTO} in its post-fusion conformation using the same construct as represented in the pre-fusion F2M_{ECTO} structures (FIG. 4A).

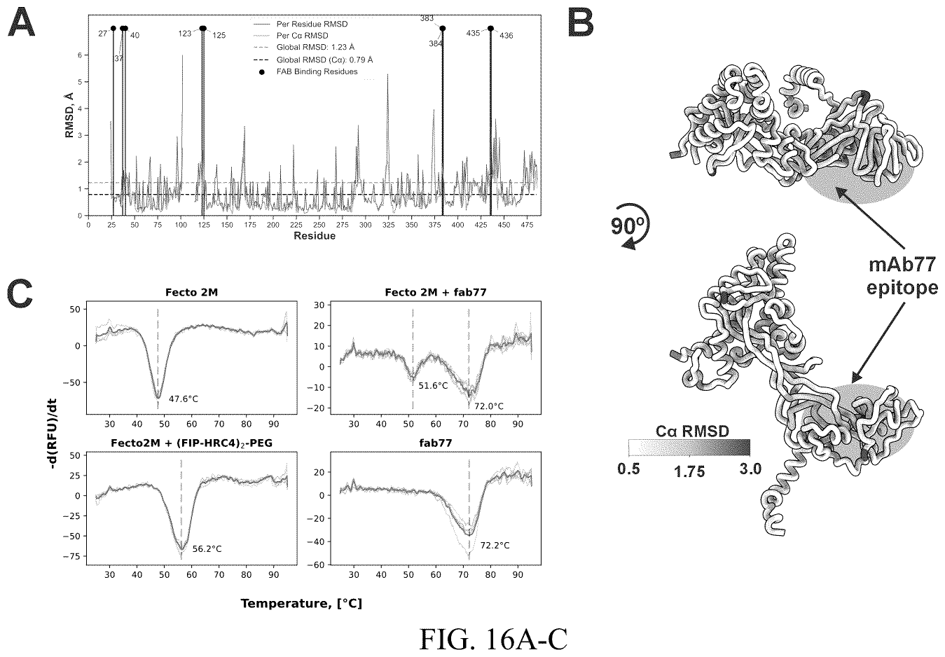

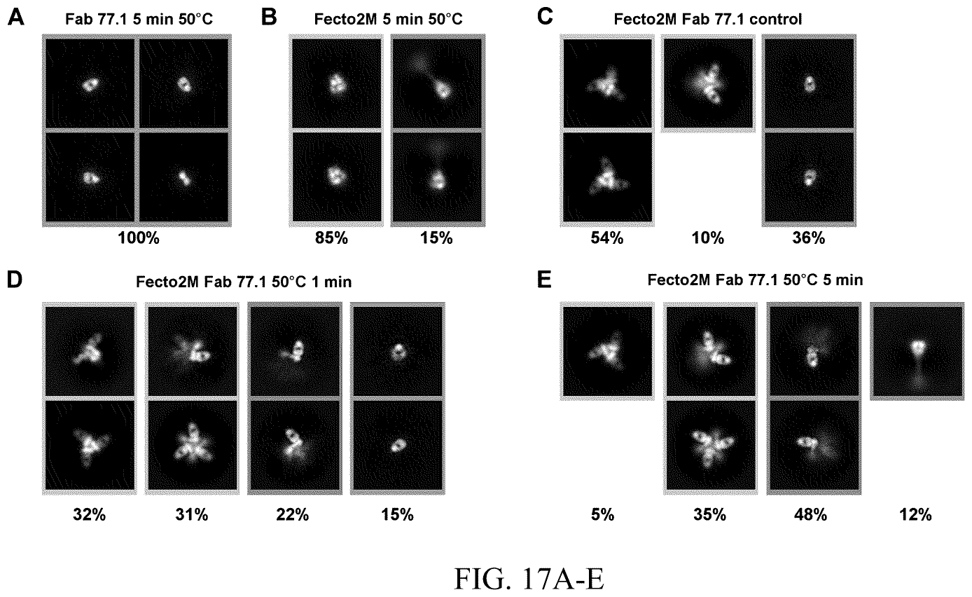

Like post-fusion structures of the F proteins of other paramyxoviruses, the MeV F_{ECTO} post-fusion structure adopts a distinct elongated shape with a refolded head domain and an extended 6-helix bundle composed of HRN and HRC (PDB: 8DMJ (unpublished),). This conformational shift leads to an approximate 20% reduction in head radius (from ~110 Å to ~93 Å) and an extension of the protein's long dimension by 60 Å (FIG. 16A, B). Despite the fusion peptide (FP) (residues 115-140) being part of the F_{ECTO} sequence, the absence of an observable density for the fusion peptide (FP) could be due to several factors: the FP's inherent structural disorder or its ability to adopt multiple conformations.

Alignment of the post-fusion structure of F2M_{ECTO} with its pre-fusion counterpart revealed that like other paramyxoviruses, domain I (residues 290-373) and N-terminal part of F2 undergo the least rearrangement during fusion and provide a structural hinge about which the movement and rotation of other domains occur (, FIG. 4C). In contrast, there is a substantial spatial and structural rearrangement of domains II (F1 residues 374-426) and I (F1 residues 115-287 and F2 residues 50-104) (FIG. 4D, E, FIG. 15 B). Although the majority of domain II retains the immunoglobulin-like fold present in the pre-fusion conformation, residues 434-446 of this domain as well as the linker connecting domain II to HRC (residues 447-456) elongate as HRC transitions from the “down” state to the “up” state as fusion proceeds. As a result, the entirety of domain II shifts 4.3 Å toward domain I.

The most profound conformational change from pre- to post-fusion occurs in the FP and HRN. Structural rearrangement within these regions includes the refolding of HRN from several disjointed helices and β-strands to one elongated helix, exposure of the buried fusion peptide, and a 52° rotation around residue T287 (FIG. 4D). Overall, the conformational transition from pre- to post-fusion involves dramatic refolding of the FP and HRN, retention of the globular domain structure, but repositioning of the domains relative to their pre-fusion positions.

The significant conformational shifts and repositioning of the three components of the tripartite mAb 77.1 epitope clarify why 77.1 is unable to recognize Fin its post-fusion state (FIG. 4F). Specifically, the spatial rearrangement between domain II and the C-terminal heptad repeat (HRC), coupled with the movement of domain II relative to domain I, leads to the dislocation of the mAb 77.1 epitope and disrupts recognition of F.

### mAb 77.1 Interrupts the Fusion Cascade, Leading to Trimer Disintegration.

The structure of mAb 77.1 in complex with F, coupled with the post-fusion F structure, clearly demonstrated the prefusion conformational specificity of the epitope. However, the cell biology mechanistic assay suggested that mAb 77.1 acts on F at an intermediate state—after fusion has been activated but before forming the six-helix bundle.

To gain a clearer understanding of the mechanism by which mAb 77.1 inhibits viral entry, Applicants sought to investigate its impact on F_{ECTO} structure and stability. Specifically, Applicants considered two main hypotheses: 1) mAb 77.1 destabilizes the prefusion state of F_{ECTO}, thereby facilitating its transition to the post-triggered state, or 2) binding of Fab 77.1 induces conformational changes in F_{ECTO}, influencing the refolding cascade. To test these hypotheses, Applicants investigated the structural effects of Fab 77.1 binding on F_{ECTO}, as well as assessed the influence of Fab 77.1 on thermal stability via DSF.

Alignment of the structures of F_{ECTO} in complex with either [FIP-HRC]₂-PEG₁₁ or Fab 77.1 demonstrated high structural conservation, with an RMSD of 0.79 Å over all Ca or 1.23 Å over all atoms. Residues involved in the mAb 77.1 epitope showed only minor differences between structures, with fusion peptide residues L123 and V125 having an RMSD of ~2.2 Å over all atoms (FIG. 16A, B). Hence, Fab binding only marginally alters the conformation of F_{ECTO}. In line with this comparison, Applicants found that Fab 77.1 increases the thermostability of F from a Tₘ^{app} of 47.6 (±0.1) °C to 51.6 °C (FIG. 16 C). While this stabilization was not as significant as that of [FIP-HRC]₂-PEG₁₁ (56.2 °C), it nevertheless suggests that mAb 77.1 does not destabilize F.

To further elucidate the mechanism of action mAb 77.1, Applicants turned to negative stain (NS) EM to directly visualize the molecular events following F activation in the presence of Fab 77.1. F_{ECTO} was complexed with excess Fab 77.1 for 4 hours at room temperature. Subsequently, the mixture was incubated at 50 °C (above the Tₘ^{app} of F but below that of the F-Fab 77.1 complex) for 1, 5, 7.5, or 10 minutes. Samples were then rapidly cooled on ice for 5 minutes and applied to negative stain grids.

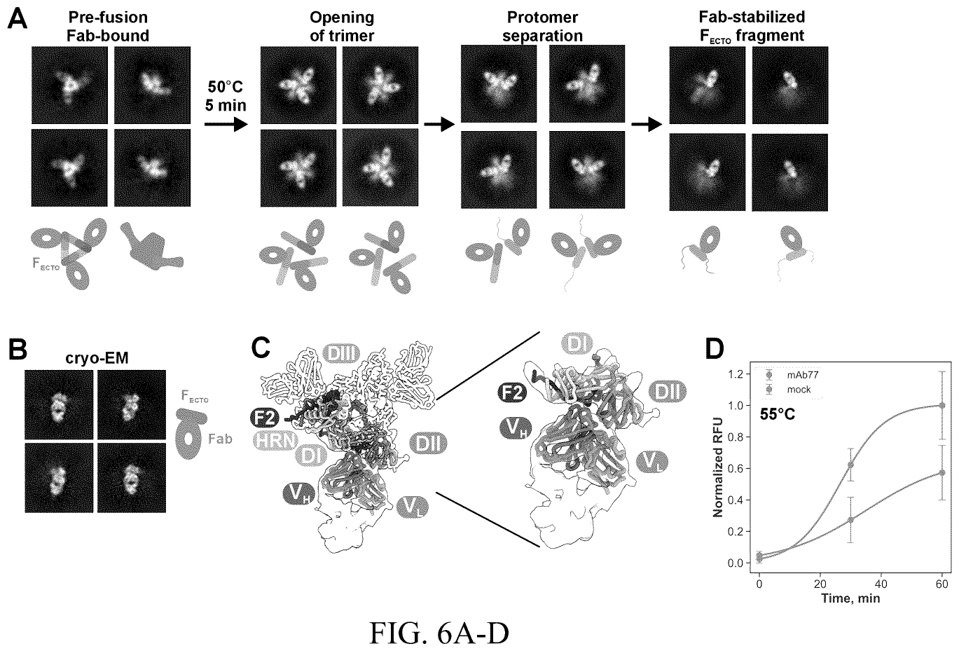

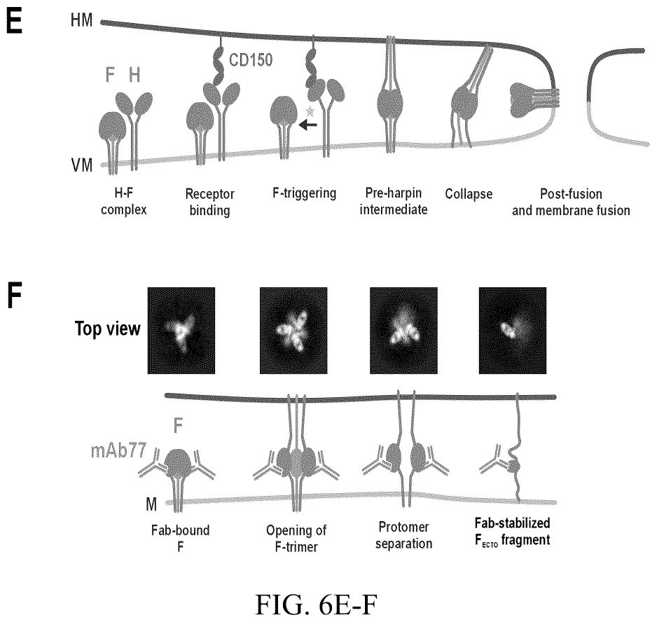

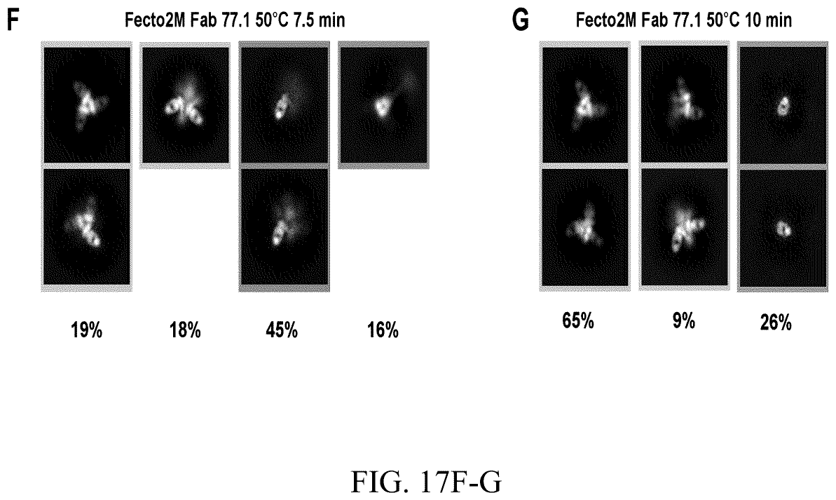

NS EM imaging of the 1, 7.5, and 10-min time points showed a mixture of states including F_{ECTO}-Fab 77.1 complex, F_{ECTO} in post-fusion conformation and new, previously unobserved conformations and those were the most apparent in the 5-min time point (FIG. 6A, FIG. 17). Specifically, Applicants observed a subset of complexes where three Fab molecules remained bound to F_{ECTO}, albeit with significant changes in the conformation of F_{ECTO} in which the head appears to adopt a relaxed, open state, leading to the separation of the protomers and potentially refolded HRN and FP domains. Additionally, another state was observed where one of the protomers had dissociated from the trimer with the remaining two protomers, each flanked by a Fab, forming a “V”-shaped structure. Lastly, Applicants also identified 2D classes where only a small rigid fragment of F_{ECTO} remained bound to Fab 77.1 (FIG. 6A).

To further validate these findings, Applicants re-examined the cryo-EM dataset of the F_{ECTO}-Fab 77.1 complex, where similar classes were noted, particularly those of Fab bound to a small fragment of F (FIG. 6B, C FIG. 11A). A map of this class was successfully resolved to 7 Å resolution. Fab 77.1 was first docked into the apparent Fab density. Then, the Fab portion of the F_{ECTO}-Fab trimer was aligned to this docked model. The density not attributable to Fab aligned well with the mAb 77.1 epitope on F_{ECTO} (i.e., domain II, part of domain I, and the N-terminal part of F2) (FIG. 6C).

To further dissect how mAb 77.1 modulates F conformational rearrangements, Applicants assessed the binding of a post-trigger-specific antibody (mAb 19GD6, anti-Trig) to cell-surface expressed F, in the presence and absence of mAb 77.1. Although the specific epitope of this mAb has yet to be elucidated, previous studies have shown it binds after triggering has occurred. F-transfected cells were incubated with mAb 77.1 or diluent media alone, heated to 55 °C for 30 or 60 mins, and stained with mAb 19GD6. The presence of mAb 77.1 led to increased detection of post-trigger F epitopes, corroborating the earlier findings from negative-stain electron microscopy (FIG. 6D). These collective results imply that the antibody's mechanism of action involves disrupting the F trimer upon activation, thereby revealing post-trigger epitopes possibly through trimer fragmentation.

Compiling these results with previously obtained data, Applicants propose a model detailing how mAb 77.1 effectively neutralizes MeV. In the canonical fusion cascade, the H-F complex engages either CD150 or Nectin-4 receptors, triggering structural changes in the H protein that lead to F protein activation (FIG. 6E). Subsequent refolding steps lead to a post-fusion conformation, driving membrane fusion. The data indicate that mAb 77.1, while binding to the pre-fusion state of F, does not prevent F activation. Structural changes in domain III and HRN and imposed conformational constraints of domain I and domain II by mAb 77.1 result in protomers separation and trimer relaxation which ultimately disrupt the F_{ECTO} trimeric structure, except for the fragment stabilized by mAb 77.1 (FIG. 6F).

The model provides a mechanistic basis for the Fab 77.1-bound pre-fusion state observed in cryo-EM data and explains the outcome of the cell biology mechanistic assay. Moreover, Applicants postulate that the post-fusion conformation observed in this study, a critical step in the canonical fusion pathway, is not part of the mAb 77.1-modulated refolding cascade. This hypothesis is supported by the absence of any post-fusion states in the cryo-EM dataset for the F_{ECTO}-Fab 77.1 complex and the presence of Fab-stabilized F_{ECTO} fragments.

### CONCLUSION

MeV remains a global concern and source of considerable morbidity and mortality despite the availability of an effective vaccine. Notably, all MeV vaccines in use involve live-attenuated virus, which poses risks to those with weakened immune systems, such as cancer patients, those undergoing immunosuppressive therapy, pregnant women, and infants below 12-15 months. Furthermore, approximately 5% of those that can safely receive the live-attenuated vaccine do not develop protective anti-MeV responses. Alternatives to vaccination, such as antibody-based therapeutics, would provide an orthogonal line of defense that could be used in a broader population. However, such molecules are not yet available.

Nearly all anti-MeV monoclonal antibodies thus far described are directed against the receptor-binding protein H. The few anti-MeV F mAbs that have been isolated belong to one of two main antigenic groups, termed I and II. Epitope I mAbs are potently neutralizing but susceptible to escape by naturally occurring MeV variants. Among epitope II mAbs, 77.4 was the most potent. Here, Applicants show the human-chimeric mAb 77.1 retains this potent inhibition of F-mediated fusion in vitro, and demonstrate that the mAb limits viral replication in vivo, even at a very low dose of 0.1 mg/kg (FIG. 1).

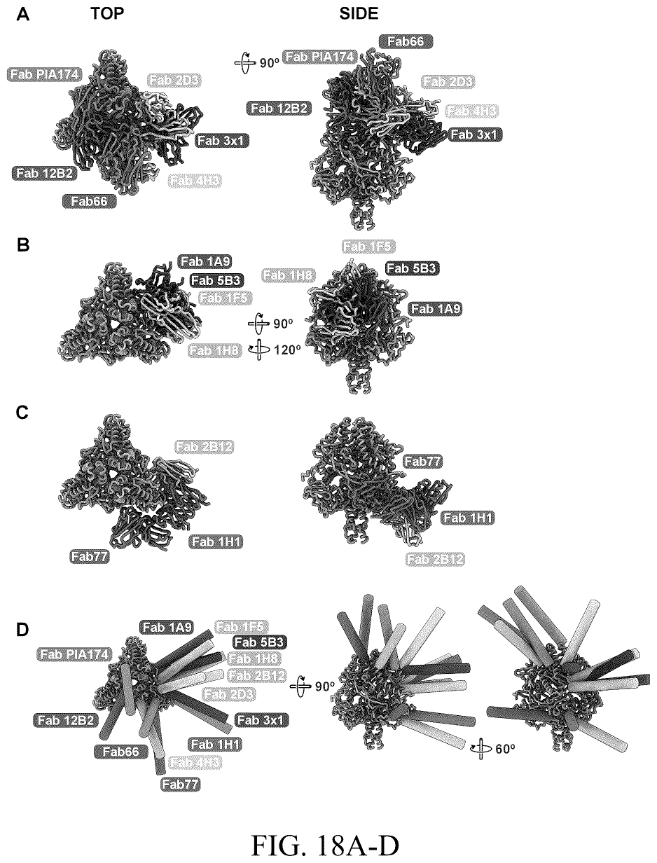

Several antibodies that bind the pre-fusion state of other paramyxovirus fusion proteins have been described (PDB ID: 7UOP, 7UPA, 7UPB, 7UPK), and for the respiratory syncytial virus (RSV), one such antibody is in clinical use (FIG. 18). Comparison of mAb 77.1 and the 12 Fab-bound F structures currently deposited in the PBD reveals there are three, rather than two, major sites that anti-F antibodies target: the apex of the head domain, the face of the trimer where two protomers meet, and the base of the head domain (FIG. 18A-D). Like the two unpublished Nipah F-targeted mAbs, 2B12 and 1H1, mAb 77.1 binds the base of the head domain. However, its quaternary epitope spans across domains and protomers, rather than encompassing only domain II, as do 2B12 and 1H1. Regardless of the epitope, the suggested, and in some cases known, mechanism of action for each of these published antibodies is to block fusion activation by locking F in the prefusion state. As such, Applicants hypothesized that mAb 77.1 inhibits viral entry similarly: by binding to pre-fusion F before it has been activated by H-receptor engagement on the target cell. This mechanism is in contrast to HRC peptide mimics, which interact with the F protein only after activation of fusion where the virus has attached to the cell membrane, but before fusion has been completed. Unexpectedly, however, the cell biology mechanistic assay demonstrated that mAb 77.1 does not block the activation of fusion and the launch of conformational change, but instead, like HRC mimics, arrests the protein at an intermediate step along the fusion pathway (FIG. 2).

Paradoxically, the high-resolution cryo-EM structure of F2M_{ECTO} bound to Fab 77.1 shows F is in the prefusion conformation instead of adopting a conformation more consistent with a fusion intermediate. Comparison of Fab 77.1-bound F2M_{ECTO} to other F_{ECTO} structures, including the [FIP-HRC₄]₂-PEG₁₁-F2M_{ECTO} complex detailed here, demonstrates little structural difference. The only exception is the fusion peptide, which is held in a slightly different position by the Fab (FIG. 16 B). Furthermore, the post-fusion structure of F2M_{ECTO}, the first available for MeV, shows that the tripartite mAb 77.1 epitope is fractured upon the collapse of HRC into the 6-helix bundle with HRN. Hence, mAb 77.1 cannot bind to post-fusion F.

Curiously, the quaternary epitope of mAb 77.1 includes the fusion peptide of F, which extends from a neighboring protomer (e.g., FB) and onto the top of domain II of FA. However, the mechanistic assay indicates that the fusion peptide is released and inserted into the host membrane. Applicants note that the fusion peptide makes a relatively small contribution to the overall epitope. Only two of the sixteen hydrogen bonds are formed between the mAb and the fusion peptide (CDR H1 Y27 and S31 with L123 and V125, respectively). Based on PISA predictions, these interactions contribute just-1.5 kcal/mol of free energy upon complex formation as compared to the −8.6 kcal/mol released over the entirety of the epitope. Hence, the fusion peptide, which is located on the edge of the epitope, could potentially be extracted, leaving the antibody anchored to the N-terminal portion of F2 and the loop between domain II and HRC. The stronger remaining interactions would likely still preclude repositioning of HRC, which requires release of the domain II-HRC loop. The structures also suggest that the fusion peptide may not be the first region to refold upon fusion triggering. Instead, refolding of HRN from several disjointed sub-sections into an elongated helix could be the driving force for the refolding that, once initiated, pulls the FP from its location atop the neighboring domain II, like a ribbon from a box.

Indeed, using NS EM, Applicants are able to capture several distinct conformations of F-Fab complexes, including a relaxed and open trimer, still bound by three Fabs, disassembled trimer with only two promoters bound by Fab 77.1 and a single Fab bound to small, structured fragment. Upon further data mining of the cryo-EM data set used to solve the trimeric F-Fab structure, Applicants also identified a population of Fab bound to an ordered fragment of F, corresponding to the epitope, minus the fusion peptide.

Based on these biochemical and structural findings, Applicants propose a model outlining how mAb 77.1 provides protection against MeV. mAb 77.1 recognizes the F protein while it is in its pre-fusion state, implying that binding could occur while the virus circulates within the host, rather than during the virus-host engagement phase, unlike HRC derived peptides. Despite this, the F protein can still be activated by the H-receptor and initiate the canonical pathway for membrane fusion. However, the rigidity introduced by the binding of mAb 77.1 to the epitope, which includes domains I and II, as well as the linker region between domain II and HRC, prevents F from following its standard refolding route. Instead, the F trimer adjusts by separating its protomers to accommodate this rigidity. As the refolding progresses to undefined intermediate states, the F protomers continue to separate until only one remains, stabilized by mAb 77.1. This stabilized state may even induce in vivo specific neutralizing antibodies against the intermediate state, a hypothesis that Applicants can test in the future.

This newly understood mechanism of antibody-mediated defense may also apply to other antibodies specific for pre-fusion states. Previous studies focusing on such antibodies against Nipah, Hendra, or Parainfluenza viruses have primarily examined binding kinetics, neutralization efficiency, and structural aspects without delving into the mechanistic actions of these antibodies. Therefore, it is plausible that some of these previously studied antibodies may function similarly to mAb 77.1 (FIG. 18 D), although the exact mechanism of action has not been explored. In the case of the HPIV3 neutralizing antibody PIA174 Applicants have shown that it blocks fusion by stabilizing the pre-fusion state of the protein and escape variants are easily obtained. Additionally, the only other antibodies shown to recognize a viral glycoprotein refolding intermediate state are the HIV-1 gp41-specific monoclonal antibodies, 2F5 and 4E10. These rare antibodies were known to neutralize by targeting a transient conformation during the gp41 conformational changes involved in membrane fusion. However, the precise epitopes and structures contributing to their specific binding to the intermediate state had been unresolved.

In conclusion, the research thoroughly characterizes a novel, highly efficacious prophylactic antibody against the MeV that uses a unique neutralization mechanism. This study is the first to provide a comprehensive analysis of the mechanism for antibody-mediated neutralization. The findings could have broader implications for understanding other antibodies that target the pre-fusion state of proteins. Furthermore, leveraging high-resolution cryo-EM reconstructions, Applicants have elucidated the two states of the MeV F protein, thereby paving the way for the next generation of MeV therapies that can be precisely tailored to accommodate the dynamic nature of F.

### Material and Methods

Plasmids. The genes of MeV IC323 or B3 hemagglutinin, fusion protein, nectin 4, and CD150 (or SLAM) proteins were codon optimized, synthesized, and sub-cloned into the mammalian expression vector pCAGGS. A codon-optimized fusion protein sequence derived from the Measles virus strain Ichinose-B95a (Taxonomy ID: 645098) was synthesized by GeneScript and subcloned into the pcDNA3.1 vector. Stabilizing point mutations (E170G and E455G) previously characterized in SSPE and MIBE Measles strains were sequentially introduced using the Q5 Site-Directed Mutagenesis kit. The ectodomain construct was generated by removing the transmembrane region and cytoplasmic tail residues (496-550) using the Q5 Site-Directed Mutagenesis kit. To generate Drosophila S2 stable cell lines, fusion protein ectodomain construct E170G/E455G was subcloned from the pcDNA vector to the pMT-Puro vector, including a C-terminal enterokinase cleavage site-twin-strep-tag via NEB HiFi DNA assembly.

Cells. HEK293T (human kidney epithelial), Vero, and Vero-SLAM/CD150 (African green monkey kidney) cells were grown in Dulbecco's modified Eagle's medium (DMEM; Life Technologies; Thermo Fisher Scientific) supplemented with 10% fetal bovine serum (FBS, Life Technologies; Thermo Fisher Scientific) and antibiotics at 37 °C in 5% CO₂. The Vero-SLAM/CD150 culture media were supplemented with 1 mg/ml Geneticin (Thermo Fisher Scientific). Drosophila S2 cells were cultured in complete Schneider's Drosophila Medium (Gibco) supplemented with 10% heat-inactivated fetal bovine serum (FBS) and 1% penicillin-streptomycin (100 U/mL penicillin and 100 μg/mL streptomycin) at 25 °C without CO₂ or Lonza Insect Xpress Medium. ExpiCHO cells were maintained in ExpiCHO Expression Medium (Gibco) supplemented with 8 mM L-glutamine in a humidified incubator at 37 °C with 8% CO₂.

In vitro viral entry: Vero-SLAM cells were plated in 96-well plates (1×10⁴ cells/well). The following day, cells were treated with serial dilutions of antibody 77.1 (mAb 77.1); N-(3-cyanophenyl)-2-phenylacetamide, referred to as 3G, and MeV F-derived HRC4 dimeric fusion inhibitory lipopeptide (i.e., [HRC-PEG4]₂-chol). Simultaneously, cells were infected with MeV WT B3-EGFP for 3 h at 37 °C (150-200 pfu/well). After 3 h, the medium was replaced with medium containing 2% of methylcellulose Avicel/2× complete medium (1:1). After 24 h, GFP-infected cells were counted using a Cytation 5 (BioTek) and analyzed with Gen5 3.11 software (min object size 25 μm, max object size 135 μm, threshold 3,000). The samples were tested in three separate experiments performed with technical duplicates in five-fold dilutions in 96 well plates. The measurements from each plate were normalized using 100 nM of mAb 77.1, 25 μM of 3G compound, and 500 nM concentration of HRC4 as 100% inhibition. Data was analyzed in OriginPro 2023, where all biological and technical replicates were fitted with logistic regression function with shared IC₅₀ and Hill slope values, and A1 and A0 (highest and lowest signals) were used to normalize the data. Based on the normalized data, the percent of inhibition was calculated using the following equation: [1−X]*100, where X is the fraction of the normalized signal.

Beta-Galactosidase (Beta-Gal) complementation-based fusion assay. The Beta-Gal complementation-based fusion assay was performed as previously described. Briefly, HEK293T cells transiently transfected with nectin 4 or CD150 receptor-bearing cells expressing the omega subunit of β-Gal are mixed with cells co-expressing glycoproteins F and H and the alpha subunit of β-Ga. Cell fusion leads to alpha-omega complementation and is measured in the presence or absence of fusion inhibitory compounds. Fusion is stopped by lysing the cells and, after addition of the substrate (®The Tropix Galacto-Star™ chemiluminescent reporter assay system, Applied Biosystems), fusion is quantified on a Tecan M1000 microplate reader. Three biological replicates were recorded, and globally fitted with a logistic function in OriginPro 2023. Obtained parameters were used to normalize the data per sample and plotted with standard error calculated from all data points.

Cell surface staining with conformation-specific anti-F mAbs. HEK293T cells transiently transfected with viral glycoprotein constructs. The transfected cells were incubated at the temperatures and times indicated in the figures. Specific MeV F conformations were detected with mouse monoclonal antibodies (mAbs) followed by Alexa-488 anti-mouse secondary antibody (Life Technologies) and DAPI (4′,6-diamidino-2-phenylindole; Thermo Fisher) staining. Plates were imaged with a Cytation5, and the relative fluorescence of antibody-bound cells was determined using BioTek Gen5.

Viral evolution assay: Vero-SLAM cells were infected with 100 PFU/well of MeV B3-EGFP in 96-well plates then incubated for 2 h at 37 °C and further treated with several concentrations of inhibitors to promote the emergence of escape variants. Viruses were collected after five days and passaged similarly several times as indicated.

In vivo experiments: Cotton rats. Inbred cotton rats (Sigmodon hispidus) were purchased from Envigo, Inc., Indianapolis. Both male and female cotton rats aged 5 to 7 weeks were used. For i.n. infection, 10⁵ TCID₅₀ of MeV (strain B3 wt EGFP) was inoculated intranasally to isoflurane-anesthetized cotton rats in a volume of 100 μl. Animals received either the indicated amount of mAb 77.1 or vehicle 12 hours prior to infection. Four days after infection, the animals were euthanized by CO₂ inhalation, and their lungs were collected and weighed. Lung tissue was homogenized with a glass dounce homogenizer. Serial 10-fold dilutions of supernatant fluids were assessed on Vero SLAM cells for the presence of infectious virus in 48-well plates using cytopathic effect (CPE) as the endpoint. Plates were scored for CPE microscopically after 7 days and the TCID₅₀ was calculated. The results are presented as TCID₅₀/g of lung tissue.

RBC fusion assay. RBC fusion assays were performed on HEK 293T cells transiently expressing MeV H_Y17H HPIV3_T193A chimera and MeV F (T461I). Cell monolayers were washed three times with serum-free medium, placed at 4 °C with 1% RBC in CO₂-independent media for 30 min, then treated with different concentrations of inhibitors and placed at 37 °C for 60 minutes. Zanamivir was added at a final concentration of 10 mM, and the cells were incubated at 4 °C for 30 min, rocked, and the liquid phase was collected in V-bottomed 96-well plates to measure reversibly bound RBCs. The cells were then incubated at 4 °C for 30 min with ACK-Lysing buffer, and the liquid phase was collected in V-bottomed 96-well plates to measure irreversibly bound RBCs. The cells were then lysed in lysis buffer and transferred to flat-bottom 96 well plates to quantify fused RBCs. The amount of RBCs in each of the three compartments (reversibly bound, irreversibly bound, and fused) was determined by absorption measurement at 410 nm.

F_{ECTO} purification: Drosophila S2 cells were transfected with a plasmid encoding the ectodomain construct (pMT-puro-Fecto2M-EK-twin-Strep-TagII) following the manufacturer's protocol. On day 1, S2 cells were seeded in a 6-well plate at a density of 1.0×10⁵-1.5×10⁵ cells/mL in 3 mL of complete Schneider's medium. On day 2, the cells were transfected with 2 μg of the plasmid using the Effectene (QIAGEN) transfection protocol. Selection with puromycin (6 μg/mL) started on day 5, at which point cells were transferred to a T25 flask. The cells were then gradually expanded to a T75 flask, with the medium replaced by Lonza Insect Xpress medium containing 6 μg/mL puromycin. Subsequently, cells were expanded to 2×1 L in 2 L shaking flasks, induced with 500 μM CuSO₄ at a 1×10⁷ cells/mL density, and harvested four days post-induction. Cells were removed from the culture medium by centrifugation at 5,000×g for 20 minutes. The medium was adjusted to pH 8.0 with 1 M HEPES, and Biolock (IBA) (2.4 mL/L of medium) and NaCl (to a final concentration of 800 mM) were added. The medium was loaded onto a StrepTrap HP 5 mL column (Cytiva), and the column was washed with high-salt HBS buffer (50 mM HEPES pH 8.0, 800 mM NaCl) until a stable UV absorbance at 280 nm was achieved. The protein was then eluted with a high-salt HBS buffer containing 5 mM d-desthiobiotin. Next, the eluted protein fractions were pooled, and the buffer was exchanged to high-salt HBS using a HiPrep 26/10 Desalting column. Finally, the purified protein was flash-frozen in liquid nitrogen. Protein concentration was estimated using the molar extinction coefficient at 280 nm (48,820 M⁻¹ cm⁻¹).

Antibody expression: Monoclonal antibody mAb 77.1.1 was produced in the ExpiCHO cell line by following the manufacturer's high-titer transfection protocol using the pTRIOZ-mAb 77.1 plasmid, which encodes the heavy and light chains of the antibody. After seven days of expression, cells were removed from the culture medium by centrifugation at 4,000×g. The medium was then adjusted to pH 8.0 with 1 M HEPES (pH 8.0) buffer and incubated overnight at 4 °C with protein A agarose beads to capture the mAb 77.1. On a subsequent day, the beads were washed with 20 ml of HBS buffer, and the bound antibody was eluted using 15 ml 0.2 M glycine (pH 2.0). The eluate was immediately neutralized by adding 10% (v/v) 1 M HEPES buffer (pH 8.0).

To generate Fab fragments, mAb 77.1.1 was digested at 1 mg/ml with 1% (w/v) cysteine-activated papain for 4 hours at 37 °C in a water bath. The enzymatic reaction was halted by adding 0.5 M iodoacetamide to a final concentration of 50 mM.

Following the digestion, the reaction mixture was diluted 10-fold with 10 mM HEPES buffer (pH 8.0) to decrease salt concentration and subsequently loaded onto a 1 mL Mono S column pre-equilibrated with 20 mM HEPES buffer (pH 8.0). The Fab fragments' flowthrough was collected, while the Fc fragments were eluted using a single step with 20 mM HEPES buffer (pH 8.0) containing 2 M NaCl. The isolated Fab fragments were concentrated, and their buffer was exchanged for HBS buffer. The Fab fragments were stored at 4 °C for further analysis and experimentation. Protein concentration was estimated using the molar extinction coefficient at 280 nm (mAb 77.1:240360 M⁻¹ cm⁻¹, Fab 77:84,270 M⁻¹ cm⁻¹).

Formation of Fecto2M-Fab 77.1 Complex: The Fecto2M and Fab 77.1 complex was prepared by incubating approximately 5 μM of Fecto2M with a three-fold molar excess of Fab 77.1 at 4 °C overnight. Following the incubation, the mixture was subjected to size-exclusion chromatography using a Superdex 200 Increase 10/300 GL column (Cytiva) in HBS buffer (50 mM HEPES, pH 8.0, and 150 mM NaCl). The fractions containing the F_{ECTO}-Fab 77.1 complex were collected, concentrated, and used on the same day.

Differential Scanning Fluorimetry (DSF) Assay: Differential Scanning Fluorimetry (DSF) was used to determine protein stability and evaluate variations in thermal denaturation profiles under distinct buffer conditions. Each analysis was conducted using a 25 μL volume containing 2.5 μg of protein and 2.5 μL of a 1% SYPRO Orange solution (Thermo Scientific), with the remaining volume composed of the buffer being tested. Stability measurements for FECTO, FECTO-Fab 77.1, FECTO-[FIP-HRC]₂-PEG₁₁ were performed in the HBS buffer. For pH screening, buffers of Sodium Acetate (pH 5.0), Bis-Tris (pH 6.0), MOPS (pH 7.0), HEPES (pH 8.0), and CAPSO (pH 9.0) were used, all at 50 mM. The salt screening was performed using 50 mM HEPES buffer (pH 8.0) with NaCl concentrations ranging from 0 to 2 M.

The samples were dispensed into a 96-well PCR plate (BIO-RAD Multiplat 96-Well PCR Plates, low profile, unskirted), and the plate was then sealed with a heat-sealing film (Bio-RAD Microseal ‘B’ PCR Plate Sealing Film) to prevent evaporation during the heating process. A thermal cycling procedure was initiated on a Bio-Rad CFX 96 thermocycler, with the temperature incrementally elevated from 25 °C to 95 °C at a rate of 1 °C per minute.

Throughout the thermal cycling, SYPRO Orange fluorescence was monitored continuously with the wavelengths for excitation and emission set at 520 nm and 540 nm, respectively. Data analysis was automatically carried out in Bio-RAD CFX Manager software, and the first derivative of the melting curve was graphed against temperature. Each experimental condition was performed in quadruplicate, and the average derivative signal was calculated. The minimum value in the averaged derivative signal represented the apparent melting temperature (Tₘ^{app}) of the protein.

Bio-layer interferometry: BLI-based kinetic analysis was conducted using the OctetRed384 system. Anti-human Fab (FAB2G, Sartorius) capture sensors were loaded with Fab at 15 μg/mL in 10 mM HEPES pH 7.4, 150 mM NaCl, 3 mM EDTA, 0.05% Tween-20, supplemented with 0.5 mg/ml BSA (1xHBSTE/BSA). Binding of F2M_{ECTO} to Fab-loaded sensors was performed with two-fold serial dilutions of F. Baseline and dissociation steps were carried out in 1×Kinetics buffer. Regeneration was carried out using 10 mM Glycine, pH 2.5. Reference sensors (those not loaded with Fab but dipped into F-containing wells) were subtracted from each dataset to account for non-specific binding of F. Fab-only sensors were used as sample references. Kinetic parameters (Kₒₙ and K_{off}) and affinities (K_{D}) were calculated from a nonlinear fit based on the 1:1 binding kinetic model of the data using the Octet Data Analysis HT software version 11 (Fortebio). Three replicates for each binding curve were performed.

Structural mapping of protein variants: The sequences for Measles morbillivirus Fusion (MeV F) proteins were acquired using a custom Python script using the BioPython library. This script was used to query the Entrez protein database for “Measles morbillivirus Fusion.” All results obtained from this process were consolidated into a single fasta file.

The sequences were then filtered based on size, retaining only those longer than 400 and shorter than 600 amino acids. Subsequently, the Needleman-Wunsch algorithm inside BioPython was employed for pairwise alignment with the F WT sequence (Uniprot: Q786F3) to further curate the sequences. Any sequences with low alignment scores (lower than 0) were removed from consideration.

After this curative process, the filtered fasta file was globally aligned using the Clustal Omega software. This global alignment was stored in a clustal format file, which was then used to calculate the sequence conservation for each position using the AL2CO program. This primary sequence conservation was visualized and graphed using Matplotlib and Seaborn Python packages.

Furthermore, structural sequence conservation was mapped onto the pre- and post-fusion structures of F_{ECTO} using ChimeraX 1.6, which also uses AL2CO. Statistics regarding the point mutations at the 77.1 epitope were computed in Python based on the Clustal alignment.

Cell Surface Biotinylation: HEK293T cells were transfected with Measles B3 F variants (wildtype-wt, 436K, 436G, 436D, 37R) or Measles H and by incubating 2 μg/well of cDNA in OPTIMEM with 4 μg/well of Lipofectamine 2000 (Invitrogen 11668019) in OPTIMEM (Gibco 31985088) for 20 minutes at room temperature and then adding to a well of a 6 well plate for 3 hours at 37 °C. After transfection, cells were incubated with DMEM (Gibco 10569044) with 10% FBS (Gibco 16000044) and 1% Penicillin-Streptomycin (Gibco 15140122) at 37 °C or 32 °C for 24 or 48 hours. Cells were then incubated at 4 °C with 3.3 mM NHS-S-S-dPEG4-biotin (Quanta Biodesign 10194) in DPBS (Gibco 14287080) for 1 hour before lysing with DH Buffer [50 mM HEPES (Gibco 15630080), 100 mM NaCl, 5 mg/mL dodecyl maltoside (Thermo Scientific 89903) in Milli-Q Water] supplemented with complete Protease Inhibitor Cocktail (1 tablet/50 mL; Roche 11836145001). Biotinylated proteins were pulled down with Streptavidin Sepharose (Invitrogen 434341) for 16 hours at 4 °C. Protein was eluted from the beads in reducing Laemmli SDS Sample Buffer (Boston BioProducts BP-110R), boiled for 10 minutes, and run on a 4-20% Novex Tris-Glycine Protein Gel (Invitrogen WXP42026BOX). The gel was transferred to nitrocellulose using iBlot quick transfer method (Invitrogen IB23001). The blot was blocked (Invitrogen WB7050), treated with Anti-Measles F HRC (GenScript; Rabbit) and alkaline phosphatase-conjugated anti-rabbit secondary antibody (Invitrogen WB7105). The blot was then developed using NBT/BCIP Substrate (Invitrogen WP20001). Analysis of the blot was performed in GelAnalyzer 23.1 and the integrated intensity of the bands was normalized to the expression level of F wt at 37 °C.

Post-triggered antibody recognition with and without 77.1: HEK293T cells were transfected using pCAGGS MeV B3 wt F. Specifically, 0.1 μg/well of plasmid was mixed with 0.3 μg/well of Lipofectamine 2000 (Invitrogen, Cat #11668019) in OPTI-MEM (Gibco, Cat #31985088). The mixture was incubated for 20 minutes at room temperature before being added to a 96-well plate and incubated for 3 hours at 37 °C. Post-transfection, the cells were cultured in DMEM (Gibco, Cat #10569044) supplemented with 10% FBS (Gibco, Cat #16000044) and 1% Penicillin-Streptomycin (Gibco, Cat #15140122) at 37 °C for 24 hours. Cells were then exposed to either 250 nM of 77.1 antibody (Creative Biolabs, Cat #Custom-211420; human origin) or left untreated, both at 4 °C. After a 30-minute incubation, the media was replaced. Subsequently, one plate was maintained at 4 °C, another was incubated at 55 °C for 30 minutes, and the last one was incubated at 55 °C for 60 minutes. Afterward, the cells were cooled to 4 °C and treated with a previously characterized mouse monoclonal antibody specific to the post-triggered state of measles F. Following a 60-minute incubation, the cells were stained with goat anti-mouse conjugated with Alexa Fluor 488 (1 μg/mL; Invitrogen, Cat #A32723) and goat anti-human conjugated with Dylight 594 (1 μg/mL; Abcam, Cat #ab97005). Cells were then counterstained with DAPI (1 μg/mL; Thermo Scientific, Cat #62248) and imaged using an Agilent Cytation 5 system. Total fluorescence per well was quantified using BioTek Gen5 Software. Background fluorescence was calculated from untreated wells and subtracted from the measurements. Data was plotted against the time of incubation at 55 °C with standard error and fitted with logistic regression function.

### Grid Preparation

Negative stain: Copper grids (400 mesh) coated with carbon film (CF400-Cu, Electron Microscopy Sciences) were used for negative staining. The grids were first subjected to glow discharge treatment using the easyGlow Glow Discharge Cleaning System (Pelco) for 15 seconds at 15 mA to enhance their hydrophilicity. Next, the protein sample concentration was adjusted to 0.01 absorbance units at 280 nm, and 3 μL of the sample was applied to the grid for a 1-minute incubation. Subsequently, the grid was blotted to remove an excess sample and washed three times with 3 μL deionized water, blotting between each washing step. The grid was incubated with 3 μL of 1% uranyl acetate solution for negative staining. After two blotting steps, a final drop of the staining solution was added, followed by a 1-minute incubation. The grid was then blotted once more to remove excess stain and allowed to air-dry.

Graphene oxide grids: In this study, Applicants used custom-made graphene oxide (GO) grids. The GO grids were prepared by diluting a commercial GO solution (2 mg/mL, Sigma-Aldrich) in deionized water to a final concentration of 0.2 mg/mL. This diluted solution was sonicated for 5 minutes in a water bath to ensure proper dispersion of GO layers. Next, large aggregates were removed via centrifugation at 500×g for 1 minute. Finally, the supernatant containing the dispersed GO was collected by transferring the upper 50% solution to a fresh 1.5 mL microcentrifuge tube.

Quantifoil R2/2 grids were washed with ethyl acetate overnight and glow-discharged under the same conditions as the negative stain grids. Following this, 3 μL of the GO solution was applied to the grids and incubated for 4 minutes. The grids were subsequently blotted, rinsed with 20 μL of deionized water, blotted once more, and air-dried for at least 10 minutes.

On the same day of grid preparation, the dried GO grids were used for the vitrification of the protein samples. The Vitrobot Mark IV plunge-freezing system was operated at 4 °C and a relative humidity of 100%. A 3 μL aliquot of the protein sample (0.2-0.5 mg/ml) was deposited onto the GO grid, followed by a 12-second incubation period. After the incubation, the excess sample was blotted away (blot force 0) for 3 seconds. The grid was immediately plunge-frozen in liquid ethane cooled by liquid nitrogen.

C-flat grids: C-flat 1.2/1.3 400 mesh grids were glow discharged like GO grids and immediately used for sample freezing. A 3 μL aliquot of 0.5 mg/ml protein sample was deposited onto the grid, immediately blotted for 6s, and plunge-frozen in liquid ethane cooled by liquid nitrogen.

### Cryo-EM Data Processing

F_{ECTO}2M-Fab 77: Movies were collected on Titan Krios operated at 300 KV using a K3 Direct electron detector (Gatan) in counting mode (0.66 Å pixel⁻¹) at a nominal magnification of ×130,000 using a defocus range of −0.5 μm to −2.0 μm with Bioquantum energy filter slit set to 20 eV. Movies (LZW compressed TIFF files) were collected in EPU (Thermo) over a 3 s exposure with an exposure rate of ~15 e-pixel⁻¹ s-1, resulting in a total exposure of ~50 e-Å⁻² (1 e-Å⁻² frame-1). Downstream processing was done using Relion, homemade Relion scripts, and cryoSPARC. Frame alignment was done in Relion 4 using MotionCor2 version 1.4.0 with 6×5 patches and a B factor of 150. CTF determination was performed using CTFFIND4 version 4.1.14 using dose-weighted micrographs.

Particles were auto-picked in Relion from randomly selected micrographs using the Laplacian-of-Gaussian algorithm, extracted at 512 px box size and Fourier-cropped to 64 px (5.28 A/px), and used for the 2D classification step. Next, particles resembling glycoprotein were selected and subjected to further rounds of 2D classification. The best classes showing different glycoprotein orientations were selected and used for the Topaz model training over 10 epochs. Finally, the optimized model was used to pick particles in the whole dataset. ~1.1 M particles were extracted in Relion, imported in cryoSPARC, and subjected to 2 rounds of 2D classification. Classes resembling glycoprotein with bound fab were selected (148k particles), and particles corresponding to Fab and Fab2 were discarded. Selected particles from cryoSPARC were transferred back to Relion using a homemade script and re-extracted at 512 px with Fourier-cropping to 160 px (2.112 A px⁻¹). Particles were again imported to cryoSPARC, where the initial model was generated. The obtained model was used for the 3D classification in Relion with 3 Classes.

During the first 25 iterations, global angular searches were performed using 15-degree sampling intervals. Once the classification reached equilibrium (followed by monitoring class distribution using Follow Relion Gracefully), the 3D classification job was resumed with local angular searches of 7.5-degree sampling intervals and a 30-degree local angular search range. This procedure was repeated with a decreasing angular sampling interval and search range (kept double the angular sampling) until there was no improvement in estimated angle accuracy. Two classes showed high-resolution features and were selected for the 3D Refine protocol (142k particles). Next, refined particles were re-extracted at 512 px with Fourier-cropping to 320 px (1.056 A px⁻¹) with re-centering coordinates. Finally, particles were refined, post-processed, and CTF and aberration refinement were performed. This procedure was repeated until there was no improvement in the reported post-processing resolution. Then, the Polishing step was applied, first by training on 10000 particles with a Fraction of Fourier pixels for the testing set to 0.5. The obtained trained parameters were used for particle polishing, and particles were extracted at 320 px (1.056 A px⁻¹). Particles were again 3D refined, and CTF and aberration refinement were performed until there was no further improvement in the reported post-processing resolution. Finally, polished particles were imported to cryoSPARC, and a non-uniform refinement protocol was used to generate the final cryo-EM map in C3 symmetry. Then, particles were C3 symmetry expanded and refined using the Local Refine protocol in cryoSPARC to obtain the final map.

F_{ECTO} [FIP-HRC₄]₂-PEG₁₁: Similarly, as for the F_{ECTO}-Fab 77 structure, FECTO [FIP-HRC₄]₂-PEG₁₁ structure was recorded and processed following the same workflow. Number of particles and workflow overview is presented in FIG. 11C.

F_{ECTO} post-fusion: F_{ECTO} was incubated in lower salt buffer (50 mM HEPES pH 8.0, 150 mM NaCl) for 1 week in 4 °C. In contrast to the other two datasets, Quantifoil R1.2/1.3 grids were used to freeze F_{ECTO} in post-fusion confirmation. 3 μL of 0.5 mg/ml FECTO was mixed with 1 μL of 0.36 mM LMNG, blotted for 6s, and frozen in liquid ethane. The data processing was similar to the other two datasets, and detailed processing is shown in FIG. 11 B.

### Model Building

For the F_{ECTO}-Fab 77.1 structure, the obtained cryo-EM map was used to dock the previously published F X-ray structure (PDB ID: 5XYW) using ChimeraX. Next, the antibody model was generated with an in-house implementation of ColabFold based on AlphaFold2. Once the models were positioned, manual model building was conducted in Coot 0.9.8.7, sourced from the CCP4 package. An automatically sharpened and density-modified map from PHENIX ResolveEM (using only half-maps) was the basis for model building. A real-space refinement protocol followed manual model building in the PHENIX package, consisting of 10 macrocycles without any enforced restraints, and increased the number of Max iterations to 800. Finally, water placement was carried out manually on a density-modified map. Water molecules were positioned where spherical density was visible at map RMSD>5.0, no more than 2.7 Å from protein atoms. Interfaces were analysed using ChimeraX and PISA server. Electrostatic maps, as well as the generation of F variants, were calculated in ChimeraX. Local map scales for F_{ECTO} were calculated in OccuPy.

A refined model of FECTO from FECTO-Fab 77.1 was used to dock in the F_{ECTO} [FIP-HRC₄]₂-PEG₁₁ map. FECTO in post-fusion conformation was first generated using homology modeling using SWISS-MODEL software and manually rebuilt in Coot. Refinement and evaluation of the model was done as described above.

Heat-Induced Refolding of FECTO-Fab 77.1: A 7.5 μM solution of F_{ECTO} in HBS containing 800 mM NaCl was combined with a 1.5-fold molar excess of Fab 77.1. This mixture was then diluted fourfold using 50 mM HEPES (pH 8.0), resulting in a final NaCl concentration of approximately 150 mM. The complex was allowed to form at room temperature over 4 hours. A 15 μL aliquot of the formed complex was transferred to a PCR tube and subjected to heat treatment in a PCR thermocycler. The incubation times tested were 1, 5, 7.5, or 10 minutes at 50 °C and 1 or 5 minutes at 55 °C. After incubation, the samples were rapidly cooled by placing the tube on ice for 5 minutes. Negative stain grids were then prepared using the described protocol.

The acquired data were processed using cryoSPARC 4.3. After motion correction and estimation of CTF values, particle picking was performed using the blob picker. Subsequent 2D classification was performed using the previously described deep 2D classification protocol to segregate the particles into distinct classes.

Sequence analysis: Approximately 1400 MeV genomic sequences were obtained from the nucleotide NCBI database. CDS regions were manually annotated and curated to include only those between 400 and 600 amino acid residues in length with pairwise alignment scores greater than 0 as compared to the F WT sequence. The resulting ~820 sequences were aligned using Clustal Omega, and the conservation score was computed using AL2CO. Conservation scores were placed on the 3D models of F_{ECTO}^{PRE} and F_{ECTO}^{POST} in ChimeraX using AL2CO. Sequence variants were determined using a homemade Python script based on the multiple sequence alignment file.

mNGS and variant calling: mNGS was performed as previously described (PMID: 29970463). Briefly, RNA was extracted from 50 μL of viral culture using the Quick-RNA Viral Kit (Zymo) and treated with TURBO DNase I (ThermoFisher). cDNA was generated from the DNase-treated RNA using Superscript IV Reverse Transcriptase (Thermo Fisher) and random hexamers (IDT), followed by second-strand synthesis via Sequenase Version 2.0 DNA Polymerase. The resulting double-stranded cDNA was then purified with the DNA Clean &amp; Concentrator Kit (Zymo). Libraries were constructed from 2 μL of cDNA using the Nextera XT kit (Illumina) and sequenced on 1x192 bp Illumina MiSeq runs. Sequencing reads were adapter and quality trimmed using Trimmomatic v0.38 (PMID: 24695404). Variants present at a frequency greater than 10% and coverage greater than 10× were identified with LAVA (github.com/greninger-lab/lava) using the MeV reference genome. All variants were manually confirmed by mapping sequencing reads to the same MeV reference strain in Geneious v11.1.4. Those variants present in intergenic region between the matrix and fusion proteins as well as in homopolymeric tracts were excluded from the analysis. Sequencing reads are available under NCBI BioProject number PRJNA594952.

Statistical analysis: All data analysis and visualizations were conducted using a Python notebook integrating SciPy, Matplotlib, Pandas, Seaborn, and Statannotations packages. Statistical significance was determined using the Mann-Whitney test. For p-value annotations, the following symbols were used: ‘ns’ indicates p<=1,* represents 0.01<p<=0.05, ** signifies 0.001<p<=0.01, and *** stands for 0.0001<p<=0.001.

Chemicals and peptides: N-(3-cyanophenyl)-2-phenylacetamide (also known as 3G) was commercially acquired from ZereneX Molecular Limited (UK). The purity of 3G was tested by high-pressure liquid chromatography (HPLC) and shown to be >95% pure.

Peptides were purchased from CPC, Ltd. Bromoacetyl cholesterol and bismaleimide cholesterol derivatives were custom made by Charnwood Molecular Ltd. Dimethylsulfoxide (DMSO), tetrahydrofuran (THF), N,N-diisopropylethylamine (DIPEA) were purchased from Sigma Aldrich, bismaleimide-PEG₁₁ from Quanta BioDesign. HPLC purification was performed on a 1100 Series Agilent HPLC system equipped with a UV diode array detector and a fraction collector using a reverse phase (RP.) Phenomenex Jupiter C4 LC column 300 Å (150×21.2 mm, particle size 5 μm). MALDI-TOF analysis was performed on Bruker UltrafleXtreme MALDI-TOF-TOF instrument.

### The Antibody's Complementarity Determining Regions (CDRs)

In the antibody against measles virus F protein, Applicants identified the CDRs in the heavy chain variable region and the light chain variable region, as follows (with the CDRs identified by underlining):

Heavy chain:
DVQLQESGPGLVKPSQSLSLTCTVSGYSITSDYAWNWIRQFPGNKLEWMG
YISYTLTTGYNPSLKSRISITRDSSKNQFFLQLNSVTTEDTATYYCARSG
WLLPYWYFDVWGAGTTVTVSS
Light chain:
DVQITQSPSYLAASPGETITINCRASKSISKYLAWYQEKPGKTNELLIYS
GSTLQSGIPSRFRGSGSGTDFTLTISSLEPEDFAMYYCQQHNEYTLTFGG
GTKLELKR

### Tables

TABLE 1
Model and map statistics

| Model | | | | | Fecto-(FIP-HRC4)₂- | |
|---|---|---|---|---|---|---|
| Composition (#) | Fecto-Fab 77.1 | | Fecto-post fusion | | PEG11 | |
| Chains | 16 | | 11 | | 13 | |
| Atoms | 16159 (Hydrogens: 0) | | 9894 (Hydrogens: 0) | | 10768 (Hydrogens: 0) | |
| Residues | Protein: 2037 | | Protein: 1256 | | Protein: 1357 | |
| | Nucleotide: 0 | | Nucleotide: 0 | | Nucleotide: 0 | |
| Water | 229 | | 134 | | 33 | |
| Ligands | BMA: 6 | | NAG: 9 | | LIG: 3 | |
| | NAG: 15 | | | | NAG: 15 | |
| | | | | | BMA: 6 | |
| | | | | | MAN: 4 | |
| Model statistics | | | | | | |
| Bonds (RMSD) | | | | | | |
| Length (Å) (# > 4sigma) | 0.003 | (0) | 0.002 | (0) | 0.006 | (0) |
| Angles (°) (# > 4sigma) | 0.548 | (0) | 0.525 | (0) | 0.685 | (1) |
| MolProbity score | 1.55 | | 1.25 | | 1.14 | |
| Clash score | 6.47 | | 4.88 | | 3.53 | |
| Ramachandran plot (%) | | | | | | |
| Outliers | 0.20 | | 0.00 | | 0.00 | |
| Allowed | 2.98 | | 1.21 | | 1.71 | |
| Favored | 96.82 | | 98.79 | | 98.29 | |
| Rama-Z (Ramachandran | | | | | | |
| plot Z-score, RMSD) | | | | | | |
| whole (N = 2013) | −0.60 | (0.18) | 0.25 | (0.23) | −0.08 | (0.21) |
| helix (N = 429) | 0.23 | (0.24) | 1.10 | (0.23) | 0.14 | (0.23) |
| sheet (N = 486) | −0.06 | (0.24) | 1.23 | (0.48) | 0.25 | (0.35) |
| loop (N = 1098) | −0.77 | (0.18) | −0.86 | (0.23) | −0.13 | (0.22) |

| Rotamer outliers (%) | 0.00 | 0.00 | 0.00 |
|---|---|---|---|
| Cbeta outliers (%) | NA | NA | NA |
| Peptide plane (%) | | | |
| Cis | 7.7/0.2 | 0.0/0.0 | 0.0/0.0 |
| proline/general | | | |
| Twisted | 0.0/0.0 | 0.0/0.0 | 0.0/0.0 |
| proline/general | | | |
| CaBLAM outliers (%) | 1.96 | 0.79 | 0.90 |
| ADP (B-factors) | | | |
| Iso/Aniso (#) | 16159/0 | 9894/0 | 10768/0 |
| min/max/mean | | | |
| Protein | 7.78/145.94/55.27 | 0.00/196.59/75.85 | 14.91/124.73/48.03 |
| Ligand | 27.94/156.21/78.74 | 102.93/151.05/126.78 | 0.20/171.70/57.90 |
| Water | 13.74/72.27/39.79 | 33.21/102.23/55.55 | 23.07/53.29/37.59 |
| Occupancy | | | |
| Mean | 1.00 | 1.00 | 1.00 |
| Data | | | |
| Box | | | |
| Supplied | 2.6 | 2.7 | 2.3 |
| Resolution (Å) | | | |
| Resolution | Masked/(Unmasked) | Masked/(Unmasked) | Masked/(Unmasked) |
| Estimates (Å) | | | |
| d FSC (half maps; | 2.56 | 2.72 | 2.34 |
| 0.143) (Å) | | | |
| d model | 2.9/(3.0) | 3.1/(3.1) | 2.6/(2.6) |
| d FSC model | 2.5/2.5/2.8/ | 2.6/2.7/3.0 | 2.2/2.3/2.5 |
| (0/0.143/0.5) (Å) | (2.5/2.6/3.0) | (2.7/2.7/3.1) | (2.3/2.3/2.7) |
| Map | −2.73/3.40/0.03 | −4.28/6.60/0.04 | −2.73/4.58/0.03 |
| min/max/mean | | | |
| Model vs. Data | | | |
| CC (mask) | 0.81 | 0.82 | 0.89 |
| CC (box) | 0.69 | 0.67 | 0.72 |
| CC (peaks) | 0.67 | 0.65 | 0.72 |
| CC (volume) | 0.79 | 0.80 | 0.87 |
| Mean CC for ligands | 0.65 | 0.54 | 0.64 |
| Data acquisition | | | |
| Microscope/camera | Thermo Titan Krios G3 300 kV/Gatan K3 + GIF Bioquantum | | |
| Magnification | 130′000 X | | |
| Exposure/total | 2.5 s/~50 e⁻/Å² | | |
| electron exposure | | | |
| Frames | 50 | | |
| Defocus range | −0.6-2.5 μm | | |
| Pixel size | 0.66/1.056 | 0.66/0.99 | 0.66/0.88 |
| (raw/final) (Å) | | | |
| Number of micrographs | 10344/7798 | 6028/5420 | 10716/5830 |
| (all/final) | | | |
| Number of particles | 142852/428556 | 52850/158562 | 100661/235003 |
| (unique/symmetry expanded) | | | |
| Box size (raw/final) (px) | 512/320 | 600/400 | 400/300 |

| | |
|---|---|
| Symmetry | Local C1 from symmetry expanded C3 |

TABLE 2
CRD regions of 77.1 involved in F binding

| CDR | Residue range | Interactions |
|---|---|---|
| CDR H-1 | 31-36 | 4 |
| CDR H-2 | 51-66 | 3 |
| CDR H-3 | 99-110 | 7 |
| CDR L-2 | 69-75 | 2 |

TABLE 3
77.1-F interface statistics per chain.

| | Interface | Δ\|G | Hydrogen | Salt |
|---|---|---|---|---|
| Interface | area (Å²) | (kcal/mol) | bonds | bridges |
| HC-F1′ | 466 | −2 | 7 | 1 |
| HC-F2′ | 283 | −4 | 4 | 0 |
| HC-F1″ | 140 | −1.5 | 2 | 0 |
| LC-F1″ | 0 | | | |
| Total | 995 | −8.6 | 15 | 1 |

TABLE 4
Hydrogen bonds and salt bridges between 77.1 and F per interface
HC-F1′ Interface

| Hydrogen | HC residue | Distance | |
|---|---|---|---|
| bond | [atom] | (Å) | F1′ residue [atom] |
| 1 | TRP 101 [N] | 3.08 | GLN 383 [OE1] |
| 2 | TYR 107 [OH] | 2.38 | ARG 435 [O] |
| 3 | ASP 32 [OD2] | 2.5 | GLN 383 [N] |
| 4 | TYR 33 [OH] | 3.54 | GLY 384 [N] |
| 5 | TYR 107 [OH] | 3.5 | ARG 435 [N] |
| 6 | TYR 107 [O] | 2.9 | ARG 436 [NH1] |
| 7 | TYR 105 [O] | 2.96 | ARG 436 [NH1] |
| 8 | ASP 109 [OD1] | 2.74 | ARG 436 [NH2] |
| Salt | HC residue | Distance | |
| bridge | [atom] | (Å) | F1′ residue [atom] |
| 1 | ASP 109 [OD2] | 2.97 | ARG 436 [NH2] |

HC-F2′ Interface

| Hydrogen | HC residue | Distance | |
|---|---|---|---|
| bond | [atom] | (Å) | F2′ residue [atom] |
| 9 | TYR 54 [OH] | 3.22 | GLY 37 [O] |
| 10 | ARG 72 [NH2] | 3 | SER 40 [O] |
| 11 | THR 55 [OG1] | 3.41 | TRP 27 [NE1] |
| 12 | THR 57 [OG1] | 3.75 | TRP 27 [NE1] |

HC-F1″ Interface

| Hydrogen | HC residue | Distance | |
|---|---|---|---|
| bond | [atom] | (Å) | F1″ residue [atom] |
| 13 | TYR 27 [OH] | 3.12 | LEU 123 [O] |
| 14 | SER 31 [O] | 3.46 | VAL 125 [N] |

LC-F1″ Interface

| Hydrogen | LC residue | Distance | |
|---|---|---|---|
| bond | [atom] | (Å) | F1″ residue [atom] |
| 15 | TYR 49 [OH] | 3.14 | ARG 435 [N] |
| 16 | GLN 55 [OE1] | 2.99 | SER 434 [OG] |

TABLE 5
Residue variants present at the 77.1 epitope with their interaction
type and number of interactions. Variant residues shown in
bold were tested for their effect on 77.1 binding.

| Residue | Original | Residue | | Total |
|---|---|---|---|---|
| Position | Residue | Variation | Interaction Type | interactions |
| 27 | W | — | Sidechain | 2 |
| 37 | G (99.88%) | R (0.12%) | Backbone | 1 |
| 40 | S | — | Sidechain/Backbone | 1 |
| 123 | L | — | Backbone | 1 |
| 125 | V | — | Backbone | 1 |
| 383 | Q | — | Sidechain/Backbone | 2 |
| 384 | G | — | Backbone | 1 |
| 434 | S | — | Sidechain/Backbone | 3 |
| 435 | R | — | Backbone | 3 |
| 436 | R (72.89%) | K (27.11%) | Sidechain | 3 |

### Example 2—Grafting 77.1 mAb

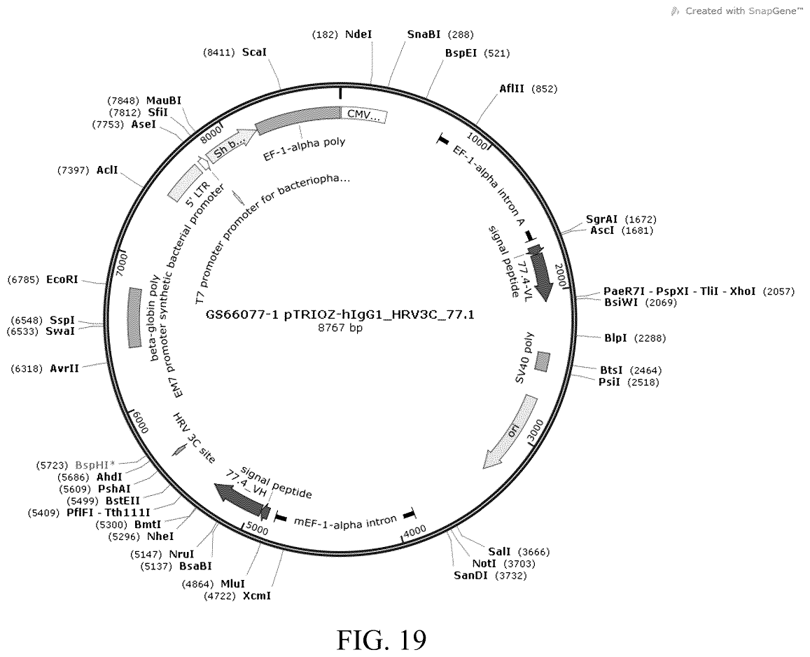

Previously, Applicants assessed the neutralization properties of mouse monoclonal antibodies that specifically target the MeV F protein in its pre-fusion state. From the most potent antibody Applicants derived a human monoclonal antibody, referred to as 77.1 mAb, and Applicants show that it has viral neutralizing activity in vitro and in vivo. The 77.1 mAb, with variable domains (VL and VH) derived from 77.4 (mouse) was cloned in pTRIOZ-hIgG1 vector (FIG. 19).

77.4 VL Sequence
GATGTTCAGATCACACAGAGCCCAAGCTACCTCGCAGCATCTCCAGGCGA
GACTATCACTATCAACTGTAGAGCCTCCAAGTCCATTTCTAAGTATCTGG
CTTGGTATCAGGAGAAGCCAGGTAAGACCAACGAGCTGCTGATCTACTCT
GGAAGCACACTCCAGTCCGGTATCCCATCCAGATTCAGAGGTTCTGGTTC
TGGCACCGATTTCACCCTGACAATCAGCTCTCTGGAGCCTGAAGATTTCG
CAATGTACTATTGTCAGCAGCATAACGAGTATACCCTGACTTTCGGTGGA
GGAACAAAGCTCGAGCTGAAGCGT
77.4 VH Sequence
GACGTGCAGCTCCAAGAGAGCGGTCCTGGTCTGGTGAAGCCTTCTCAAAG
CCTGTCCCTGACCTGTACCGTTTCTGGCTATAGCATCACTTCTGACTACG
CCTGGAACTGGATTAGGCAGTTTCCTGGTAACAAGCTGGAATGGATGGGC
TACATTAGCTACACCCTGACTACAGGTTACAATCCCTCTCTGAAGTCTAG
GATTTCTATCACTCGCGACTCCTCTAAGAACCAGTTCTTCCTGCAACTGA
ATAGCGTGACAACAGAAGATACTGCTACATACTACTGTGCAAGATCCGGT
TGGCTGCTGCCTTACTGGTACTTCGATGTGTGGGGTGCCGGCACAACCGT
GACTGTCAGCTCC

### 77.1 mAb Inhibits Spread of MeV Wild Type (Wt) and Central Nervous System (CNS)-Adapted MeV Variant

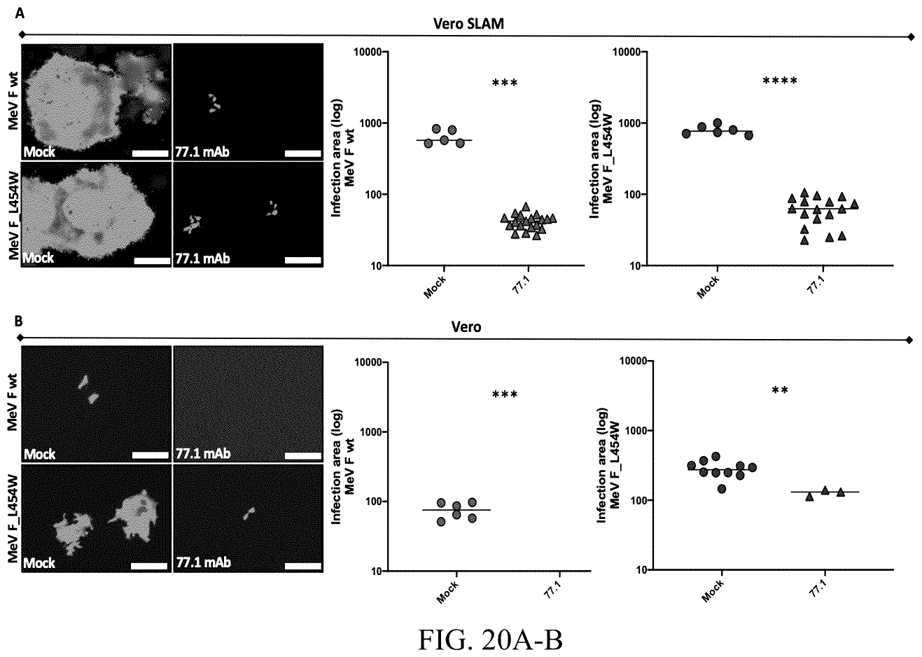

The antiviral activity of 77.1 mAb was assessed in MeV B3-eGFP-F wt and F_L454W (CNS-adapted variant) live virus infection (FIG. 20). Since the virus bearing L454W F spreads in the absence of a known MeV entry receptor, Applicants compared the inhibitory efficacies in Vero cells expressing the known receptor SLAM/CD150 (Vero-SLAM) (FIG. 1 Panel A) and Vero cells (FIG. 20 Panel B) that express no known MeV receptor. After a 120-min infection with either MeV F_wt or F_L454W viruses, cells were treated with the antibody and visualized at several time points (24, 48, and 72 h). Without treatment, the two viruses spread and formed large syncytia after 72 h in Vero-SLAM cells. In Vero cells (without receptor), MeV F_wt did not spread, while MeV F_L454W formed syncytia that were smaller than those formed in the presence of CD150/SLAM. The 77.1 mAb treatment blocked both viruses in the presence or absence of receptor.

### In Vivo Prophylaxis and Post-Exposure Efficacy of 77.1 mAb

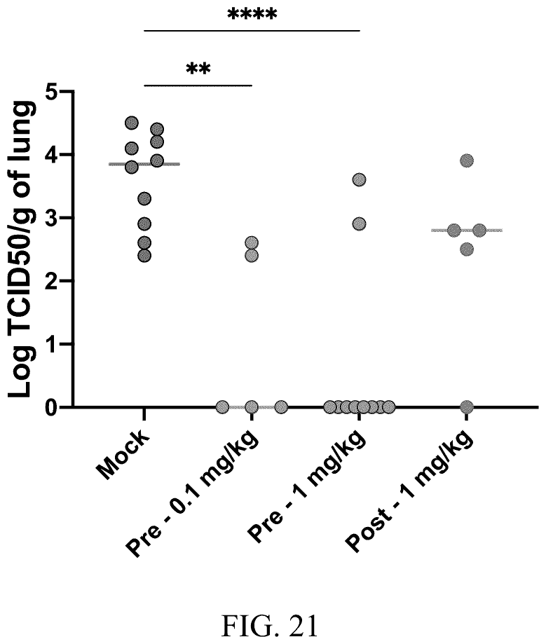

Antiviral efficacy for MeV in vivo was assessed in cotton rats (CR) as an infection model. The 77.1 mAb was administered subcutaneously 12 hours before infection (0.1 mg/kg or 1 mg/kg) or 24 hours post infection (1 mg/kg). Four days after infection, the animals were euthanized and the viral titers in the animals' lungs were quantified. Treatment with 77.1 mAb reduced the viral titer in the lungs compared with the untreated control (FIG. 21).

### Materials & Methods

Recombinant 77.1 mAb Expression and Purification (Creative Biolabs, CBL). The gene for the 77.1 (human monoclonal antibody) mAb derives from 77.4 mAb (mouse monoclonal antibody) and it was synthesized using PCR-based methods and cloned into the pTRIOZ-hIgG1 vector. The expression vectors of the antibody were transiently transfected and expressed in mammalian cells with chemically defined culture media. The mAb was purified by affinity chromatography, then ultrafiltration, and then subjected to 0.2 micron sterile filtration.

Viral spread inhibition in vitro. Vero or Vero-SLAM cells were plated in 12-well plates (2×10⁵ cells/well). The following day, cells were infected either with MeV B3-eGFP-F wild type and MeV B3-eGFP-F L454W (200 PFU/well for Vero-SLAM and 500 PFU/well for Vero cells) for 2 h at 37 °C. The medium was replaced with complete medium and humanized monoclonal antibody referred to as 77.1 mAb (100 μg/ml for Vero-SLAM and 10 ug for VERO cells). After 72 h pictures were obtained using a Cytation 5 Cell Imaging Multi-Mode Reader (BioTek). Areas of infection were measured using BioTek Gen5 software on images randomly acquired from one experiment in duplicate. (Unpaired t test with Welch's correction).

In vivo experiments in cotton rats. Inbred cotton rats (Sigmodon hispidus) were purchased from Envigo, Inc., Indianapolis. Both male and female cotton rats aged 5 to 7 weeks were used. For i.n. infection, 10⁵ TCID₅₀ of MeV B3-eGFP-F wild type in PBS was inoculated intranasally to isoflurane-anesthetized cotton rats in a volume of 100 μl. Four days after infection, the animals were euthanized by CO₂ inhalation, and their lungs were collected and weighed. Lung tissue was minced with scissors and homogenized with a glass dounce homogenizer. Serial 10-fold dilutions of supernatant fluids were assessed for the presence of infectious virus in 48-well plates using cytopathic effect (CPE) in Vero SLAM cells as the endpoint. Plates were scored for CPE microscopically after 7 days. The TCID₅₀ was calculated as described previously.

### Example 3—Thermostable Measles Fusion Glycoprotein as a New Vaccine Strategy

This grant can assess the efficacy of thermostable forms of the measles fusion protein to elicit neutralizing antibodies and induce a protective immune response in vivo. The proposed work can address two Specific Aims:

1. Engineering thermostable measles F protein immunogen. The preliminary results illustrate that while the two initial stabilizing mutations yield prefusion F, additional mutations further anchor F and offer a remarkable level of thermostability. Hypotheses: Applicants hypothesize that these stabilized, pre-fusion measles fusion proteins can elicit strong neutralizing antibodies. Second, Applicants hypothesize that immunization with full-length, stabilized measles F protein can result in superior protection over immunization with ectodomain alone [e.g., by the inclusion of membrane-proximal external region (MPER) and absence of an immunogenic, but non-neutralizing “stump/base” at the bottom]. In this aim, Applicants can produce and characterize stabilized (full-length and ectodomain) measles fusion proteins with the improved thermostability substitutions (at CUIMC/LJI), and measure neutralizing antibodies against full-length and ectodomain forms of these measles fusion protein, including antiviral activity, structures and functions (at CUIMC/LJI).

2. Evaluate the protection afforded by anti-F protein immune responses. Hypothesis: Applicants propose that stabilized measles fusion can induce protective immunity comparable to the current live vaccine (without risk of vaccine-enhanced disease). Proof of concept is first demonstrated by passive administration of anti-F neut. Ab.

1. Passive immunization with neutralizing antibodies (at CUIMC/OSU).

2. Assessment of in vivo immunization with stabilized measles fusion proteins (at CUIMC/OSU/LJI).

### Research Strategy

### A. Significance

A1. Re-emergence of measles virus (MeV) in high-income countries. Infection by MeV remains a major cause of morbidity and mortality among young children worldwide, despite the availability of safe and effective MeV vaccines. MeV was presumed to be eliminated in the U.S. in 2000 (elimination means ≥12 months without transmission). However, reintroduction of the virus has resulted in a resurgence of cases in recent years, causing repeated outbreaks. Outbreaks in the U.S. and other countries with high vaccination coverage are often attributed to unvaccinated individuals. For example, the influence of anti-vaccination proponents in (social) media lead to vaccine hesitancy among parents, resulting in pockets of unvaccinated children. However, a significant number of cases also occur in previously vaccinated people, since up to 5% of vaccine recipients do not develop adequate protective antibodies, even after the recommended two-dose vaccination regimen, and also because vaccine-elicited immunity likely wanes over time.

There is no effective antiviral regimen currently available for MeV, and insufficient coverage (defined as ≤95% vaccination rate by WHO) can lead to a rise in incidence in vulnerable populations. Indeed, MeV has already undergone a global resurgence: estimated global MeV deaths rose from 89,780 in 2016 to 207,500 in 2019. The situation may worsen since the SARS-CoV-2 pandemic reduced routine childhood vaccination coverage and may expose more vulnerable individuals to infection. Without global eradication, a significant increase in number of MeV cases can be unavoidable.

Of particular concern during this resurgence in MeV cases are the rising number of individuals with compromised immune systems, such as cancer patients, transplant recipients, persons with autoimmune disorders on immunosuppressive therapies, and human immunodeficiency virus (HIV)-infected individuals, in addition to pregnant women. In such immunocompromised people, MeV vaccination is often risky and inefficient; antibody responses are impaired, and even pre-existing immunity wanes. Measles resurgence in other parts of the world, combined with increased vaccine hesitancy in the U.S., increases the risk of future measles outbreaks in the U.S, thus posing a risk to vulnerable patient populations (see CDC reports)

A2. MeV pathogenesis. MeV is the most infectious virus known to humans, transmits via the air, and causes systemic infection. The wild-type (wt) virus can use two cellular receptors: CD150 expressed by subsets of immune cells and nectin-4 expressed by epithelial cells. MeV infection of a susceptible host starts in the respiratory tract. Alveolar macrophages and dendritic cells expressing the cellular receptor CD150 are the initial targets. The first MeV-infected cells then transmit the virus to bronchus-associated lymphoid tissues and/or draining lymph nodes, where the virus replicates and is disseminated to CD150-positive B and T lymphocytes, with subsequent viremia. This stage of the disease leads to widespread infection of immune cells in primary, secondary, and tertiary lymphoid tissues. Infection of epithelial cells, which happens at the late stage of the viral life cycle in the host, is associated with aerosol and droplet transmission.

In healthy individuals, MeV infection paradoxically elicits both a strong MeV-specific immune response and a transient immune suppression. Studies in non-human primates have implicated that MeV preferential infection of memory lymphocytes is a direct cause of T- and B-lymphocyte depletion, causing the ‘immune amnesia’ in which pre-existing immune memory is partially erased and replaced by MeV-specific and bystander lymphocytes. These observations were recently corroborated in an observational MeV cohort study in unvaccinated children. Most virus-infected cells are cleared by cellular immunity within two to three weeks after infection. However, viral genomes can persist for several months after infection, even in the face of protective immunity.

As a result of viral persistence, central nervous system (CNS) complications may occur soon after acute MeV infection in acute encephalomyelitis (AME), or years after infection in subacute sclerosing panencephalitis (SSPE). Despite presumably functional cell-mediated immunity and high antiviral antibody titers, immune control of CNS infection is not achieved in patients suffering from SSPE. The third form of MeV-induced CNS disease-progressive infectious encephalitis or measles inclusion body encephalitis (MIBE)-occurs in immunosuppressed patients several months following MeV infection. MeV-associated CNS complications are highly lethal. Due to the perceived elimination of MeV from populations in high-income countries over the last few decades, there has been little incentive to develop novel intervention strategies. Further, there are currently no licensed antivirals for acute or persistent MeV infections.

A3. Scientific premise: Mechanism of MeV virus membrane fusion and its inhibition: the “fusion complex” in the CNS. This application builds on the recently published data and on several new findings from Applicant's laboratories. The MeV hemagglutinin (H, the receptor binding protein) and fusion (F) envelope glycoproteins work together to mediate virus attachment and entry into target cells. Applicants refer to the H/F pairs of MeV as the viral “fusion complex” since these proteins, in the wild-type virus, act in concert. F is synthesized as a precursor (F₀) that is cleaved within the cell to yield the pre-fusion F complex comprising three C-terminal F₁ subunits associated covalently with three N-terminal F₂ subunits. This trimeric F structure is kinetically trapped in a metastable conformation, primed for fusion activation upon engagement of the H glycoprotein by the cell-surface receptor (discussed in A.2). After receptor engagement by H, the pre-fusion F undergoes a structural transition, extending and inserting its hydrophobic “fusion peptide” into the target cell. During entry, F refolds into a “trimer of hairpins” post-fusion structure that brings together the N-terminal heptad repeat (HRN) and the C-terminal heptad repeat (HRC), triggering subsequent fusion of viral and cell membranes. Before refolding to the post-fusion state, the F protein undergoes a series of intermediate states. One of these intermediates is the putative target of antiviral HRC-derived peptides. Applicants have shown that in MeV field isolates from the CNS, the fusion complex of MeV can mediate viral spread in the CNS in the absence of canonical receptors, and that this specific CNS adaptation is due to mutations in the MeV F protein. The combination of acquired mutations in the F protein derived from the CNS includes a balance between those mutations that lead to decreased thermostability and others that partially “compensate” for the decreased stability. Applicants previously reported that the introduction of these compensatory mutations alone into the wild-type fusion protein increases the fusion protein stability.

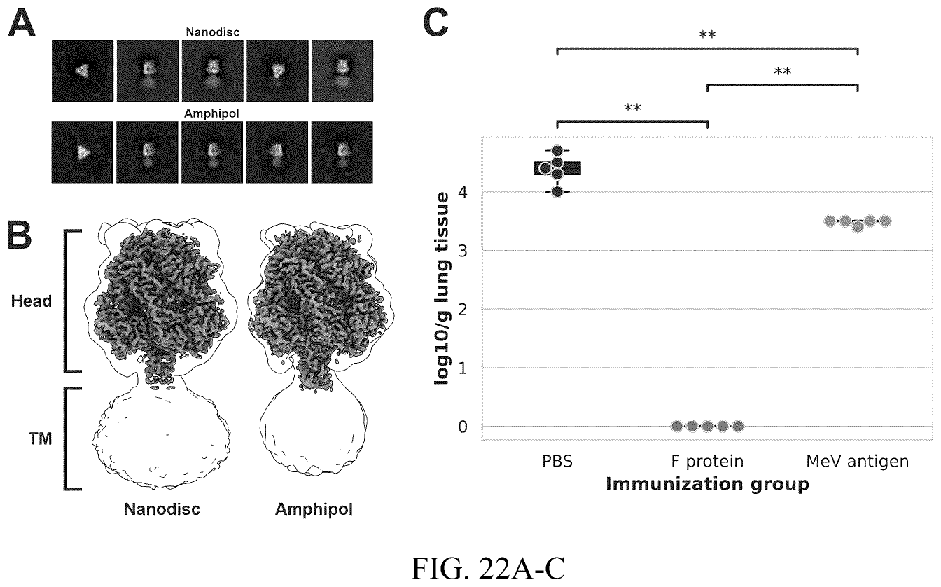

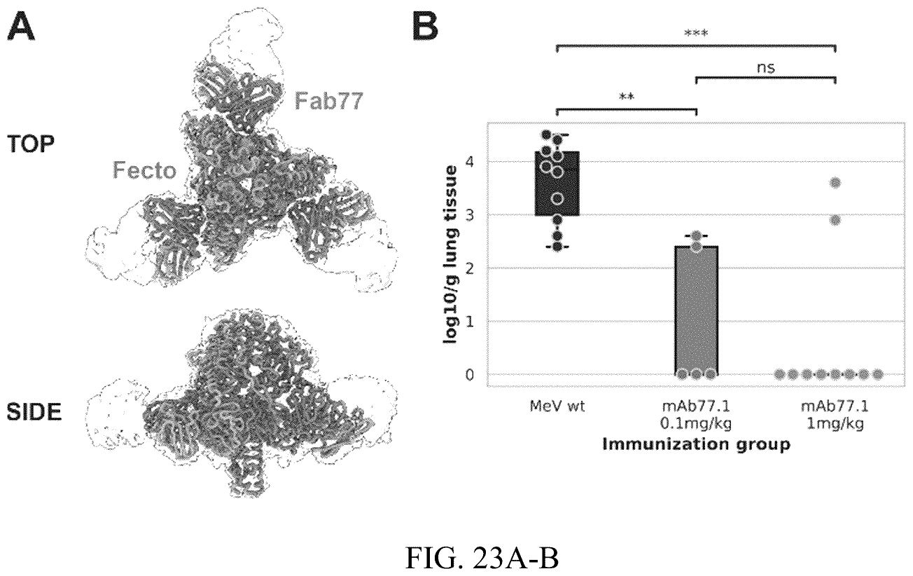

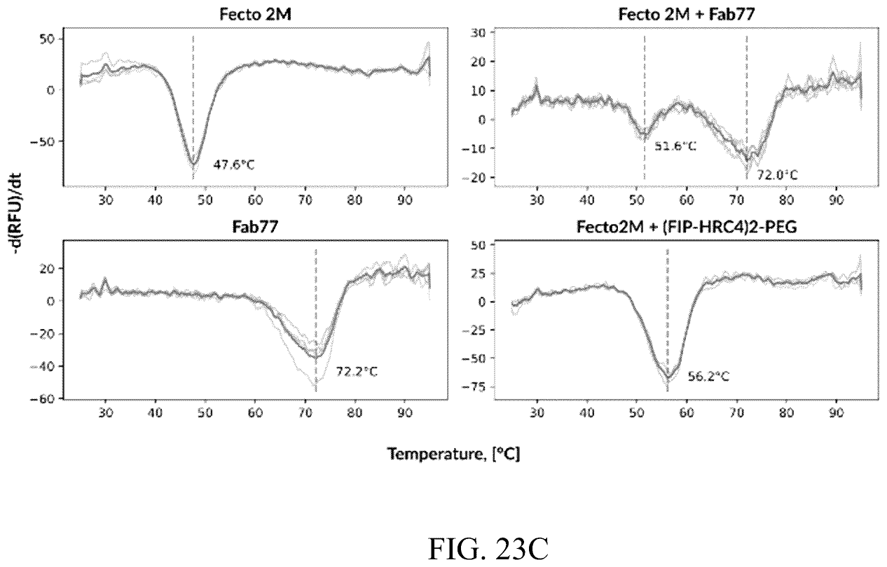

Based on these results, Applicants produced MeV F proteins with an abolished cleavage site containing the first two identified stabilizing mutations (E170G and E455G), in full-length and ectodomain versions. Applicants describe the full-length protein in FIG. 22 and the ectodomain version in FIG. 23.

The full-length MeV fusion protein bearing the mutations E170G and E455G maintains the pre-fusion state both in nanodisc and amphipol (FIG. 22A). In the stabilized full-length structure (FIG. 22B), the trans-membrane and intracytoplasmic domains are not yet ordered, but their presence is confirmed. Immunization with this full-length version resulted in protective immunity in vivo: Animals received 5 μg of the stabilized full-length MeV F twice. Immunized animals were protected against the subsequent viral challenge (FIG. 22C) and developed neutralizing antibodies (not shown). No vaccine-enhanced disease after the subsequent challenge with the virus was observed (not shown).

In addition to the full-length MeV F, Applicants have also produced an ectodomain form of the stabilized MeV F protein (FIG. 23). In FIG. 23A, Applicants report the structural information of the extra cytoplasmic domain (ectodomain, aa 1 to 495) of the MeV F in complex with the variable region (Fab) of a monoclonal antibody (mAb77.1, the humanized version of a mouse mAb 77.4 Applicants previously described (Mathieu, et al., 2021)) which is protective in vivo (FIG. 23B). The ectodomain is thermostable (FIG. 23C), and its thermostability is further improved by a previously described fusion inhibitor (Bovier, et al., 2021). Note that Fab 77 (77.4 is the mouse mAb and 77.1 is the humanized version) recognizes the prefusion state, and arrests fusion at the pre-hairpin intermediate state.

Based on the success of these two initial mutations (E170G and E455G), Applicants explored other stabilizing substitutions as well. Applicants report here (Table 6) that when these mutations are combined in the wild-type F background, their effect is additive to the point that the most stable F protein can be exposed at 60 °C for a prolonged time and still maintain the pre-fusion state (assessed by recognition of pre-fusion antibody). For the experiment in Table 6, cells were transfected with F containing the indicated substitutions and 24 hours post-transfection, cells were exposed to either 55 °C or 60 °C over time. The presence of the pre-fusion epitope assessed and the time in minutes at which 50% of the pre-fusion epitope was still observed (TS50) are reported.

TABLE 6
Thermal stability of the MeV F mutant proteins detected by the binding
of a mouse monoclonal antibody (mAb 77.4) specific for the pre-fusion
conformation of F. Data are reported as an average of three independent
experiments and are expressed as the time (minutes) at 55 °C or 60 °C that
decrease the fraction of pre-fusion epitope to 50% (TS₅₀) compared
to time 0 (100%, after overnight at 37 °C).

| | Thermal stability | Thermal stability |
|---|---|---|
| | at 55 °C TS₅₀ | at 60 °C TS₅₀ |
| MeV F (mutations) | in minutes | in minutes |
| wt | 10 | 2 |
| E455G | 21 | 18 |
| E170G/E455G | 60 | 25 |
| E170G/E455G/G506E | >120 <180 | >30 |
| E170G/Y442H/E455G/G506E | 180 | >30 |

Based on the data from Table 6 and more recent findings, Applicants can introduce additional mutations with the aim of obtaining highly stable and highly immunogenic MeV fusion protein. Note that the wild-type F are in post-fusion state (data not shown).

In summary, Applicants have obtained structural data for both full-length and ectodomain forms of stabilized MeV fusion F, both in complex with and without a neutralizing antibody, and demonstrated the preliminary efficacy of the stabilized fusion proteins as a safe, recombinant subunit vaccine strategy.

A4. Current gaps and medical need: passive immunotherapy and vaccination modalities. Applicants propose developing both an active immunization strategy (FIG. 22C) and passive immunization strategy (FIG. 23B), based on educating the immune system to target the pre-fusion state of measles F.

The availability of the live attenuated vaccine means that most of the U.S. population has not been exposed to wild-type MeV. In the absence of exposure, the level of neutralizing sera in the overall population (herd) is decreasing over time, leaving highly immunized countries actually susceptible to larger outbreaks. This increasing susceptibility poses a particular threat to those who are too young or too vulnerable immunologically to be protected by the live vaccine. “America's success in eliminating measles produced a paradoxical increase in the vulnerability” (Schuchat, et al., 2016).

The existence of an efficacious, live-attenuated MeV vaccine has almost led to the successful eradication of MeV. However, several political and socioeconomic factors have prevented complete eradication. The growing number of immuno-compromised people, who are most at risk of MeV infection, cannot be vaccinated themselves and are at risk during MeV outbreaks. A safer form of the vaccine, from stabilized recombinant subunit, is needed for these individuals.

Successful proof-of-concept for the stabilized, recombinant F approach can be found in the related respiratory syncytial virus (RSV). Several stabilized forms of fusion protein are in the clinical pipeline for evaluation of efficacy and safety, and one was recently approved for clinical use (see FDA news release May 3, 2023). In this application, Applicants assess a stabilized MeV F proteinaceous vaccine and test whether the unique stabilization strategy has the additional advantage of thermostability: maintaining the right structure and shape, even at high temperatures. This thermostability would also be a boon to MeV global eradication where poor or inconsistent cold chain compromises vaccine access and delivery, an issue that has not been solved by the dry formulation of the current vaccine. A cold chain is a temperature-controlled supply chain that includes all vaccine-related equipment and procedures. The cold chain begins with the cold storage unit at the manufacturing plant, extends to the transport and delivery of the vaccine and correct storage at the provider facility, and ends with administration of the vaccine to the patient. With this project, Applicants expect to fill specific unmet needs for people at risk for MeV infection, both at home and abroad, who cannot be vaccinated or for whom a thermostable vaccine would improve access.

### A5. Scientific Rigor and Consideration of Relevant Biological Variables.

Scientific rigor: All experiments can be performed with robust sample numbers and repeated multiple times on different days. Appropriate statistical tests can be applied to evaluate efficacy, and all virological readouts. Orthogonal assays can be used to confirm the results. All reagents can be validated as outlined in “Authentication.” Detailed experimental protocols and primary data can be maintained and available to interested parties. Positive and negative controls (e.g., infected vs. non-infected, immunized vs. naive) are included in all experiments, as evidenced in the prior body of research and preliminary data.

Relevant biological variables: Some proposed experiments can be performed in cell culture systems, where the gender for established cell lines is not a variable. Where primary cells are used as test systems, the gender of origin can be noted and tracked as an experimental variable (the same applies for human induced pluripotent stem cells-hiPSC), and in vivo experiments can include both males and females.

### B. Innovation

This proposal embodies three levels of innovation:

1) Mechanistic: The in vitro mechanistic experiments revealed that several mutations, which arose to counterbalance F-destabilizing mutations in CNS-adapted fusion complexes, effectively stabilize the prefusion MeV virus F. By combining the stabilizing mutations in the protein, Applicants successfully obtained a novel, thermostable, prefusion MeV F, which can be made in full-length and ectodomain forms and have both been confirmed by cryo-EM.

2) Conceptual: A previous attempt to stabilize the MeV F in its pre-fusion state involved disulfide stapling of MeV F ectodomain. However, this artificial disulfide F protein aggregates even at 4 °C (data not shown). Here, instead of artificially introduced mutations, Applicants used a combination of natural mutations isolated in human lethal cases of MeV encephalitis from viruses adapted to the human central nervous system. Among the natural mutations are some that decrease and others that increase thermal stability. By combining only the naturally evolved stability-increasing mutations, Applicants created a remarkably thermostable form of MeV F (see Table 6; stable at 60 °C). Additionally, Applicants can test the hypothesis that inclusion of the membrane-proximal external region (MPER), which is present only in the full-length protein, can lead to more potent neutralization activity than immunization with ectodomain alone, as has been shown for RSV, HIV, and Influenza.

3) Translational: The stabilized full-length MeV F elicits a protective immune response in a validated animal model for vaccine evaluation (cotton rat). Applicants propose that the new forms of MeV F incorporating the additional stabilizing mutations can retain immunogenicity (see Tables 6 and 7) over time and temperature to obtain a shelf and temperature-stable vaccine. Additionally, Applicants have developed a fusion inhibitor that can further stabilize the pre-fusion F protein (FIG. 23C), which could be used as an additional pre-fusion state stabilizer. Finally, Applicants propose that Applicants can obtain several human mAbs, for post-exposure prophylaxis as an alternative to the currently use of IgG preparation purified from hyperimmune sera. In contrast, monoclonal antibodies preparations consist of IgG only, have defined targets and optimized neutralizing activity per amount protein.

### C. Approach

C1.0 Engineering thermostable MeV F protein immunogen. Hypothesis: Applicants hypothesized that pre-fusion MeV fusion protein would elicit strong neutralizing antibodies. Applicants propose that immunization with full-length MeV stabilized F protein can result in superior protection vs. immunization using ectodomain forms of MeV, by (i) the inclusion of membrane-proximal external region (MPER) antibody epitopes, (ii) superior quaternary structure, and (iii) lack of potentially distracting “stump” at the very base of the ectodomain.

Rationale: The current MeV vaccine is based on live-attenuated MeV that has been proven safe and efficacious to prevent MeV infection and disease in the immune-competent host. Using this vaccination strategy, MeV infections could be controlled in the early 2000s. However, the resurgence of MeV infection in advanced countries, the persistently incomplete vaccine coverage in developing countries, and the growing number of vulnerable immunocompromised individuals without vaccination who can no longer rely on herd immunity for protection, in aggregate underscore the need for an additional vaccine that complements and solves the shortcomings of the current vaccine. The two major drawbacks of the current live-attenuated vaccine strategy are that (1) severely immunocompromised people cannot be vaccinated with the live vaccine and (2) that distribution of the vaccine requires a cold-chain and careful handling during reconstitution. Additionally, the current vaccine elicits mostly antibodies against hemagglutinin (H): 80% of the human sera antibodies are against H, and only 20% against F, and those anti-F antibodies that are elicited by the current vaccine are poorly neutralizing. Applicants believe that this demonstrates a gap in vaccine coverage. Applicant's preliminary data show that antibodies against pre-fusion F are neutralizing and offer strong in vivo protection (FIGS. 22 and 23). During CNS infections (which are most often associated with lethal outcomes) antibodies blocking F protein may be most effective by preventing viral spread early on. Applicants hypothesize that a neutralizing anti-F response can be beneficial. Here, Applicants can test the hypothesis that antibodies elicited by immunization with F are protective. Applicants propose to develop a strategy based on the use of a stable pre-fusion F protein that could be stored and transported at room temperature and which could be safely used in the immune-compromised population. This strategy could boost existing immunity and develop de novo protection in those who have not been able to receive any current vaccine. F could be combined with H in an eventual vaccine; here, Applicants begin by testing the hypotheses that a stabilized F can lead to protective immunity, as has been observed for RSV.

Based on the published and unpublished data (see Table 6), Applicants have identified several mutations that stabilize the pre-fusion state of MeV F and render the recombinant subunit thermostable. In aim C1, Applicants can prepare stabilized F proteins, determine the efficacy of these highly stabilized F as immunogens, and compare full-length to ectodomain versions to evaluate the role of the membrane-proximal, transmembrane, and intracytoplasmic domains as neutralizing epitopes. For the related respiratory syncytial virus (RSV), a stabilized pre-fusion Fectodomain, lacking the membrane-proximal, transmembrane and intracytoplasmic domains, and stabilized using introduced disulfide bonds, has been recently approved for clinical use (see FDA site), providing a strong rationale for the strategy here. For RSV, only the pre-fusion state of the F protein induced neutralizing antibodies, whilst the post-fusion state does not. This finding is in stark contrast to the related human metapneumovirus (HMPV), for which neutralizing antibodies in vivo are directed against both the pre- and post-fusion state of the HMPV F. Here, Applicants can also determine whether MeV neutralization requires antibodies only against the pre-fusion state of F (as in the RSV case) or if both pre- and post-fusion states contain valid neutralizing epitopes (as in the HMPV case).

Strategy and Plan: In the published and unpublished data (see, FIGS. 22, 23 and Table 6), Applicants have shown that several naturally occurring amino-acid mutations in the MeV F protein stabilize the pre-fusion state of MeV F. These naturally occurring mutations are superior in their stabilizing properties compared to the previous artificial disulfide bonds introduced into the MeV F ectodomain. In the preliminary data presented here, Applicants show that the combination of two previously published natural mutations (E170G and E455G) supports production of pre-fusion MeV F (FIGS. 22 and 23), and that this stabilized prefusion F can be efficiently used to induce protective immunity in vivo (FIG. 22C). Newer data (Table 6) show that two additional mutations further stabilize the pre-fusion F. In this aim, Applicants can produce full-length and ectodomain versions of MeV F with these new mutations and assess their stability over time (up to 12 months) and temperature (up to 60 °C).

Applicants have also shown that a humanized anti-pre-fusion F antibody is efficacious in vivo (see FIG. 23B). Using the current and newly generated MeV F protein (full-length and ectodomain versions), Applicants can elicit fully human monoclonal antibodies and assess the antiviral activity in ex vivo models that represent the main targets of MeV infection and complication (immune system, lung epithelia, and central nervous tissue) using both wt and central nervous system (CNS)-derived recombinant virusesto ensure that the antibodies Applicants are eliciting are neutralizing in the appropriate target tissue and could block CNS-adapted viruses. Additional viral evolution in the three ex vivo models described above can test the barrier to resistance to the neutralizing antibodies. Applicants hypothesize that the full-length version of the stabilized MeV F can induce stronger immunity (in terms of neutralizing activity and with a higher barrier to resistance) compared to the ectodomain version. In C1.1 and C1.2a, cryo-EM, and biochemical characterization can determine the precise structure of the stabilized F and determine the specific epitopes recognized by the neutralizing antibodies. In C2, the efficacy of the neutralizing antibodies and the vaccine strategy can be assessed in vivo in a relevant animal model for MeV infection.

C1.1 Production and characterization (biochemical and structural) of stabilized (full-length and ectodomain) MeV fusion proteins (at CUIMC/LJI). Hypothesis: Highly stabilizing mutations in the MeV F can allow for a long shelf life even in the absence of cold-chain. Applicants can utilize a total of six different MeV F: Wild-type F in ectodomain and full-length (post-fusion); E170G/E455G in ectodomain and full-length (pre-fusion); and two additional combinations of stabilizing mutations to be selected here, also in ectodomain and full-length (pre-fusion). The first four (blue highlight in Table 7) have been described and characterized above and are ready to be used (see FIGS. 22 and 23). The fifth and sixth pre-fusion F proteins can be selected based on the results of this subaim. Applicants can begin with the four available MeV F proteins (pre-vs. post-fusion; ectodomain vs. full-length) to determine whether only pre-fusion Felicits/selects neutralizing antibodies, or if the post-fusion state can also induce neutralizing immunity. In Table 6 Applicants show a series of mutations that, when combined, further stabilized the MeV F. Applicants can produce F proteins from this set of mutations as follows: E170G/Y442H/E455G ectodomain and full-length; and E170G/Y442H/E455G/G506E full-length (yellow highlight in Table 7). The ectodomain ends at aa 495; therefore, the G506E stabilizing mutations can be used only for the full length. For the next versions of F, Applicants can test whether a V459I that Applicants have also recently identified to stabilize F offers improved stability when combined with the other mutations. Applicants can produce the F proteins that incorporate stabilizing mutations as described in Table 7. Applicants can then assess the stability of the different F proteins and based on stability data (tested as in FIG. 23C; requiring small-scale preparation), Applicants can produce 6 new F proteins (three full-length and three ectodomains) plus the two original ones (blue highlight in Table 7) for a total of 8 different proteins. The wt F proteins adopt the post-fusion conformation and can be tested only in immunization studies. Applicants can prepare multiple aliquots of the F proteins. Aliquots can be either lyophilized or left in solution. Both groups can be kept at different temperatures (−80, −20, 4, 30, 40, 50, 60 °C) and for several times (1, 3, 6, 9, 12 months). Applicants expect −80 °C to be the control for maintaining stability, and thus, Applicants can have several aliquots at −80 °C to use for each time point and temperature. At the indicated time points and temperatures, Applicants can assess the amount of pre-fusion vs. post-fusion both structurally (using single particle negative stain and/or cryo-EM as in FIGS. 22 and 23) and using conformation-specific monoclonal antibodies. Preference can be given to the two MeV F proteins that show the highest stability of pre-fusion epitope after incubation at high temperature and for the longest time (see metrics in milestone).

TABLE 7
F proteins to be produced and tested. F
proteins Applicants have already in hand,
tested in CRYO-EM and ready to use are
wt and E170G/E455G. The E170G/E455G full-
length and ectodomain are described in
FIGS. 22 and 23. E170G/Y4442H/E455G and
E170G/Y442H/E455G/G506E have been assessed
for stability (see Table 6).

| | Ectodomain | |
|---|---|---|
| MeV F (mutations) | (aa 1-495) | Full Length |
| wt | X | X |
| E170G/E455G | X | X |
| E170G/Y4442H/E455G | X | X |
| E170/E455G/V459I | X | X |
| E170G/Y4442H/E455G/V459I | X | X |
| E170G/E455G/V459I/G506E | Stops before aa 506 | X |
| E170G/Y442H/E455G/G506E | Stops before aa 506 | X |
| E170G/Y442H/E455G/V549I/G506E | Stops before aa 506 | X |

Immunogenicity in mice: The most stable F proteins (one full-length and one ectodomain version), together with the wt F in its post-fusion state, as well the E170G/E455G pre-fusion full-length and ectodomain F that Applicants have already produced, can be used here and in C2.2 to assess their potential to elicit protective immunity in vivo. A total of 6 F proteins can be used for immunization: [3 full length and 3 ectodomain to include: one each post-fusion (wt), one each of the original 2 pre-fusion (E170G/E455G), plus 2 selected from the stability studies]. Mice can be immunized twice intramuscularly (i.m.) with 1 and 5 μg of protein. In FIG. 22 Applicants used 5 μg of F protein given twice 21 days apart (day 0+21) to generate a strong immune response. Adjuvant (for both mice and cotton rats): In the current experiments, Applicants used alum as an adjuvant. AS03 from GSK would be ideal. Applicants can attempt to obtain AS03 from GSK, or alternatively, Applicants can commercially acquire a similar formulation (i.e, AddaS03™, InvivoGen). Two weeks after last immunization (day 35) the neutralizing activity of the sera can be tested using live virus (both wt and CNS-adapted variants). Sera can also be analyzed by ELISA for binding reactivity to the antigens used for immunization. Number of animals required: 7 proteins (6 F proteins+mock control)×10 (5 each sex)×2 doses (1 and 5 μg)×3 treatments (kept at −80°, kept at 4° for 1 month in solution, and incubated at the highest temperature and longest time and still be at 90% pre-fusion)=420 mice. Note that in the case of the wt F, proteins can be in post-fusion state all the time and serve as control. If the sera are not neutralizing, the antigen and the immunized mice cannot be progressed further.

C1.2. Neutralizing antibodies against MeV F: antiviral activity, structural and biological characterization (at CUIMC/LJI). In this sub aim, Applicants can obtain monoclonal antibodies (mAb) by either mouse immunization (from C1.1) and subsequent antibody humanization (as done above for 77.1), or by screening a de-identified human healthy donor library of hybridoma (which is currently frozen at CUIMC, from PBMC of an individual who was previously infected with MeV wt virus and now has a high neutralization titer). The overall objective is to obtain fully human monoclonal antibodies.

C1.2a mAbs against MeV F. Hypotheses: (1) Full-length MeV F can elicit more potent antibodies than the truncated ectodomain. (2) Post-fusion epitopes are not neutralizing. These hypotheses can be tested directly via mouse immunization and indirectly by assessing neutralizing activity of human hybridoma derived from PBMC.
(i) Mouse: After assessing the neutralizing activity of the sera, a total of 12 mice from C1.1, two mice per antigen (one male and one female) with the most neutralizing sera against live virus can be used for hybridoma production.
(ii) Human: Applicants have prepared a library of human hybridomas from a de-identified human donor with high neutralizing titer for MeV. The antibodies against F can be analyzed in this aim in order to address the specific hypotheses about protection from anti-F and anti-pre-fusion vs. anti-post fusion F.

The supernatant fluids from these hybridomas (murine and human) can be assessed for inhibition of viral entry. The supernatant fluids from human hybridoma (which may contain a majority of anti-H antibodies) can be further screened using a specific fusion assay mediated by MeV F in the absence of MeV H. This can allow detection of specific F mAb and exclude H specific antibodies. The binding reactivity of the supernatant antibodies to the pre-fusion, or post-fusion, full-length or ectodomain antigens used for immunization (see C1.1) can be assessed by ELISA. Applicants can use these results to determine if neutralization activity correlates with the recognition of pre-fusion and/or post-fusion F in cell-based ELISA, as described in. Briefly, cells expressing F (full-length wild-type, full-length E170G/E455G, and the most stable full-length F selected in C1.1 from Table 7) can be kept at 4 °C (a condition which maintains the pre-fusion state) or incubated at 55 °C for 60′ (a condition which drives wild-type F into the post-fusion state). The supernatant fluids from the hybridomas can be assessed for their ability to bind to the resulting F on the cell surface. The data from these screens can indicate which hybridoma offers the most potent neutralizing activity and the viral target to which it binds (e.g., Fin pre-fusion, and Fin post-fusion). The most potent mouse and human antibodies identified can be sequenced. Mouse mAb can be humanized as Applicants have done for 77.4 to obtain the 77.1 human antibody (FIG. 23). Synergy-additivity-antagonism between mAbs can be assessed. The most potent humanized mouse- and human antibodies can be scaled up and tested (1) for potency ex vivo (as Applicants have done before) and (2) imaged using single particle cryo-EM to map their epitopes (see FIG. 23 for proof-of-concept using 77.1). The mechanism of action can also be assessed using established assays. Based on the ex vivo potency and epitope recognition, Applicants can select 8-10 mAbs for viral evolution studies in C1.2b.

C1.2b Ex vivo viral evolution under selective pressure. Hypothesis: Assessing the determinants of viral resistance early on in the drug discovery process can identify the most promising candidates prior to in vivo testing.

Rationale: Applicants have previously described a murine mAb 186a that although potent, was specific only for a subset of MeV strains. Applicants have also described a mAb, 77.4, with a broader spectrum of activity, including efficacy against CNS-adapted MeV variants. Applicants have humanized the broadly active mAb 77.4 into a fully human version termed 77.1 (FIG. 23). Consistent with its broad activity, Applicants have been unable to generate resistance mutations to 77.1 in cell culture despite several attempts. Applicants want to determine the potential for the new mAbs to be identified here to elicit resistance. Applicants propose here ex vivo (using B-cells, lung organoids, and brain organoids) virus evolution to search for resistance mutations. The use of the ex vivo models has two main advantages: (1) the potential for viral evolution in a more authentic tissue, and (2) the time course of viral evolution can be of several weeks (especially for the organoids) allowing more time for the virus to evolve.

Strategy and overall plan: Applicants can attempt to elicit MeV variants resistant to the mAb in B-cells, lung organoids, and brain organoids. Applicants can use wt virus and a virus bearing the L454W F (as example of CNS-adapted virus) mutation which spread most effectively in brain organoids and in vivo. This knowledge can be used to identify the mAb(s) least likely to elicit resistance (for C2.1), and those able to block CNS-adapted variants, a feature important to avoid mAbs that may induce CNS-adapted viruses. Applicants can correlate escape mutation sites, lack of escape mutation emergence, epitope, and the form of F best recognized by the mAb (pre/post-fusion; stabilized or not), together with the data from C2.2 in order to select F best suited to passive immunization. Cotton rats can be used to assess the in vivo pathogenesis of viral variants that arise under the selective pressure of mAb treatment (e.g., whether resistance to mAb leads to a more pathogenic virus or not). The 8 mAb that performed best in C1.2a (e.g., lowest IC₅₀ and IC₉₀ ex vivo, with the most diverse epitope recognition) can be tested here for efficacy.

Generation of MeV variants resistant to mAb: Applicants can attempt to elicit MeV viruses resistant to the inhibitory effect of mAbs using protocols routinely performed in the lab. In these experiments, Applicants can use the B3 strain, which is the currently circulating strain of wt virus, labelled with GFP. Briefly, several dilutions of MeV B3-eGFP wt and B3-eGFP L454W (both viruses are in hand) can be passaged ex vivo in the presence of several concentrations of mAb (ranging between 5× and 20× the IC₅₀) for four days in the B-cell ex vivo model, or for several weeks in the lung organoid and brain organoid models. Note that mAbs can be added after the initial infection to allow the viral polymerase complex to replicate and produce phenotypic variants for selection. Resistant viruses would spread even in the presence of mAb; spread of virus can be monitored by fluorescence microscopy. Yields of virus can be determined in immunoassays and/or by RT-qPCR. As the virus spreads in the presence and absence of antibody, the concentration of the mAb can be gradually increased, to obtain a population of resistant viruses. This strategy of applying selective pressure for viral evolution is similar to the informative experiments performed in the lab for neuraminidase-resistant variants, small molecule inhibitor-resistant variants, and antibody resistant viruses.

Analysis of resistant variants in vitro: Mutant, resistant viruses can be plaque purified (if possible-see potential pitfalls below), then analyzed for their phenotype (plaque formation, resistance to inhibition by other small molecules and/or fusion inhibitory peptides). Applicants can determine whether the fitness of each variant is similar to that of the parent virus, or whether the variants require the presence of inhibitor for viability. Viral RNA can be extracted and sequenced. Applicants can analyze resistant virus mutants by high-throughput sequencing and obtain complete genomic sequences. Sequences of the plaque-purified viruses and viruses from ex vivo growth can be compared to population-derived sequences generated during the duration of the selection experiments. This way Applicants can avoid neglecting potentially important sub-populations that may co-exist during or after the selection process. The method enables specific selection of viral sequences from complex sample types, yielding sequencing depth and coverage through orders of magnitude increases in specific read numbers.

If F (and/or H, or other viral proteins e.g., the matrix protein) contain mutations, Applicants can introduce the mutated genes into the expression vectors and evaluate the glycoprotein functions in the functional assays (F stability, F-triggering, fusion assays). If multiple mutations are found, site-specific mutagenesis can be used to introduce the mutations into the F (or H) background, and singly mutated genes can be analyzed for their phenotypes using the same in vitro assays. Location and conservation of the mutations can tell the extent to which the resistance mechanism(s) for different mAb are similar. If the mutants derived from the different mAbs are markedly different, Applicants can analyze the contributions of the specific mutations (in either F or H, or other genes if mutated) to dissect each contribution. Applicants also have the reverse genetics system in hand in case Applicants want to introduce selected mutations into the live virus.

Analysis of resistant variants in vivo: If Applicants identify resistant variants that grow well in vitro and ex vivo, Applicants can assess their in vivo fitness. Resistant variants' pathogenicity can be compared in vivo to the parent MeV virus. Applicants can use 6 adult cotton rats (CR) to assess the in vivo fitness of each resistant variant. Applicants anticipate testing a maximum of 5 variants plus control for a total of 6 viruses, (6 animals per group, n=36 CR, 18 each sex).

Viral titer: Virus isolation can be done from lungs as described above and in. Virus titration can be performed by plaque assay. Samples can also be sequenced to assess viral evolution in vivo.

Anticipated results and potential pitfalls: Applicants already have four of the six MeV F proteins required for the work proposed here and in C2.2. In C1.1, Applicants can express additional F proteins based on the mutations in Tables 6 and 7 and select the additional two for C2.2. If stabilization at high temperature for prolonged times (e.g., 90% pre-fusion stability) is not possible by simply using the mutations from Table 6, Applicants can also add a specific fusion inhibitor that can further stabilize the pre-fusion ectodomain of F protein (FIG. 23C). Applicants also have in hand one fully human antibody (mAb 77.1) for C1.2b and C2.1. Applicants anticipate generating additional mAbs, but propose to start C1.2b with mAb 77.1 while Applicants wait for the new mAbs identified in C1.2a. Applicants present ample evidence that single-particle cryo-EM work can proceed seamlessly and that specific epitope recognition can be identified (FIGS. 22 and 23). Applicants suggest that Applicants can correlate epitope recognition to antiviral potency and test the working hypothesis that mAbs that recognize the membrane-proximal domain (present in the full-length, but not ectodomain F) can be potent and offer a higher barrier to resistance. Applicants have a set of functional assays that can identify the specific mechanism of action (e.g., F-stabilization or destabilization, or blockage of transient intermediates). Since the recombinant MeV viruses are in hand and ex vivo models are established, the efficacy study should proceed seamlessly. Dose-response assays can determine the dose-response in cell lines and ex vivo of all the mAb to move forward into ex vivo efficacy studies. The potency of the mAbs and their likelihood of eliciting resistance can be revealed using relevant ex vivo models. Since Applicants have already used these models, Applicants anticipate minimal technical difficulties. Applicants considered the possibility that the matrigel in the lung organoid model might prevent full diffusion of the antibody; in this case Applicants could make a single fragment antibody to facilitate diffusion or use human airway epithelial cells as in the previous work.

Mutations arising under the selective pressure of mAbs ex vivo should be highly informative about the relationship between viral fitness in authentic host models and the role of F-mediated entry, i.e., which features of the MeV glycoproteins are conducive to fitness. Data on the molecular basis of resistance can derive from the analysis of mutations in plaque-purified resistant viruses as well as the population analyses of potential viral quasi-species generated during the selection process. Selection in organoids may differ from selection in B-cells, and the unbiased approach can capture this eventuality as well.

The sequence alterations that arise through H and F mutation can be linked to functional alterations, and this information used to understand resistance mechanisms. For human parainfluenza virus type 3, enhanced fusion kinetics led to partial resistance to peptide inhibitors in vitro. However, Applicants showed that mutations that increase fusion kinetics have a concomitant negative impact on growth in natural host tissue (human airway) and such resistance mutations are likely to significantly reduce fitness in vivo. Applicants propose that by assessing these mechanisms early on in antiviral development, Applicants can avoid advancing antiviral strategies that could lead to more pathogenic viruses. It is possible that no infectious particles can be recovered from the ex vivo models (since MeV tend to spread via cell-to-cell infection in this tissues) and in this case Applicants can simply use the sequence data and introduce selected mutations in the reverse genetics system to obtain recombinant viruses bearing the mutated viral genes. Determining the ease of generation of variants and the fitness of MeV-containing resistance mutations can permit Applicants to predict the likelihood of evolution of clinically relevant resistant MeV (in future work). Applicants also consider the possibility that no resistance can be elicited. This is unlikely, since in the presence of small molecules, resistant variants of MeV have previously arisen. However, lack of resistance would suggest that the fitness cost is too high to generate a viable variant resistant to that specific mAb, and this mAb would therefore be an ideal candidate to move forward. The information gained here can be used to identify mAbs with the lowest likelihood of eliciting resistance, and to infer the best antigen for active immunization.

Discussion: From aim C1.1, Applicants can determine the stability and longevity of the stabilized MeV F proteins, as well as their potential to induce neutralizing antibodies. Applicants can determine whether neutralization activity is endowed only by the pre-fusion state of the F protein, or if the post-fusion state can also elicit neutralization activity (disproving the working hypothesis). In C2.2, the same question can be asked in the context of viral infection in vivo. The hypothesis that the membrane-proximal domain and/or other determinants in the full-length F may elicit an additional or superior neutralization activity can be tested here in terms of neutralizing antibodies and can be correlated with the in vivo protection data in C2.1 (as shown in the preliminary data above see FIGS. 22 and 23). The stabilized F protein that elicits the most potent response and that is most stable can be identified.

Applicants can also obtain a set of diverse mAbs directed against several epitopes of the F protein. By assessing mAbs in ex vivo tissue, Applicants can identify viral inhibitors that are active in authentic tissue, and Applicants can also identify ideal probes for use in future studies of pathogenesis. In C1.2b, Applicants can attempt to elicit viral resistance to the neutralizing antibodies obtained in C1.2a, to select the most promising neutralizing antibodies to move to C2.1. Since Applicants propose that ideally the final use of the mAbs can be in a cocktail, synergy and non-overlapping epitope binding are ideal features. Applicants expect to move to C2.1 a total of 4 mAbs (77.1 plus additional 3). The data from Aim C1 can directly inform the work of aim C2.1, and therefore aims C1 and C2 are interconnected. However, the availability of mAb 77.1 can allow aim C2.1 to proceed even in the unlikely eventuality that no better mAbs can be identified in C1. Aim C2.2 can start with the four available F proteins and await the results of C1 for the addition of two F proteins of potentially greater stability.

C2. Evaluate the protection afforded by anti-F protein immune responses. Hypothesis: Applicants propose that immunization with stabilized MeV F can induce protective immunity comparable to the current live vaccine (without risk of vaccine-enhanced disease).

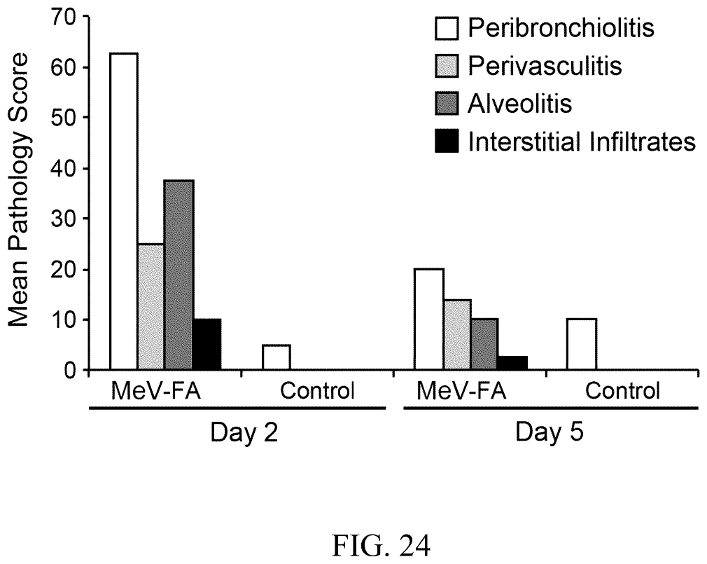

Rationale: Applicants have shown that immunization with pre-fusion, full-length MeV F efficiently protects cotton rats from MeV infection (see FIG. 22) and that a specific mAb against the prefusion state of F is highly efficacious in pre-exposure prophylaxis (see FIG. 23). Importantly no hyperresponsiveness (enhanced disease) was observed in the preliminary immunization studies. Absence of enhancement is critical to avoid the safety issues caused by vaccination with formalin-inactivated MeV (FIG. 24). Here, in aim C2.1, Applicants can evaluate mAb against prefusion F in passive immunization, assessing whether specific mAbs against the prefusion state of F are superior to general polyclonal human hyperimmune serum, the current standard of treatment, in blocking MeV infection. Next, in aim C2.2, Applicants can evaluate activity against pre-fusion F in active immunization, comparing immunization with stabilized, pre-fusion F to the current live attenuated vaccine in both immune-competent and immune-suppressed animals. Of paramount importance is that the proposed strategy does not cause hyperresponsiveness (a.k.a. vaccine-enhanced) disease, a feature which can be carefully tested here.

Virus-induced lung damage or vaccine-enhanced disease. Immunization of children with formalin (FA)-inactivated MeV virus has led to more severe disease after infection with wildtype virus (so-called atypical or vaccine-enhanced disease). Atypical measles was characterized histologically by severe pneumonitis. The analysis of lung damage after primary infection or vaccine-enhanced disease after challenge of immunized cotton rats can be performed stepwise. On day 4 after infection, the left lung lobe is secured for virus titration. The right lung lobe is inflated with formalin, fixed for 3 days, processed and embedded in paraffin. After processing, slides are stained with H&E. For vaccine-enhanced disease, lung pathology is scored on a scale from 0 (nothing) to 100 (severe) for alveolitis, peribronchitis, perivasculitis and interstitial infiltrates. Using this method, Applicants have obtained data for vaccine-enhanced disease after immunization with FA-inactivated MeV in cotton rats. In Applicant's hands, immunization of cotton rats with FA-inactivated MeV leads to the development of T cells as well as neutralizing antibodies which decline towards the time of challenge (data not shown). After MeV challenge, viral titers in lung tissue were 100-fold reduced (2.6±0.3 log10 in immunized versus 4.7±0.2 log10/g lung tissue in naïve animals). However, histologically severe inflammation was observed on days 2 and 5 (FIG. 24). If in the experiments proposed here, histological damage is observed, Applicants can establish a more detailed cytokine profile similar to that already established for atypical disease after RSV vaccination in cotton rats. Based on the RSV study, Applicants can measure IFN alpha, beta, and gamma; TNF alpha; TGF beta; and IL-6 by qPCR (LightCycler RNA amplification kit with SYBR green I (Roche) using plasmids containing cloned cotton rat cytokine genes as standards. Data can be analyzed using LightCycler software version 3. Quantification can be based upon fit point analysis with arithmetic baseline adjustment. Melting peak and melting curve analyses can be performed using the polynomial calculation method.

Justification of the proposed animal model: MeV naturally infects only humans and NHPs. Although Applicants have previously used small animal models to test in vivo efficacy of fusion inhibitory peptides, including a suckling mouse model transgenic for human CD150, none of these models recapitulates the complex pathogenesis of morbillivirus infections. In another approach, Applicants have used cotton rats (CR), susceptible to wild-type MeV infection. While neither model is ideal for MeV pathogenesis, cotton rats are suitable for MeV vaccination studies (both active and passive) because the virus grows in the respiratory tract, and they develop antibody and T cell responses, allowing measurement of the reduction of virus growth and development of antibodies. Therefore, Applicants propose that this model can likely reflect the efficacy of the proposed strategies and their potential shortfalls (e.g., vaccine-enhanced disease, see FIG. 24).

C2.1. Passive immunization with neutralizing antibodies (at CUIMC/OSU/La Jolla). Applicants have shown that the first humanized monoclonal antibody is highly efficacious in preventing MeV infection in cotton rats (see FIG. 23B). Here Applicants can test additional mAbs and conditions to evaluate the efficacy of the strategy in pre-exposure prophylaxis (C2.1a), post-exposure treatment (C2.1b), and in severely immunocompromised hosts (C2.1c).

Strategy and Plan: Testing in vivo antiviral efficacy of new human monoclonal antibodies directed toward MeV F protein can be performed using a tiered approach, compliant with 3R (Replacement, Reduction and Refinement) principles in animal experimentation, with tiers being separated by Go/No Go decisions. In C2.1.a, Applicants can perform a prophylaxis study. Antibodies that do not achieve adequate in vivo protection compared to the 77.1 (see FIG. 23) cannot be advanced to C2.1b. In C2.1b, Applicants can assess post-exposure antiviral efficacy by dosing antibody after exposure. Primary outcome parameters can be levels of viremia in the lungs. Antibodies that do not show efficacy in these experiments cannot be advanced to C2.1c. In C2.1c, Applicants can assess prophylaxis and therapeutic efficacy in immune compromised animals. Primary outcome parameters can be identical to those used in C2.1b. The work in aim 2 can start with the humanized anti-MeV F 77.1 (see FIG. 23) and can proceed with additional 3 antibodies selected in C1.2.

C2.1a. Pre-exposure prophylactic efficacy. Groups of three (immunocompetent) cotton rats can be dosed with anti-MeV F antibody (using the humanized 77.1 mAb used in FIG. 23B as a positive control). Antibodies can be i.p. injected 1 day before inoculation with recombinant B3-eGFP wt virus. Four days after inoculation, the animal can be sacrificed, and viral titers can be assessed. The experiment can be performed twice to bring the number of animals per treatment group to six. The repeat experiment can not be performed if all treatments fail in the first experiment (i.e., the dose or time point does not prevent infection). Number of animals required: 4 antibodies (3 new mAbs plus mAb 77.1 as positive control)×6 (3 each sex)×2 doses (0.2 mg/kg body weight and 1.0 mg/kg body weight)=48 CR.

C2.1b. Early post-exposure treatment. Groups of three animals can be inoculated with recombinant B3-eGFP wt virus and treated with the most potent mAb (based on C2.1a, the one most effective at the lowest dose) and can be inoculated 1 or 2 days after infection. Sampling and follow-up can be identical to C2.1a, and the complete experiment can be performed twice to generate groups of six. Number of animals required: 4 Ab (2 most potent mAb from C2.1a+77.1+immune serum)×6 (3 for each sex)×1 dose (highest efficacious dose from C2.1a)×2 (time points)=48 CR.

C2.1c. Prophylactic and therapeutic efficacy in immunocompromised animals. Cotton rat immune suppression: The protocol for immune suppression (I-S) in cotton rats is well established and has been extensively used for testing immunoprophylaxis for RSV. Briefly, CR can be treated with intraperitoneal (i.p.) injections of cyclophosphamide at a dose of 50 mg/kg three times weekly for 21 days prior to viral challenge, and I-S can continue for an additional 6 days (sufficient for the proposed experiments). To monitor the level of I-S during these experiments, CR can be bled for white blood cell counts and differential cell counts one day before viral challenge.

Prophylactic and therapeutic efficacy can be assessed by comparing viral titers four days post-infection (same as described in C2.1a) between mock- and antibody-treated animals. The experiment can be performed twice to bring the number of animals per group to six. Number of animals required: 4 samples (2 most potent mAbs from C2.1a+77.1+mock control)×6 animals (3 for each sex)×1 (route)×2 (time points, before and after infection)=48 CR.

Anticipated results and potential pitfalls: In this sub aim Applicants can define the prophylactic and therapeutic efficacy of passive immunotherapy in the cotton rat animal model (immune competent and immune suppressed) using antibody specific for MeV F. The primary outcome parameters are virological data obtained from the treated animals combined with the assessment of infection as in FIG. 23B. Applicants expect that prophylactic administration of the antibody can protect against infection (C2.1a) and can determine whether the new mAbs are superior to the 77.1 described in FIG. 23B. In C2.1b, Applicants can test whether passive immunoprophylaxis is also effective in a post-exposure regimen. In C2.1c, Applicants can test whether passive immunoprophylaxis is effective in immunosuppressed animals. Note that the main objectives of this sub-aim are to provide evidence that the strategy is effective in (i) prophylaxis, (ii) early post-exposure treatment, and (iii) in immune-compromised hosts. The overarching goal of this sub-aim is to establish a passive treatment based on human mAbs to provide a well-defined composition without the inherent variability of depending on human hyperimmune serum obtained from healthy donors. A cocktail is likely an ideal eventual treatment. Here, Applicants can begin by testing them individually to assist in cocktail component selection. In future work Applicants propose to test such a cocktail of mAbs against MeV F that recognize different epitopes.

C2.2. Assessment of in vivo immunization with stabilized MeV F proteins (at CUIMC/OSU/LJI). Applicants have shown that the full-length pre-fusion MeV F protein in its pre-fusion state, given twice at 5 μg, induces protective immunity in immune-competent cotton rats (see FIG. 22C). Here Applicants can test additional MeV F derived from aim C1.1. Applicants propose to compare a total of six MeV F proteins (four in pre-fusion and two in post-fusion, from aim C1.1, note that two pre-fusion (E170G/E455G, FIGS. 22 and 23) and two post-fusion (wt) are already in hand and one full length in pre-fusion has shown to be protective, (see also Tables 6 and 7) given twice at two dosages (1 and 5 μg) in (C2.2a). The F that elicits the most potent neutralization without adverse effects can be tested in an additional range of dosages with the aim of decreasing to a single inoculation (C2.2b). In parallel, the F with the best protection can be assessed in severely immune compromised hosts (C2.2c). In addition to the virological data, sera from the cotton rats before and after immunization can be evaluated for neutralization activity and epitope recognition.

C2.2a. Pre- and post-fusion F protein immunization efficacy. Groups of three (immunocompetent) cotton rats can be dosed with MeV Fin either the pre- or post-fusion state after i.p. administration, using the current live attenuated MeV vaccine as a positive control. The immunization can use two dosages (1 and 5 μg) and can be repeated twice (as Applicants have done for FIG. 22). Two weeks after the last immunization, animals can be infected and sacrificed four days post-infection. Viral titer can be assessed as in FIGS. 22 and 23. Sampling of naïve animals can be identical to that of the immunized animals, and infection can be detected based on virological data (including seroconversion). Sera of the animals can be collected before immunization, before infection, and after infection, and the neutralization titer can be measured. The experiment can be performed twice to bring the number of animals per treatment group to six. The repeat experiment cannot be performed if all treatments fail in the first experiment (i.e., the dose or time point does not prevent infection). Half of the lung can be used to assess viral titer, and the other half for the presence of hyper-responsiveness (FIG. 24). Number of animals required: 8 (6 F proteins+mock control+MeV antigen as in FIG. 22)×6 (3 each sex)×2 (doses: 1 and 5 μg)=96 CR+6 CR with live attenuated vaccine=102 CR. Post-fusion F can be used here as well even in the eventuality that non-neutralizing sera is produced in mice in C1.1. This is to avoid missing the possibility that post-fusion F vaccination may result in vivo protection despite not inducing neutralizing antibody.

C2.2b. Frequency of administration and stability testing. Groups of three animals can be inoculated with the two most efficacious F proteins (based on C2.2a, one full-length and one ectodomain, each the most effective at the lowest dose, highest neutralization titer, and without negative effect, based on the histology and cytokines and chemokine profiles). Additionally, Applicants aim to assess whether a single dose would suffice to generate a response. Sampling and follow-up can be identical to C2.2a, and the complete experiment can be performed twice to generate three groups. Number of animals required: 5 (4 fusion proteins+mock control)×6 (3 each sex)×2 doses (5 μg, once or twice)×3 (F preparations: kept at −80, kept at room temperature, and incubated at the highest temperature and longest time based on C1.1)=180+6 with live attenuated vaccine=186 CR).

C2.2c. Efficacy in immunocompromised animals. Immunization efficacy can be assessed by comparing viral titer four days post-infection between mock- and immunized animals. The experiment can be performed twice to bring the number of animals per group to six. Number of animals required: 2 (most efficacious F protein from C2.2a and C2.2b+mock control)×6 (3 each sex)=12 CR.

Note that the total number of animals listed is a maximal number (480). Applicants expect that certain immunization, dosage or frequency may fail at the first set and cannot be carried over to the next level of testing.

Anticipated results and potential pitfalls: In this sub aim, Applicants can define the efficacy of active immunization in the cotton rat animal model using MeV F in either the pre- or post-fusion state, in either full-length or ectodomain versions. The primary outcome parameters are virological data obtained from the immunized animals combined with the assessment of infection, neutralizing activity of the sera collected from the animals and lack of hyperresponsiveness (see, FIG. 24). Based on the earlier observation, there is no evidence of hyperimmune response, but it is possible that by increasing the dosage (to test activity of a single-shot immunization), this could be observed, and in this case, Applicants would return to the safer lower dosage. Applicants expect that administration of the pre-fusion state can be superior to the post-fusion state. Applicants expect some of the pre-fusion F described in Tables 6 and 7 to be superior to the current two-substitution F used in FIGS. 22 and 23. These experiments can determine if immunization with recombinant F is as, more, or less protective than the current live attenuated vaccine. Many of the antibodies elicited against the live-attenuated vaccine are elicited against H, and antibodies against both H and F may be desirable in the final second-generation vaccine. This focused program analyzes F in order to begin with the fundamental questions of whether anti-F antibody is equally or more protective compared to the current vaccine. While a dry formulation of the current vaccine that does not require cold chain exists the issue of the use of live attenuated vaccine persists for immune-compromised people. In C2.2c, Applicants can test whether the strategy could be efficacious in immune-suppressed animals. It is particularly important to measure the response in the immune-suppressed animals because they would represent an indication of efficacy in the immune-compromised population. The main goal of this sub aim is to establish an active immunization strategy that could be given to both immune-compromised and -competent people and would not require cold-chain for distribution. How long the immunity derived from F immunization can last in comparison to the current vaccine can be addressed in future work, once Applicants determine the most effective subunit vaccination strategy in this application.

Aim 1: Generate truncated and full-length pre-fusion F obtain structures to identify candidates that elicit more potent immune responses vs. first-generation stabilized proteins by a Y3Q2 Go/No-Go. Quantifiable metrics: Antigen stability/production quality: >10 mg/liter; stability at 4 °C (>90% retention of activity at 30 days). If go, Applicants can test stability at 40 °C (>45% retention of activity at 30 days). Stability can be assessed for lyophilized and solution forms of the antigen. Antigen epitope display: Retains all key complex, conformational epitopes (by ELISA or BLI) that are defined using neutralizing or protective mAbs. Immunogenicity characteristics: Elicits >1/10,000 titer of serum IgG binding to immunogen; >0.5% of cells produce IFNγ after peptide restimulation (T cell response); no reactogenicity in mice and cotton rat (weight loss of <5% after immunization). If stabilized immunogens do not meet these metrics, Applicants can focus on using the current stabilized F by a Y3Q4 Go/No-Go.

Isolate and characterize the mouse-directed mAb response, identify potent nAbs, structures of F complexed with mAbs, and identify the best mAbs or cocktail in cotton rats. Quantifiable metrics of prioritized mAbs: Neutralizing activity: EC_{50<10} ng/ml. Binding affinity: K_{D}<5 nM. Protein stability/quality: >100 mg/liter; stability at 4 °C or −80 °C (>90% activity at 30 days). Synergy: Combinations of mAbs can show synergy or additive inhibition (no antagonism). Isolation of mAb with high genetic barriers to resistance must occur by a Y2Q4 Go/No-Go. Structures must be generated by a Y3Q4 Go/No-Go.

Aim 2: Efficacy of the best mAbs or cocktail in cotton rats. Quantifiable metrics: Efficacy in cotton rats: Reduction in virus growth in nose and lung by >2 logs prophylactically and therapeutically. In vivo protection must be demonstrated by a Y3Q4 Go/No-Go. Eliciting protective immunity in vivo: Identify the optimal form (ectodomain or full-length) of MeV F and choose among the top antigens for protecting cotton rats with novel MeV candidate vaccines. Eliciting high titer response in cotton rats by Y3Q4 Go/No-Go. Protection of cotton rats from MeV challenge [measuring if 1 μg enough or higher (e.g.,5 μg) is necessary] by Y4Q3Go/No-Go. Quantifiable metrics: Reduction of viral titers at least 100-fold in lung tissue. Absence of vaccine-enhanced disease by Y4Q4 Go/No-Go. Is one dose enough (highest dose possible without negative effect can be tested) Y5Q3 Go/No-Go.

### Example 4—MeV F Protein Stabilization Strategy Based on Viral Evolution

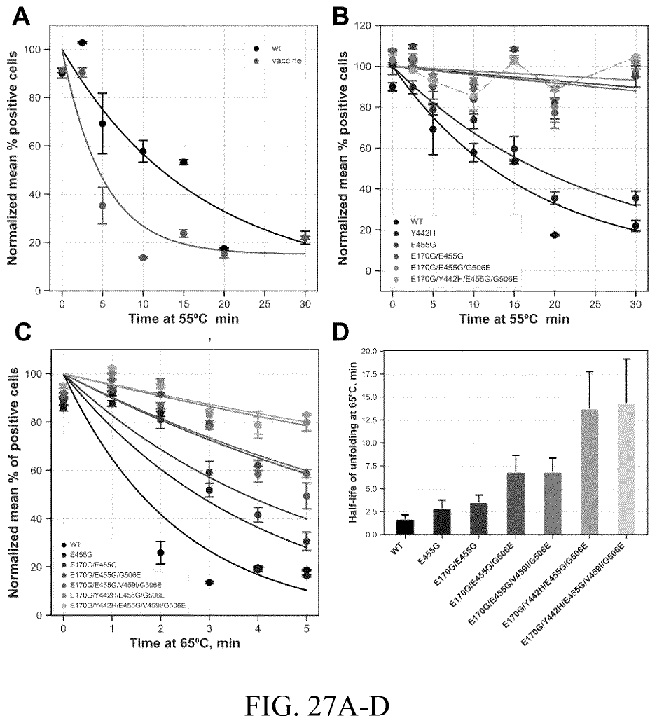

The current live measles virus was obtained via passage of wt virus on cells not expressing the native cellular receptors (CD150 and Nectin-4), resulting in alteration in the H-F fusion complex. Applicants previously observed that the vaccine-derived F is more easily activated than the wt F when paired with the vaccine-derived H bound to the cellular receptor CD46 (Jurgens, et al., 2015). Applicants asked whether this proficiency to promote fusion could inversely correlate with the thermal stability of the vaccine-derived F protein. The F protein from vaccine strain Edmonston-Enders (Genebank: AF266287.1), previously known as Moraten, is characterized by a longer signal peptide due to the insertion of Met-Ser-Ile residues before the initial methionine (M) of the wt strain. Additionally, this strain harbors three-point mutations at positions R263G, S362Y, and T429I compared to wt. In FIG. 27A, Applicants exposed the wt and vaccine F proteins at 55 °C over time and assessed the presence of the pre-fusion state. The presence of the pre-fusion epitope was measured by cell-based fluorescent high-content image analysis, as described in previous work. Applicants found that after 10 minutes at 55 °C the vaccine Fin pre-fusion state could not be detected anymore, while 50% of wt F pre-fusion epitope was still present. These data indicate that the wt F protein is significantly more thermostable than the vaccine-derived F. Applicants hypothesized that by introducing in the wt F background mutations that increase the temperature at which the pre-fusion epitope is still present, Applicants would obtain a full-length F (F_{FL}) that could be solubilized and used as a subunit vaccine. To obtain stable purified MeV F_{FL} proteins, Applicants started with previously described mutations (2M, E170G, and E455G) and combined these with additional stabilizing substitutions selected during viral evolution (Y442H, V459I, and G506E). Applicants first assess the thermostability of full-length MeV containing various combinations of stabilizing mutations on cells transfected with F-containing the indicated mutations. These F proteins expressing cells were then exposed to 55 °C and 65 °C for selected time intervals (see FIG. 27B-D). The stability half-lives are shown in FIG. 27D and Table 8. The data shows that when expressed on the cell surface, the F bearing E170G and E455G was highly stable and that the F bearing all the stabilizing mutations (E170G, Y442H, E455G, V459I, and G506E) was the most stable. Note that references in this application to antibody 77 refer to either of antibody 77.1 and antibody 77.4, which are interchangeable for purposes of this disclosure.

Table 8. Thermal stability of the MeV F mutant proteins detected by the binding of a mouse monoclonal antibody (mAb 77.4 (Mathieu, et al., 2021)) specific for the pre-fusion conformation of F in cell-based ELISA. Data is reported as an average of three independent experiments with the standard error and were fitted with single exponential function fit (Eq 1), and stability is expressed as the half-life (minutes) at 65 °C (Eq 2).

f(x)=A₁·e^{-x/t1}+y₀ Eq 1

t1/2=t1·ln(2) Eq 2

| | |
|---|---|
| | Stability half-life at |
| MeV F (mutations) | 65 °C ± standard error |

| | | |
|---|---|---|
| wt | 1.64 ± 0.50 | min |
| E455G | 2.82 ± 0.93 | min |
| E170G/E455G (F_{FL} 2M) | 3.47 ± 0.83 | min |
| E170G/E455G/G506E | 6.77 ± 1.86 | min |
| E170G/E455G/V459I/G506E | 6.78 ± 1.55 | min |
| E170G/Y442H/E455G/G506E | 13.70 ± 4.07 | min |
| E170G/Y442H/E455G/V459I/G506E | 14.30 ± 4.82 | min |

The MeV Vaccine Strategy Relies on Purifying a Full-Length MeV F Protein that is Stable in the Pre-Fusion State.

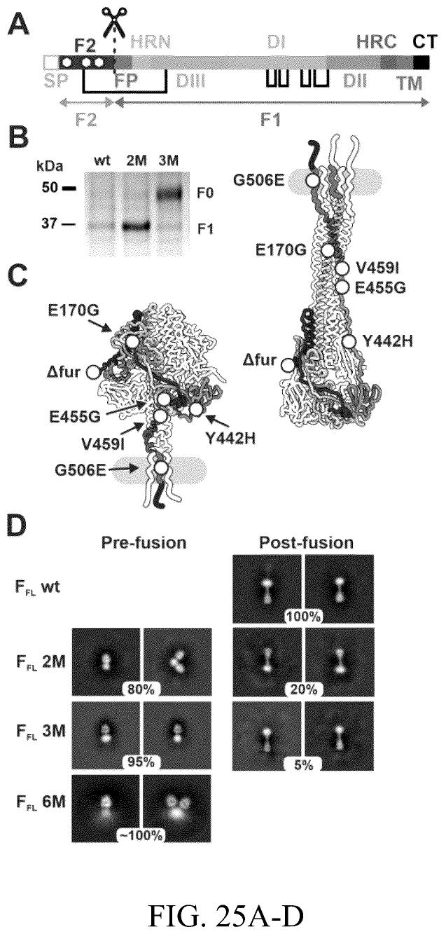

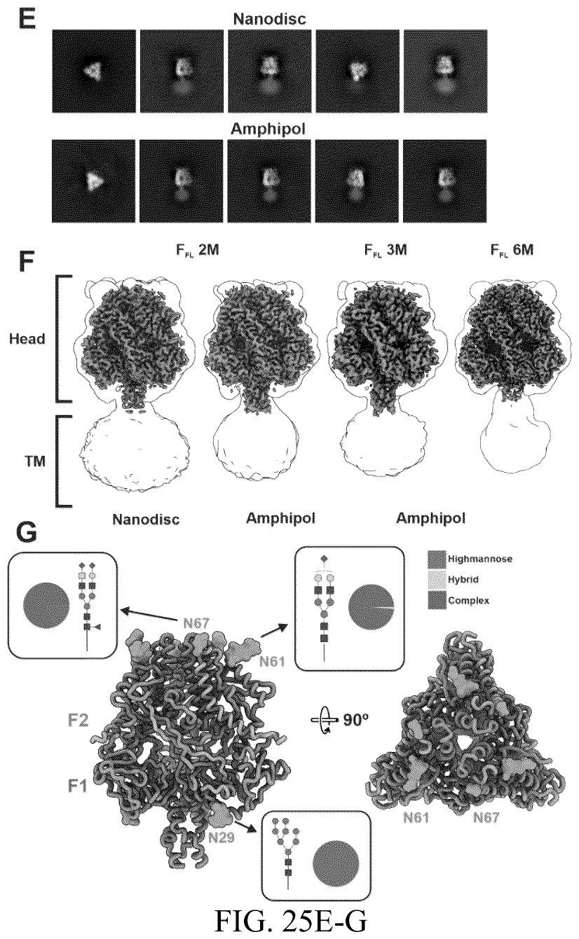

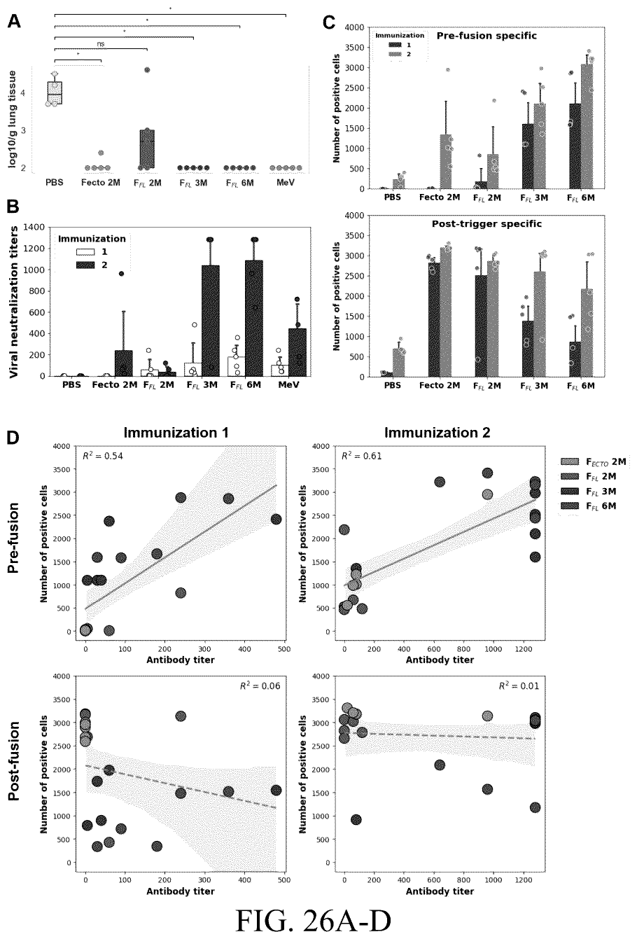

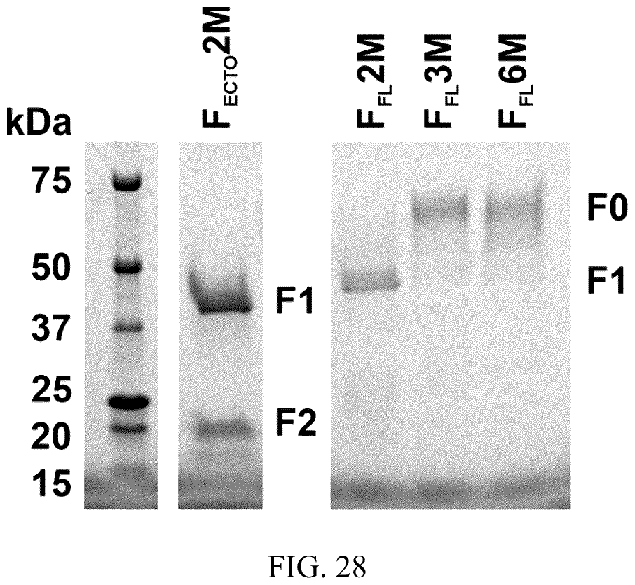

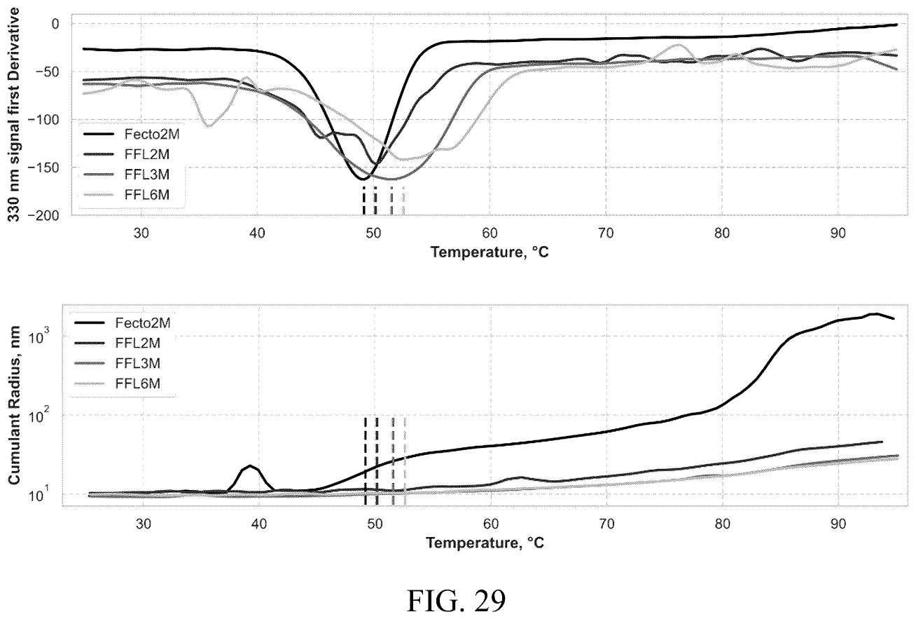

Based on the cell surface thermostability findings described above, Applicants designed MeV F proteins with the two previously identified stabilizing mutations (E170G and E455G) as well as with three additional stabilizing mutations (Y442H, V459I, and G506E). Applicants also introduced a mutation that removed the furin cleavage site (Δfur) from each of the F constructs. By removing the cleavage site, Applicants prevented the exposure of the fusion peptide (FP). These MeV F_{FL} constructs were named F_{FL} 2M (E170G and E455G), F_{FL} 3M (Δfur, E170G and E455G), and F_{FL} 6M (Δfur, E170G, Y442H, E455G, V459I, and G506E) (FIG. 26A-C and FIG. 28A, gel analysis of these constructs). The structural integrity and stability of the purified constructs were determined using negative stain electron microscopy (NS EM) and nano differential scanning fluorimetry (nano-DSF) coupled with dynamic light scattering (DLS). This analysis revealed the additional point mutations provided further stabilization of F. Their stabilization in pre-fusion conformations were also supported by reduced proportions of particles in post-fusion conformation as well as increased apparent melting temperature (Tₘ³) and reduced hydrodynamic radius in the function of temperature (FIG. 25D, FIG. 29).

A comparative analysis of the F_{FL} 2M construct with the F_{FL} wild type (wt) demonstrated that approximately 80% of the particles remained in the pre-fusion conformation (FIG. 25D). Abolishing the furin cleavage site further increased the stability, resulting (F_{FL} 3M) in 95% of the particles maintaining a pre-fusion state (FIG. 25D). The mechanism underlying the formation of the post-fusion conformation for Δfur constructs remains elusive, albeit the human parainfluenza virus type 3 Δfur F protein structure was also in the post-fusion state indicating that uncleaved F can fold to a post-fusion state. In addition, Applicants observed that full-length constructs demonstrated smaller hydrodynamic radius upon thermal unfolding which was further improved by abolishing the Furin cleavage site (FIG. 29). For the F_{FL} 6M construct, NS EM analysis did not detect any particles in a post-fusion state. In addition, the F_{FL} proteins exhibited a tendency to incorporate multiple copies within a single detergent micelle. This phenomenon was particularly notable in the F_{FL} 6M construct, where the insertion of multiple F proteins into a single micelle was more pronounced. However, it is important to note that Applicants did not observe the formation of an F-protein array, a structural arrangement previously reported for the Nipah F protein.

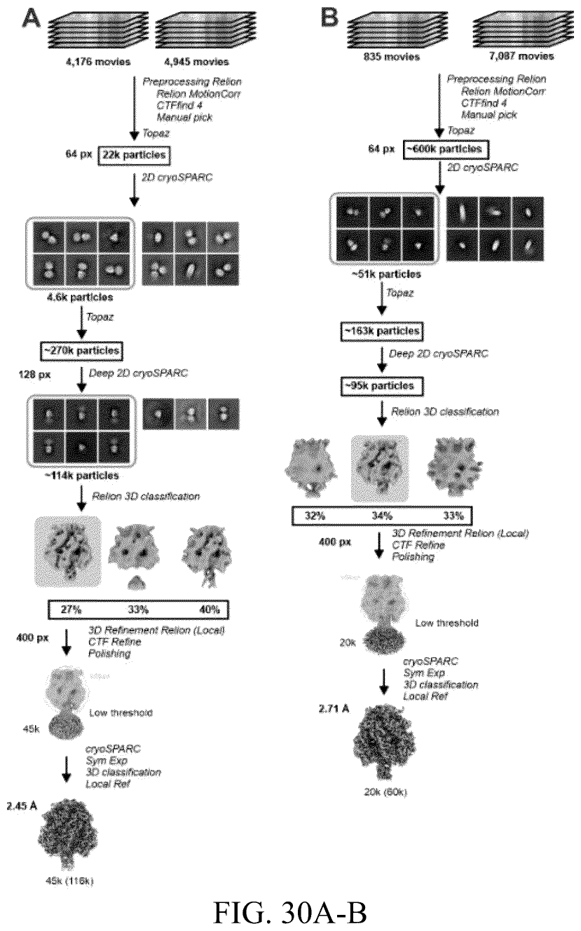

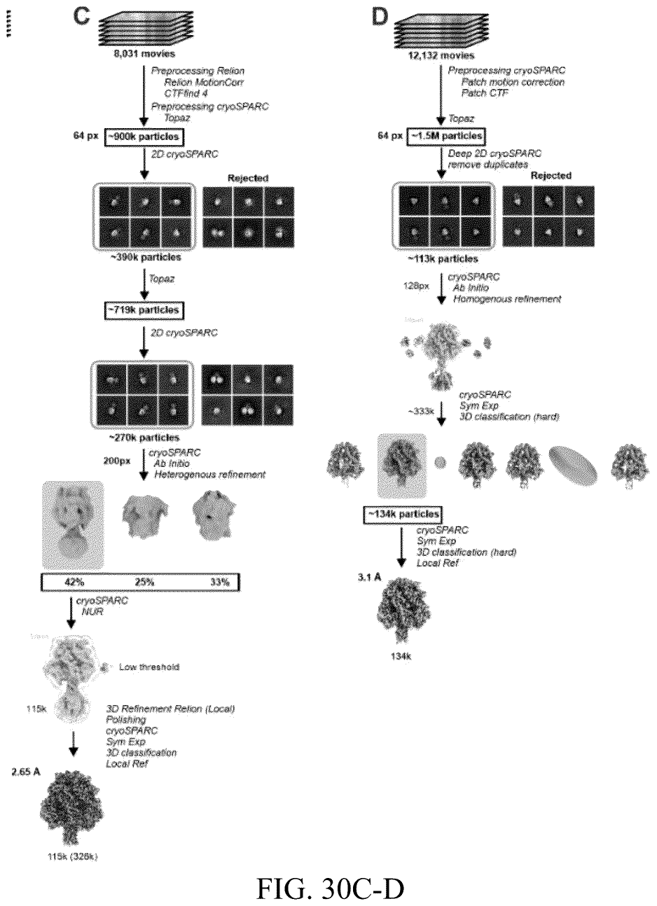

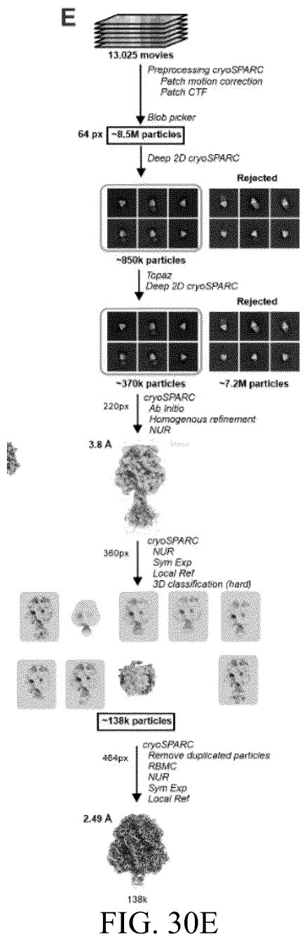

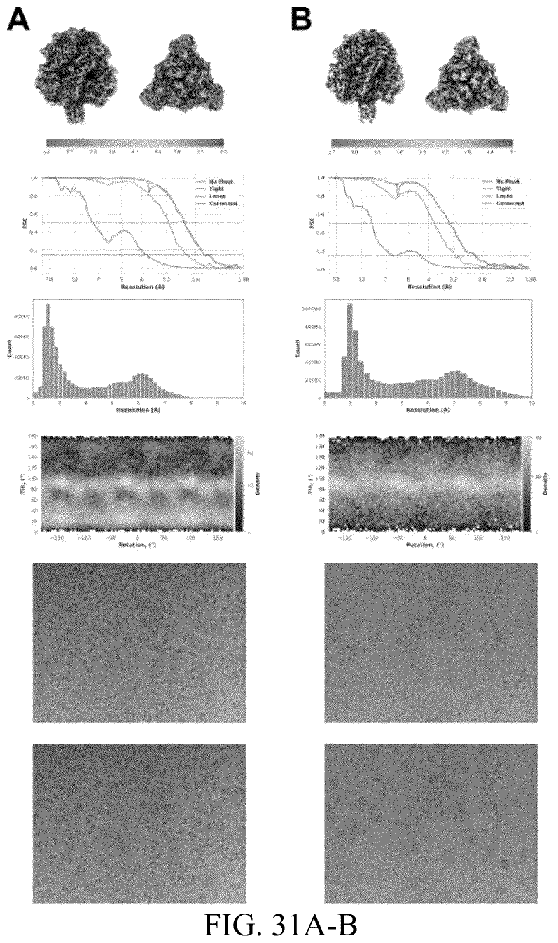

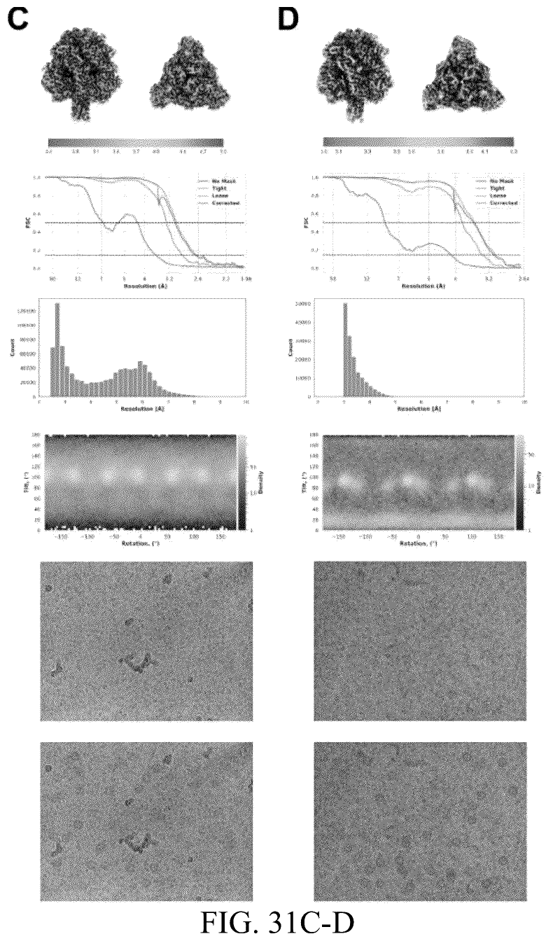

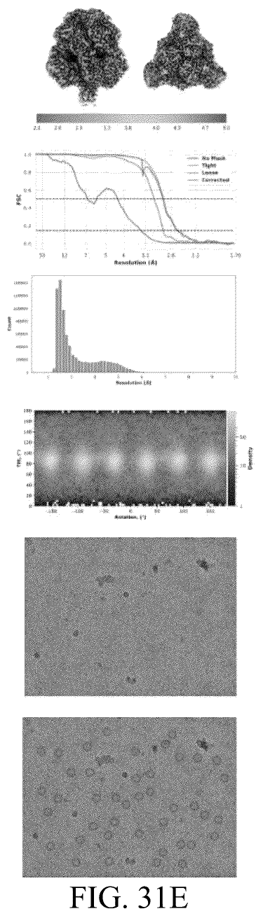

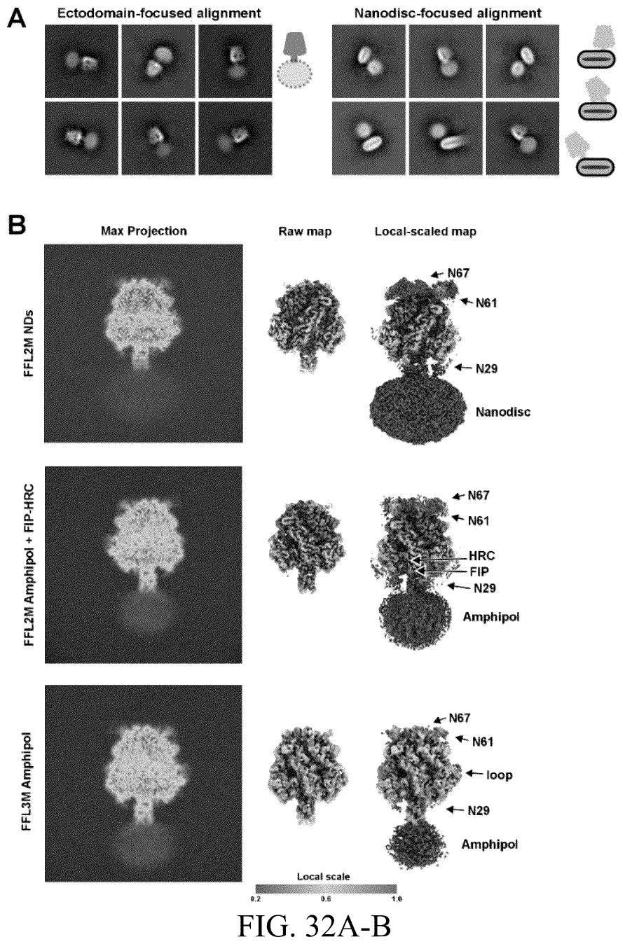

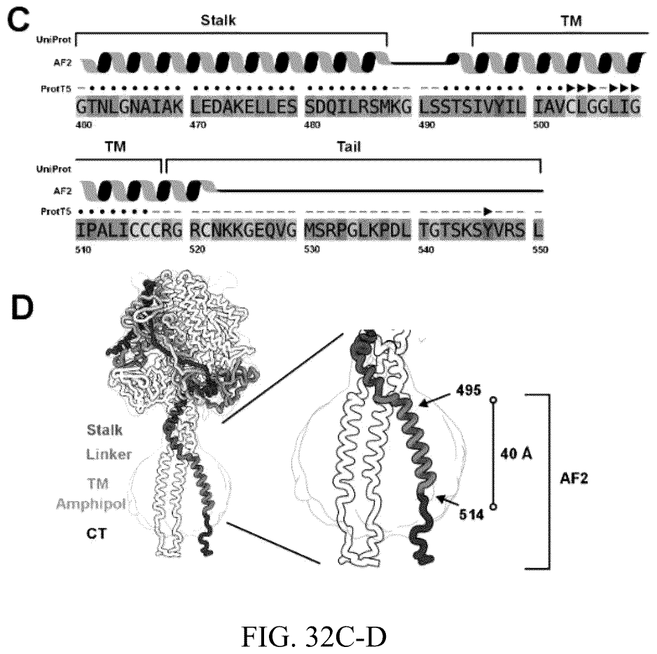

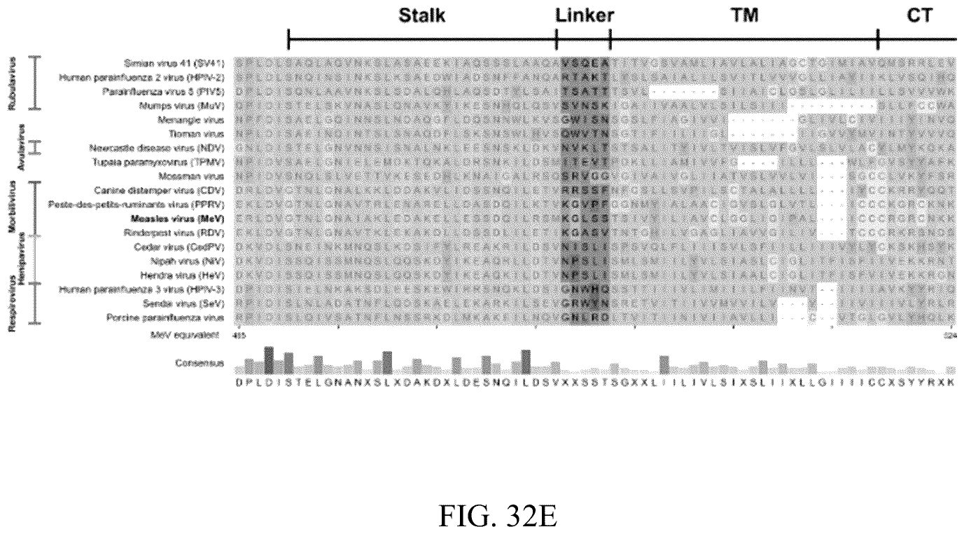

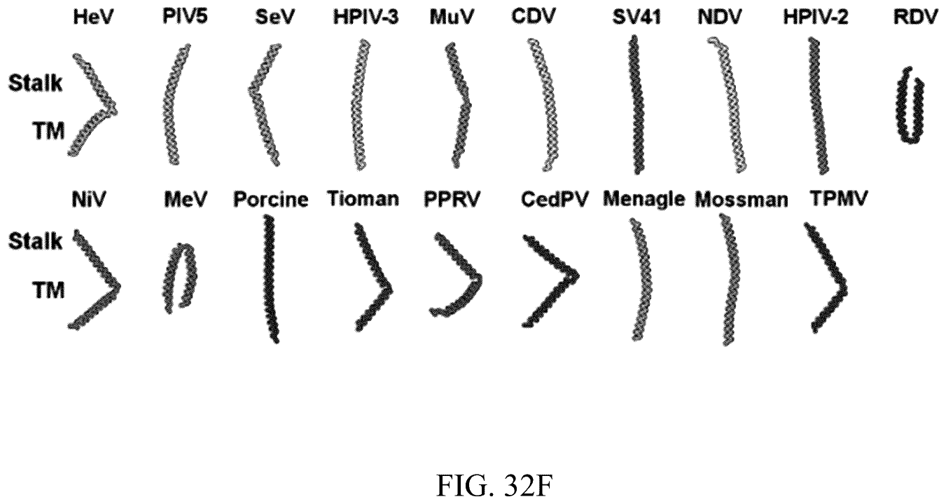

Applicants used nanodisc to stabilize the transmembrane region for high-resolution structure determination. The proteins were vitrified, and cryo-EM data acquisition was conducted as previously described [Science in press]. Initial structural analysis of the F_{FL} 2M construct within nanodiscs failed to provide detailed insights into the transmembrane region, despite the application of focused refinement and classification strategies (FIG. 29A, FIG. 30A). To overcome this limitation, Applicants employed two alternative approaches: stabilizing the glycoprotein with the MeV F pre-fusion stabilizing peptide ([FIP-HRC]₂-PEG₁₁) and -replacing nanodiscs with amphipol to reduce positional heterogeneity of the transmembrane region (FIG. 25E-F). Nevertheless, these methods could not resolve the transmembrane region of the F protein either (FIG. 25E-F). Applicants observed a high conformational heterogeneity of the head domains in relation to the nanodisc in the 2D classes, limiting the structural reconstruction to just the head domain (FIG. 32A). Focused classification on transmembrane region and various flexibility addressing pipelines also did not succeed in providing any information on the transmembrane region, implying that either structural heterogeneity is too high, or the transmembrane (TM) region of MeV F lacks a defined quaternary structure, compared to other viral proteins, such as the influenza HA protein.

Applicants noted similar observations for other full-length constructs that were resolved in amphipol: F_{FL} 3M and F_{FL} 6M (FIG. 32B). They were also missing a resolvable density for the transmembrane region (FIG. 32B).

The analysis of the F_{FL} 2M, F_{FL} 3M, and F_{FL} 6M constructs in amphipol revealed partial density in the 488-491 residue region. This observation suggests a discontinuity in the stalk helix prior to the commencement of the transmembrane helix (FIG. 32B). The residues in this region are likely contributors to increased flexibility, potentially leading to the interruption of the helical structure between stalk and transmembrane region. Supporting this hypothesis, the computational modeling using AlphaFold2 (AF2) and secondary structure predictions from the protein large language model ProtT5 indicated that the 488-491 region is non-helical, delineating a clear division between the stalk and transmembrane helices (FIG. 32C). Consequently, Applicants constructed a theoretical model of the transmembrane region, based on the partial density and AF2 predictions (FIG. 32D). The obtained hybrid model suggests that the flexible loop situated between the stalk and transmembrane region may impede the formation of a three-helix bundle within the lipid bilayer. This model also provides an explanation for the observed high flexibility of the F protein in the 2D classes within nanodiscs and amphipol, attributable to the loop in the membrane-proximal region of F.

Next, Applicants aligned the F protein sequences from representative members of the Paramyxoviridae family and identified the presence of similar flexible linkers in various viral species (FIG. 32E). Based on this multiple sequence alignment, Applicants extracted sequences corresponding to the stalk, loop, and transmembrane regions of other paramyxoviruses (FIG. 32E). These sequences were then used for secondary structure predictions using AF2 (FIG. 32F). The analysis revealed that the separation between the stalk and transmembrane regions is a relatively common feature in the Paramyxoviridae family. This structural characteristic is not only observed in MeV but is also predicted in other paramyxoviruses, including Tupaia Paramyxovirus (TPMV), Cedar Virus (CedPV), Peste des Petits Ruminants Virus (PPRV), Nipah Virus (NiV), Hendra Virus (HeV), Rinderpest Virus (RDV), Sendai Virus (SeV). Conversely, there is no apparent helix separation in Parainfluenza Virus 5 (PIV5), Canine Distemper Virus (CDV), Simian Virus 41 (SV41), Newcastle Disease Virus (NDV), Human Parainfluenza Virus 2 (HPIV-2), porcine paramyxovirus, Menangle, and Mossman (FIG. 32F). The specific functional role of this flexible loop remains to be elucidated. However, it might play a role in expanding the range of interaction between the F protein and the host membrane, potentially enhancing the angular range of the interaction between the virus and the host membrane.

In earlier work, Applicants described the ectodomain of the F protein as exhibiting a superhelical structure in the stalk region, with additional coiling starting at residue 474. This characteristic is also evident in the F_{FL} structures, where the secondary structure in this region mirrors that of the ectodomain construct. This observation suggests that ectodomain constructs lacking the trimerization domain adopt a conformation similar to that of the F_{FL} protein. Furthermore, the comparison between the previously resolved ectodomain and the F_{FL} structures obtained in this study revealed no significant structural variations, except for minor differences observed in the flexible loop region including residues 165-172.

### MeV F Glycosylation

To explore the potential impact of glycosylation patterns on epitope availability, Applicants used mass spectrometry to characterize such patterns present on F_{FL}. While Applicants previously expressed F ectodomain in insect cells and obtained the glycosylation patterns present in Drosophila S2 expressed protein, here Applicants expressed F in mammalian HEK293F cells to yield a glycosylation pattern analogous to that found on the native virus. Applicants found that N29 is exclusively high mannose, possibly due to obstructed access of the site after protein folding. For N61 and N67, Applicants identified complex-type glycans; N61 has only a single N-Acetylneuraminic acid and N67 with a fucose at first N-acetylglucosamine (FIG. 25G). The glycosylation patterns obtained from HEK293F cells are in agreement with the previously shown patterns Applicants showed for S2 expressed cells. In summary, MeV F has 7.6 kDa of glycans per single protomer, resulting in ~12% glycan mass fraction. This is relatively low compared to other viral glycoproteins such as HIV gp160 (42%), Lassa GP (33%), Mumps (22%), or Nipah (20%).

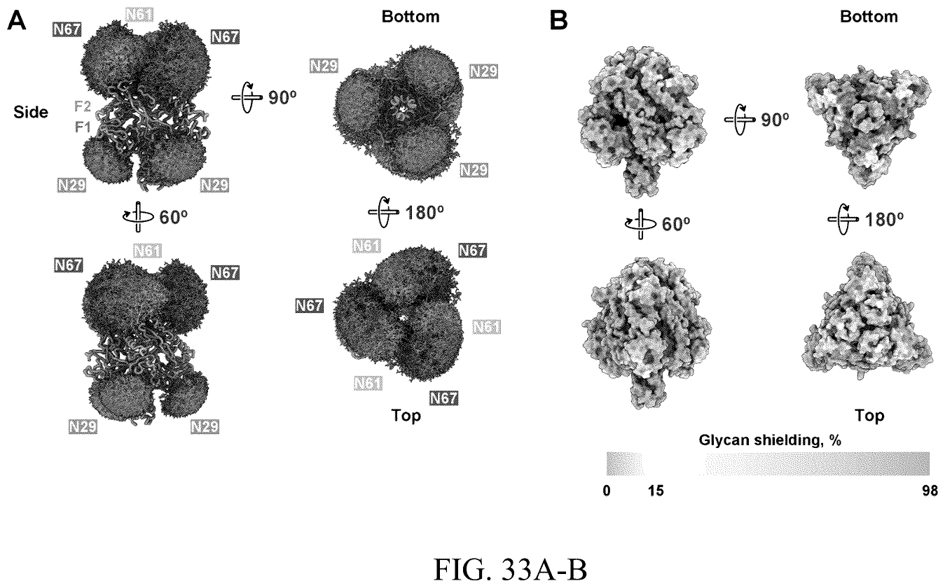

Additionally, Applicants explored the extent of shielding afforded by the three glycans present on MeV F. The apex of F is predominantly shielded by N61 and N67, leaving only a fraction of the apex accessible to neutralizing antibodies (FIG. 33A). The spatial arrangement of these glycans results in a complete exposure of the glycoprotein's face, rendering it a potentially viable target for antibodies (FIG. 33A). The proximity of the glycans to the glycosylation site is closely linked to the degree of protection that these glycans, with shielding percentages ranging from 15 to 98% (FIG. 33B).

### F_{FL} as Vaccine

Previous vaccine development efforts, particularly for the Respiratory Syncytial Virus (RSV), have demonstrated that full-length glycoprotein constructs afford considerable protection. However, recent advancements in RSV vaccine research have led to the approval of vaccines that use the ectodomain of the F protein stabilized in its pre-fusion conformation. Given the increased in vitro stability of full-length constructs of the MeV constructs, which potentially reveal more immunologically relevant epitopes compared to their ectodomain counterparts, Applicants wanted to explore the in vivo immunogenicity of the F mutants characterized above, Applicants immunized cotton rats using the three different F proteins (F_{FL} 2M, F_{FL} 3M, and F_{FL} 6M, 5 μg/animal/dose for two doses) or with a live virus vaccine (Edmonston-Zagreb MeV vaccine strain, as a comparator; FIG. 26). Following the immunization (see specific protocol in methods), animals were challenged with wt B3-eGFP measles virus. Immunization with each of the three F proteins resulted in protective immunity in vivo. Immunized animals were protected against the subsequent viral challenge (FIG. 26A). Interestingly, in assessing the development of neutralizing antibodies after the first and second immunization (FIG. 26B), Applicants found that immunization with of the most stable F proteins (F_{FL} 3M or F_{FL} 6M, see FIG. 25D) resulted in higher neutralization titers compared to the immunization with MeV vaccine. Applicants then proceeded to assess whether the serum antibodies from the cotton rats were specific for the pre-fusion or post-triggered F protein (FIG. 26C) using an assay similar to that used for the data in Table 8 and published work. The two most stable F proteins (F_{FL} 3M and F_{FL} 6M) mainly induced antibodies that target the pre-fusion F. Based on this and the robust viral neutralization activity shown in FIG. 26B, Applicants demonstrated that proteins in pre-fusion state induce strong humoral immunity.

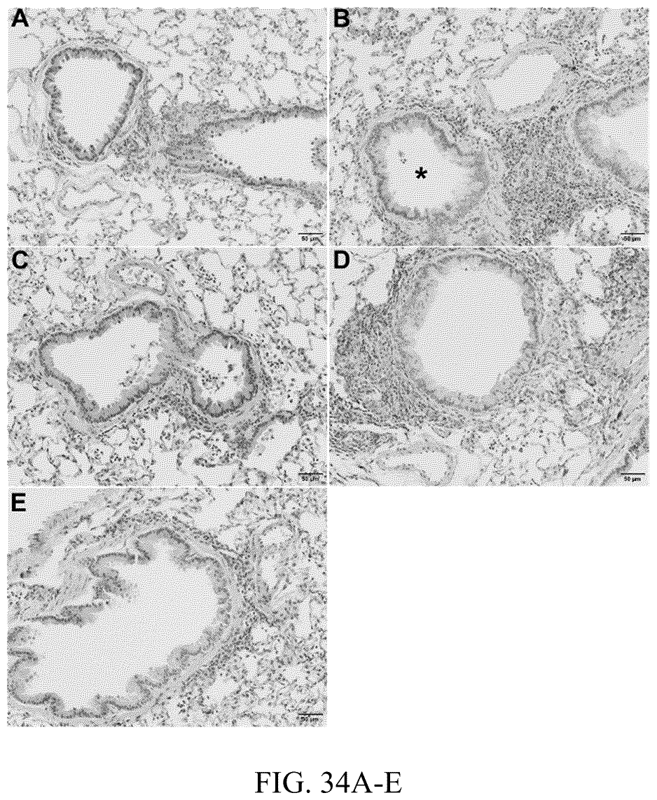

Because atypical measles or vaccine-enhanced disease has been observed in children immunized with formalin (FA)-inactivated MeV, Applicants assessed the risk of enhanced disease following vaccination with the subunit protein and subsequent viral challenge. Atypical measles has been described in animal models histologically as severe pneumonitis. Applicants performed histological analysis of the lungs of vaccinated cotton rats similar to previously described analysis and assessed lung damage after primary infection or after challenge of immunized cotton rats in the experiment shown in FIG. 26. On day 4 after infection, the left lung lobe was harvested for virus titration (FIG. 26A). Right and accessory lobes were stained with hematoxylin and eosin (H&E). All lung sections were evaluated histologically for peribronchiolitis, perivasculitis, interstitial pneumonitis, and alveolitis. Each parameter was scored using semi-quantitative scales modified from studies of vaccine-enhanced respiratory syncytial virus disease studies. In the animals vaccinated with the subunit proteins, no vaccine-enhanced disease was observed by histological analysis after the subsequent challenge with the virus (See, FIG. 34). The quantity and severity of peribronchiolar inflammatory infiltrates did not differ between animals vaccinated with the subunit proteins and those vaccinated with Edmonston-Zagreb, and substantial perivasculitis and interstitial pneumonitis were not observed in any vaccinated animals (Table 9).

TABLE 9
Semiquantitative histopathologic scoring of inflammatory lesions following measles
challenge for severity (0-3) and quantity (0-4) of inflammatory infiltrates.

| | Treatment Group; mean (standard deviation) | | | | | |
|---|---|---|---|---|---|---|
| | Peribronchioloar | | Perivascular | | Interstitial | Alveolar |
| | Infiltrates | | Infiltrates | | Infiltrates | Infiltrates |
| | Quantity | Severity | Quantity | Severity | Quantity | Quantity |
| Vehicle | 0.75 | 0.75 | 0 | 0 | 0 | 0 |
| | (0.5) | (0.5) | (0) | (0) | (0) | (0) |
| Edmonston- | 2 | 1.4 | 1.2 | 1.2 | 0.2 | 0.2 |
| Zagreb | (1) | (0.55) | (0.45) | (0.45) | (0.45) | (0.45) |
| F_{FL} 2M | 1.8 | 1.2 | 0.8 | 0.8 | 0.4 | 0.6 |
| | (0.84) | (0.45) | (0.84) | (0.84) | (0.55) | (0.55) |
| F_{FL} 3M | 1.2 | 1.2 | 0.8 | 0.8 | 0 | 0 |
| | (0.45) | (0.45) | (0.45) | (0.45) | (0) | (0) |
| F_{FL} 6M | 1.8 | 1.4 | 0.6 | 0.6 | 0 | 0 |
| | (0.45) | (0.55) | (0.55) | (0.55) | (0) | (0) |

### DISCUSSION

The data presented show that Applicants have acquired structural data for the full-length constructs of stabilized MeV F protein and demonstrated the efficacy of the stabilized F proteins as a recombinant subunit vaccine strategy with an excellent safety profile in cotton rats. Applicants propose developing an active immunization strategy that is based on guiding the immune system to target the pre-fusion state of MeV F. While this strategy is specifically tailored for the individual who cannot get vaccinated with the current live vaccine, it could also be used to boost immunity in previously immunized people. The thermostability of the subunit vaccine described here could also represent an important feature for the global eradication of measles since the recombinant subunit vaccine could be easily transported and stored without the need for cold chain distribution, which could be limited in some rural areas.

The availability of a live attenuated vaccine means that most of the U.S. population has not been exposed to wild-type MeV. In the absence of live virus exposure, the level of neutralizing antibodies in the overall population (herd) decreases over time, increasing the susceptibility of even countries with high vaccination coverage to larger outbreaks. This increasing susceptibility poses a particular threat to those who are too young or too vulnerable immunologically to be immunized by the live vaccine. The live-attenuated MeV vaccine nearly led to the successful eradication of MeV in the late 20th century. Despite this significant progress, measles outbreaks continue to occur. The growing number of immune-compromised individuals, who are most at risk of MeV infection and who cannot be vaccinated themselves, are at the highest risk of infection during MeV outbreaks. An alternative vaccine in the form of a stabilized recombinant subunit is an ideal and urgently needed substitute for these individuals.

Additionally, the current live vaccine mostly elicits antibodies against hemagglutinin (H): 80% of the human sera antibodies are specific for H while only 20% are specific for F, and the anti-F antibodies are not strongly neutralizing. While the lack of neutralization activity of the anti-F antibodies cannot be directly correlated to the vaccine F stability, Applicants speculate it could contribute to the shortcomings of the anti-F sera. These factors demonstrate a gap in vaccine efficacy. Based on the prior characterization of MeV F mutations observed in patients, Applicants selected a combination of amino acid substitutions that stabilized the pre-fusion state of MeV F, eliciting neutralizing antibodies that appeared to be even more effective in protecting from MeV infection in animal model (FIG. 26). The data show that immunization with pre-fusion F protein results in in vivo protection and elicits potent neutralizing antibodies without the need for anti-H (FIG. 26). The neutralizing titer from animals immunized with the most stabilized F proteins is higher than the titer induced by the live MeV vaccine. For the related respiratory syncytial virus (RSV), stabilized pre-fusion F ectodomains alone have been recently approved for clinical, providing a strong rationale for the strategy. Several stabilized forms of RSV fusion protein are in clinical evaluation of efficacy and safety, and two (Abrysvo, Pfizer and Arexvy, GSK) are in clinical use. For RSV, only the pre-fusion state of the F protein induces neutralizing antibodies, whilst the post-fusion state does not. This finding starkly contrasts with the related human metapneumovirus (HMPV), for which neutralizing antibodies in vivo are directed against both the pre- and post-fusion states of the HMPV F. In the instant case, Applicants show that MeV neutralization correlates with antibodies against the pre-fusion state of F (as in the RSV case) (FIG. 26D). Notably, immunization with the F_{FL} 2M (with lower F in pre-fusion state stability), resulted in low neutralizing titer and yet it offered in vivo protection. Applicants speculate the in vivo protection may be due to cell-mediated immunity, a hypothesis that can be tested in future work.

The F thermostability would represent a boon to MeV global eradication, particularly in countries where a lack of or insufficient cold chains compromise vaccine access and delivery. This issue has not been solved by the dry formulation of the current live vaccine. Applicants also propose that an F-based vaccine could be utilized in children younger than one year old (when usually the live vaccine is administered) without risking low efficacy due to the presence of fusion protein specific maternal antibodies elicited by the live vaccine and as a boost in cases of waning immunity. These hypotheses can be tested in follow-up experiments. Applicants expect this strategy will fill specific unmet needs for those at risk for MeV infection who cannot be vaccinated or for whom a thermostable vaccine would improve access.

### Methods

Plasmids. The genes of Measles morbillivirus (MeV) strains IC323, B3, Schwarz hemagglutinin protein (H), fusion protein (F), human nectin 4, and human CD150 proteins were codon optimized, synthesized, and sub-cloned into the mammalian expression vector pCAGGS (Epochlifescience). A codon-optimized fusion protein sequence derived from the Measles virus strain Ichinose-B95a (Taxonomy ID: 645098, Uniprot ID: Q786F3) was synthesized by GeneScript and subcloned into the pcDNA3.1 vector. The FL expression constructs were generated by cloning the F ORF into a pCG plasmid (Addgene ID #84817 (10.1038/gt.2014.125)) and adding a C-terminal twin Strep-Tag II using the NEBuilder HiFi DNA Assembly. Stabilizing point mutations previously characterized in SSPE and MIBE Measles strains, including E170G, Y442H, V459I, E455G, G506E and Δfur, were introduced sequentially using the Q5 Site-Directed Mutagenesis kit. The Δfur mutation changes the furin cleavage site from RHKR to NHNR, as previously described.

Cells. HEK293T (human kidney epithelial), Vero, and Vero-SLAM/CD150 (African green monkey kidney) cells were grown in Dulbecco's modified Eagle's medium (DMEM; Life Technologies; Thermo Fisher Scientific) supplemented with 10% fetal bovine serum (FBS, Life Technologies; Thermo Fisher Scientific) and antibiotics at 37 °C in 5% CO₂. The Vero-SLAM/CD150 culture media were supplemented with 1 mg/ml Geneticin (Thermo Fisher Scientific).

HEK 293F cells were cultured in a humidified incubator at 37 °C with a 5% CO₂ atmosphere in FreeStyle™ 293 Expression Medium (Gibco). Cells were passaged every 3 to 4 days to maintain optimal cell density and split to 0.5 M/ml in 30 ml medium. Cells were maintained and used up to 25th passage.

Protein expression and purification. HEK293F cells were expanded to a volume of at least 500 mL, to a density of approximately 1×10⁶ cells/mL on the day of transfection. Transfection was performed using the pCG-F_{FL} variant construct at a concentration of 1 μg/mL, using polyethylenimine (PEI, Polysciences) at a 3-fold excess relative to the DNA amount, following a standard protocol. The cells were then cultured for five days, and on the morning of the fifth day, they were centrifuged at 4000 g in a 500 mL centrifuge tube. The resulting cell pellet was resuspended in a volume equivalent to one-tenth of the initial culture (50 mL) in ice-cold HBSE buffer (50 mM HEPES, pH 8.0; 150 mM NaCl; 1 mM EDTA). To this suspension, 200 μL of protease inhibitor cocktail (PIC), 200 μL of Biolock, and 1.5 μL of Benzonaze were added, and the mixture was kept on ice. The cell suspension was then transferred to a 15 ml douncer and homogenized by gently moving the piston up and down at least ten times. The homogenized cells were collected into a 50 mL Falcon tube, to which NP-40 (ThermoFisher) was added to achieve a final concentration of 2%. The cells were incubated at 4 °C for 2 hours with gentle rocking at approximately 1 Hz. This solubilized solution was then transferred into 30 mL tubes and the insoluble fraction was pelleted by centrifugation at 38,000 g using the SA 20.1 rotor at 4 °C for 30 minutes. The supernatant was collected and incubated overnight with 1 mL of Streptactin resin (Qiagen), previously equilibrated with HBSE, at 4 °C with rocking. This mixture was then diluted to 50 mL with HBSE to reduce the final NP-40 concentration. The following day, the mixture was applied to a gravity Bio-Rad column and the resin was washed with 10 mL of HBSE containing 0.06% NP-40 to remove excess NP-40 and wash out contaminants. For nanodisc assembly protein was eluted in HBSE containing 0.06% NP-40 and 5 mM d-desthiobiotin (Sigma). Alternatively, for amphipol embedding, 1 ml of HBSE containing 0.06% NP-40 was added and 30 μL of 100 mg/ml Amphipol (Anatrace) was added and resin slurry was mixed to allow uniform mixing of the amphipol. The mixture was incubated for 2 h at 4 °C with rocking. Once incubation was finished, resin was washed with 3 times with 2 ml of HBSE and protein was eluted with HBSE with 5 mM d-desthiobiotin. The expression yields of FL constructs varied, ranging from 0.2 mg to 0.8 mg per liter of cell culture.

MSPD1 purification. MSP1D1 purification was done based on previously reported protocol. Briefly, the pMSP1D1 plasmid obtained from Addgene was used to heat-shock transform E. coli BL21 (DE3) cells (NEB). The cells were then cultured in 1 mL of LB at 37 °C with shaking at 200 rpm for 1 hour. The transformed cells were subsequently plated on kanamycin-containing agar plates (50 μg/mL) (ThermoFisher) and incubated overnight at 37 °C. A single colony from this plate was used to inoculate 50 mL of LB with kanamycin (50 μg/mL), which was then incubated overnight at 37 °C. This starter culture was added to 2 L Terrific Broth (TB) containing 50 μg/mL kanamycin. The culture was grown at 37 °C until an OD₆₀₀ of 2.5 was reached, induced with 1 mM IPTG, and then the temperature was reduced to 28 °C one hour post-induction. Cells were harvested four hours after induction. For protein purification, the cell pellet was resuspended in lysis buffer (50 mL, 20 mM PBS pH 7.4 with 1% Triton X-100 added post-resuspension) and left on ice for 30 minutes. Lysis was achieved using M-110P pressure cell disruptor (Microfluidics) at 18k PSI at 4 °C. The lysate was then centrifuged (37,000×g, 20 min) and the supernatant applied to a pre-equilibrated 5 ml Ni-NTA resin (Qiagen) at 4 °C. The column was washed with 40 mM Tris, 0.3 M NaCl, 1% Triton X-100 (Anatrace), pH 8.0; followed by 40 mM Tris, 0.3 M NaCl, 50 mM Na-Cholate, 20 mM Imidazole, pH 8.0; and then 40 mM Tris, 0.3 M NaCl, 50 mM Imidazole, pH 8.0. Elution was done using a buffer containing 40 mM Tris, 0.3 M NaCl, 0.4 M Imidazole, 40 mM Na-Cholate, pH 8.0. Protein purity was identified by SDS-PAGE, and concentration was measured using UV spectroscopy at 280 nm, based on a theoretical extinction coefficient of 21430 M⁻¹ cm⁻¹.

Nanodisc assembly. The concentration of the eluted F_{FL} constructs, in HBSE buffer supplemented with 0.06% NP-40 and 5 mM D-desthiobiotin, was determined by measuring the absorbance at 280 nm. Typical concentration values were around 0.1 mg/mL. Due to potential inaccuracies in protein concentration measurements due to the presence of NP-40, nanodisc assembly was conducted with a 5-fold molar excess of nanodiscs relative to the protein trimer. Lipids, specifically POPC and POPG (Avanti), were mixed in an 80:20 molar ratio, extracted from chloroform solutions, and transferred to a glass test tube. The lipids were then dried under an argon stream for 15 minutes. To ensure complete removal of chloroform, the dried lipids were further washed with 1 mL of diethyl ether and subsequently dried for an additional 10 minutes. Then, lipids were resuspended in a 30 mM DDM solution in water, followed by vigorous vortexing until fully solubilized. This solution underwent two freeze-thaw cycles alternating between liquid nitrogen and 37 °C, and was then sonicated for 15 minutes in a water bath to ensure homogenous, clear lipid suspension. The lipid mixture was added to the protein solution at a 300-fold molar excess relative to the protein, and the mixture was incubated for 15 minutes. Subsequently, a 5-fold molar excess of MSPD1 relative to the protein was added, followed by another 15-minute incubation. To remove NP-40, 10% of the total volume of wet Biobeads SM2 (Bio-Rad) was added, and the mixture was incubated overnight with rocking at 1 Hz. The Biobeads were replaced the following day until no change in absorbance at 280 nm was detected, indicating the successful removal of NP-40. Empty nanodiscs were separated from the protein-embedded nanodiscs through size exclusion chromatography using a Superdex 200 10/300 GL column (GE Healthcare). The successful incorporation of the protein into the nanodiscs was verified using SDS-PAGE gel electrophoresis and NS EM.

Protein concentration determination. Protein concentration was determined using absorbance measurements at 280 nm using Nanodrop 2000. The spectrophotometer was blanked on water and the buffer correction was done externally. For molar concentrations given extinction coefficients were used: F_{FL} (all variants): 51,800 cm⁻¹ M⁻¹, F_{ECTO} (all variants): 48,820 cm⁻¹ M⁻¹.

Stability measurements using nano-differential scanning fluorimetry (nano-DSF) and dynamic light scattering (DLS). Stability measurements, expressed as apparent melting temperature and changes in hydrodynamic radius as a function of temperature, were conducted using the Nanotemper Prometheus Panta. Three 10 μL capillaries, each filled with 0.2 mg/mL of protein for each construct, were subjected to a temperature ramp of 1 °C/min from 25 °C to 95 °C. Protein unfolding was monitored through changes in the nano-DSF fluorescent signal, with the first derivative used to determine the apparent melting temperature. Simultaneously, DLS signals were recorded. Data analysis was performed using PR.Panta 1.7.4 software (Nanotemper), and the average of processed data, including the first derivative of the 330 nm signal and the cumulant fit of the DLS data, were plotted using Python.

Grid preparation. The freezing procedure and grid types were consistent for all protein constructs, following the protocol outlined previously (Science in press). Briefly, Quantifoil R2/2 grids had been washed with ethyl acetate the day before freezing. On the day of freezing, graphene oxide (GO, Sigma) a was prepared fresh and applied to the grid. Specifically, 3 μL of a 0.2 mg/ml GO solution was applied and allowed to incubate for 3 minutes, followed by blotting and washing with 20 μL of ddH2O, after which the grid was left to dry for 10 minutes. Within the Vitrobot Mark IV, protein samples at a concentration of 0.15-0.25 mg/ml were applied to the grid at 4 °C and 100% humidity, incubated for 15 seconds, followed by blotting for 3 seconds before being flash-frozen in liquid ethane cooled with liquid nitrogen.

Structure determination F_{FL}. All samples were collected using a Titan Krios microscope operating at 300 kV with a Gatan K3 camera and energy filter set to 20 eV, resulting in a magnification of 130,000× and a pixel size of 0.66 Å/px. The exposure settings were 50 ē/Å2 with an exposure rate of 15 ē/px/s. The approach described in (Science in press) was followed, and recorded movies in TIFF format were motion corrected using Relion 4's own algorithm. CTF values were estimated using CTFfind 4. Initial particle picking was performed using LoG picker inside Relion and followed by 2D classification and Topaz picking. Particles were extracted with binning to 64 pixels, and 2D classification was carried out in cryoSPARC. Particles were then moved back to Relion for initial model generation and 3D classification. The 3D classification was continued until class equilibrium was reached, as monitored by Follow Relion Gracefully program (Science in press +Zyla, D. (2024)). Follow_Relion_gracefully (Version v5) [Computer software]. https://doi.org/10.5281/zenodo.10465899]. Only classes with expected features were selected, and once the Nyquist resolution in the binned data was achieved, particles were unbinned to 128 pixels for another round of 3D classification. The selected 3D class was run in a 3D refinement protocol and the final particle set was unbinned to ~3/4 of the original box size. Further processing involved Refine 3D, CTFrefine, and Baysian Polishing in Relion. Finally, the final particle stacks were imported to cryoSPARC, where Non-Uniform Refinement, symmetry expansion to C3, and Local Refinement were used.

Glycoproteomics LC-MS/MS measurements. The experiments were performed as previously described with minor modifications, as follows. For each sample and protease digestion, approximately 50 ng of peptides were run by online reversed phase chromatography on an Dionex UltiMate 3000 (Thermo Fisher Scientific) coupled to a Thermo Scientific Orbitrap Fusion mass spectrometer. A Poroshell 120 EC C18 (50 cm×75 μm, 2.7 μm, Agilent Technologies) analytical column and a ReproSil-Pur C18 (2 cm×100 μm, 3 μm, Dr. Maisch) trap column were used for peptide separation. Duplicate samples were analyzed with two different mass spectrometry methods, using identical LC-MS parameters and distinct fragmentation schemes. In one method, peptides were subjected to Electron Transfer/Higher-Energy Collision Dissociation (HCD) fragmentation. In the other method, all precursors were subjected to HCD fragmentation, with additional EThcD fragmentation triggered by the presence of glycan reporter oxonium ions. A 90 min LC gradient from 0% to 44% acetonitrile was used to separate peptides at a flow rate of 300 nl/min. Data was acquired in data-dependent mode. Orbitrap Fusion parameters for the full scan MS spectra were as follows: a standard AGC target at 60 000 resolution, scan range 350-2000 m/z, Orbitrap maximum injection time 50 ms. The ten most intense ions (2+ to 8+ ions) were subjected to fragmentation. For the EThcD fragmentation scheme, the supplemental higher energy collision dissociation energy was set at 27%. MS2 spectra were acquired at a resolution of 30,000 with an AGC target of 800%, maximum injection time 250 ms, scan range 120-4000 m/z and dynamic exclusion of 16 s. For the triggered HCD-EThcD method, the LC gradient and MS1 scan parameters were identical. The ten most intense ions (2+ to 8+) were subjected to HCD fragmentation with 30% normalized collision energy from 120-4000 m/z at 30,000 resolution with an AGC target of 100% and a dynamic exclusion window of 16 s. Scans containing any of the following oxonium ions within 20 ppm were followed up with additional EThcD fragmentation with 27% supplemental HCD fragmentation. The triggering reporter ions were: Hex(1) (129.039; 145.0495; 163.0601), PHex(1) (243.0264; 405.0793), HexNAc(1) (138.055; 168.0655; 186.0761), Neu5Ac(1) (274.0921; 292.1027), Hex(1)HexNAc(1) (366.1395), HexNAc(2) (407.166), dHex(1)Hex(1)HexNAc(1) (512.1974), and Hex(1)HexNAc(1)Neu5Ac(1) (657.2349). EThcD spectra were acquired at a resolution of 30,000 with a normalized AGC target of 400%, maximum injection time 250 ms, and scan range 120-4000 m/z.

Glycoproteomics data analysis. The acquired data was analysed using Byonic (v4.5.2) against a custom database of recombinant F_{ECTO} and the proteases used in the experiment, searching for glycan modifications with 12/24 ppm search windows for MS1/MS2, respectively. Up to ten missed cleavages were permitted using C-terminal cleavage at F/Y/W/M/L for chymotrypsin, or T/A/S/V for alpha lytic protease. For N-linked analysis, carbamidomethylation of cysteine was set as fixed modification, oxidation of methionine/tryptophan as variable common 1, and hexose on tryptophan as variable rare 1. N-glycan modifications were set as variable common 2, allowing up to max. 2 variable common and 1 rare modification per peptide. All N-linked glycan databases from Byonic were merged into a single non-redundant list to be included in the database search. All reported glycopeptides in the Byonic result files were manually inspected for quality of fragment assignments (with scores ≥200). All glycopeptide identifications from both EThcD and HCDpdEThcD runs were merged into a single non-redundant list per sequon. Glycans were classified based on HexNAc content as truncated (≤2 HexNAc; <3 Hex), paucimannose (2 HexNAc, 3 Hex), oligomannose (2 HexNAc; >3 Hex), hybrid (3 HexNAc) or complex (>3 HexNAc). Byonic search results were exported to mzIdentML format to build a spectral library in Skyline (v22.2.0.312) and extract peak areas for individual glycoforms from MS1 scans. The full database of variable N-linked glycan modifications from Byonic was manually added to the Skyline project file in XML format. Reported peak areas were pooled based on the number of HexNAc, Fuc or NeuAc residues to distinguish truncated, paucimannose, oligomannose, hybrid, and complex glycosylation, or the degree of fucosylation and sialylation, respectively.

Glycan shielding. Glycan shielding was assessed using the GlycoSHIELD program, with the F_{EL}2M structure serving as the template. For each glycosylation site, the nearest related glycan was assigned: N29 with high mannose, N61 with complex glycan lacking fucose, and N67 with complex glycan containing fucose. The combined structure was used to calculate solvent accessibility using the provided scripts, and figures were generated using ChimeraX.

Statistical analysis. All data analysis and visualizations were conducted using a Python notebook integrating SciPy, Matplotlib, Pandas, Seaborn, and Statannotations package (W. Mc Kinney, Pandas: A foundational python library for data analysis and statistics (2011). www.dlr.de/sc/portaldata/15/resources/dokumente/pyhpc2011/submissions/pyhpc2011_subm ission_9.pdf. F. Charlier, M. Weber, D. Izak, E. Harkin, M. Magnus, J. Lalli, L. Fresnais, M. Chan, N. Markov, O. Amsalem, S. Proost, A. Krasoulis, getzze, S. Repplinger, Trevismd/Statannotations: V0.5 (2022; zenodo.org/record/7213391)). Statistical significance was determined using the Mann-Whitney test. For p-value annotations, the following symbols were used: ‘ns’ indicates p<=1,* represents 0.01<p<=0.05, ** signifies 0.001<p<=0.01, and *** stands for 0.0001<p<=0.001.

Cell surface staining with conformation-specific anti-F mAbs and sera from in vivo immunization. HEK293T cells transiently transfected with viral glycoprotein constructs. The transfected cells were incubated at the temperatures and times indicated in the figures. Specific MeV F conformations were detected with mouse monoclonal antibodies (mAbs) followed by Alexa-488 anti-mouse secondary antibody (Life Technologies) and DAPI (4′,6-diamidino-2-phenylindole; Thermo Fisher) staining. Sera samples were tested starting from 1:50 and following two-fold dilutions. Thereafter, cells were washed with PBS and incubated for 1 h on ice with protein G Alexa-488 conjugate (1:1000; Life Technologies), washed with PBS, and fixed for 10 min on ice with 4% paraformaldehyde (PFA) with a 1:1000 dilution of DAPI (4′,6-diamidino-2-phenylindole; Thermo Fisher) for 60 min. Plates were imaged with a Cytation5, and the relative fluorescence of antibody-bound cells was determined using BioTek Gen5.

Viral rescue. HEK293T cells were transfected using Lipofectamine 2000 on 6-well plates coated with poly-D-lysine. The transfection mixture included cDNA constructs coding for viral proteins N (GS6558900-1), P (GS59482), and L (GS58944), all sourced from Epoch Life Science according to the design specifications. Additionally, cDNA for T7 polymerase (GS58929) and the genome for the MeV B3 eGFP wt virus (GS68838) were included. Cells were incubated overnight at 37 °C in Opti-MEM medium. The medium was replaced with DMEM supplemented with 10% FBS after the overnight incubation. Cells were then subjected to a heat shock treatment by incubating them at 42 °C in a water bath for 3 hours. Subsequently, the cells were returned to a 37 °C incubator for 48 hours. After this incubation, the HEK293T cells were detached and transferred to Vero-SLAM cells in T-75 flasks to facilitate the amplification of the rescued virus. The virus underwent four passages and was titrated to a concentration of 10⁶ plaque-forming units per milliliter (PFU/ml). The virus sample was then sequenced and used in subsequent in vivo experiments. Described in (Science in press).

Neutralization assay (NT). Two-fold serum dilutions with a starting dilution of 1:10 were incubated with 25 TCID₅₀ MeV wt B3-eGFP virus for 1 hour at 37 °C and plated in duplicate onto 10⁴ Vero-huCD150 cells in MEM with 2% newborn calf serum in a 96 well plate. Three days later, viral titers were determined microscopically by cytopathic effect and eGFP expression.

In vivo experiments: Cotton rats. Inbred cotton rats (Sigmodon hispidus) were purchased from Inotiv, Inc., Indianapolis. Both male and female cotton rats aged 5 to 7 weeks were used. Animals received subcutaneously either 5 ug of the indicated proteins, 10⁴ TCID₅₀ of the MeV live vaccine (Edmonston-Zaghreb) or the vehicle. The animals were immunized twice and two weeks after the last immunization were challenged. For intranasal infection, 10⁵ TCID₅₀ of MeV (strain B3 wt eGFP) was inoculated intranasally to isoflurane-anesthetized cotton rats in a volume of 100 μl. Four days after infection, the animals were euthanized by CO₂ inhalation. All right lung lobes and accessory lung lobes were collected for histologic evaluation. Left lung lobes were collected, weighed, and homogenized with a glass Dounce homogenizer for viral titration. Serial 10-fold dilutions of supernatant fluids were assessed on Vero-huCD150 (SLAM) cells for the presence of infectious virus in 48-well plates using cytopathic effect (CPE) as the endpoint. Plates were scored for CPE microscopically after 5 days, and the tissue culture infectious dose 50 (TCID₅₀) was calculated. The results are presented as TCID₅₀/g of lung tissue.

Histology preparation and evaluation. Right cranial, right middle, right caudal, and accessory lung lobes were perfused and fixed in 10% neutral buffered formalin. Standardized sections were trimmed and paraffin embedded. 4-μm thick sections were stained with H&E and graded histologically with a semi-quantitative scoring system for inflammatory infiltrates. The quantity of peribronchiolar and perivascular infiltrates were evaluated based on the proportion of profiles with associated inflammatory infiltrates (0%=grade 0; 0-25%=grade 1; 25-50%=grade 2; 50-75%=grade 3; >75%=grade 4). Severity of peribronchiolar and perivascular infiltrates were evaluated on a severity scale based on the most severely affected profile (no affected profiles=grade 0; interrupted cuff=grade 1; complete cuff <5 cell layers thick=grade 2; complete cuff >5 cell layers thick=grade 3). Interstitial and alveolar infiltrates were graded on quantity of alveolar septae or alveolar spaces, respectively, with inflammatory infiltrates (0%=grade 0; 0-10%=grade 1; 10-33%=grade 2; 33-66%=grade 3; >66%=grade 4).

### Example 5—MeV Conformation Specific Antibodies

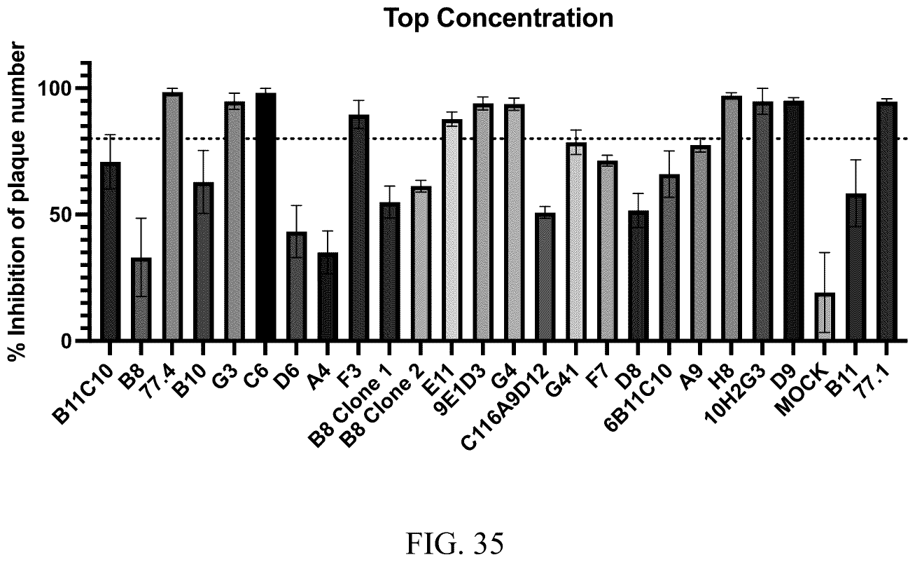

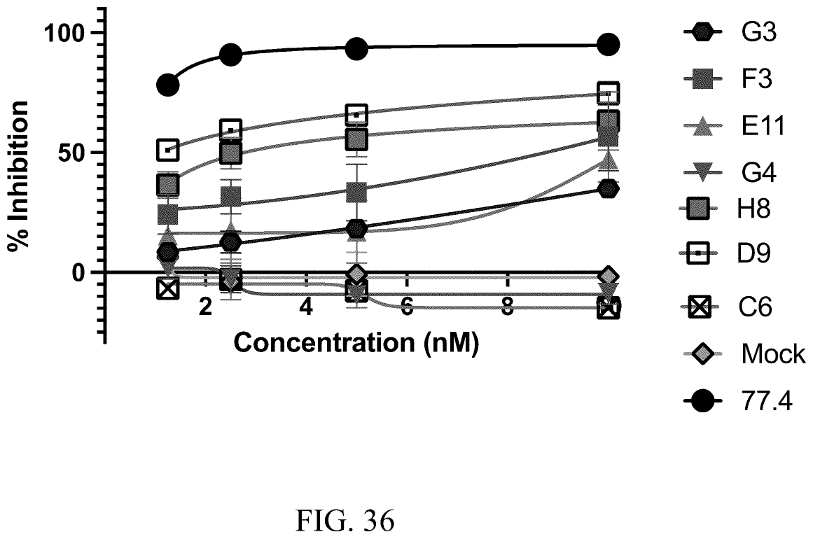

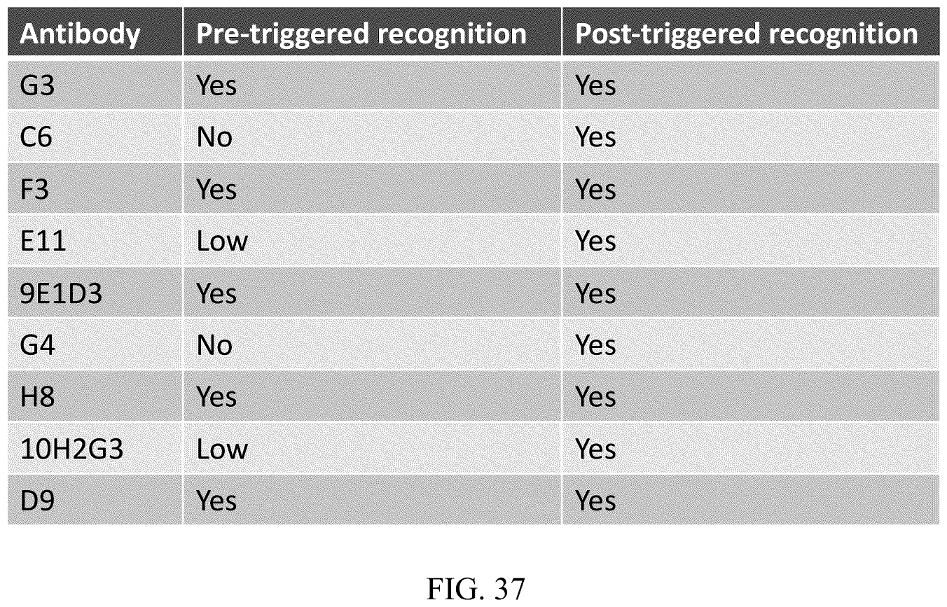

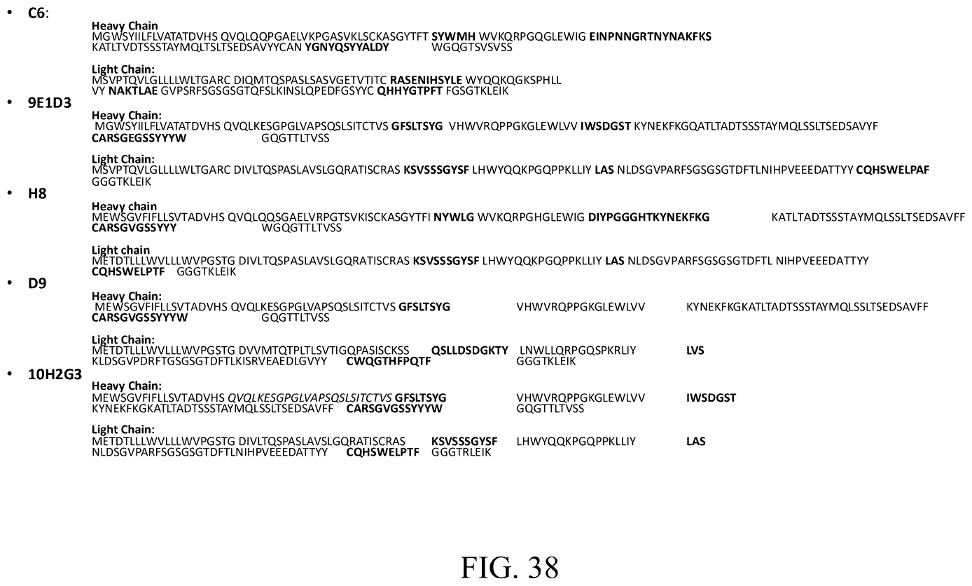

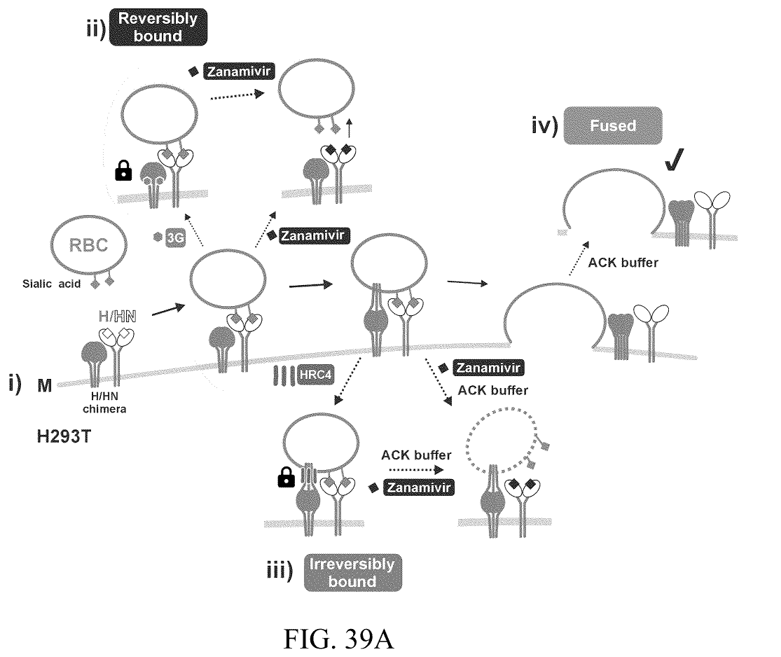

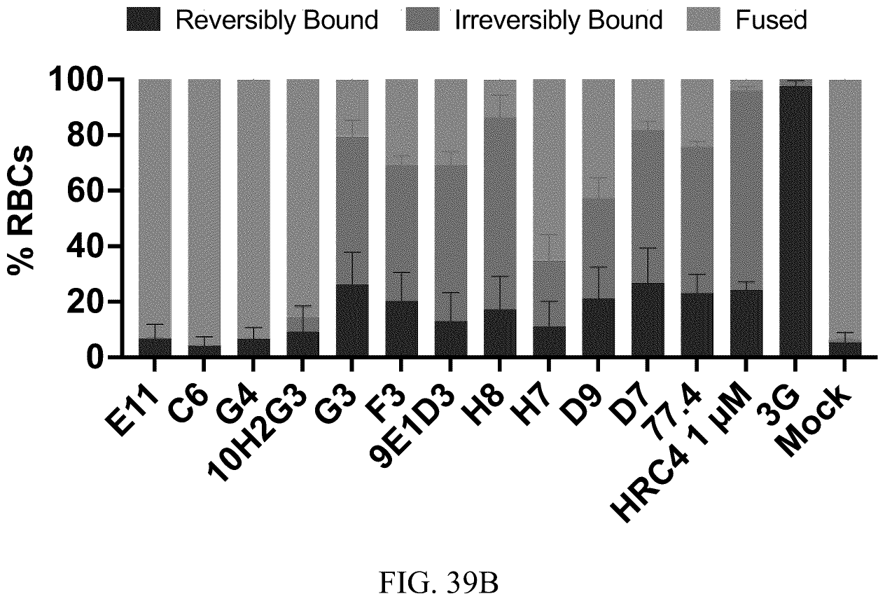

FIGS. 35-39 show analysis of MeV conformation specific antibodies. Applicants analyzed the following antibodies (CDRs are in bold):

C6:
Heavy Chain:
(SEQ ID NO: 17)
MGWSYIILFLVATATDVHS QVQLQQPGAELVKPGASVKLSCKASGYTFT SYWMH
WVKQRPGQGLEWIG EINPNNGRTNYNAKFKS
KATLTVDTSSSTAYMQLTSLTSEDSAVYYCAN YGNYQSYYALDY WGQGTSVSVSS
Light Chain:
(SEQ ID NO: 18)
MSVPTQVLGLLLLWLTGARC DIQMTQSPASLSASVGETVTITC RASENIHSYLE
WYQQKQGKSPHLLVY NAKTLAE GVPSRFSGSGSGTQFSLKINSLQPEDFGSYYC
QHHYGTPFT FGSGTKLEIK
9E1D3:
Heavy Chain:
(SEQ ID NO: 19)
MGWSYIILFLVATATDVHS QVQLKESGPGLVAPSQSLSITCTVS GFSLTSYG
VHWVRQPPGKGLEWLVV IWSDGST
KYNEKFKGQATLTADTSSSTAYMQLSSLTSEDSAVYF CARSGEGSSYYYW
GQGTTLTVSS
Light Chain:
(SEQ ID NO: 20)
MSVPTQVLGLLLLWLTGARC DIVLTQSPASLAVSLGQRATISCRAS KSVSSSGYSF
LHWYQQKPGQPPKLLIY LAS NLDSGVPARFSGSGSGTDFTLNIHPVEEEDATTYY
CQHSWELPAF GGGTKLEIK
H8:
Heavy chain:
(SEQ ID NO: 21)
MEWSGVFIFLLSVTADVHS QVQLQQSGAELVRPGTSVKISCKASGYTFI NYWLG
WVKQRPGHGLEWIG DIYPGGGHTKYNEKFKG
KATLTADTSSSTAYMQLSSLTSEDSAVFF CARSGVGSSYYY WGQGTTLTVSS
Light chain:
(SEQ ID NO: 22)
METDTLLLWVLLLWVPGSTG DIVLTQSPASLAVSLGQRATISCRAS KSVSSSGYSF
LHWYQQKPGQPPKLLIY LAS NLDSGVPARFSGSGSGTDFTL NIHPVEEEDATTYY
CQHSWELPTF GGGTKLEIK
D9:
Heavy Chain:
(SEQ ID NO: 23)
MEWSGVFIFLLSVTADVHS QVQLKESGPGLVAPSQSLSITCTVS GFSLTSYG
VHWVRQPPGKGLEWLVV KYNEKFKGKATLTADTSSSTAYMQLSSLTSEDSAVFF
CARSGVGSSYYYW GQGTTLTVSS
Light Chain:
(SEQ ID NO: 24)
METDTLLLWVLLLWVPGSTG DVVMTQTPLTLSVTIGQPASISCKSS
QSLLDSDGKTY LNWLLQRPGQSPKRLIY LVS
KLDSGVPDRFTGSGSGTDFTLKISRVEAEDLGVYY CWQGTHFPQTF GGGTKLEIK
10H2G3:
Heavy Chain:
(SEQ ID NO: 25)
MEWSGVFIFLLSVTADVHS QVQLKESGPGLVAPSQSLSITCTVS GFSLTSYG
VHWVRQPPGKGLEWLVV IWSDGST
KYNEKFKGKATLTADTSSSTAYMQLSSLTSEDSAVFF CARSGVGSSYYYW
GQGTTLTVSS
Light Chain:
(SEQ ID NO: 26)
METDTLLLWVLLLWVPGSTG DIVLTQSPASLAVSLGQRATISCRAS KSVSSSGYSF
LHWYQQKPGQPPKLLIY LAS NLDSGVPARFSGSGSGTDFTLNIHPVEEEDATTYY
CQHSWELPTF GGGTRLEIK

### Example 6—Screening for Neutralizing Conformation-Specific Measles f Antibodies

Measles virus (MeV) is a highly infectious respiratory virus and a re-emerging global public health threat. Inadequate vaccine coverage, decline in herd immunity, and vulnerable populations of immune compromised people who cannot be vaccinated have created an increased need for effective interventions. Monoclonal antibodies can both act as valuable antiviral therapeutics and aid in structural and mechanistic studies of the virus. Most neutralizing antibodies against measles are against the receptor binding protein (MeV H). Applicants aimed to elicit neutralizing monoclonal antibodies against various conformations of the measles fusion protein (MeV F). Antibodies that recognize the pre-fusion, post-fusion, or some intermediate state of MeV F may have different mechanisms of neutralization and may also aid in capturing different states of MeV F for structural analysis. Applicants generated monoclonal antibody producing hybridomas from mice vaccinated with stabilized soluble MeV F and characterized them. Applicants screened the antibodies for neutralization of live virus, recognition of pre-fusion and post-fusion MeV F, and mechanism of fusion inhibition using in vitro functional assays. Among neutralizing antibodies, the ones that recognize only the pre-fusion state or the pre- and post-fusion state all blocked fusion by stabilizing the F protein in an extended intermediate state after its activation. On the other hand, neutralizing antibodies that primarily recognized the post-fusion state did not block fusion in the in vitro experiment despite neutralizing live virus. Screening antibodies for conformational specificity as well as mechanism may provide a strategy for selecting or specifically eliciting the most potent neutralizing mAbs for MeV treatment or prophylaxis.

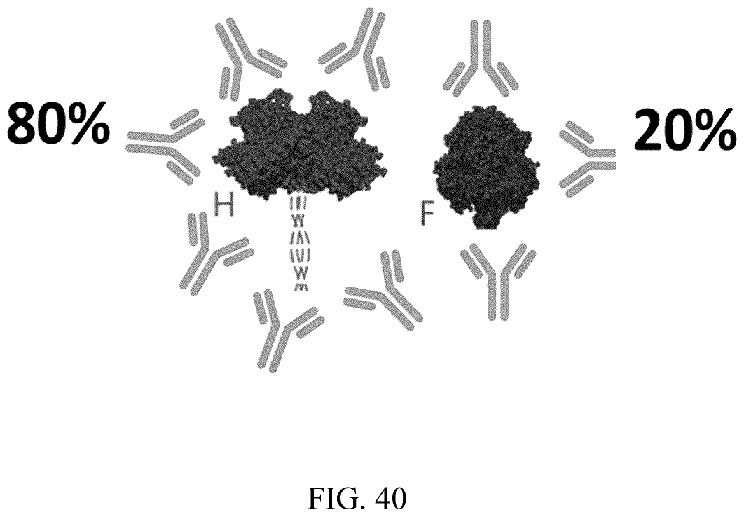

The hemagglutinin and fusion proteins are the main targets of neutralizing antibodies. The current vaccine elicits mostly antibodies against hemagglutinin: 80% of human sera antibodies are against H, and only 20% against F, and those anti-F antibodies are poorly neutralizing. Applicants hypothesize that the pre-fusion MeV fusion protein would elicit strong neutralizing antibodies. Applicants have identified several anti-F antibodies that effectively block viral entry. Applicants present here structural and cell biology data that show the epitope recognized and the mechanisms of action (FIG. 40).

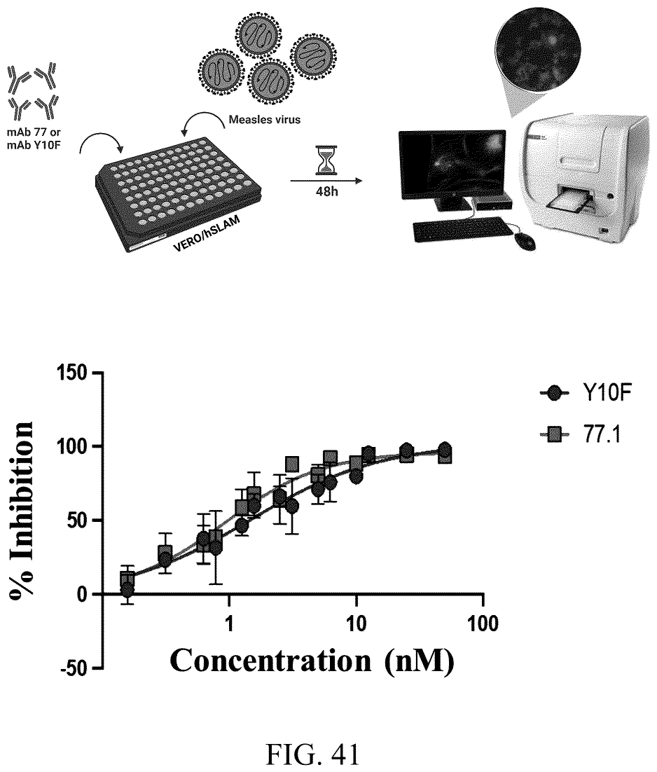

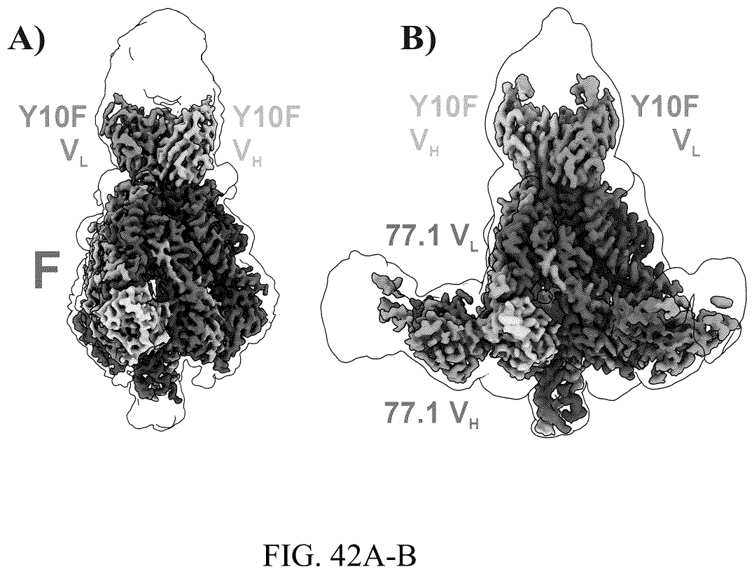

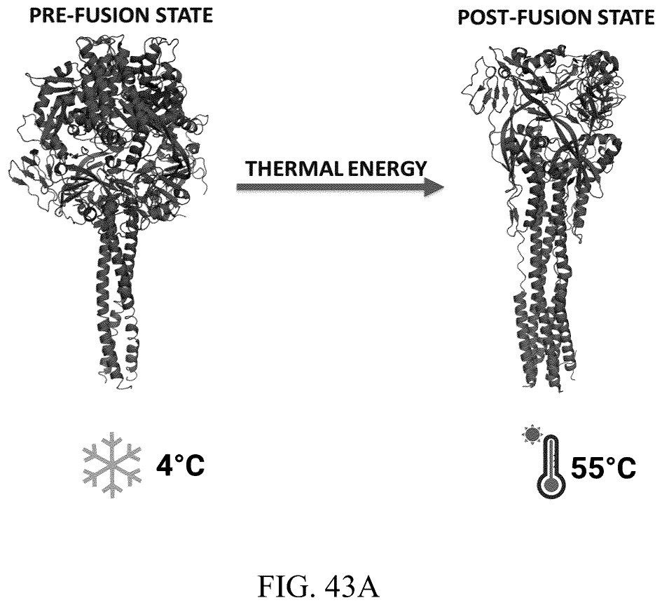

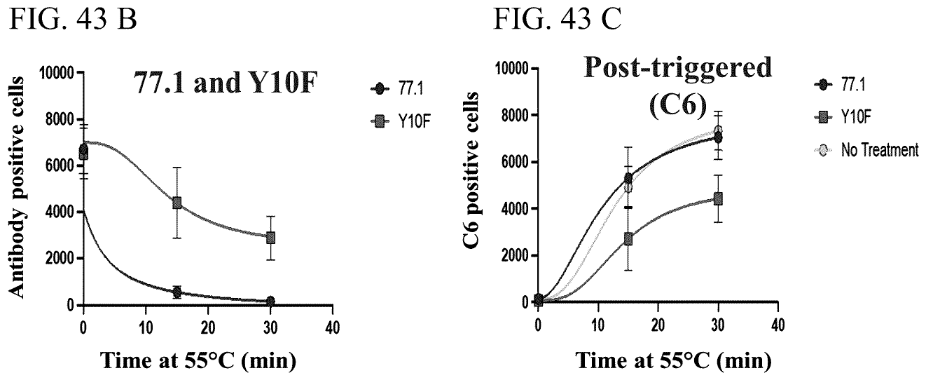

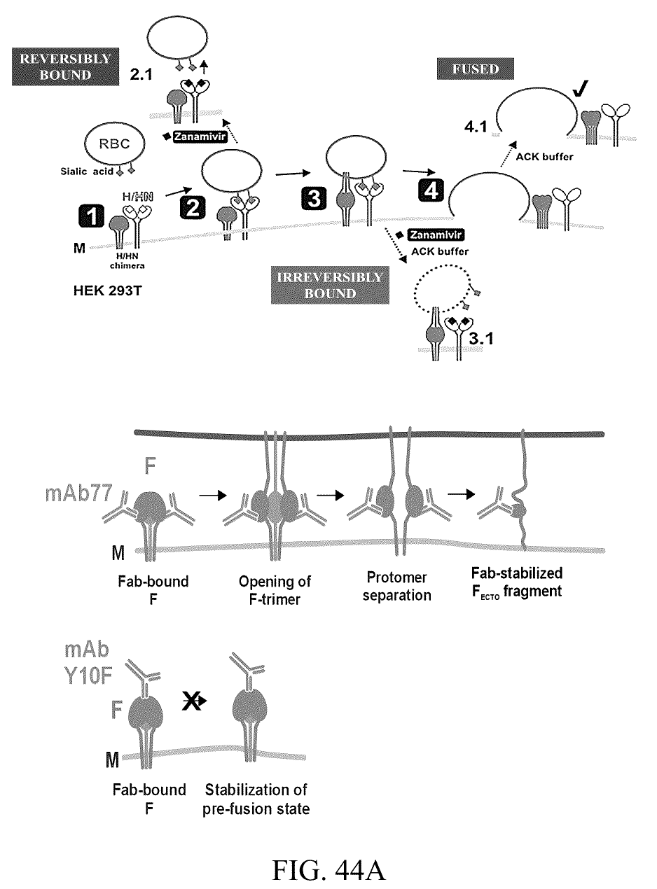

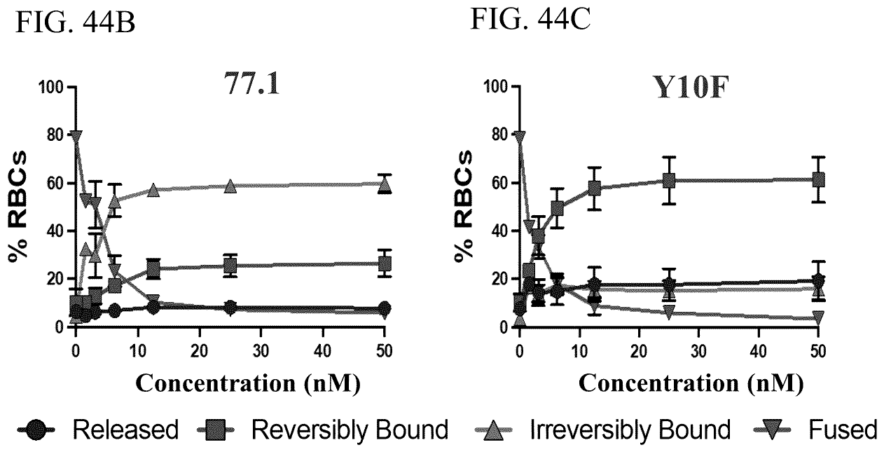

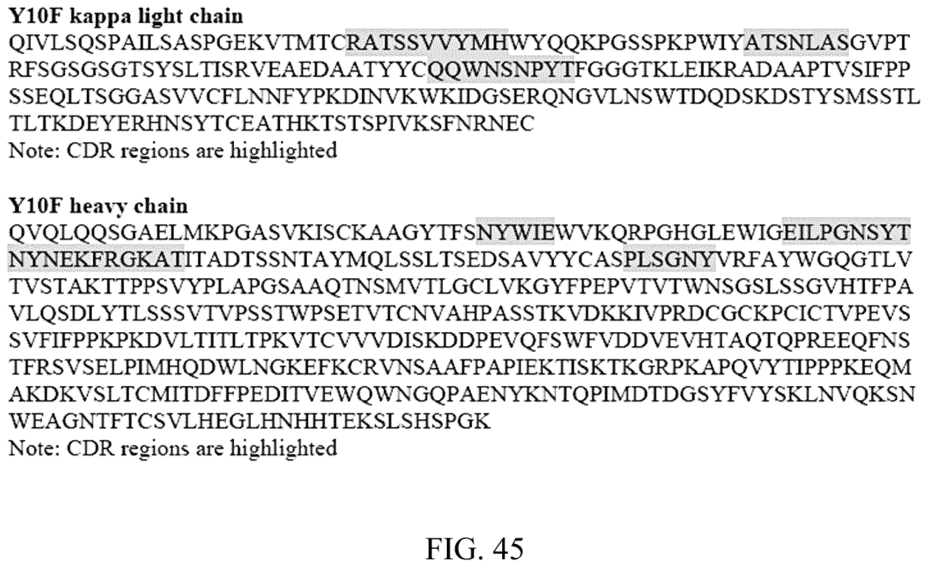

mAb 77.1 and Y10F are both effective in inhibiting MeV entry in a nanomolar range (FIG. 41). Applicants show the structural basis of epitope recognition (FIG. 42). The spatial distribution of epitopes on the F protein allows this antibody combination to bind two out of three available epitopes simultaneously. Applicants analyzed MeV F and show that the post-triggered epitope is reduced in the presence of Y10F (FIG. 43). The data indicates that Y10F prevents MeV F from undergoing to post-triggered state. Applicants show that MeV F mediated fusion is blocked by 77.1 and Y10F mAbs via distinct mechanisms (FIG. 44).

The sequence of Y10F is:

Y10F kappa light chain:
(SEQ ID NO: 15)
QIVLSQSPAILSASPGEKVTMTCRATSSVVYMHWYQQKPGSSPKPWIYAT
SNLASGVPTRFSGSGSGTSYSLTISRVEAEDAATYYCQQWNSNPYTFGGG
TKLEIKRADAAPTVSIFPPSSEQLTSGGASVVCFLNNFYPKDINVKWKID
GSERQNGVLNSWTDQDSKDSTYSMSSTLTLTKDEYERHNSYTCEATHKTS
TSPIVKSFNRNEC
Y10F heavy chain:
(SEQ ID NO: 16)
QVQLQQSGAELMKPGASVKISCKAAGYTFSNYWIEWVKQRPGHGLEWIGE
ILPGNSYTNYNEKFRGKATITADTSSNTAYMQLSSLTSEDSAVYYCASPL
SGNYVRFAYWGQGTLVTVSTAKTTPPSVYPLAPGSAAQTNSMVTLGCLVK
GYFPEPVTVTWNSGSLSSGVHTFPAVLQSDLYTLSSSVTVPSSTWPSETV
TCNVAHPASSTKVDKKIVPRDCGCKPCICTVPEVSSVFIFPPKPKDVLTI
TLTPKVTCVVVDISKDDPEVQFSWFVDDVEVHTAQTQPREEQFNSTFRSV
SELPIMHQDWLNGKEFKCRVNSAAFPAPIEKTISKTKGRPKAPQVYTIPP
PKEQMAKDKVSLTCMITDFFPEDITVEWQWNGQPAENYKNTQPIMDTDGS
YFVYSKLNVQKSNWEAGNTFTCSVLHEGLHNHHTEKSLSHSPGK
Note: CDR regions are underlined

### Example 7—a Neutralizing Antibody Prevents Postfusion Transition of Measles Virus Fusion Protein

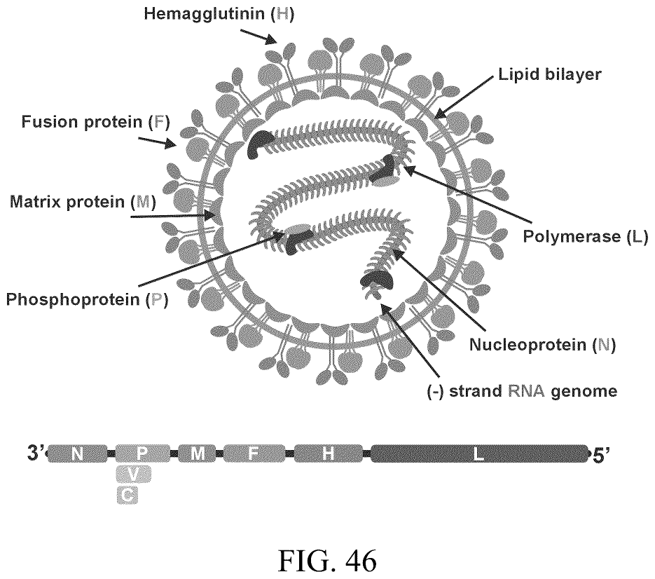

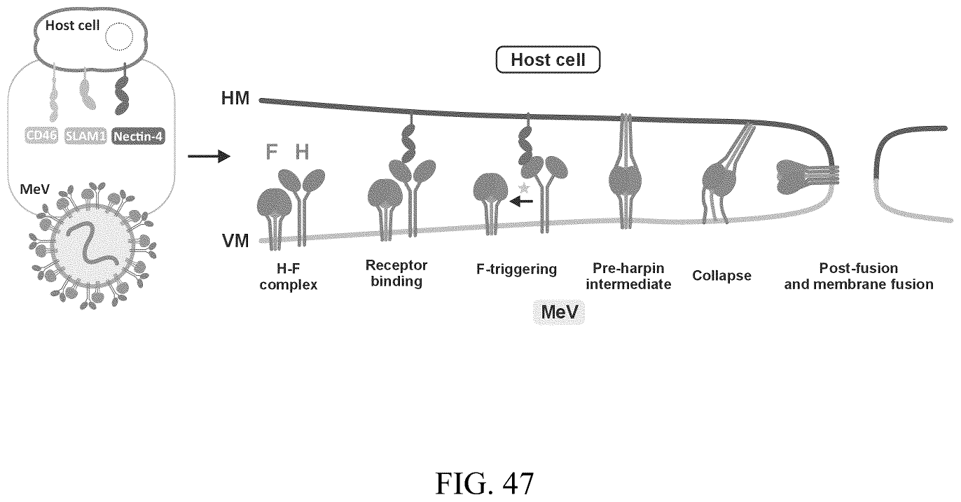

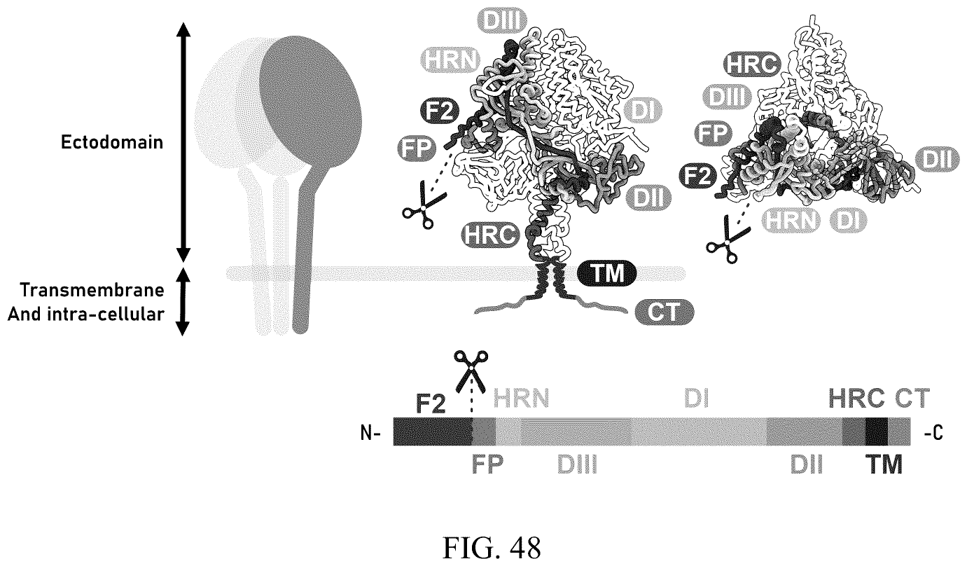

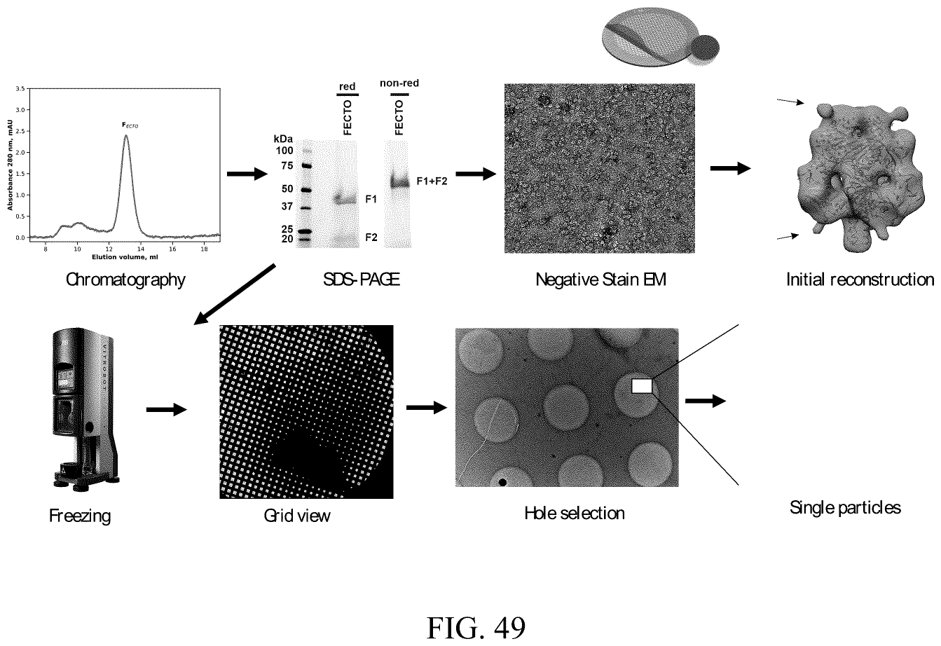

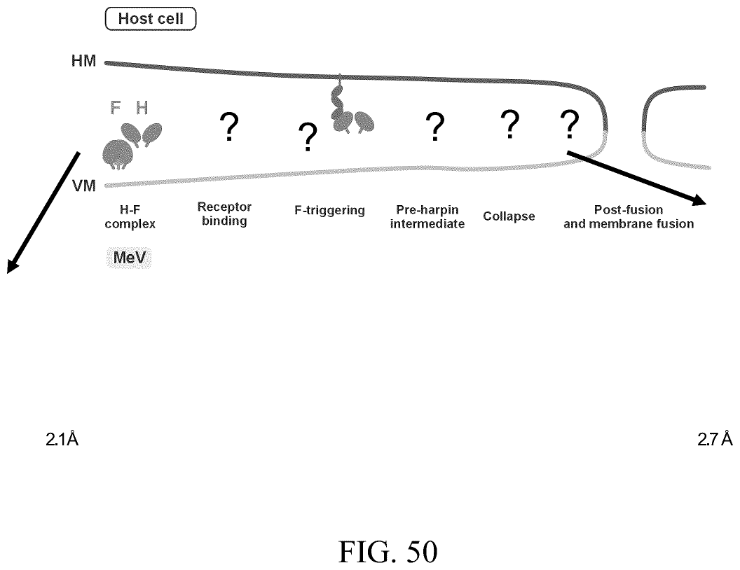

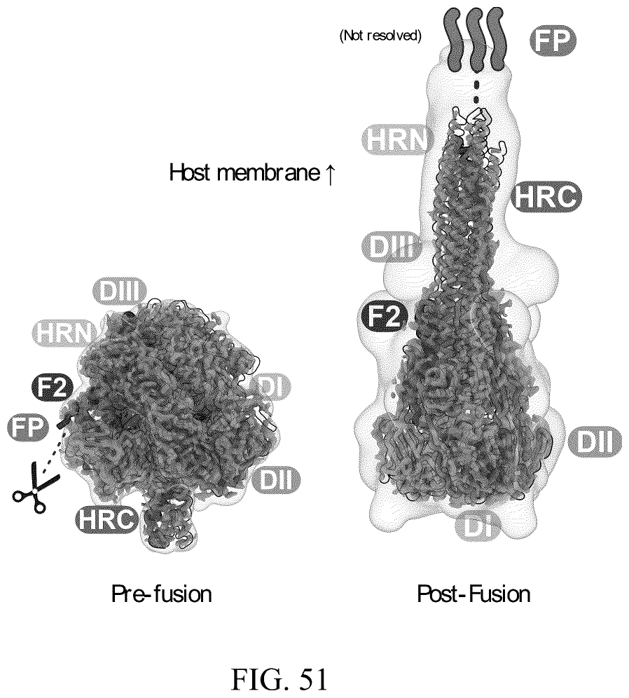

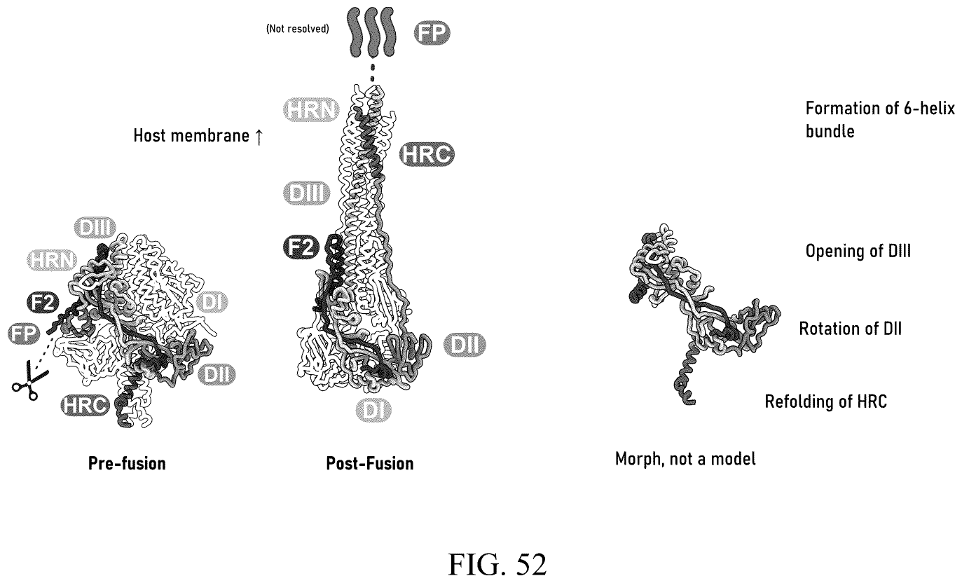

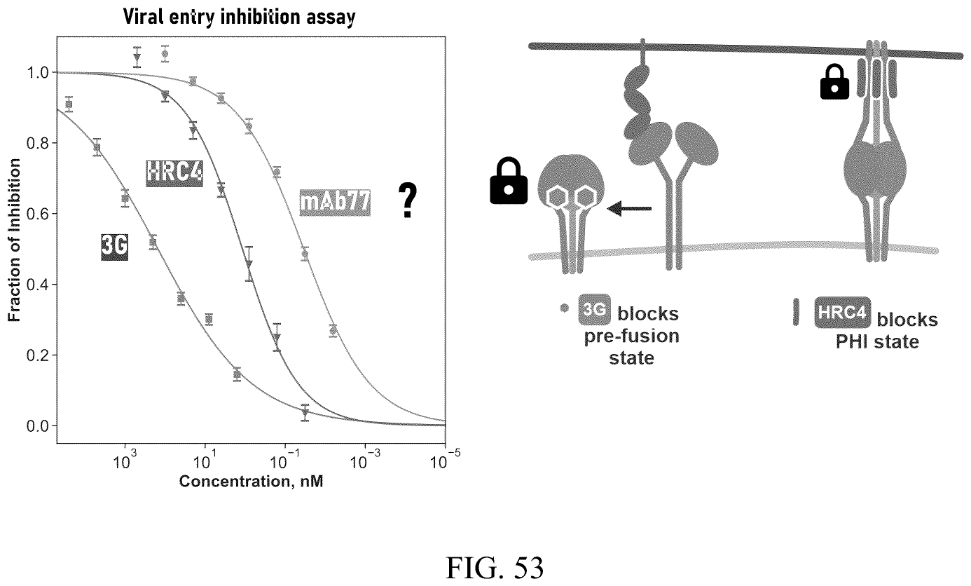

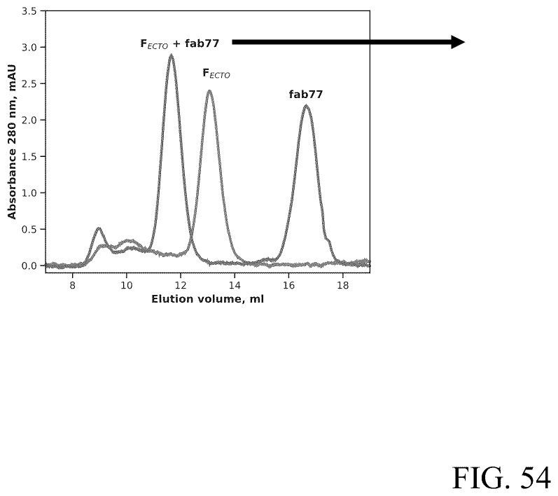

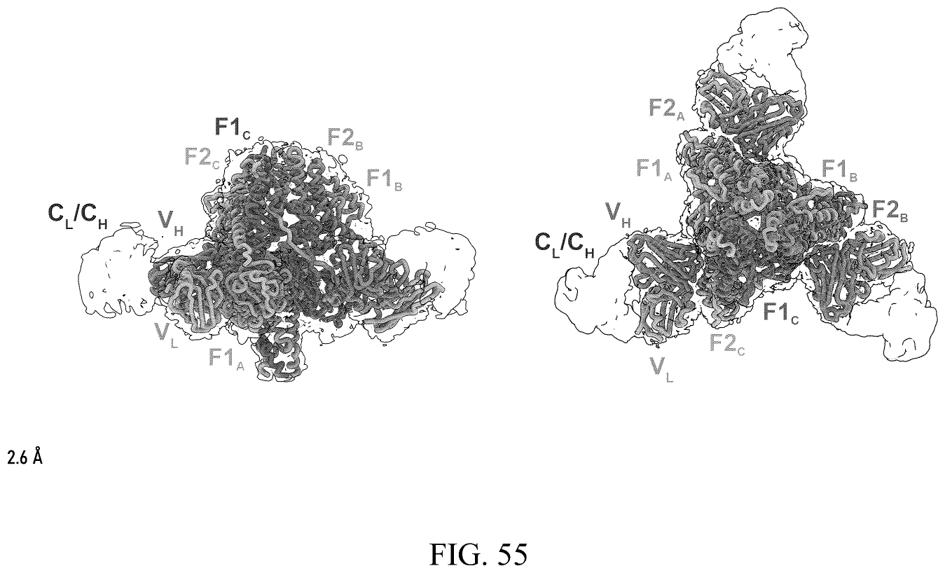

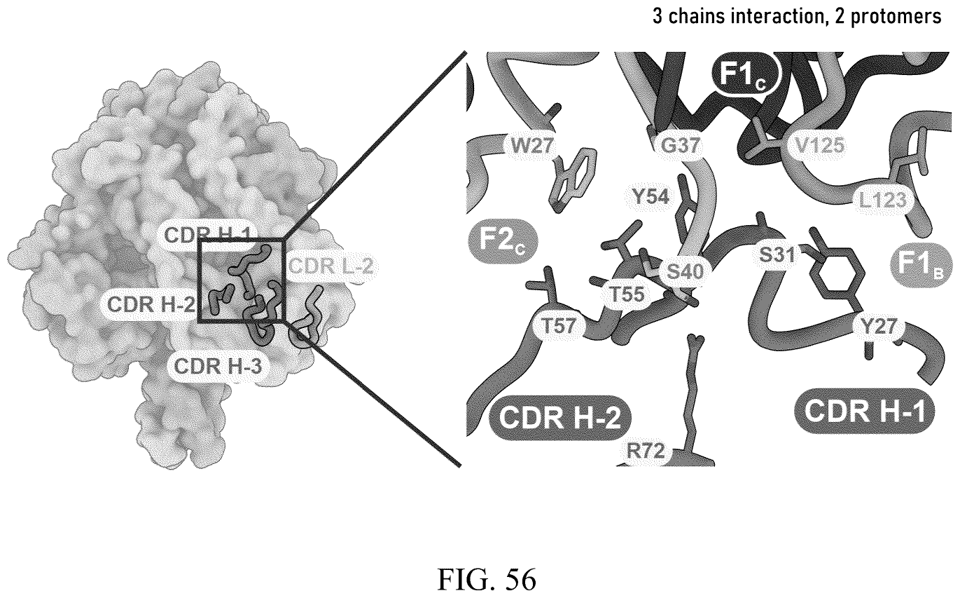

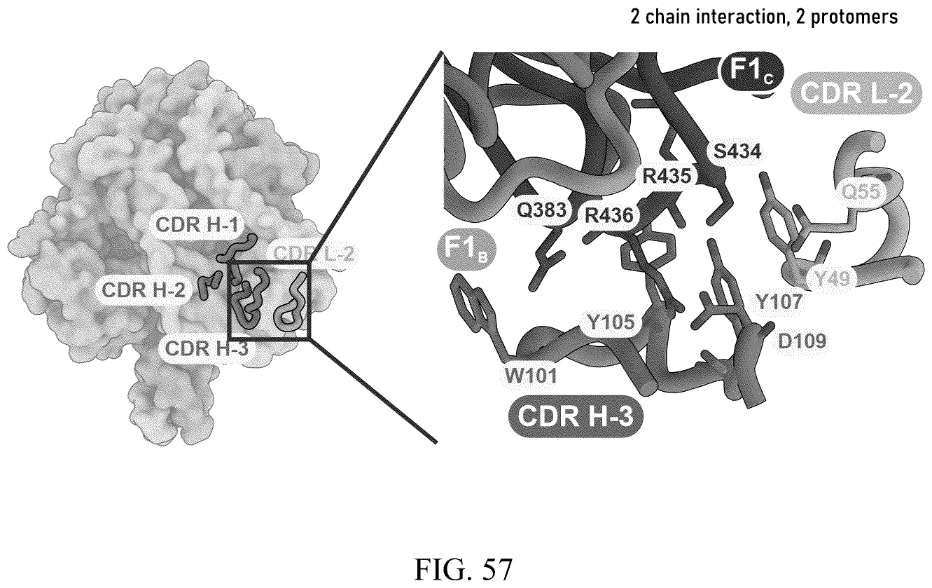

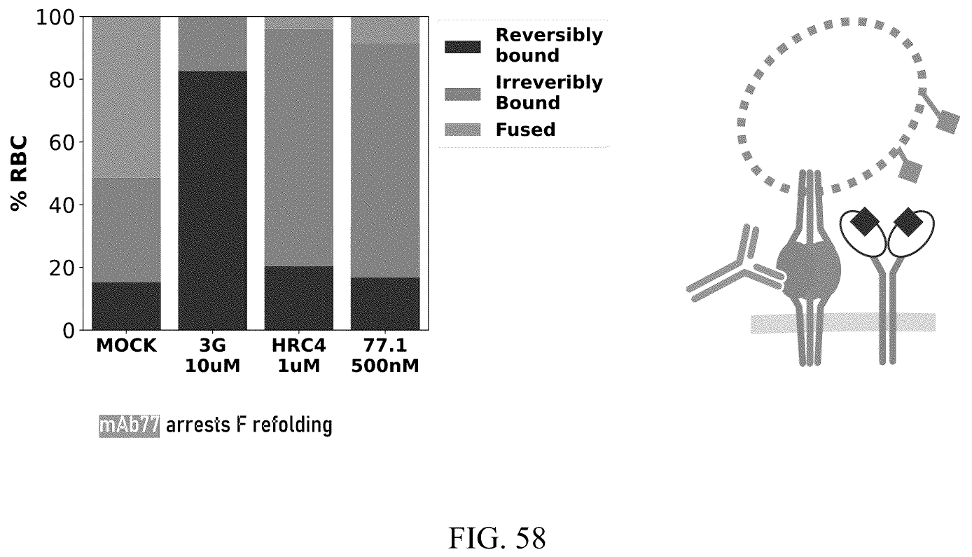

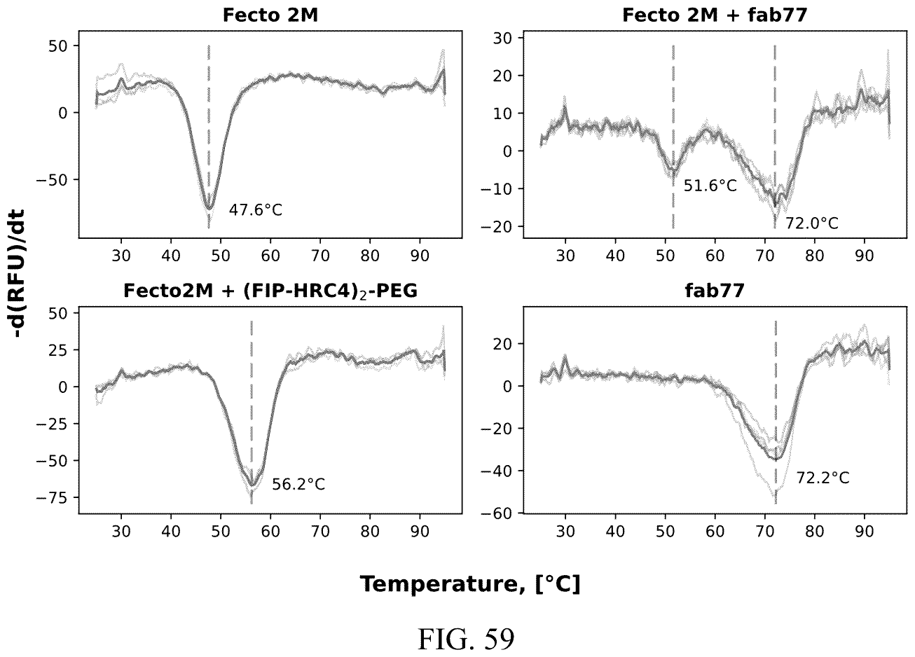

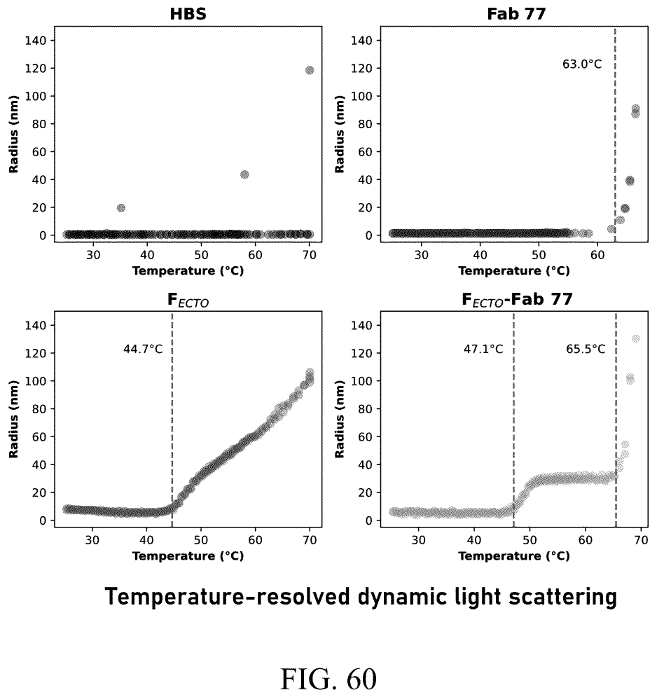

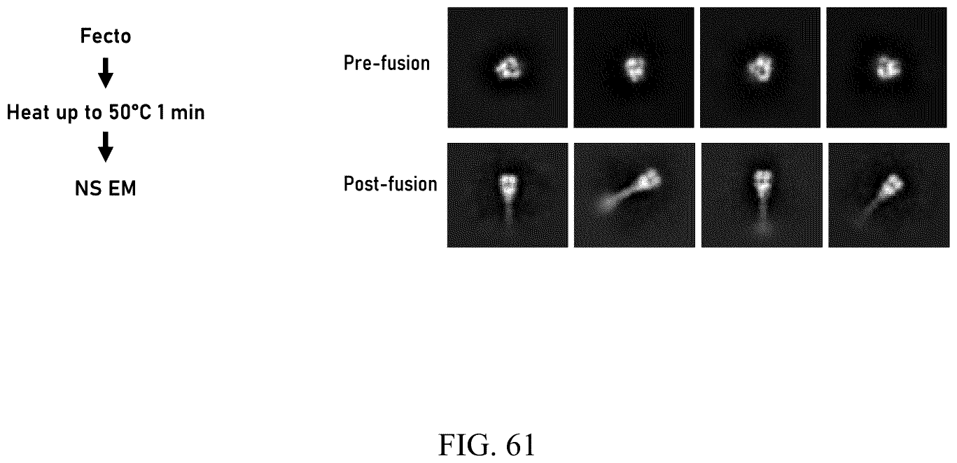

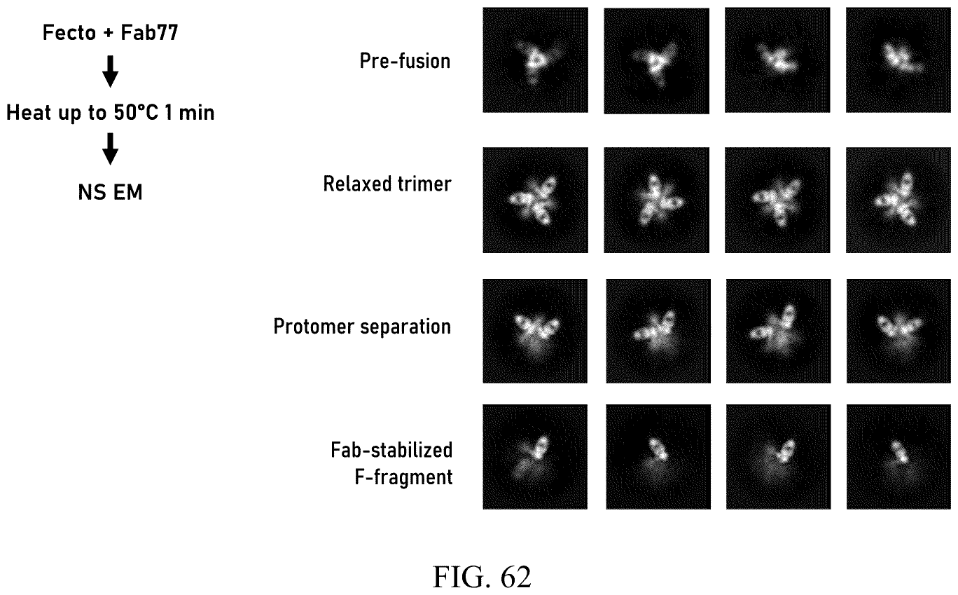

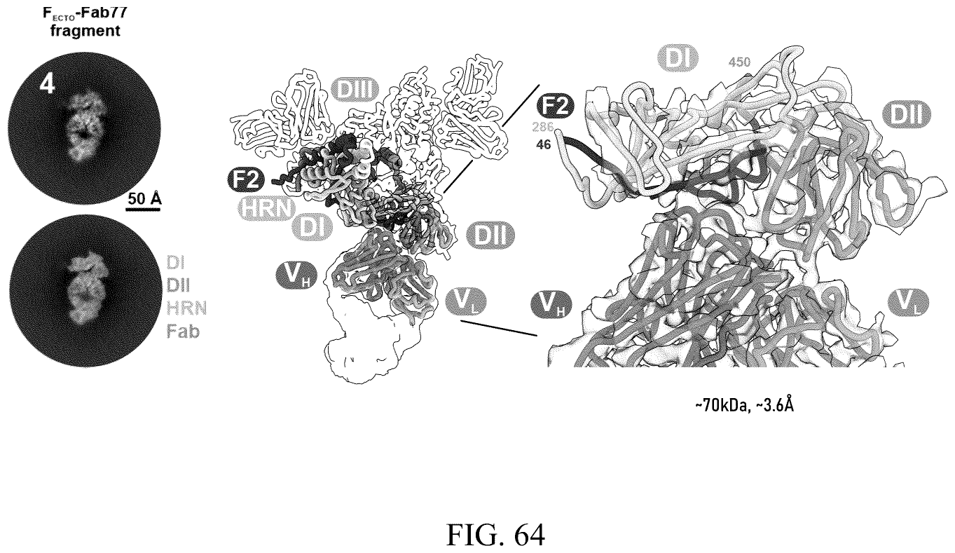

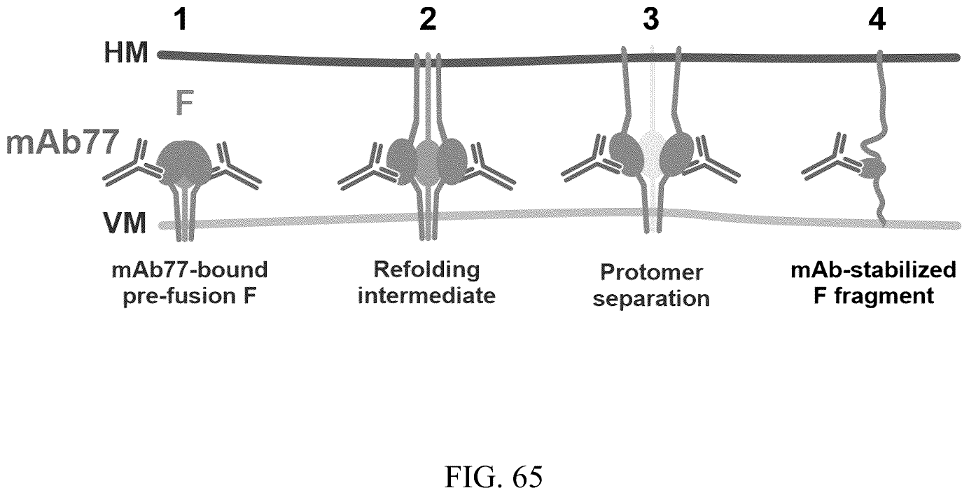

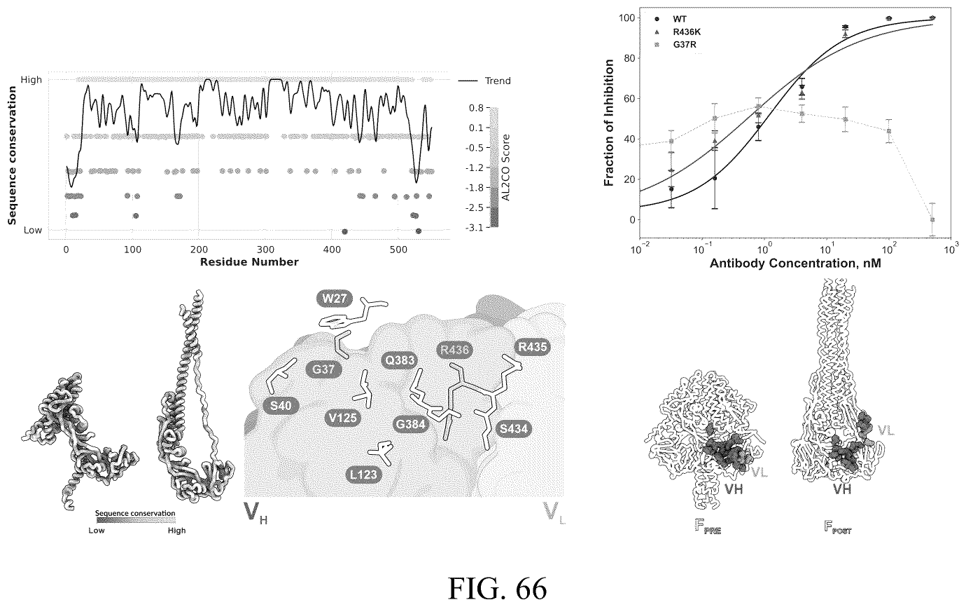

Measles (MeV) is a paramyxoviruse and causes various infections in vertebrates (FIG. 46). Measles is still a serious threat. R₀ of 12-18, is the most infectious known human disease. There are more childhood deaths than any other vaccine-preventable disease. There are 2500× more deaths in low-income countries. >95% vaccination coverage is required for herd immunity. Vaccination campaigns are compromised by COVID-19 and sociological reasons. Additionally, not everyone can be vaccinated. There are no available measles specific treatments and drugs. Developing antiviral strategies for measles is crucial. FIG. 47 shows measles virus entry. FIG. 48 shows MeV architecture and structure (see, also, Hashiguchi T, Fukuda Y, Matsuoka R, et al. Structures of the prefusion form of measles virus fusion protein in complex with inhibitors. Proc Natl Acad Sci USA. 2018; 115(10):2496-2501). MeV glycoproteins refold from pre-fusion and post fusion (FIG. 50, 51, 52). Applicants tried to stop the process by targeting the MeV fusion (F) protein in its prefusion state and inhibit fusion and viral infection, such as with mAb77 (see, also, Mathieu C, Ferren M, Harder O, et al. Single-chain variable fragment antibody constructs neutralize measles virus infection in vitro and in vivo. Cell Mol Immunol. 2021; 18(7):1835-1837). mAb77 works better than previously described inhibitors (FIG. 53). Applicants determined the structure of F with Fab77 (FIG. 54-57). Using the red blood cell fusion assay described herein, Applicants determined that mAb77 arrests F in fusion intermediate state (FIG. 58). Applicants determined that Fab77 only marginally stabilizes F (47.6 vs 51.6 °C) (FIG. 59), but completely changes F refolding behavior because Fab77 stabilizes F refolding intermediate (FIG. 60). Applicants determined how Frefolds (FIG. 61-63). Applicants show that the Fab keeps the epitope stable (FIG. 64). In summary, Applicants obtained a novel MeV F construct that allows the study of viral fusion, characterized pre-post fusion states of F elucidating conformational changes happening during refolding, described a novel neutralizing antibody against MeV, and elucidated a unique neutralization mechanism that halts the refolding before the membrane merging (FIG. 65). Additionally, the mAb77 epitope is highly conserved (FIG. 66).

### Example 8—a Neutralizing Antibody Prevents Postfusion Transition of Measles Virus Fusion Protein

INTRODUCTION: Declining vaccination rates have resulted in a global resurgence of measles virus (MeV) and a clear need for effective treatments for those who cannot or have not yet been vaccinated or those in whom vaccines do not elicit neutralizing immunity. A promising approach involves neutralizing antibodies directed at the viral fusion protein F, but no such neutralizing antibodies are yet structurally characterized or approved.

RATIONALE: This study aims to elucidate the mechanism of action of monoclonal antibody (mAb) 77 and assess its potential as a therapeutic for MeV infection.

RESULTS: Applicants find that mAb 77 potently neutralizes MeV infection in vitro and significantly reduces replication and viral loads in vivo compared with mock-treated animals. A mechanistic assay revealed that mAb 77 effectively prevents complete membrane fusion, but it does so without blocking F activation, the initiation of fusion, or the formation of the extended intermediate.

To understand the mechanism of neutralization of mAb 77, Applicants engineered a stabilized F ectodomain (FECTO) with two naturally occurring point mutations identified in cases of MeV encephalitis. These two point mutations allowed expression of F in the prefusion conformation without exogenous trimerization domains while still permitting later folding of this F into its postfusion state. A 2.11-Åresolution cryo-electron microscopy (cryo-EM) structure of MeV FECTO in its prefusion state demonstrated that the two stabilizing mutations did not affect the overall structure, and a further structure of the postfusion F at 2.7-A-resolution provided the endpoint structure needed to model intermediates in the refolding pathway.

A third cryo-EM structure, of FECTO in complex with Fab 77 (2.6-Å resolution), revealed the antibody epitope. mAb 77 simultaneously bridges domains I and II, the N-terminal part of F2, and the linker in between domain II and C-terminal heptad repeat, and on the edge of the epitope also recognizes the fusion peptide. This conformational epitope is only present in the prefusion form of F.

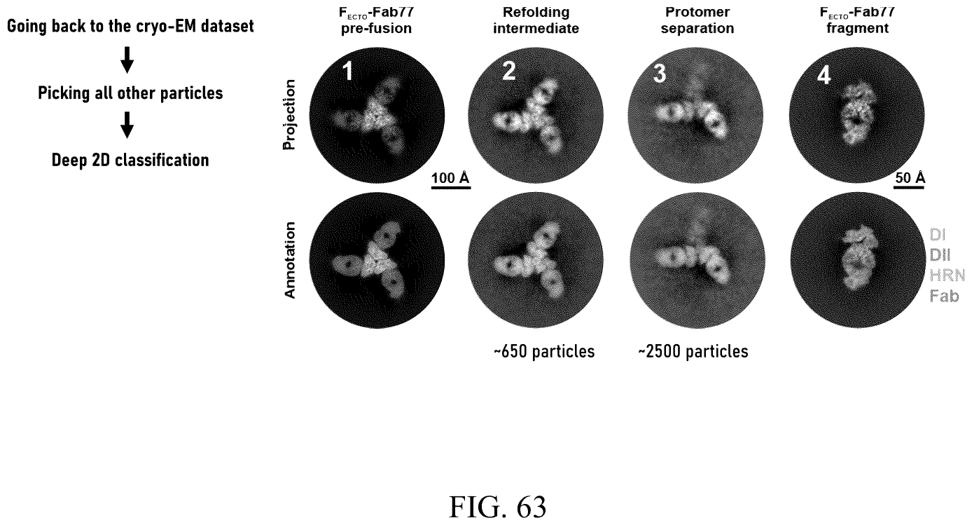

To reconcile this prefusion-bound structure with mechanistic information that mAb 77 neutralized at a post-triggering state, Applicants sought to understand intermediates in refolding and the stage at which mAb 77 blocked the fusion process. A deeper analysis of the cryo-EM dataset of the FECTO-Fab 77 complex uncovered subpopulations of particles in a series of two dimensional (2D) classes that illustrated snapshots in refolding (triggering, extended intermediate, trimer separation) and also allowed visualization of a trimeric, refolding-intermediate state arrested by bound Fab 77. A fourth structure, of this Fab 77-stabilized F fragment, was resolved to 3.6-Å resolution, revealing that the antibody still binds domains I and II and the N-terminal heptad repeat (HRN) in their prefusion arrangement and that anchoring of these regions together arrests the process of refolding at this intermediate stage. See, FIGS. 63-65.

CONCLUSION: The study reveals in vitro neutralization and in vivo protection by mAb 77, as well as the cryo-EM structure of mAb 77 in complex with MeV F and its mechanism of action, information that is important for potential therapeutic use against MeV infection. Applicants showed that mAb 77 specifically recognizes MeV F in the prefusion conformation and allows initial F activation and refolding but also that it interrupts the refolding process to prevent conformational changes required for membrane fusion and viral entry. These findings suggest a model for understanding not only antibody neutralization of MeV, but also other viruses for which antibodies are thought to interrupt fusion but for which structures are not yet available.

### Example 9—a Neutralizing Antibody Prevents Postfusion Transition of Measles Virus Fusion Protein

Measles virus (MeV) presents a public health threat that is escalating as vaccine coverage in the general population declines and as populations of immunocompromised individuals, who cannot be vaccinated, increase. There are no approved therapeutics for MeV. Neutralizing antibodies targeting viral fusion are one potential therapeutic approach but have not yet been structurally characterized or advanced to clinical use. Applicants present cryo-electron microscopy structures of prefusion F alone [2.1-angstrom (Å) resolution], F complexed with a fusion-inhibitory peptide (2.3-Å resolution), F complexed with the neutralizing and protective monoclonal antibody (mAb) 77 (2.6-Å resolution), and an additional structure of postfusion F (2.7-Å resolution). In vitro assays and examination of additional EM classes show that mAb 77 binds prefusion F, arrests F in an intermediate state, and prevents transition to the postfusion conformation. These structures shed light on antibody-mediated neutralization that involves arrest of fusion proteins in an intermediate state.

Measles virus (MeV) is an enveloped, single-stranded negative-sense RNA virus that is highly transmissible [basic reproduction number (R₀) of 12 to 18] by the respiratory route. MeV belongs to the Paramyxoviridae family and causes considerable morbidity and mortality despite the availability of safe and effective MeV vaccines. Measles accounts for more childhood deaths than any other vaccine-preventable disease. MeV was presumed to be eliminated in the US (defined as ≥12 months without transmission) in 2000. However, the reintroduction of the virus in recent years has resulted in a resurgence and repeated outbreaks. There were 9 million cases worldwide in 2021, >10.5 million in 2022, and between 2022 and 2023, the World Health Organization (WHO) European region observed more than a 30-fold increase in cases, with 40 of 53 member states affected. American vaccine coverage is now below the >95% required for herd immunity [see Centers for Disease Control and Prevention reports]. Although outbreaks are usually attributed to the unvaccinated, cases also occur in people who were previously vaccinated. Up to 5% of vaccine recipients do not develop adequate protective antibodies, even after the recommended two dose vaccination regimen, and even in those who do develop protective antibodies, vaccine elicited immunity likely wanes over time. Even mild cases of measles infection can result in immune “amnesia” and loss of preexisting antibodies against other infections, leaving measles-convalescent individuals more vulnerable to future diseases. Rare but severe complications of measles can lead to lethal MeV encephalitis.

MeV infects through coordinated action of the hemagglutinin (H) and fusion (F) envelope glycoproteins to mediate virus attachment to two cellular receptors, SLAM1 (or CD150) and Nectin-4, and entry into target cells. Vaccination results in neutralizing antibodies against these two proteins. Earlier work suggested that most neutralizing antibodies are directed against H and that anti-F antibodies elicited by the current vaccine are poorly neutralizing. A better understanding of the structures of F protein, the conformational changes of F that drive fusion, and whether antibody-mediated inhibition of conformational change can result in in vitro neutralization and in vivo protection against MeV are needed.

F is initially synthesized as a precursor molecule (F0), which is subsequently cleaved into two subunits: the N-terminal F2 and the C terminal F1. These subunits remain linked by a disulfide bond. Three F1 and F2 subunits assemble to form a prefusion F complex on the viral surface. This trimeric complex is kinetically trapped in the metastable state. Under physiological conditions, fusion activation requires the engagement of the H glycoprotein by the cell surface receptor. Once this activation occurs, the prefusion F protein undergoes a substantial conformational change, elongating and embedding the hydrophobic fusion peptide into the membrane of the target cell. During the fusion process, Frefolds into a stable six-helix bundle postfusion structure formed by the tight association of the N-terminal heptad repeat (HRN) and the C-terminal heptad repeat (HRC). This entire process brings the viral and cellular membranes in close proximity and leads to membrane merging. However, before adopting the low-energy postfusion state, the F protein adopts a range of intermediate conformations, which require rearrangement of its domains.

Targeting the intermediate state of F is a potential antiviral approach. Although neutralizing monoclonal antibodies (mAbs) offer lifesaving treatments for several viruses, none are currently available to prevent or treat measles. Furthermore, no structural information is yet available for any antibody against MeV that could be used to define epitopes or activities that prevent or treat infection. The mAb for which structure and activities are documented here is one of the very few neutralizing mAbs described against the MeV envelope fusion (F) protein.

### Human Chimeric mAb 77 Potently Inhibits MeV Infection

Applicants previously isolated and sequenced an F-targeted neutralizing mouse antibody mAb 77.4 and demonstrated that it inhibits MeV infection by blocking fusion. To facilitate future use as a human therapeutic, Applicants grafted the 77.4 variable regions into a human immunoglobulin G1 (IgG1) framework to obtain a chimeric antibody, now called mAb 77, and assessed its in vitro and in vivo efficacy.

Applicants first evaluated the antiviral activity of mAb 77 using an in vitro viral infection assay in which Vero-human CD150 (Vero-huCD150/SLAM) cells were treated with serial dilutions of mAb 77. Two distinct MeV F viral entry inhibitors were included as additional controls. One inhibitor, N-(3-cyanophenyl)-2-phenylacetamide [compound 3G], binds to prefusion F and blocks H-mediated fusion activation. The other inhibitor is an HRC-derived peptide called HRC4, which binds to HRN in the prehairpin intermediate and blocks the conformational transition to the six-helix bundle. After treatment, cells were infected with the MeV wild-type (WT) B3-eGFP (enhanced green fluorescent protein) virus. The mAb 77 potently inhibited infection with a median inhibitory concentration F1 (IC50) of 0.034 nM (5.1 ng/ml) (FIG. 1A), which was substantially better than both HRC4 and 3G, which blocked fusion with IC50 values of 1.17 and 150.12 nM, respectively.

To test mAb 77 for in vivo efficacy against MeV exposure, Applicants used the cotton rat model of measles virus infection. Cotton rats were treated with 0.1 mg/kg (n=5) or 1 mg/kg (n=10) of mAb 77 intraperitoneally 12 hours before challenge with 105 TCID50 (50% tissue culture infectious) dose of MeV WT B3-GFP. Antibody pretreatment resulted in a significant reduction in viral replication as compared with mock-treated animals that had an average titer of 3.6 log 10±0.8 TCID50 per gram of lung tissue (FIG. 1B). Three of the animals pretreated with 0.1 mg/kg mAb were virus negative, and two had reduced virus titers in lung tissue (2.4 and 2.6 log 10 TCID50per gram of lung tissue). Eight of the animals treated with 1 mg/kg mAb were virus-negative, and two had reduced virus titers (mean of 2.9 and 3.6 log 10 TCID50 per gram of lung tissue).

### mAb 77 Prevents Conformational Changes Required for Fusion

The F protein is primed to undergo substantial conformational change and refolding to drive the fusion of the viral and host membranes. These structural changes are triggered by a signal from the stalk region of the H protein, which is activated once the globular head of the H protein binds to its cellular receptor. As a first step in F conformational rearrangement, the HRN (contained in domain III of the F1 head region) refolds from three short helices and beta-hairpin motifs into a single ~100-Ålong helix. This assembled and now-extended helix projects the fusion peptide, toward and into the host membrane. This extended F conformation is often called the “preharpin intermediate”. The subsequent sequence of events remains elusive but, by analogy with other viruses, likely involves substantial rearrangement and repositioning of the stalk region containing the HRC. This movement enables the HRC to collapse onto the HRN into a six-helix bundle that draws the viral and host membranes together to promote fusion.

Previous studies demonstrated that murine mAb 77.4 inhibits the fusion of viral and host cell membranes, but the step at which it functions remains unclear (Mathieu, et al., 2021). The mAb 77 antibody could (i) stabilize the F prefusion conformation to prevent F from triggering, (ii) allow triggering but stabilize the intermediate state to prevent subsequent refolding and membrane fusion, or (iii) impair F refolding by another mechanism.

To elucidate the stage of the fusion process at which mAb 77 acts, Applicants used an assay that measures fusion between transfected human embryonic kidney 293T (HEK293T) cells with red blood cells (RBCs) by quantifying the amount of hemoglobin released upon lysis (FIG. 2A and B) (Bovier, et al., 2021). An integral component of this assay is the ability to release receptor engagement by H so that anchoring of RBCs by H can be distinguished from anchoring by F. As RBCs do not display either of the MeV receptors for WT H (i.e., CD150/SLAM or nectin-4), Applicants instead used a previously characterized chimeric protein that contains the MeV H stalk fused to the sialic acid receptor-binding head domain of paramyxovirus human parainfluenza virus 3 (HPIV3) HN. The interaction of the HHN chimeric protein with the sialic acid displayed on the RBC surface can be reversed through treatment with the small molecule zanamivir. Hence, the H-HN chimera enables both reversible attachment to RBCs and MeV H-driven triggering of F. H-HN and F transfected cells were first incubated with RBCs at 4 °C to permit sialic acid binding but not fusion. The cell mixture was then treated with either media or different viral entry inhibitors, and fusion was allowed to proceed for 60 min at 37 °C. Treatment with diluent media alone resulted in fusion of ~50% of total RBCs with the HEK293T cells (FIG. 2, A and B). The remaining RBCs had either begun, but not completed, fusion (irreversibly bound through F) or had not yet initiated fusion (reversibly bound through HN only). Treatment with the 3G inhibitor prevented nearly all F-mediated anchoring, indicating, as previously reported, that this inhibitor prevents F activation. Meanwhile, treatment with the HRC4 peptide inhibitor resulted in most RBCs being irreversibly bound, indicating that F was activated but could not complete fusion, as previously described. Treatment with mAb 77 had the same effect as HRC4, where most RBCs were irreversibly bound but not fused. Applicants conclude from these results that, unlike 3G, mAb 77 does not completely prevent the activation of F, nor does it inhibit the insertion of the fusion peptide into target cells. It does, however, prevent the collapse of the prehairpin intermediate into the six-helix bundle required for fusion, suggesting that mAb 77 remains associated with an intermediate form of F.

### Natural Substitutions Allow for Production of MeV F in the Prefusion State

To structurally elucidate the mechanism of action of mAb 77, Applicants first established a protein purification protocol using a stabilized MeV F construct. Expression of the WT MeV F ectodomain (amino acids 1 to 495) results in the production of F in its postfusion conformation only. Previous structural characterization of MeV F was achieved by engineering four cysteines in the stalk region, which results in disulfide linkages between the three protomers. However, the protein produced from this construct aggregated, even at lower concentrations. As such, Applicants instead generated a MeV F ectodomain that incorporates two naturally occurring point mutations, Glu170→Gly (E170G) F3 and E455G (FIG. 3A). These mutations were derived from human central nervous system isolates of MeV and stabilized F in the prefusion state. When paired, stabilization was further enhanced. This F ectodomain (F_{ECTO}) was ~95% cleaved by furin, and ~85% of the resulting particles were in the prefusion conformation. Mass spectrometry revealed that all three predicted N-glycosylation sites were occupied with oligomannose and (core-fucosylated) paucimannose glycans, as expected for the insect cell expression system.

Although F_{ECTO} can be purified in the prefusion state, the protein refolds to the postfusion state over time. To determine how to maintain the prefusion conformation, Applicants used differential scanning fluorimetry (DSF) to systematically explore the impact of pH and salt concentration on the apparent melting temperature (Tm app) of F_{ECTO}. Although pH had little influence on the Tm app of F_{ECTO}, higher salt concentration increased the Tm app from 44.6° to 54.8 °C, at 60 and 1460 mM NaCl, respectively (FIG. S2F). Hence, buffering conditions of 800 mM NaCl and 50 mM HEPES pH 8.0 were used in the subsequent purification of F_{ECTO}. Applicants saw no F_{ECTO} in the postfusion conformation, even after >2 weeks of storage in this buffer.

### Structure of Prefusion F

Applicants determined a 2.11-A-resolution cryo-electron microscopy (cryo-EM) structure of F in its unbound, prefusion state to investigate the influence of the introduced stabilizing mutations (FIG. 3B). This high resolution structure of the natural F_{ECTO} closely resembles the previous cysteine-stabilized prefusion F ectodomain with two exceptions. First, loop 248-265 adopts a different conformation, likely attributable to previous crystal contacts. Second, the alpha helices of the HRC in the ectodomain stalk resolved here begin to supercoil around each other starting at residue 473, whereas those in the Cys-stabilized construct remain linear and do not supercoil.

Prefusion F in Complex with Inhibitor

Applicants next solved the structure of F_{ECTO} in complex with [FIP-HRC]2-PEG11, a tandem peptide conjugate of two copies each of fusion inhibitor peptide (FIP)-HRC, linked by a PEG₁₁ moiety (FIG. 3C). As this inhibitor contains both FIP, which stabilizes prefusion F, and an HRC mimic, which binds to the pre-hairpin intermediate, it could theoretically capture either the pre- or post triggered state of F, provided the correct state is available for binding. Applicants obtained a high resolution structure of the F_{ECTO}-[FIP-HRC]2-PEG11 complex at 2.3-Å resolution (FIG. 3C). Similar to the previous structure of MeV Cys-stabilized F in complex with a related peptide inhibitor, here Applicants find that FIP stabilizes F by occupying the hydrophobic pocket created by F1 residues from all three protomers, an interaction that locks MeV F in the prefusion state. All F_{ECTO} particles adopt a prefusion state with apparent density for FIP but no density for the HRC-derived peptide. The lack of observable alternative conformations of F and the lack of HRC density together suggest that a fusion-intermediate state was not available for HRC to bind stably.

Prefusion F in Complex with mAb 77

Applicants cell-based assays indicate that binding of mAb 77 locks F into an intermediate state of refolding, after release of the fusion peptide, but before collapse of the trimer into the six-helix bundle required to complete fusion. The Fab of mAb 77 (Fab 77) binds F_{ECTO} with a dissociation constant (Kd) of 360 pM (0.36 nM), which is consistent with its potent in vitro neutralization. To elucidate the binding epitope of mAb 77 and further discern its mechanism of action, Applicants obtained a F_{ECTO}-Fab 77 complex and determined its high-resolution structure by cryo-EM.

The final map of the F_{ECTO}-Fab 77 complex was resolved to 2.6-Å resolution, showing three copies of Fab 77 binding to the prefusion F_{ECTO} trimer (FIG. 3E). The antibody epitope lies at the base of the trimer and encompasses regions in both F1 and F2 from one protomer plus an additional F1 from a neighboring protomer (FIG. 3, C, D, and F). The heavy chain of Fab 77 contributes the bulk of the interaction, burying 889 Å2 of surface area on F_{ECTO}, whereas the light chain buries 106 Å2. The predominant interactions between the antibody and F_{ECTO} occur through the heavy-chain complementarity-determining region (CDR) 3, with additional contributions from CDR H1, CDR H2, and CDR L2 (FIG. 3F).

CDR H2 contacts F2A residues W27, G37, and S40. CDR H1 residues Y27 and S31 form hydrogen bonds with the backbone of residues L123 and V125, located in the fusion peptide of F1B in the neighboring protomer (FIG. 3E). Meanwhile, CDR-H3 occupies a shallow cleft between domains I and II of F1B, forming six hydrogen bonds and one salt bridge. F1B residue R436 forms hydrogen bonds to the backbone oxygens of CDR H3 residues Y105 and Y107 and a salt bridge with CDR H3 D109 (FIG. 3G). CDR L2 residues Y49 and Q55 also bind to this region, making a hydrogen bond with the backbone nitrogen of F1B residue R435 and the hydroxyl group of S434 (FIG. 3H). Together, this extensive network of 16 hydrogen bonds and one salt bridge anchor domains I and II of F1 in the prefusion state. Despite the glycan being closely located to the Fab 77 epitope at N29, no interactions were observed between Fab 77 and N29.

### F Postfusion Structure

The structure of F_{ECTO} in complex with Fab 77 unequivocally demonstrates that this mAb recognizes the prefusion conformation of F and anchors the fusion peptide through interactions with CDR H1. However, the RBC fusion assay (FIG. 2) suggests that mAb 77 prevents viral infection by arresting the fusion process after the fusion peptide has been released from the trimer and inserted into the host cell membrane.

To reconcile the structural finding that the Fab 77 epitope is in prefusion F, with the experimental data suggesting that the Fab 77 stabilizes a transitional state after activation, Applicants determined the cryo-EM structure of MeV F in its postfusion state. Direct structural comparison of the pre- and postfusion states from the same protein sequence would provide a more comprehensive understanding of the conformational dynamics of F_{ECTO} and clarify how mAb 77 might selectively interact with the prefusion form or reveal conservation of the epitope in the postfusion form.

As mentioned above, storage of F_{ECTO} in a high-salt buffer promotes conformational stability, whereas storage in a low-salt buffer induces spontaneous refolding to the postfusion state. Hence, Applicants incubated F_{ECTO} in low-salt buffer (150 mM NaCl) and solved a 2.7-Å-resolution cryo-EM structure of its postfusion conformation, using the same construct as represented in F4 the prefusion F_{ECTO} structures (FIG. 4, A and B).

Like postfusion structures of other paramyxovirus F proteins, the MeV F_{ECTO} postfusion structure adopts a distinct elongated shape with a refolded head region and an extended six-helix bundle composed of HRN and HRC (FIG. 4, A and B). This conformational shift reduces the head radius by around 20% (from ~110 Å to ~93 Å) and extends the protein's long dimension by 60 Å. The fusion peptide (FP; residues 115 to 140) is part of the F_{ECTO} sequence, but Applicants could not observe the corresponding density for the fusion peptide, likely owing to its inherent flexibility.

Alignment of the postfusion structure of F_{ECTO} with its prefusion counterpart revealed that domain I (residues 290 to 373) and the N terminal part of F2 have the lowest degree of rearrangement during fusion and provide a structural hinge about which the movement and rotation of other domains occur (FIG. 4C). In contrast, domains II (F1 residues 374 to 426) and III (F1 residues 115 to 287 and F2 residues 50 to 104) show substantial spatial and structural rearrangement (FIG. 4, D and E). Although most of domain II retains the immunoglobulin-like fold present in the prefusion conformation, residues 434 to 446 of this domain, as well as the linker connecting domain II to HRC (residues 447 to 456), elongate as HRC transitions from the “down” state to the “up” state as fusion proceeds. As a result, all of domain II shifts 4.3 Å toward domain I.

The most profound conformational change between the pre- to postfusion state occurs in the fusion peptide and HRN. Structural rearrangement within these regions includes refolding of HRN from several disjointed helices and -strands to one elongated helix, exposure of the buried fusion peptide, and a 52° rotation around residue T287 (FIG. 4D). Overall, the conformational transition from pre- to postfusion involves drastic refolding of the fusion peptide and HRN, as well as retention of the globular domain structure with repositioning of the domains relative to their prefusion positions.

The major conformational shifts and repositioning of the three distinct components of the tripartite mAb 77 epitope clarify why this antibody cannot recognize F in its postfusion state (FIG. 4F). Specifically, the spatial rearrangement between domain II and the HRC, coupled with the movement of domain II relative to domain I, lead to the dislocation of the mAb 77 epitope and loss of recognition of postfusion F.

### Preservation of the mAb 77 Binding Interface in MeV Variants

Although MeV has been generally considered to be serologically monotypic, genetic characterization of WT viruses identified eight clades (A to H) that can be further divided into 22 genotypes plus one additional proposed genotype. To evaluate whether natural genetic variation in MeV might affect mAb 77 binding, Applicants examined sequence conservation across the available MeV F protein sequences in the National Center for Biotechnology Information (NCBI) database. Of the 550 residues in MeV F, 319 are fully conserved among the variants, but 231 (42%) have single or multiple amino acid substitutions (FIG. 5, A and B). F5 Within the mAb 77 epitope, however, only two residues, G37 and R436, vary across the range of known MeV sequences (FIG. 5C). The G37R mutation is extremely rare, occurring in only one instance (0.12% of sequences), but R436K occurs in >25% of F sequences. Binding of mAb 77 observed for WT F bearing an R436K mutation is similar, but mAb 77 did not bind F carrying the rare G37R mutation (FIG. S8, A to C). In addition, the G37Rmutation also decreased F-mediated fusion activity, which may be one reason for the low natural incidence of this substitution in circulating viruses (FIG. 5D).

### mAb 77 Interrupts the Fusion Cascade, Leading to Trimer Disintegration

The structure of mAb 77 in complex with F, coupled with the postfusion structure of F, clearly demonstrated that the epitope targeted by mAb 77 is specific to the prefusion conformation. However, the mechanistic fusion assay suggested that mAb 77 acts on F at an intermediate state, after F has been triggered and fusion activated, but before the six-helix bundle has formed.

To further illuminate the mechanism by which mAb 77 inhibits viral entry, Applicants investigated its impact on F_{ECTO} structure and stability. Specifically, Applicants considered two (not mutually exclusive) hypotheses: (i) mAb 77 induces a conformational change in the epitope of F, potentially leading to the destabilization of the trimer; or (ii) mAb 77 stabilizes both prefusion F_{ECTO} and an intermediate state, halting the refolding cascade from two arms. To test these hypotheses, Applicants compared the structure of F in the absence and presence of Fab 77 and then assessed the impact of Fab 77 on F thermal stability using DSF and temperature-resolved dynamic light scattering (DLS). These complementary approaches enabled Applicants to assess the overall stability of F and F-Fab 77 complexes (DSF) and monitor temperature-induced changes in protein conformation (DLS).

Alignment of the structures of F_{ECTO} alone and in complex with Fab 77 demonstrated high structural conservation, with a root mean square deviation (RMSD) of 0.75 Å over all Ca or 1.24 Å over all atoms. Residues involved in the mAb 77 epitope showed only minor differences between structures, with fusion peptide residues L123 and V125 having an RMSD of ~2 Å over all atoms. Hence, binding of Fab 77 only marginally altered the conformation of F_{ECTO}.

DSF analysis demonstrated a modest increase in the thermostability of F when bound to Fab 77 (Tm app of 47.6 °C versus 51.6 °C in the unbound and bound state, respectively; FIG. S9C). This stabilization was more limited than that seen for [FIP-HRC]2-PEG11 (56.2 °C) but nevertheless suggests that binding of mAb 77 does not inherently destabilize F_{ECTO}. Meanwhile, although DLS analysis also demonstrated that the temperature at which the F_{ECTO}-Fab 77 complex begins to unfold (Tm onset) is similar to that of F alone, Applicants found that the conformational behavior of the complex is markedly different. In the absence of Fab 77, the hydrodynamic radius (Rh) of F_{ECTO} is ~5.5 nm up to 44.7 °C, beyond which there is a steady temperature-dependent increase in Rh, likely reflective of the transition to the postfusion state, followed by aggregation. Conversely, a clear plateau phase is observed for the F-Fab 77 complex, which remains arrested at an Rh of ~30 nm between 50° and 65 °C. This plateau is only surpassed above 65 °C, when Fab 77 presumably unfolds and releases F. These data suggest that mAb 77 only modestly enhances the stability of prefusion F but readily inhibits its collapse to the postfusion conformation through stable association with a fusion intermediate.

To visualize this potential intermediate(s) directly, Applicants revisited the F_{ECTO}-Fab 77 cryo-EM dataset, this time incorporating previously rejected Fab-bound classes. Through extensive two-dimensional (2D) classification, Applicants identified several additional 2D class projections showing distinct Fab-induced F_{ECTO} conformations F6 (FIG. 63-65). Included in these projections was a complex of three Fabs bound to an F_{ECTO} trimer that is conformationally distinct from that of the prefusion trimer. In this case, a part of the head region appears to be drawn into the center of the F trimer, suggesting that a three-helix bundle would form upon release of the fusion peptide and refolding of the HRN. Applicants additionally observed a class in which one F_{ECTO} protomer was dissociated from the trimer, leaving a “V”-shaped structure formed by the remaining two protomers, each bound by a Fab 77 molecule. Finally, Applicants identified a 2D class in which Fab 77 is bound to a small F_{ECTO} fragment (FIG. 63-65). Similar 2D classes could be identified using time-resolved negative stain electron microscopy (NS-EM) analysis, in which F_{ECTO}-Fab 77 complexes were incubated for 1 to 10 min at 50 °C before grid preparation.

The scarcity of particles and views in the cryo-EM dataset hindered efforts to obtain 3D reconstructions for the trimeric refolding intermediate and separated protomer complexes. However, Applicants successfully determined the structure of the Fab 77-stabilized F_{ECTO} fragment to 3.6-Å resolution (FIG. 63-65). This structure reveals that Fab 77 is bound to F_{ECTO} domains I and II as well as the N-terminal segment of F2, in a manner similar to that of Fab 77 bound to prefusion F_{ECTO} (FIG. S11G). The lack of density for the fusion peptide suggests that this region has been released from the Fab 77 epitope, a possibility that is consistent with the results of the biochemical and cell-based assays.

### DISCUSSION

Most mAbs described thus far against MeV are directed against the receptor binding protein H. The limited set of mAbs identified for the fusion protein F fall into two principal antigenic categories, I and II. Category I mAbs are potently neutralizing but susceptible to escape by naturally occurring MeV variants. Category II mAbs typically exhibit less potent neutralization and protection in mice yet offer greater resilience against escape mutants. Where these antibodies specifically bind on the fusion protein is currently unknown, as the precise epitopes have not been characterized. mAb 77 (mAb 77) is category II but has high neutralization potency in vitro and protection in vivo.

Several antibodies that bind the prefusion state of other paramyxovirus fusion proteins have been described, and for the paramyxovirus respiratory syncytial virus (RSV), two such antibodies are in clinical use. In contrast to MeV, other paramyxovirus fusion proteins have three, rather than two, major epitope classes: the apex of the head domain, the trimer interface where two protomers meet, and the base of the head domain. Two mAbs that target Nipah virus F, 2B12 and 1H1, bind the base of the head domain that encompasses primarily domain II with single interactions with either domain I (2B12) or the cathepsin L cleavage site (1H1). In this study, Applicants show that mAb 77 against MeV also binds the base of the head domain, but its quaternary epitope spans across three domains and three polypeptide chains (FIG. 3, E and G). Applicants hypothesized that mAb 77, like the Nipah mAbs 2B12 and 1H1, inhibits viral entry by binding to prefusion F before it has been activated by H-receptor engagement on the target cell. This mechanism differs from that of HRC peptide mimics, which interact with the F protein only after activation of fusion, when the virus has attached to the cell membrane, but before fusion has been completed. Unexpectedly, however, the mechanistic assays demonstrated that mAb 77 does not block any initiation of conformational change. Instead, like HRC mimics, mAb 77 allows initial triggering but arrests the protein at an intermediate step along the fusion pathway (FIG. 2).

Curiously, the quaternary epitope of mAb 77 includes, at its edge, the fusion peptide of F, which extends from a neighboring protomer (e.g., FB) onto the top of domain II of FA. However, the results of the mechanistic assay indicate that the fusion peptide is released and inserted into the host membrane, despite mAb 77 binding. Applicants note that the fusion peptide makes a relatively small contribution to the overall epitope. Indeed, only two of the 16 hydrogen bonds between mAb 77 and Finvolve the fusion peptide (CDR H1 Y27 and S31 with L123 and V125, respectively). Based on PISA (Proteins, Interfaces, Structures and Assemblies) server predictions, these interactions contribute just-1.5 kcal/mol of free energy upon complex formation, in contrast to the −8.6 kcal/mol discharged across the entire epitope. Hence, the fusion peptide, located on the edge of the epitope, could potentially be extracted even though mAb 77 remains anchored to the N terminal portion of F2 and the loop between domain II and HRC. The stronger remaining interactions would likely still preclude HRC repositioning, which requires release of the domain II-HRC loop. Furthermore, the structures suggest that the fusion peptide is not necessarily the first region to refold upon fusion triggering. Instead, the refolding of HRN from several disjointed subsections into an elongated helix could be the driving force for the refolding that, once initiated, pulls the FP from its location atop the neighboring domain II, like a ribbon from a box. This possibility is consistent with previous studies on refolding of the parainfluenza virus 5 F protein examined by oxidative footprinting and mass spectrometry.

On the basis of these biochemical and structural findings, Applicants propose a model of mAb 77-mediated protection against MeV that harmonizes the cell-based mechanistic studies with the biophysical and structural observations (FIG. 63-65). In this model, (i) mAb 77 anchors F in a prefusion state through extensive interactions with two adjacent protomers; (ii) receptor engagement by H triggers refolding of F-HRN and release of the fusion peptide from the mAb 77 epitope; and (iii) continued binding of F by mAb 77 prevents complete refolding by anchoring F2 to domains I, II, and the linker in between. As a result, the binding of mAb 77 arrests an intermediate between pre- and postfusion states.

It is not yet clear whether mAb 77-mediated stabilization of prefusion F would completely inhibit F triggering in a natural viral infection or whether the primary mechanism of action for this mAb is to prevent the completion of fusion. The temperature-dependent DSF and DLS experiments demonstrated that mAb 77 enhances the stability of prefusion F up to ~50 °C, after which the complex adopts a conformational species distinct from that of apo-F. Meanwhile, the RBC-fusion assays indicated that Fis activated at 37 °C, even in the presence of mAb 77. These seemingly contradictory results may be explained by the stabilizing mutations present in recombinant F_{ECTO} used for DSF and DLS experiments versus the destabilized, full-length F used in the cell-based assay. It is also unclear whether the intermediate states captured by NS- and cryo-EM reflect the structural trajectory of apo-F or whether they are distinct states induced or enforced by mAb binding. Nevertheless, the analyses demonstrate that in addition to stabilization of prefusion F, mAb 77 also prevents fusion by remaining bound to a post-triggered F.

This dual mechanism of antibody-mediated defense may also apply to other antibodies that are specific for prefusion states. Several previous studies focusing on similar antibodies against Nipah, Hendra, or parainfluenza viruses have primarily examined binding kinetics, neutralization efficiency, and structure but did not extensively explore mechanisms of activity. As a contrasting case, Applicants have shown that the HPIV3-neutralizing antibody PIA174 stabilizes the prefusion state of the protein. The only other antibodies shown to recognize a refolding intermediate state of a viral fusion protein are the HIV-1 gp41-specific mAbs 2F5 and 4E10. These rare antibodies were known to neutralize by targeting a transient conformation during gp41-mediated membrane fusion. However, structures illuminating their specific binding to the intermediate state are as yet unresolved. The structures presented here, of the beginning and ending states of MeV F at high resolution, with additional lower-resolution views of captured intermediates along the path, illuminate a mechanism of neutralization by conformational arrest of the fusion protein at an intermediate, posttriggered state. This work paves the way for the next generation of measles virus therapeutics and illuminates a mechanism that may be shared by antibodies against other viral pathogens.

### Materials and Methods

Plasmids. The genes of Measles morbillivirus (MeV) strains IC323 or B3 hemagglutinin protein (H), fusion protein (F), human nectin 4, and human CD150 (huCD150/SLAM) proteins were codon optimized, synthesized, and subcloned into the mammalian expression vector pCAGGS (Epoch Life Science). A codon-optimized F protein sequence derived from the Measles virus strain Ichinose-B95a (Taxonomy ID: 645098) was synthesized by GenScript and subcloned into the pcDNA3.1 vector. Stabilizing point mutations (E170G and E455G) previously characterized in SSPE and MIBE measles strains were sequentially introduced using the Q5 Site-Directed Mutagenesis kit (NEB). The ectodomain construct was generated by removing the transmembrane region and cytoplasmic tail residues (496 to 550) using the Q5 Site-Directed Mutagenesis kit. To generate Drosophila S2 stable cell lines, the F protein ectodomain construct E170G/E455G (F_{ECTO}) was subcloned from the plasmid cloning DNA (pcDNA) vector to the pMT-puro vector, including a C-terminal enterokinase cleavage site and a dual StrepII tag via NEB HiFi DNA assembly.

Cells. HEK293T (human kidney epithelial), Vero, and Vero-huCD150 (African green monkey kidney) cells were grown in Dulbecco's modified Eagle's medium (DMEM; Life Technologies; Thermo Fisher Scientific) supplemented with 10% fetal bovine serum (FBS, Life Technologies; Thermo Fisher Scientific) and antibiotics at 37 °C in 5% CO₂. The Vero-huCD150 culture medium was supplemented with 1 mg/ml of geneticin (Thermo Fisher Scientific). Drosophila S2 cells were cultured at 25 °C without CO2 in complete Schneider's Drosophila Medium (Gibco) supplemented with 10% heat-inactivated FBS and 1% penicillin-streptomycin (100 U/ml penicillin and 100 mg/ml streptomycin) or in serum-free Lonza Insect Xpress Medium. ExpiCHO cells were maintained in ExpiCHO Expression Medium (Gibco) supplemented with 8 mM L-glutamine in a humidified incubator at 37 °C with 8% CO2.

In vitro viral infection. Vero-huCD150 cells were plated in 96-well plates (1 Å~104 cells per well). The next day, cells were treated with serial dilutions of mAb 77, compound 3G, or MeV F-derived HRC4 dimeric fusion inhibitory lipopeptide (i.e., [HRC-PEG4]2-chol). Simultaneously, cells were infected with MeV WT B3-eGFP for 3 hours at 37 °C [150 to 200 plaque-forming units (PFU) per well]. After 3 hours, the medium was replaced with medium containing 2% of methylcellulose Avicel/2 Å~ complete medium (1:1). After 24 hours, eGFP-infected cells were counted using a Cytation 5 (BioTek) and analyzed with Gen5 3.11 software (min object size: 25 mm; max object size: 135 mm; threshold: 3000). The samples were tested in three experiments performed with technical duplicates in fivefold dilutions of 96-well plates. The measurements from each plate were normalized using 100nMof mAb 77, 25 mM of compound 3G, or 500 nM concentration of HRC4 as 100% inhibition. Data were analyzed in OriginPro 2023, where all biological and technical replicates were globally fitted with logistic regression function with shared IC₅₀ and Hill slope values, and A1 and AO (highest and lowest signals) were used to normalize the data. Percent inhibition was calculated on the basis of the normalized data using the following equation: [1−X]*100, where X is the fraction of the normalized signal.

β-Galactosidase (β-Gal) complementation-based fusion assay. The β-Gal complementation-based fusion assay was performed as previously described. Briefly, HEK293T cells transiently transfected with nectin 4 or huCD150 and the b-Gal omega subunit are mixed with cells coexpressing glycoproteins F and H and the b-Ga alpha subunit. Cell fusion leads to alpha-omega complementation and is measured in the presence or absence of fusion inhibitory compounds. Fusion is stopped by lysing the cells and, after addition of the substrate (The Tropix Galacto-Star chemiluminescent reporter assay system, Applied Biosystems), fusion is quantified on either a Tecan M1000 microplate reader or a Biotek Cytation 5. Three biological replicates were recorded and globally fitted with a logistic function in OriginPro 2023. Obtained parameters were used to normalize the data per sample and plotted with standard error calculated from all data points.

Cell surface staining with conformation-specific anti-F mAbs. HEK293T cells were transiently transfected with viral glycoprotein constructs. The transfected cells were incubated at the temperatures and times indicated in the figures. Specific MeV F conformations were detected with mouse mAbs followed by Alexa-488 anti-mouse secondary antibody (Life Technologies) and DAPI (4′,6-diamidino-2-phenylindole; Thermo Fisher) staining. Plates were imaged with a Cytation5, and the relative fluorescence of antibody-bound cells was determined using BioTek Gen5.

Viral rescue. HEK293T cells were transfected using Lipofectamine 2000 in six-well plates coated with poly-D-lysine. The transfection mixture included cDNA constructs coding for viral proteins N (GS6558900-1), P (GS59482), and L (GS58944), all sourced from Epoch Life Science according to the design specifications. Additionally, cDNA for T7 polymerase (GS58929) and the genome for the MeV B3 eGFPWT virus (GS68838) were included. Cells were incubated overnight at 37 °C in Opti-MEM medium. The medium was then replaced with DMEM supplemented with 10% FBS. Cells were then subjected to a heat shock treatment by incubation at 42 °C in a water bath for 3 hours. Subsequently, the cells were returned to 37 °C for 48 hours. The cells were then detached and co-incubated with Vero-huCD150 cells in T75 flasks to facilitate rescued virus amplification. The virus underwent four passages and was titrated to a concentration of 106 PFU/ml. The virus sample was then sequenced and used in subsequent in vitro and in vivo experiments.

In vivo experiments: Cotton rats. Inbred cotton rats (Sigmodon hispidus, cotton rat strain “Hsd: Cotton Rat” without genetic modifications) were purchased from Envigo, Inc., Indianapolis. Male and female cotton rats aged 5 to 7 weeks were used for experiments. For intranasal infection, 105 TCID50 of MeV (strain B3 wt eGFP) was inoculated intranasally to isoflurane-anesthetized rats in a volume of 100 ml. Animals received intraperitoneally either the indicated amount of mAb 77 or vehicle 12 hours before infection. Four days after infection, animals were euthanized by CO₂ inhalation, and their lungs were collected and weighed. Lung tissue was homogenized with a glass dounce homogenizer. Supernatants were serially diluted and titrated for infectious virus on Vero-huCD150 cells in 48-well plates. Plates were scored microscopically for cytopathic effect (CPE) after 7 days, and the tissue culture infectious dose 50 (TCID50) was calculated. The results are presented as TCID50 per gram of lung tissue.

RBC fusion assay. To investigate the specific stage at which mAb 77 affects the fusion process, Applicants used a previously characterized assay involving RBCs and HEK293T cells transfected with a chimeric version of MeV H and MeV F bearing a T461I mutation. The chimeric version of H (H-HN) contains the stalk region of MeV H to maintain interaction with MeV F fused to the sialic acid receptor-binding domain of HPIV3 HN protein. The interaction between H-HN and sialic acid receptors on RBCs can be reversed through treatment with zanamivir, which enables the differentiation of F-mediated anchoring from H-mediated anchoring. The T461I mutation was introduced into the F protein to reduce its stability and enhance fusion activation. Each of these specific proteins have been used in previous studies.

Transfected HEK293T cells were incubated with a 1% v/v of RBCs in CO₂-independent media for 30 min at 4 °C to allow for receptor binding without initiating the fusion process.

After this incubation period, the cells were washed three times with serum-free medium to remove unbound RBCs. The cells were then exposed to either diluent medium or mAb 77. MeV-F targeted inhibitors with known functions were also included as controls. These included compound 3G, which prevents H mediated fusion activation by binding to prefusion F, and a peptide derived from the heptad repeat C (HRC) region, named HRC4, which obstructs the conformational transition necessary for fusion by interacting with the HRN in the prehairpin intermediate state. After these treatments, the cell mixtures were warmed to 37 °C for 60 min to activate F mediated fusion of the RBCs and 293T cells. The outcomes of these interactions were classified into three groups: group 1: reversibly bound RBCs; group 2: irreversibly bound but not fused RBCs; and group 3: RBCs that had successfully fused with HEK293T cells. Each population was quantified through measurement of hemoglobin as detailed below (see also FIG. S1A for an experimental schematic).

The assay was performed in poly-D-lysine biocotated 24-well plates on which HEK293T cells were plated and transfected with the viral glycoproteins. The plates were then treated overnight at 37 °C with 25 mU per well of exogenous neuraminidase to prevent cell-to-cell fusion and maintain an intact cell monolayer. The next day, the plates were transferred to melting ice (4 °C) for 30 min, washed with cold CO₂-independent media, and then incubated with RBCs (1% v/v) for 30 min. After this incubation, the unbound RBCs were washed from the cell surface, the treatments were added, and the cells were incubated at 37 °C. Afterward, the plates were transferred to melting ice (4 °C) for assessment of the different populations of RBCs (reversibly bound, irreversibly bound, and fused). Reversibly bound RBCs (group 1) were separated from the cells expressing the viral glycoproteins by treating the wells with 10mMzanamivir for 30 min at 4 °C. The addition of zanamivir allowed the resuspension of the RBCs in the supernatant fluids, whereas the cells expressing the viral glycoproteins remained attached to the plastic in the well. The supernatant fluids containing the reversibly bound RBC were moved into V-bottomed 96-well plates and spun down. The RBCs remained at the bottom of the V-bottomed well, and the supernatant was removed and replaced with ACK-Lysing buffer (ammonium-chloride-potassium, from Gibco A10492-01) to released in solution the hemoglobin. Irreversibly bound RBCs (group 2) were measured by incubating the cells (minus the removed, reversibly bound RBCs) with ACK Lysing buffer (ammonium-chloride-potassium, from Gibco A10492-01) for 30 min at 4 °C. ACK buffer selectively lyses (unfused) RBCs by disrupting the internal ionic balance, resulting in the release of hemoglobin directly into the media. The hemoglobin was released in the supernatant fluids that are transferred to 96-well plate for hemoglobin quantification. The remaining cells (still bound to the plastic in the well) were fusions of RBC and HEK239 Ts (group 3) and are ACK buffer insensitive. To release the hemoglobin, this population of cells were lysed with 50 mM HEPES (Gibco Cat #15630080), 100 mM NaCl, and 5 mg/ml dodecylmaltoside (Thermo Scientific Cat #89903) (DH buffer) and clarified by centrifugation. Lysate was then moved to flat-bottomed 96-well plates for hemoglobin quantification.

The level of hemoglobin was quantified for each group by measuring the absorbance of each lysate at 410 nm. The amount of hemoglobin in each group was added together to represent 100%. Each group was displayed as a fractional proportion of this normalized value.

F_{ECTO} purification. Drosophila S2 cells were transfected with a plasmid encoding the F ectodomain construct (pMT-puro-F_{ECTO}2M-EK-twin-Strep-TagII) following the manufacturer's protocol (referred to throughout the text as F_{ECTO} for simplicity). On day 1, S2 cells were seeded in a six-well plate (1.0×10⁵ to 1.5×10⁵ cells/ml) in 3 ml of complete Schneider's medium. On day 2, the cells were transfected with 2 mg of the plasmid using the Effectene (QIAGEN) transfection protocol. Selection with puromycin (6 mg/ml) started on day 5, at which point cells were transferred to a T25 flask. The cells were then gradually expanded to a T75 flask, with the medium replaced by Lonza Insect Xpress medium containing 6 mg/ml puromycin. Subsequently, cells were expanded to 2×1 L in 2 L shaking flasks, induced with 500 mMCuSO4at 1×10⁷ cells/ml, and harvested four days postinduction. Cells were removed from the culture medium by centrifugation at 5000 g for 20 min. The medium was adjusted to pH 8.0 with 1 M HEPES, and Biolock (IBA) (2.4 ml per liter of medium) and NaCl (to a final concentration of 800 mM) were added. The medium was loaded onto a StrepTrap HP 5 ml column (Cytiva), and the column was washed with high-salt HBS (50mMHEPES pH 8.0, 800 mM NaCl) until a stable UV absorbance at 280 nm was achieved. The protein was then eluted with high-salt HBS containing 5 mM d-desthiobiotin (Sigma). Next, the eluted protein fractions were pooled, and the buffer was exchanged for high-salt HBS using a HiPrep 26/10 Desalting column (Cytiva). Finally, the purified protein was flash-frozen in liquid nitrogen. Protein concentration was estimated using the molar extinction coefficient at 280 nm (48,820 M⁻¹ cm⁻¹).

Antibody expression. mAb 77) was produced in ExpiCHO cells by following the manufacturer's high-titer transfection protocol using the pTRIOZ-mAb 77 plasmid, which encodes the antibody heavy and light chains. After seven days of expression, cells were removed from the culture medium by centrifugation at 4000 g. The medium was then adjusted to pH 8.0 with 1 M HEPES (pH 8.0) and incubated overnight at 4 °C with protein A agarose beads to capture the mAb 77. On a subsequent day, the beads were washed with 20 ml of HBS, and the bound antibody was eluted using 15 ml 0.2 M glycine (pH 2.0). The eluate was immediately neutralized by adding 10% (v/v) 1 M HEPES (pH 8.0). To generate antibody fragments (Fabs), mAb 77 was digested at 1 mg/ml with 1% (w/v) cysteine-activated papain for 4 hours at 37 °C in a water bath. The enzymatic reaction was halted by adding 0.5Miodoacetamide to a final concentration of 50 mM. After digestion, the reaction mixture was diluted 10-fold with 10 mM HEPES (pH 8.0) to decrease salt concentration and loaded onto a 1 ml Mono S column preequilibrated with 20 mM HEPES (pH 8.0). The Fabs' flowthrough was collected, and the Fc fragments were eluted using a single step with 20 mM HEPES (pH 8.0) containing 2 M NaCl. The isolated Fabs were concentrated, and their buffer was exchanged for HBS. The Fabs were stored at 4 °C for further analysis and experimentation. Protein concentration was estimated using the molar extinction coefficient at 280 nm (mAb 77: 240360M⁻¹ cm⁻¹; Fab 77:84,270M⁻¹ cm⁻¹).

Formation of F_{ECTO}-Fab 77 Complex. The F_{ECTO} and Fab 77 complex was prepared by incubating ~5 mMof F_{ECTO} with a threefold molar excess of Fab 77 at 4 °C overnight in HBS (50 mM HEPES, pH 8.0, and 150 mM NaCl). After the incubation, the mixture was subjected to size-exclusion chromatography using a Superdex 200 Increase 10/300 GL column (Cytiva) in HBS. The fractions containing the F_{ECTO}-Fab 77 complex were collected, concentrated, and used on the same day.

Differential scanning fluorimetry assay. DSF was used to determine protein stability and evaluate variations in thermal denaturation profiles under distinct buffer conditions. Each analysis was conducted using 25 ml containing 2.5 mg of protein and 2.5 ml of a 1% SYPRO Orange solution (Thermo Scientific), with the remaining volume composed of the buffer being tested. Stability measurements for F_{ECTO}, F_{ECTO}-Fab 77, F_{ECTO}-[FIP-HRC]2-PEG11 were performed in HBS. For pH screening, buffers contained 50 mM sodium acetate (pH5.0), Bis-Tris (pH 6.0), MOPS (pH 7.0), HEPES (pH 8.0), or CAPSO (pH 9.0). The salt screening was performed using 50 mMHEPES (pH 8.0) with NaCl concentrations ranging from 0 to 2 M.

The samples were dispensed into a 96-well polymerase chain reaction (PCR) plate (BIO-RAD Multiplat 96-Well PCR Plates, low profile, unskirted), and the plate was then sealed (Bio-RAD Microseal ‘B’ PCR Plate Sealing Film) to prevent evaporation during the heating process. A thermal cycling procedure was initiated on a Bio-Rad CFX 96 thermocycler, with the temperature incrementally elevated from 25 °C to 95 °C at a rate of 1 °C per min.

Throughout thermal cycling, SYPRO Orange fluorescence was monitored continuously with the wavelengths for excitation and emission set at 520 and 540 nm, respectively. Data analysis was automatically carried out in Bio-RAD CFX Manager software, and the first derivative of the melting curve was graphed against temperature. Each experimental condition was performed in quadruplicate, and the average derivative signal was calculated. The averaged derivative signal minimum represented the protein's apparent melting temperature (Tm app).

Dynamic light scattering. DLS experiments were performed using a Dynapro PlateReader-2 (Wyatt) instrument with Corning 384-well plates with clear bottom. Four independent replicates were recorded for each sample, and 25 ml of protein at 0.05 mg/ml was added to each well. The temperature ramp was set to increase at 0.2 °C per min from 25 °C to 70 °C, resulting in a 1 °C ramp per cycle. The autocorrelation function for each replicate was fitted using regularization fit in DYNAMICS 7.8.1.3 software (Wyatt), and the radius of the dominant mass species was plotted against temperature. Ten percent of the total signal change was used to calculate the temperature onset, which was manually estimated for the obtained curves. For the Fab 77 fitting, the maximum signal was fixed at double the last measured point. The resulting plots were generated using Matplotlib.

Biolayer interferometry. Biolayer interferometry (BLI)-based kinetic analysis was conducted using the OctetRed384 system (Sartorius). Anti-human Fab (FAB2G, Sartorius) capture sensors were loaded with Fab at 15 mg/ml in 10 mM HEPES (pH 7.4), 150mMNaCl, 3 mMEDTA, 0.05% Tween-20, 0.5 mg/ml BSA (1xHBSTE/BSA). Binding of FECTO to Fab-loaded sensors was performed using twofold serial dilutions of FECTO. Baseline and dissociation steps were carried out in 1xHBSTE/BSA. Regeneration was carried out using 10 mM Glycine (pH 2.5). Reference sensors (those not loaded with Fab but dipped into F_{ECTO} containing wells) were subtracted from each dataset to account for nonspecific F_{ECTO} binding. Fab-only sensors were used as sample references. Kinetic parameters (Kₒₙ and K_{off}) and affinities (Kd) were calculated from a nonlinear fit based on the 1:1 binding kinetic model of the data using the Octet Data AnalysisHT software version 11 (Sartorius). Three replicates for each binding curve were performed.

Structural mapping of protein variants. The sequences for MeV F proteins were acquired using a custom Python script using the BioPython library. This script was used to query the Entrez protein database for “Measles morbillivirus Fusion.” All results obtained from this process were consolidated into a single FASTA file.

The sequences were then filtered according to size, retaining only those longer than 400 and shorter than 600 amino acids. Subsequently, the Needleman-Wunsch algorithm inside BioPython was used for pairwise alignment with the F WT sequence (UniProt: Q786F3) to curate the sequences further. Any sequences with low alignment scores (<0) were removed from consideration.

After this curative process, the filtered FASTA file was globally aligned using the ClustalOmega software. This global alignment was stored in a clustal format file, which was then used to calculate the sequence conservation for each position using the AL2CO program. Primary sequence conservation was visualized and graphed using Matplotlib and Seaborn Python packages.

Structural sequence conservation was mapped onto the pre- and postfusion structures of F_{ECTO} using ChimeraX 1.6, which also uses AL2CO. Statistics regarding the point mutations at the mAb 77 epitope were computed in Python on the basis of the Clustal alignment.

Cell surface biotinylation. HEK293T cells were seeded in a six-well plate and transfected with measles B3 F proteins (WT, 436K, 436G, 436D, 37R) or measles H, by mixing 2 mg per well of plasmid in OPTIMEM with 4 mg per well of Lipofectamine 2000 (Invitrogen Cat #11668019) in OPTIMEM (Gibco Cat #31985088) for 20 min at room temperature and incubating with cells for 3 hours at 37 °C. After transfection, cells were incubated with DMEM (Gibco Cat #10569044) with 10% FBS (Gibco Cat #16000044) and 1% penicillin-streptomycin (Gibco Cat #15140122) at 37° or 32 °C for 24 or 48 hours. Cells were then incubated at 4 °C with 3.3 mM NHS-S-S-dPEG4-biotin (Quanta Biodesign Cat #10194) in DPBS (Gibco Cat #14287080) for 1 hour before lysing with DH Buffer [50 mM HEPES (Gibco Cat #15630080), 100 mM NaCl, 5 mg/ml dodecylmaltoside (Thermo Scientific Cat #89903) in Milli-Q Water] supplemented with complete Protease Inhibitor Cocktail (1 tablet/50 ml; Roche Cat #11836145001). Biotinylated proteins were incubated with Streptavidin Sepharose beads (Invitrogen 434341) for 16 hours at 4 °C. Protein was eluted from washed beads in reducing Laemmli SDS Sample Buffer (Boston BioProducts Cat #BP-110R), boiled for 10 min, and run on a 4-20% Novex Tris-Glycine Protein Gel (Invitrogen Cat #WXP42026-BOX). The gel was transferred to nitrocellulose using an iBlot quick transfer method (Invitrogen IB23001). The membrane was blocked (Invitrogen Cat #WB7050) and treated with Anti-Measles F HRC (GenScript; Rabbit) and alkaline phosphatase-conjugated anti-rabbit secondary antibody (Invitrogen Cat #WB7105). The blot was then developed using NBT/BCIP Substrate (Invitrogen Cat #WP20001). Analysis of the blot was performed with GelAnalyzer 23.1, and the integrated intensity of the bands was normalized to the expression level of F WT at 37 °C.

### Grid Preparation

### Negative Stain

Copper grids (400 mesh) coated with carbon film (CF400-Cu, Electron Microscopy Sciences) were used for negative staining. The grids were first subjected to glow discharge treatment using the easiGlow Glow Discharge Cleaning System (Pelco) for 15 s at 15 mA to enhance hydrophilicity. Next, the protein sample concentration was adjusted to 0.01 absorbance units at 280 nm, and 3 ml of the sample was applied to the grid for a 1-min incubation. Subsequently, the grid was blotted to remove an excess sample and washed three times with 3 ml deionized water, blotting between each washing step. The grid was incubated with 3 ml 1% uranyl acetate solution for negative staining. After two blotting steps, a final drop of the staining solution was added, followed by a 1-min incubation. The grid was then blotted once more to remove excess stain and allowed to air-dry.

### Graphene Oxide Grids

Graphene oxide (GO) grids were prepared inhouse by diluting a commercial GO solution (2 mg/ml, Sigma-Aldrich) in deionized water to a final concentration of 0.2 mg/ml. This diluted solution was sonicated for 5 min in a water bath to ensure proper dispersion of GO layers. Next, large aggregates were removed by centrifugation at 500 g for 1 min. Finally, the supernatant containing the dispersed GO was collected by transferring the upper 50% solution to a fresh 1.5 ml microcentrifuge tube.

Quantifoil R2/2 grids were washed with ethyl acetate overnight and glow-discharged under the same conditions as the negative stain grids. Then, 3 ml of the GO solution was applied to the grids and incubated for 4 min. The grids were blotted, rinsed with 20 ml deionized water, blotted once more, and air-dried for at least 10 min.

On the same day of grid preparation, dried GO grids were used for protein sample vitrification. The Vitrobot Mark IV plunge-freezing system was operated at 4 °C and a relative humidity of 100%. A 3-ml aliquot of the protein sample (0.2 to 0.5 mg/ml) was deposited onto the GO grid, followed by a 12-s incubation period. After the incubation, the excess sample was blotted away (blot force 0) for 3 s. The grid was immediately plunge-frozen in liquid ethane cooled by liquid nitrogen

### C-Flat Grids

C-flat 1.2/1.3 400 mesh grids were glow discharged and immediately used for sample freezing. A 3-ml aliquot of 0.5 mg/ml protein sample was deposited onto the grid, immediately blotted for 6-s, andplunge-frozen in liquid ethane cooled by liquid nitrogen.

### Cryo-EM Data Processing

### F_{ECTO}-Fab 77

Movies were collected on a Titan Krios operated at 300 KV using a K3 Direct electron detector (Gatan) in counting mode (0.66 Å pixel−1) at a nominal magnification of Å~130,000 using a defocus range of −0.5 to −2.0 mm with the BioQuantum energy filter slit set to 20 eV. Movies (LZW compressed TIFF files) were collected in EPU (Thermo) over a 3-s exposure with an exposure rate of ~15 e-pixel−1 s−1, resulting in a total exposure of ~50 e-Å−2 (1 e-Å−2 frame-1). Downstream processing was done using Relion, homemade Relion scripts (github.com/dzyla/cryoem_notebook; github.com/dzyla/Follow_Relion_gracefully), and cryoSPARC (93-96). Frame alignment was done in Relion 4 using MotionCor2 version 1.4.0 with 6×5 patches and a B factor of 150 (97). CTF determination was performed using CTFFIND4 version 4.1.14 using dose-weighted micrographs.

Particles were auto-picked in Relion from randomly selected micrographs using the Laplacian-of-Gaussian algorithm, extracted at 512 px box size and Fourier-cropped to 64 px (5.28 Å px⁻¹), and used for the 2D classification step. Next, particles resembling glycoprotein were selected and subjected to additional rounds of 2D classification. The best classes showing different glycoprotein orientations were selected and used for Topaz model training over 10 epochs with ~10,000 particles (99). Finally, the optimized model was used to pick particles in the whole dataset. Approximately 1.1M particles were extracted in Relion, imported into cryoSPARC and subjected to two rounds of 2D classification. Classes resembling glycoprotein with bound Fab were selected (148,000 particles), and particles corresponding to Fab only were discarded. Selected particles from cryo-SPARC were transferred back to Relion using a homemade script and reextracted at 512 px with Fourier-cropping to 160 px (2.112 Å px⁻¹). Particles were again imported to cryoSPARC, where the initial model was generated. The obtained model was used for the 3D classification in Relion with three classes.

During the first 25 iterations, global angular searches were performed using 15° sampling intervals. Once the classification reached equilibrium [followed by monitoring class distribution using Follow Relion Gracefully], the 3D classification job was resumed with local angular searches of 7.5° sampling intervals and a 30° local angular search range. This procedure was repeated with a decreasing angular sampling interval and search range (kept double the angular sampling) until there was no improvement in estimated angle accuracy. Two classes showed high-resolution features and were selected for the 3D Refine protocol (142,000 particles). Next, refined particles were reextracted at 512 px with Fourier-cropping to 320 px (1.056 Å px⁻¹) with recentering coordinates. Finally, particles were refined and postprocessed, and CTF and aberration refinement were performed. This procedure was repeated until there was no improvement in the reported postprocessing resolution. Then, the Polishing step was applied, first by training on 10,000 particles with the fraction of Fourier pixels for testing set to 0.5. The obtained trained parameters were used for particle polishing, and particles were extracted at 320 px (1.056 Å px⁻¹). Particles were again 3D refined, and CTF and aberration refinement were performed until there was no further improvement in the reported postprocessing resolution. Finally, polished particles were imported into cryoSPARC, and a nonuniform refinement protocol was used to generate the final cryo-EM map in C3 symmetry. Then, particles were C3 symmetry expanded and refined using the Local Refine protocol in cryoSPARC to obtain the final map. The final resolution reported in the manuscript was calculated in cryoSPARC at Fourier shell correlation=0.143 for all maps.

### F_{ECTO}-[FIP-HRC]2-PEG11

The F_{ECTO}-[FIP-HRC]2-PEG11 structure was recorded and processed following the same workflow as the FECTO-Fab 77 structure. The number of particles and workflow overview is presented in FIG. S5B.

### F_{ECTO}

The F_{ECTO} dataset was acquired as described above, with minor modifications. An increased electron exposure of 70 e-/Å2 was used for data acquisition. Initial data processing was conducted in cryoSPARC version 4.3 Live, where 4.6M particles were extracted from 7980 micrographs through blob picking. A deep 2D classification protocol in cryoSPARC was applied to these particles, from which the best candidates were selected for training of the Topaz model. After Topaz extraction, 12M particles were extracted at a box size of 420 px and Fourier cropped to 64 px.

Through intensive deep 2D classification, a final dataset comprising 1.7M particles of high quality was obtained. These particles were then subjected to an initial 3D reconstruction to recenter 3D coordinates, after which they were reextracted at a box size of 420 px and Fourier-cropped to 300 px. A 3D reconstruction from Homogeneous refinement yielded a resolution of 2.19 Å in C3 symmetry. After symmetry expansion to C3, the particles were classified into 10 distinct classes through 3D classification in cryoSPARC, with a high-resolution limit set at 2.0 Å. The class with the highest resolution, 2.11 Å, was selected for further processing. Local and global CTF refinements were performed, and the final refinement was done using a local refinement protocol. Detailed processing is shown in FIG. S5A.

Postfusion F_{ECTO}. F_{ECTO} was incubated in lower salt buffer (50 mM HEPES pH8.0, 150mMNaCl) for1 week at 4 °C. Quantifoil R1.2/1.3 grids were used to freeze FECTO in postfusion confirmation. F_{ECTO} (3 ml of 0.5 mg/ml) was mixed with 1 ml 0.36mMLauryl Maltose Neopentyl Glycol (LMNG), blotted for 6 s, and frozen in liquid ethane. The data processing was similar to the FECTO-Fab 77 dataset. For the postfusion conformation, data processing involved selecting particles on the basis of size and shape, specifically top views of ~70 Å in diameter and extended side views. The detailed processing is illustrated in FIG. S5D.

Fab 77-stabilized F_{ECTO} fragment and refolding intermediates. To determine the structure of the F_{ECTO} fragment stabilized by Fab 77, Applicants reprocessed the F_{ECTO}-Fab 77 dataset in cryoSPARC 4.4. Corrected movies were imported, and Patch CTF was used to estimate the contrast transfer function values. A permissive blob picker (80 to 120 Å, 0.2 minimum separation distance) yielded 5.6M particles, which underwent Deep2D classification. Classes corresponding to F_{ECTO}-Fab 77 and unresolved junk classes were rejected, and the remaining particles were used for multiple rounds of Deep2D classification. Finally, 17,000 particles showing intermediate states of refolding were used to train the Topaz model. After extraction, particles were divided into those containing the Fab 77-stabilized F_{ECTO} fragment and intermediate refolding steps. For refolding intermediates, ~35,000 particles were selected, and ~700 corresponded to the intermediate refolding step, ~2000 to the protomer-separated state, and ~230,000 to the Fab 77-stabilized F_{ECTO} fragment. The latter were reextracted at 340 px and Fourier-cropped to 160 px. Extracted particles were again subjected to Deep2D classification using a high-resolution information cutoff at 3 Å. Only the best-looking classes were selected (94,000), and the initial model was calculated with high-resolution information up to 3 Å. Homogeneous, nonhomogeneous, and local refinements were used to calculate the final map, including the whole Fab fragment. The map region corresponding to the constant region of the Fab was removed in ChimeraX, and a soft binary mask was calculated using Relion. The final map was calculated using the local refinement procedure with a focused mask centered on the Fab 77-F_{ECTO} fragment interface by adjusting the fulcrum coordinates.

Model building. For the F_{ECTO}-Fab 77 structure, the obtained cryo-EM map was used to dock the previously published F x-ray structure [Protein Data Bank (PDB) ID: 5XYW] using ChimeraX. Next, the antibody model was generated with an inhouse implementation of ColabFold based on AlphaFold2. Once the models were positioned, manual model building was conducted in Coot 0.9.8.7, sourced from the CCP4 package. An automatically sharpened and density-modified map from PHENIX ResolveEM (using only half-maps) was the basis for model building. A real-space refinement protocol followed manual model building in the PHENIX package, consisting of 10 macrocycles without any enforced restraints, and increased the number of Max iterations to 800. Finally, water placement was carried out manually on a density-modified map. Water molecules were positioned where spherical density was visible at map RMSD>5.0, no more than 2.7 Å from protein atoms. Interfaces were analyzed using ChimeraX and PISA server. Electrostatic maps, as well as the generation of F variants, were calculated in ChimeraX. Local map scales for F_{ECTO} were calculated in Occupy.

F_{ECTO} in the postfusion conformation was first generated using homology modeling using SWISS-MODEL software and manually rebuilt in Coot. Refinement and evaluation of the model was done as described above.

In the case of the F_{ECTO} fragment stabilized by Fab 77, the structure of F_{ECTO}-Fab 77 was fitted into the obtained map. Residues located within this map were retained, and portions of the model not encompassed by the map were removed. Subsequently, the remaining model underwent relaxation with Isolde, manual Coot adjustments, and PHENIX refinement.

Heat-induced refolding of F_{ECTO}-Fab 77. A 7.5-mM solution of F_{ECTO} in HBS containing 800 mM NaCl was combined with a 1.5-fold molar excess of Fab 77. This mixture was then diluted fourfold using 50mMHEPES (pH 8.0), resulting in a final NaCl concentration of ~150 mM. The complex was allowed to format room temperature over 4 hours. A 15-ml aliquot of the formed complex was transferred to a PCR tube and subjected to heat treatment in a PCR thermocycler. The incubation times tested were 1, 5, 7.5, or 10 min at 50 °C and 1 or 5 min at 55 °C. After incubation, the samples were rapidly cooled by placing the tube on ice for 5 min. Negative stain grids were then prepared using the described protocol.

The acquired data were processed using cryoSPARC 4.3. After motion correction and estimation of CTF values, particle picking was performed using the blob picker. Subsequent 2D classification was performed using the previously described deep 2D classification protocol to separate particles into distinct classes.

Sequence analysis. Approximately 1400 MeV genomic sequences were obtained from the nucleotide NCBI database. Coding sequence regions were manually annotated and curated to include only those between 400 and 600 amino acid residues in length with pairwise alignment scores greater than zero as compared with the F WT sequence (UniProt: Q786F3). The resulting ~820 sequences were aligned using Clustal Omega, and the conservation score was computed using AL2CO (89). Conservation scores were placed on the 3D models of F_{ECTO}^{PRE} and F_{ECTO}^{POST} in ChimeraX using AL2CO. Sequence variants were determined using a Python script based on the multiple sequence alignment file.

Glycoproteomics sample preparation. For glycoproteomics analysis, a SP3 sample preparation protocol was applied (110). Recombinant measles F_{ECTO} was denatured in the buffer with 2% SDS, 200 mM Tris/HCl, 10 mM tris(2-carboxyethyl) phosphine, pH 8.0, at 95 °C for 10 min followed with 30 min reduction at 37 °C for 30 min. Samples were next alkylated by adding 40 mM iodoacetamide and incubated in the dark at room temperature for 45 min. Three micrograms of recombinant F_{ECTO} was used for each protease digestion. Samples were split in half for parallel digestion with chymotrypsin (Sigma) and alpha lytic protease (Sigma). Sera-Mag beads (GE Healthcare) were used at a bead/protein ratio of 10:1 (w/w) for protein binding. Ethanol was added to the protein-beads mixture reaching final concentration 80% to enhance binding. The mixture was incubated at RT for 10minat 1000 rpm in a ThermoMixer. The supernatant was removed, and protein-bound beads were washed three times with 80% ethanol. Measles F_{ECTO} was digested on beads overnight at 37 °C in 25 ml 50 mM ammonium bicarbonate at 1000 rpm in a ThermoMixer. The supernatant was taken for MS analysis.

Glycoproteomics liquid chromatography with tandem mass spectrometry (LC-MS/MS) measurements. The experiments were performed as previously described with minor modifications, as follows. For each sample and protease digestion, ~50 ng of peptides were run by online reversed phase chromatography on an Dionex UltiMate 3000 (Thermo Fisher Scientific) coupled to a Thermo Scientific Orbitrap Fusion mass spectrometer. A Poroshell 120 EC C18 (50 cm by 75 mm, 2.7 mm, Agilent Technologies) analytical column and a ReproSil-Pur C18 (2 cm by 100 mm, 3 mm, Dr. Maisch) trap column were used for peptide separation. Duplicate samples were analyzed with two different mass spectrometry methods, using identical LC-MS parameters and distinct fragmentation schemes. In one method, peptides were subjected to electron transfer/higher-energy collision dissociation (HCD) fragmentation. In the other method, all precursors were subjected to HCD fragmentation, with additional EThcD fragmentation triggered by the presence of glycan reporter oxonium ions. A 90 min LC gradient from 0% to 44% acetonitrile was used to separate peptides at a flow rate of 300 nl/min. Data were acquired in data-dependent mode. Orbitrap Fusion parameters for the full scan MS spectra were as follows: a standard automatic gain control (AGC) target at 60,000 resolution, scan range 350 to 2000 m/z, Orbitrap maximum injection time 50 ms. The 10 most intense ions (2+ to 8+ ions) were subjected to fragmentation. For the EThcD fragmentation scheme, the supplemental higher energy collision dissociation energy was set at 27%. MS2 spectra were acquired at a resolution of 30,000 with an AGC target of 800%, maximum injection time 250 ms, scan range 120 to 4000 m/z and dynamic exclusion of 16 s. For the triggered HCD-EThcD method, the LC gradient and MS1 scan parameters were identical. The 10 most intense ions (2+ to 8+) were subjected to HCD fragmentation with 30% normalized collision energy from120 to 4000 m/z at 30,000 resolution with an AGC target of 100% and a dynamic exclusion window of 16 s. Scans containing any of the following oxonium ions within 20 ppm were followed up with additional EThcD fragmentation with 27% supplemental HCD fragmentation. The triggering reporter ions were: Hex(1) (129.039; 145.0495; 163.0601), PHex(1) (243.0264; 405.0793), HexNAc(1) (138.055; 168.0655; 186.0761), Neu5Ac(1) (274.0921; 292.1027),Hex (1)HexNAc(1) (366.1395), HexNAc(2) (407.166), dHex(1)Hex(1)HexNAc(1) (512.1974), and Hex (1)HexNAc(1)Neu5Ac(1) (657.2349). EThcD spectra were acquired at a resolution of 30,000 with a normalized AGC target of 400%, maximum injection time 250 ms, and scan range 120 to 4000 m/z.

Glycoproteomics data analysis. The acquired data were analyzed using Byonic (v4.5.2) against a custom database of recombinant F_{ECTO} and the proteases used in the experiment, searching for glycan modifications with 12/24 ppm search windows for MS1/MS2, respectively. Up to 10 missed cleavages were permitted using C-terminal cleavage at F/Y/W/M/L for chymotrypsin, or T/A/S/V for alpha lytic protease. For N-linked analysis, carbamidomethylation of cysteine was set as fixed modification, oxidation of methionine/tryptophan as variable common 1, and hexose on tryptophan as variable rare 1. N-glycan modifications were set as variable common 2, allowing up to maximum two variable common and one rare modification per peptide. All N-linked glycan databases from Byonic were merged into a single nonredundant list to be included in the database search. All reported glycopeptides in the Byonic result files were manually inspected for quality of fragment assignments (with scores ≥200). All glycopeptide identifications from both EThcD and HCDpdEThcD runs were merged into a single nonredundant list per sequon. Glycans were classified on the basis of HexNAc content as truncated (≤2 HexNAc; <3 Hex), paucimannose (2 HexNAc, 3 Hex), oligomannose (2 HexNAc; >3 Hex), hybrid (3 HexNAc) or complex (>3 HexNAc). Byonic search results were exported to mzIdentML format to build a spectral library in Skyline (v22.2.0.312) and extract peak areas for individual glycoforms from MS1 scans. The full database of variable N-linked glycan modifications from Byonic was manually added to the Skyline project file in XML format. Reported peak areas were pooled according to the number of HexNAc, Fuc, or NeuAc residues to distinguish truncated, paucimannose, oligomannose, hybrid, and complex glycosylation, or the degree of fucosylation and sialylation, respectively.

Statistical analysis. All data analysis and visualizations were conducted using a Python notebook integrating SciPy, Matplotlib, Pandas, Seaborn, and Statannotations packages (112-114). Statistical significance was determined using the Mann-Whitney test. For P-value annotations, the following symbols were used: “ns” indicates P≤1,* represents 0.01<P≤ 0.05, ** signifies 0.001<P≤0.01, and stands for 0.0001<P≤0.001.

Chemicals and peptides. Chemicals and peptides used here were previously described (Bovier, et al., 2021). The description is reported here with minimal modification. N-(3-cyanophenyl)-2-phenylacetamide (also known as 3G) was commercially acquired from ZereneX Molecular Limited (UK).

Peptides were purchased from CPC, Ltd. Bromoacetyl cholesterol and bismaleimide cholesterol derivatives were custom-made by Charnwood Molecular Ltd. Dimethylsulfoxide (DMSO), tetrahydrofuran (THF) and N,Ndiisopropylethylamine (DIPEA) were purchased from Sigma Aldrich, and bismaleimide-PEG11 was from Quanta BioDesign. HPLC purification was performed on an 1100 Series Agilent HPLC system equipped with a UV diode array detector and a fraction collector using a reverse phase (RP.) Phenomenex Jupiter C4 LC column 300 Å (150mmby 21.2 mm, particle size 5 mm). MALDI-TOF analysis was performed on Bruker ultrafleXtreme MALDI-TOF-TOF instrument.

### Example 10—Structural and Mechanistic Basis for Antibody Neutralization of the Measles Fusion Protein

Measles virus (MeV) is a highly contagious pathogen and a major global health threat. Resurgent infections, driven by insufficient vaccine coverage, waning herd immunity, and the vulnerability of immunocompromised populations, highlight the urgent need for effective countermeasures. While most vaccine-elicited antibodies target the hemagglutinin (H) protein, antibodies against the fusion (F) protein are also potent inhibitors. However, the specific target sites on this class I fusion protein, which undergoes dramatic conformational changes during entry, remain insufficiently characterized. Here, Applicants characterize four mAbs targeting distinct F conformations. Structural analyses map antibody interactions, revealing that three neutralizing mAbs recognize the metastable prefusion conformation, while a non-neutralizing mAb binds only the post-triggered state. Biophysical and functional assays define distinct mechanisms of action: neutralization occurs either by stabilizing the prefusion protein or by preventing the extended intermediate from completing fusion. Uniquely, Applicants detect a novel mechanism where an antibody prematurely triggers F activation but blocks the subsequent refolding required for viral entry. These findings provide the first detailed mapping of neutralizing epitopes on MeV F, establishing a framework for the rational design of F-targeted interventions.

### INTRODUCTION

Measles is a highly contagious viral disease that has reemerged globally in recent years. Achieving widespread vaccine-induced immunity is critical to prevent outbreaks, particularly as unvaccinated and immunocompromised individuals remain at high risk for severe disease. Although the measles vaccine has an excellent long-term safety and efficacy record, neither global nor American vaccination rates have reached the 95% threshold required for herd immunity. Consequently, the recent resurgence in measles cases reflects a failure to vaccinate rather than vaccine failure. Over the past five years, declining vaccination rates have been fueled by disruptions in routine immunization programs during the COVID-19 pandemic and by the increasing impact of vaccine hesitancy or active anti-vaccination sentiments.

These developments increase the vulnerability of individuals who cannot be vaccinated due to their early age or medical contraindications. Post-exposure prophylaxis (PEP) with total immunoglobulin remains the only recommended option for these individuals; however, its reliability is threatened by declining MeV-specific antibody titers within the donor pool. With measles elimination efforts stalled and a growing number of high-risk individuals, there is an urgent need for effective antiviral therapies. However, currently, there is no antiviral treatment approved for measles. Passive immunization using monoclonal or polyclonal antibodies has long been a cornerstone in preventing and treating other viral infections, particularly in individuals who are unable to receive active vaccination. However, only a limited number of monoclonal antibodies (mAbs) have received clinical approval for the treatment of those viral infectious diseases, and none are available for measles.

Antibodies can exert antiviral effects by targeting viral glycoproteins critical for host cell entry. In MeV, this process involves two envelope proteins: the H protein, which binds host receptors, and the F protein, which mediates membrane fusion. MeV Fis a class I trimeric fusion protein undergoing substantial conformational rearrangements from prefusion to postfusion states, a feature shared with many other enveloped viruses. Fusion is a dynamic, multistep process: following H-F interaction and receptor engagement, F transitions through an extended “pre-hairpin” intermediate, during which its heptad repeat regions (HRN and HRC) span the viral and host membranes. These regions then fold into a six-helix bundle (6HB), completing fusion and enabling viral entry.

MAbs targeting MeV H and F offer several advantages: they can neutralize the virus early in infection, avoid the toxicity and off-target effects of small molecules, and provide protection for immunocompromised or vaccine-ineligible individuals. Historical studies suggest that vaccine-induced virus neutralizing antibodies primarily target the H protein, but natural infection elicits robust neutralizing antibodies against both H and F, indicating that Fis a promising target for mAbs development. Strategies to block viral entry include targeting: (i) H protein binding to hSLAMF1 (CD150) or Nectin-4 receptors, (ii) F activation via H-F interaction, (iii) conformational transitions exposing the F fusion peptide, and (iv) F refolding and pore expansion. Applicants have generated and characterized a panel of novel chimeric (mouse Fab/human Fc) mAbs targeting MeV F, including the previously characterized antibody 77.1. Applicants present here structures and functions of four antibodies selected for distinct epitope specificity and mechanisms of action. Three of these mAbs exhibit potent neutralization against the two currently circulating wild-type MeV strains, providing a strong foundation for antiviral development. The data demonstrate that the MeV F protein contains multiple neutralization-sensitive epitopes that can be targeted by potent mAbs. By combining antibodies with distinct mechanisms broad and synergistic inhibition can be achieved, raising the barrier to resistance. A cocktail of long-acting humanized anti-F antibodies could serve as a critical countermeasure for exposed health care workers or individuals unable to receive vaccination, for outbreak control, and as a complement to existing PEP strategies.

### Results.

### Neutralization Activity and F Protein Reactivity of Chimeric Monoclonal Antibodies Against MeV.

Applicants generated and characterized a panel of mouse monoclonal antibodies (mAbs) targeting the MeV F protein. The Fab regions of four of these antibodies were grafted onto a common human Fc backbone. Here, Applicants present the functional and structural properties of these chimeric mAbs, comprising three novel antibodies (Y10F, H8, and C6) and the previously characterized 77.1 (Zyla, et al., 2024) for comparison.

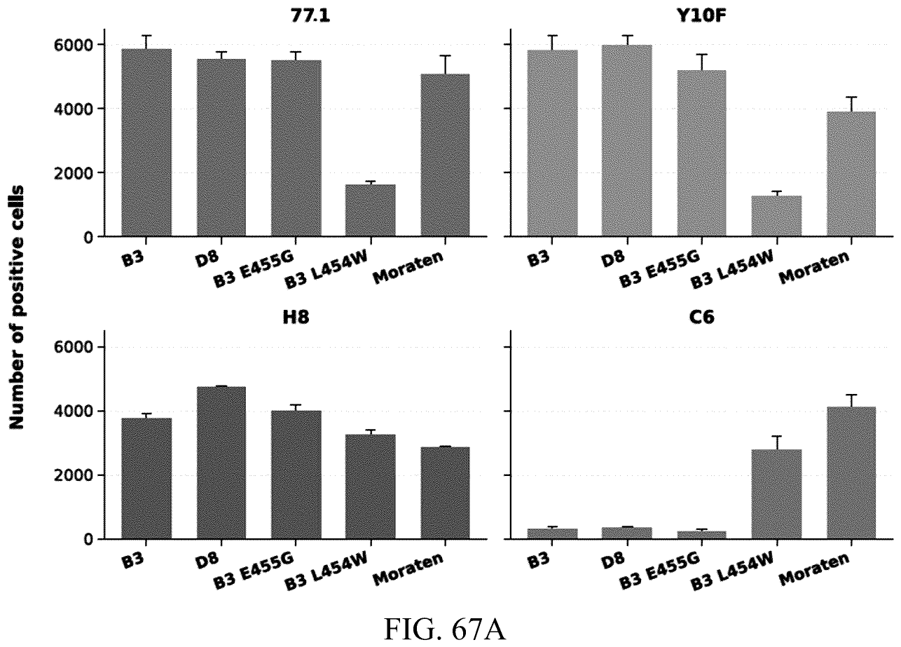

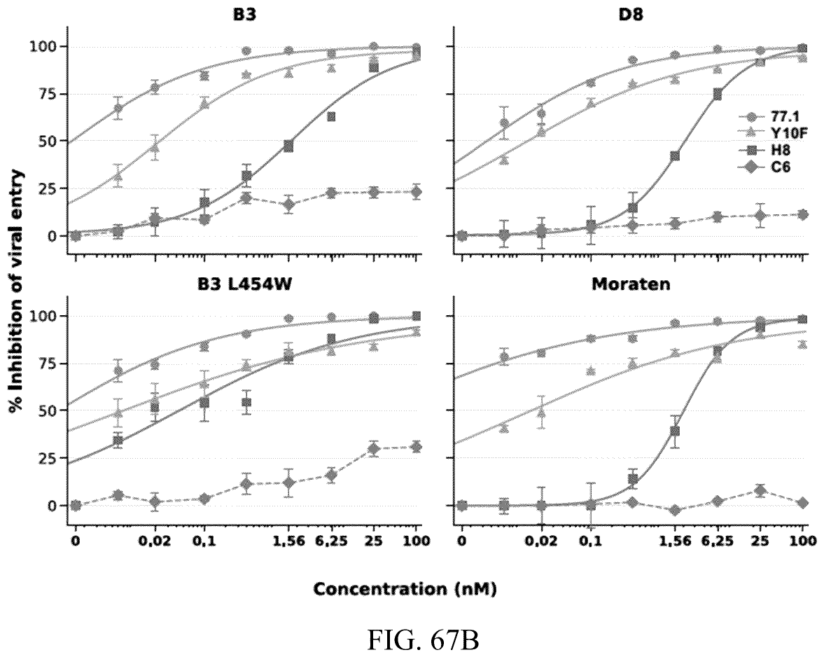

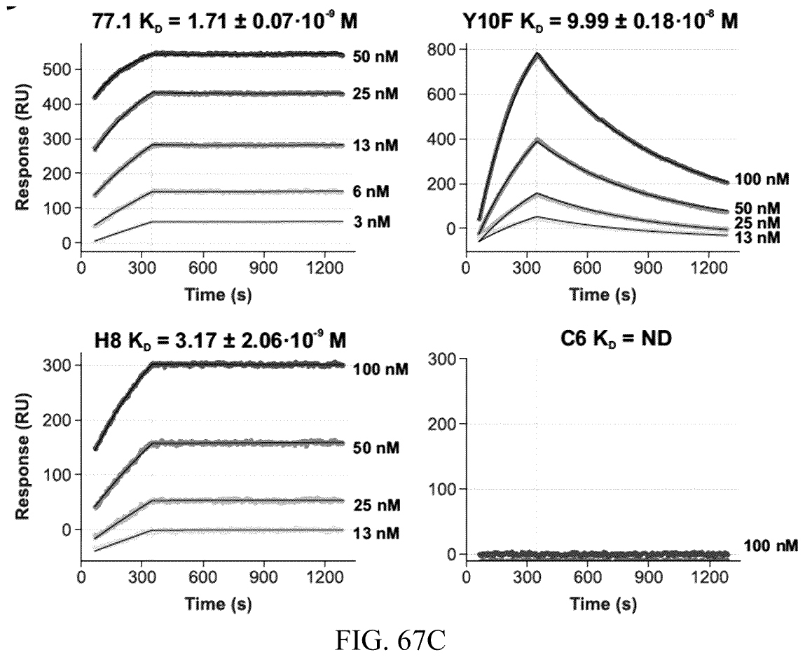

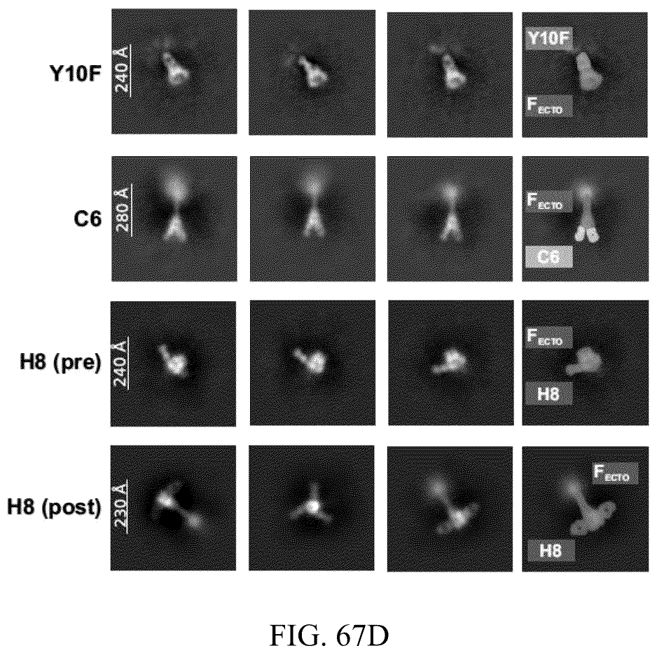

Applicants first assessed the ability of these antibodies to recognize a variety of F glycoproteins, including F from two currently circulating wild-type strains (B3 and D8), the vaccine strain (Moraten), and two previously characterized B3 F variants (L454W F), which is characterized by a destabilized prefusion conformation, and E455G F, which contains a stabilized prefusion conformation (FIG. 67A). MAbs 77.1 and Y10F recognized each of the currently circulating wild-type F proteins, the Moraten-vaccine F, and the prefusion-stabilized E455G variant. Recognition of B3 F carrying the destabilizing L454W mutation, however, was markedly reduced. In contrast, mAb H8 recognized all F proteins tested, including both pre- and postfusion forms. mAb C6 exhibited a different pattern: it did not recognize wild-type or stabilized E455G F variant and instead, it recognized only the Moraten-vaccine F and the destabilized L454W variant, consistent with its specificity for post-triggered conformations.

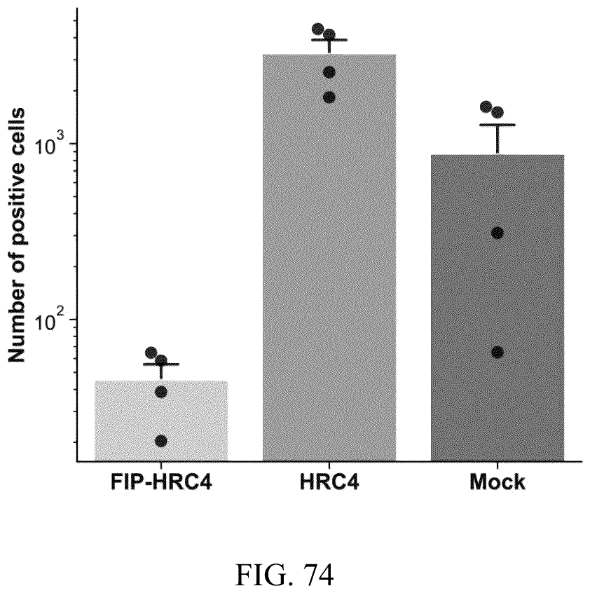

Neutralization potency, measured by plaque reduction neutralization tests (PRNT) against four recombinant viruses (B3, D8, B3 F-L454W, and vaccine strain Moraten), revealed that 77.1 and Y10F were the most potent antibodies (FIG. 67B). 77.1 exhibited IC₉₀ values ranging from 0.15-0.45 nM, while Y10F values ranged from 1.3-42 nM. H8 was less potent overall (IC₉₀: 11-63 nM) but showed higher activity against the neuropathogenic F-destabilized B3 F-L454W variant (23 nM) compared to Y10F (42 nM). In contrast, C6 exhibited no measurable neutralizing activity (using a specific mechanistic assay described in FIG. 74 Applicants determined that the C6 epitope is exposed prior to complete refolding into the postfusion conformation, indicating that C6 is a post-trigger-specific antibody).

Applicants next quantified antibody binding to purified, stabilized, soluble prefusion F ectodomain (F_{ECTO}) using high-throughput surface plasmon resonance (SPR) on a Carterra LSA (FIG. 67C). Unlike constructs locked by disulfide bridges, this F protein relies on natural substitutions to stabilize the prefusion state, which allows it to undergo conformational transitions, but at a more gradual, controlled rate than the wild-type. Three antibodies (77.1, H8, Y10F) bound this purified, prefusion MeV F. In contrast, the post-trigger-specific control mAb C6 showed no detectable binding to this prefusion-stabilized F, as expected for its post-triggered conformation-specific recognition.

Applicants observed distinct kinetic signatures across the three binders. mAb 77.1 showed the highest affinity (K_{D}=1.71±0.03×10⁻⁹M). H8 showed an intermediate association and had the slowest dissociation, with a K_{D} of 3.17±2.06×10⁻⁹ M. Antibody Y10F displayed the slowest association rate (kₐ=1.79×10⁴ M⁻¹s⁻¹), and dissociated more quickly, yielding a lower overall affinity (K_{D}=9.99±0.18×10⁻⁸ M). These trends indicate that Y10F forms a shorter-lived complex than the other two characterized antibodies (FIG. 67C and Table 10).

To visualize the epitopes of each antibody, Applicants performed negative-stain EM (NS-EM) on Fab-F_{ECTO} complexes (FIG. 67D). The structure of mAb 77.1, at the base of prefusion F, was previously described. For the new antibodies, class averages showed that Y10F engages the prefusion apex of F, whereas H8 can bind both prefusion and postfusion conformations. In contrast, C6 only produced apparent postfusion complexes. Taken together, these results indicate that three mAbs that bind prefusion epitopes are neutralizing (77.1, Y10F, H8), while the mAb that binds only a post-trigger epitope is not neutralizing (C6) (see also FIG. 74).

TABLE 10
Fitting parameters of antibody kinetics data from LSA.

| Antibody | H8 | Y10F | 77.1 | C6 |
|---|---|---|---|---|
| kₐ (M⁻¹s⁻¹) | 23640 ± 810 | 17900 ± 300 | 121000 ± 2000 | ND |
| k_{d} (s⁻¹) | (7.48 ± 4.82) × | (1.790 ± 0.009) × | (2.07 ± 0.09) × | ND |
| | 10⁻⁵ | 10⁻³ | 10⁻⁴ | |
| K_{D} (M) | (3.17 ± 2.06) × | (9.99 ± 0.18) × | (1.71 ± 0.07) × | ND |
| | 10⁻⁹ | 10⁻⁸ | 10⁻⁹ | |
| R² | 0.99967 | 0.99942 | 0.99987 | ND |

ND = Not determined

### Stability of the F Protein in Antibody Complexes.

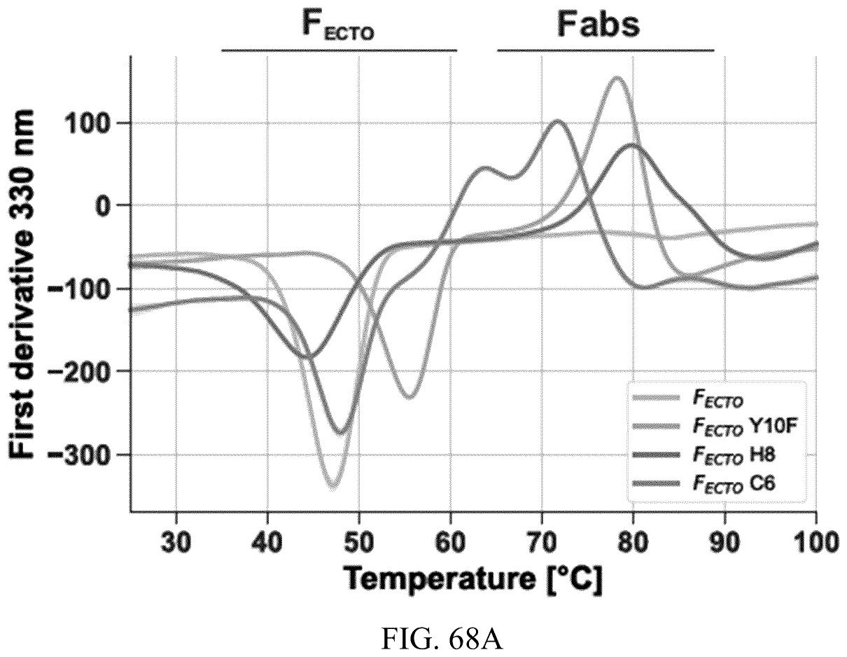

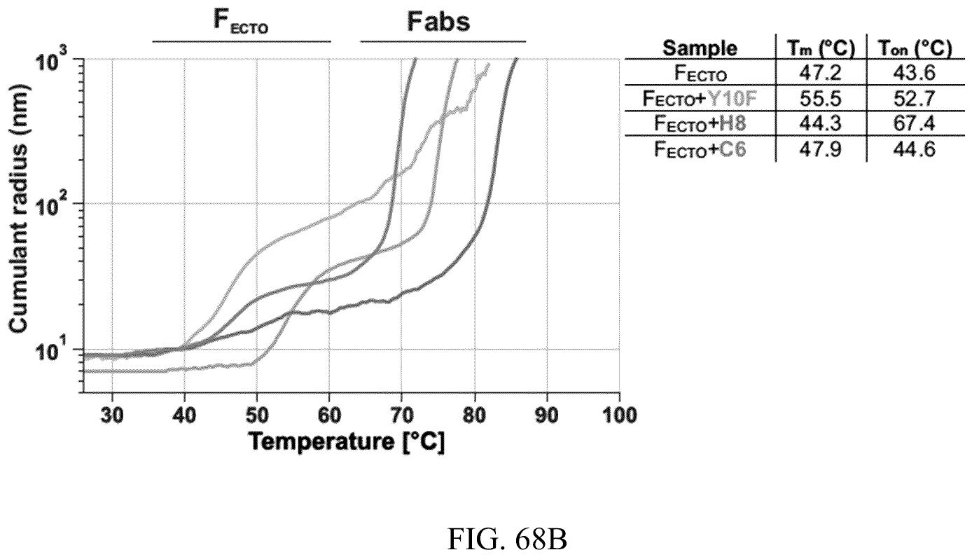

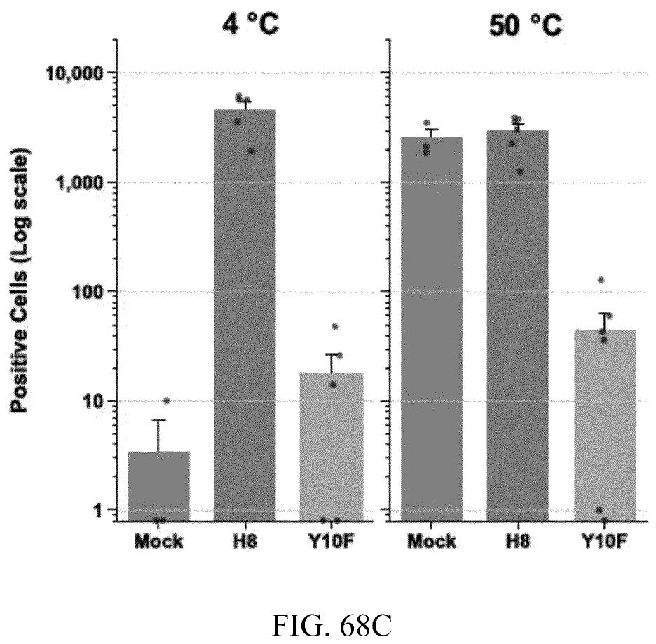

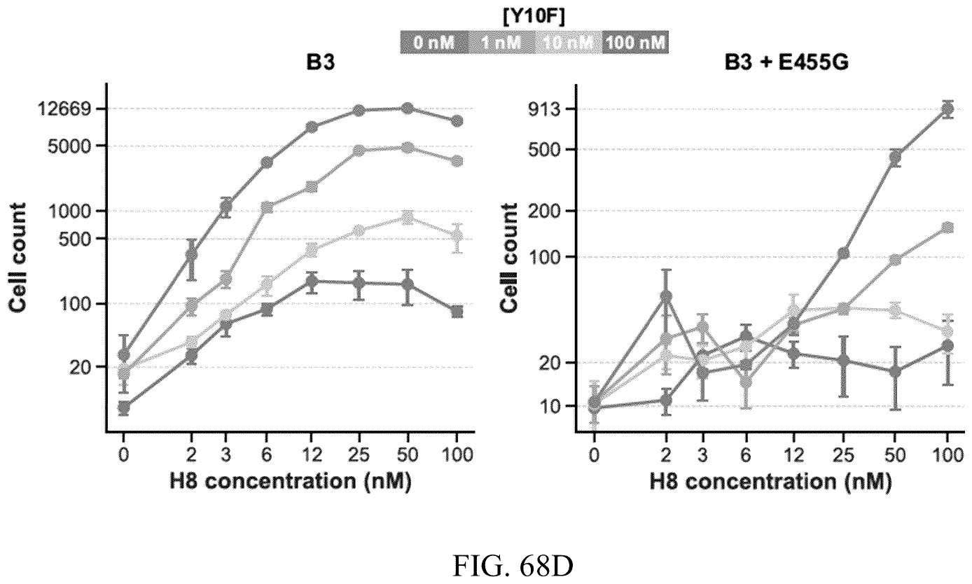

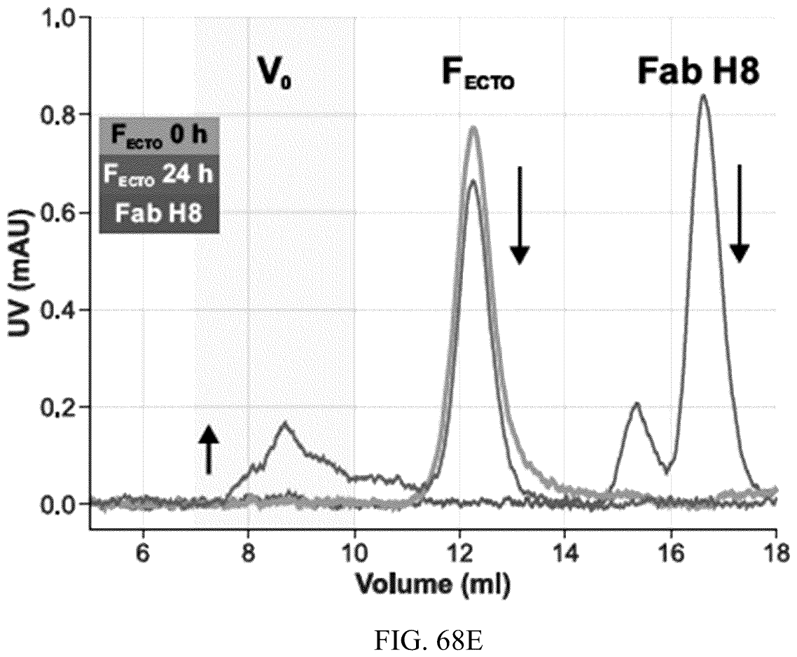

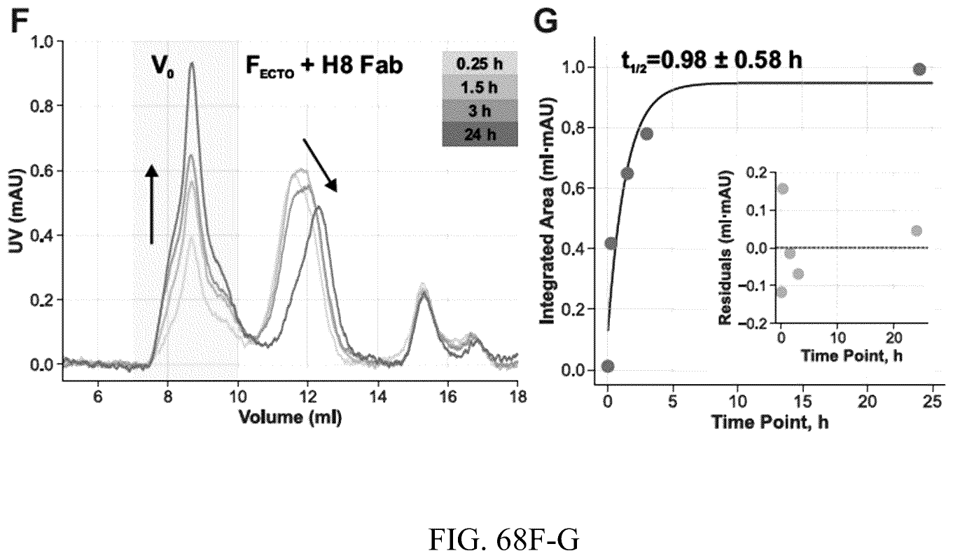

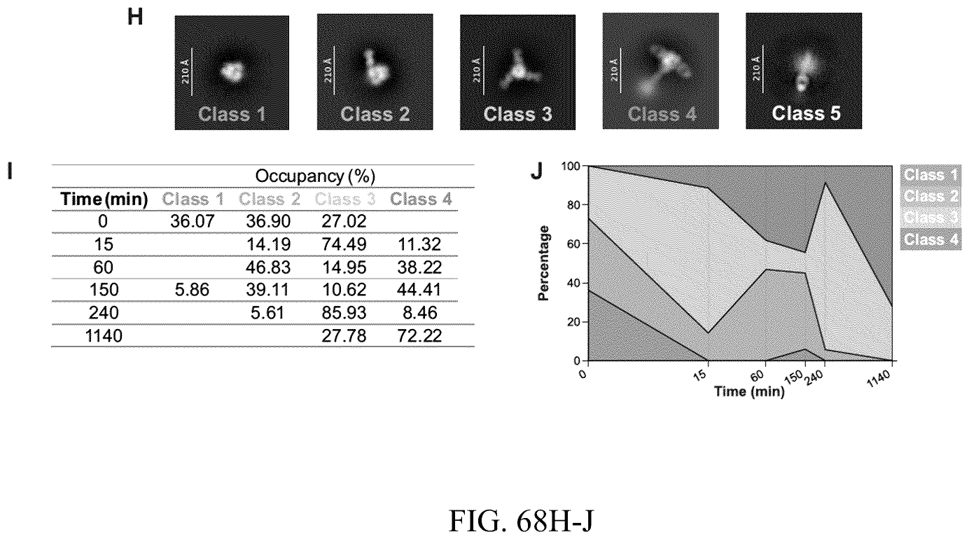

To assess how antibody binding affects the stability of the prefusion F_{ECTO}, Applicants performed nano differential scanning fluorimetry (nanoDSF; NanoTemper Prometheus Panta) (FIG. 68A). F_{ECTO} was incubated with a threefold molar excess of Fab over F trimer for 30 min at room temperature. F_{ECTO} alone exhibited a melting temperature (Tm) of 47.2 °C, consistent with prior measurements. The presence of the post-trigger-specific C6 had a negligible effect on the F_{ECTO} Tₘ (~0.7 °C). Although prefusion-specific antibodies (Y10F and H8) altered F_{ECTO} thermal stability, they did so in distinct ways. Specifically, Y10F increased it by 8.3 °C to 55.5 °C. In contrast, H8 decreased the F_{ECTO} Tₘ by 2.9 °C to 44.3 °C. The Fab melting transitions could be observed at higher temperatures, above the unfolding temperature of the F_{ECTO}-Fab complex (FIG. 68A).

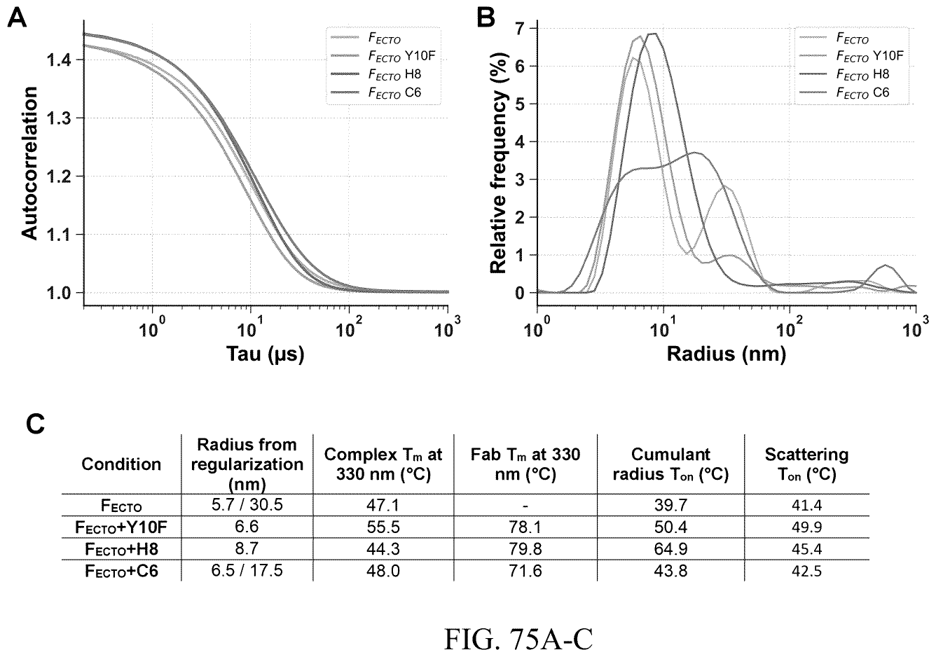

In parallel, Applicants monitored the cumulant (hydrodynamic) radius as a function of temperature (FIG. 68B, FIG. 75). The F_{ECTO} trimer alone maintained a radius of ~9 nm until reaching an aggregation onset temperature (Tₒₙ) of ~39.7-41.4 °C, after which the radius increased continuously. The sample containing the post-trigger-specific mAb C6 displayed a nearly identical aggregation profile (Tₒₙ=44.6 °C), consistent with its inability to bind the prefusion ectodomain.

In contrast, the neutralizing antibodies distinctively modulated these hydrodynamic properties. The F_{ECTO}-Y10F complex appeared more compact initially and maintained this stability up to 52.7 °C, delaying the onset of aggregation relative to the protein alone. The F_{ECTO}-H8 complex exhibited a unique profile: despite H8 lowering the melting temperature of F_{ECTO} (Tm=44.3 °C, FIG. 68A), the complex did not undergo immediate aggregation. Instead, it maintained a stable radius until 67.4 °C, a temperature corresponding to the thermal unfolding of the Fab itself. Collectively, these analyses demonstrate that while C6 has no impact on stability, Y10F stabilizes the prefusion trimer, and H8 prevents the formation of large aggregates despite lowering the activation barrier.

Mechanistically, the data suggest opposing modes of action for Y10F and H8: Y10F stabilizes the prefusion conformation and raises the energy barrier for activation, thereby preventing fusion triggering, whereas H8 lowers the activation barrier and promotes premature triggering of the fusion machinery.

To validate these mechanistic insights in the context of native membrane-anchored F, Applicants performed cell-surface staining with the post-trigger-specific mAb C6 after preincubating cells at either 4 °C or 50 °C (the latter to thermally induce F triggering) in the presence of Y10F or H8 (FIG. 68C). Because C6 recognizes an epitope exposed only upon F triggering (FIG. 74), its binding serves as a proxy for F activation. At 4 °C, C6 bound F in the presence of H8, but not in the presence of Y10F. At 50 °C, C6 showed no binding to F in the presence of Y10F. These findings corroborate the biophysical data, supporting a model in which Y10F raises the conformational activation threshold of F, thereby maintaining the prefusion state, while H8 lowers this barrier and drives premature activation.

Applicants next asked whether the stabilizing effect of Y10F could block H8-mediated F activation. For the experiment shown in FIG. 68D, cells expressing either wild-type B3 For the B3 E455G F mutant (which require more energy for activation) were incubated overnight at 37 °C. The following day, Y10F was added for 30 min (at the indicated final concentration), after which H8 was added to achieve final concentrations of 100, 50, 25, 12.5, 6.25, 3.13, or 1.56 nM. Cells were then transferred to 37 °C for 60 min. Following incubation, cells were stained with the conformation-specific mAb C6 to quantify exposure of the post-trigger epitope. In the absence of Y10F, H8 induced a dose-dependent increase in F triggering, which was more pronounced for the B3 F wt than for the stabilizing E455G mutant. In contrast, at the highest concentrations of Y10F, H8-induced F triggering was also completely abolished for the B3 F wt.

To define the kinetics of H8-induced triggering in purified protein, Applicants performed time-resolved size-exclusion chromatography and followed the accumulation of signal in the void volume. F_{ECTO}, incubated alone at room temperature for 24 h, showed only a small, gradual increase in the 7-10 mL region, indicating slow, spontaneous refolding toward the postfusion state (FIG. 68E). In contrast, F_{ECTO} incubated with H8 Fab displayed a much stronger, time-dependent increase in this void-volume peak, accompanied by loss of the intact complex peak (FIG. 68F). Plotting the integrated void-volume signal as a function of incubation time revealed a single-exponential behavior, yielding an estimated half-life of 0.98±0.58 h for H8-induced triggering (FIG. 68G).

To visualize the structural species that arise during this reaction, Applicants next analyzed time-resolved negative-stain electron microscopy datasets of F_{ECTO}-H8 mixtures. Across all six time points, Applicants repeatedly identified four main classes: prefusion F_{ECTO} without antibody (class 1), prefusion F_{ECTO} bound by a single H8 Fab (class 2), a top view of F bound by three Fabs (class 3), and a side view of Fin the postfusion conformation with three bound Fabs (class 4, FIG. 68H). A fifth, highly heterogeneous class contained H8 bound to F fragments, free Fab, and other mixed species and was therefore excluded from quantitative analyses. Notably, classes 3 and 4 did not always appear together in the same datasets, suggesting that they may represent distinct conformational endpoints or intermediates rather than 2 distinct views of a single class. Over time, the occupancy of classes 1 and 2 decreased and became negligible at later time points, whereas classes 3 and 4 increasingly dominated the particle population (FIG. 681-J). This shift in class distribution aligns with the size-exclusion data and supports a reaction pathway in which H8 converts prefusion F into multi-Fab-bound, post-trigger-like conformations and the postfusion state.

### Cryo-EM Mapping of Y10F and H8 Epitopes on the F Protein.

To define the antibody epitopes on F, Applicants determined cryo-EM structures of the F_{ECTO} in complex with Y10F alone, with Y10F and 77.1 bound simultaneously, and with H8 alone.

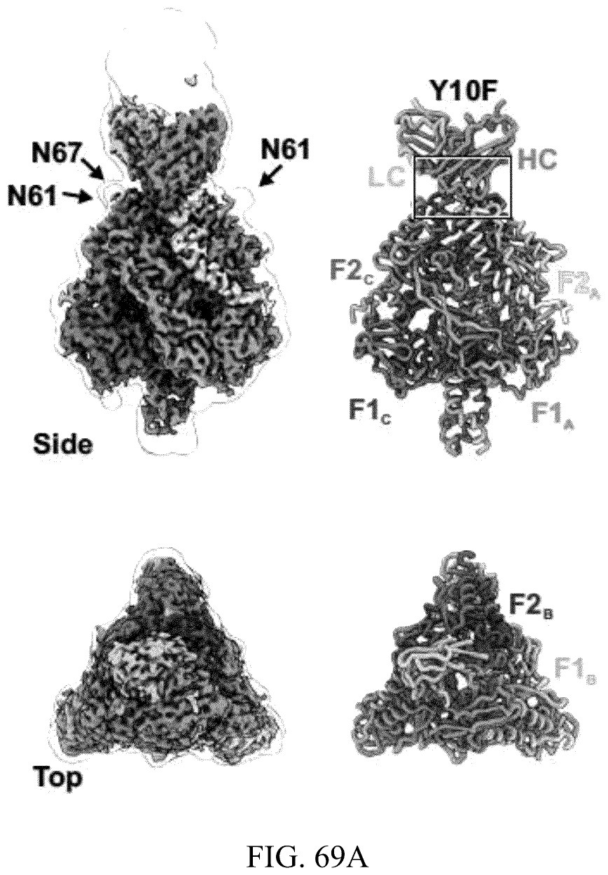

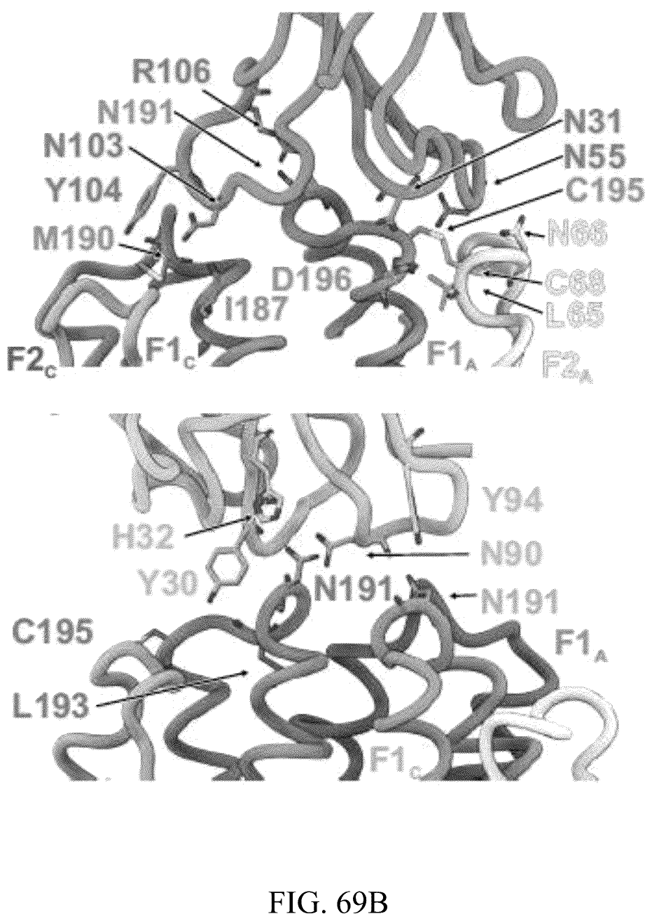

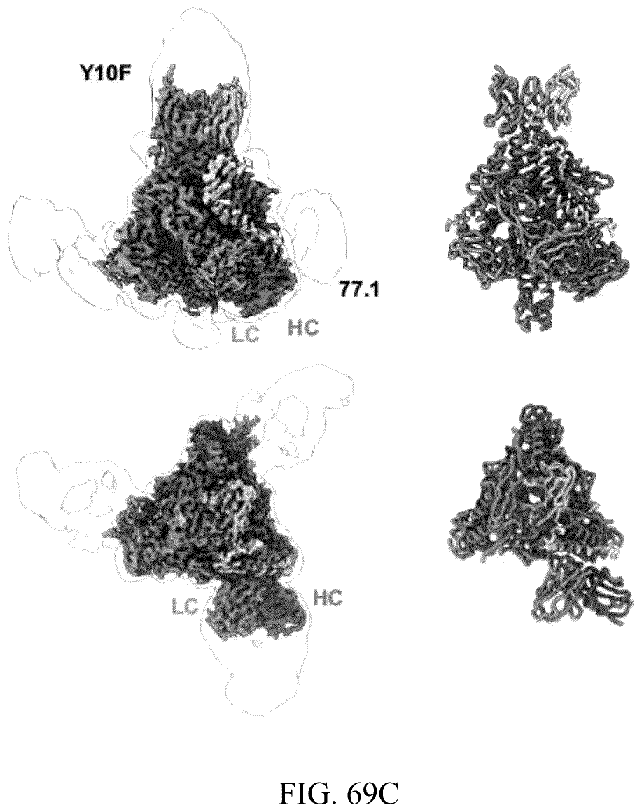

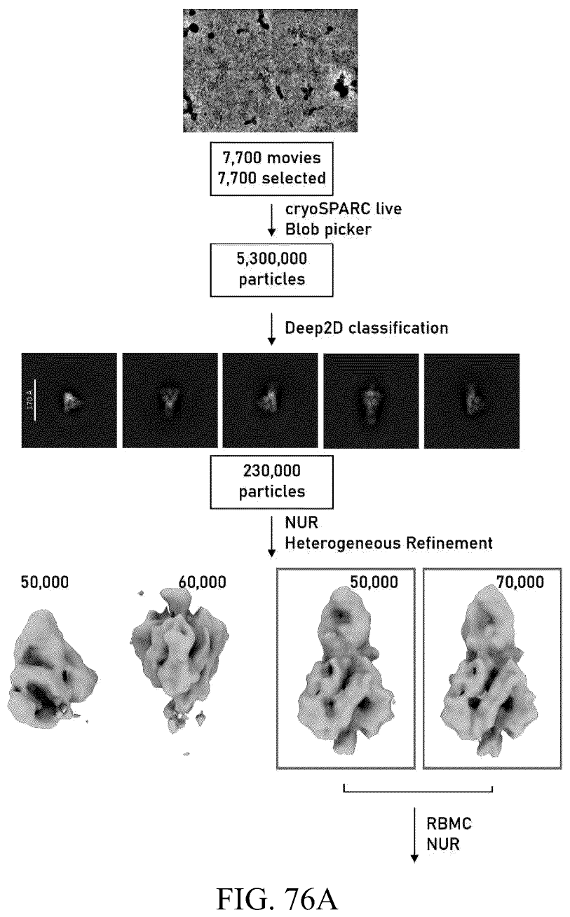

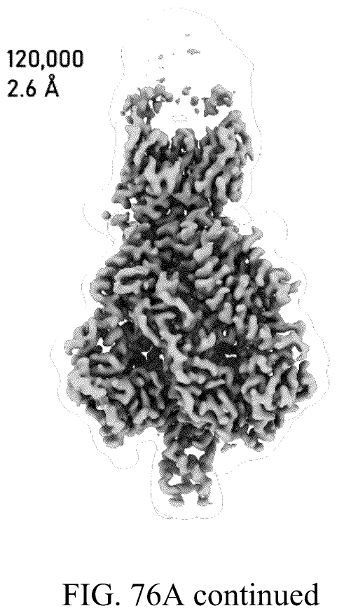

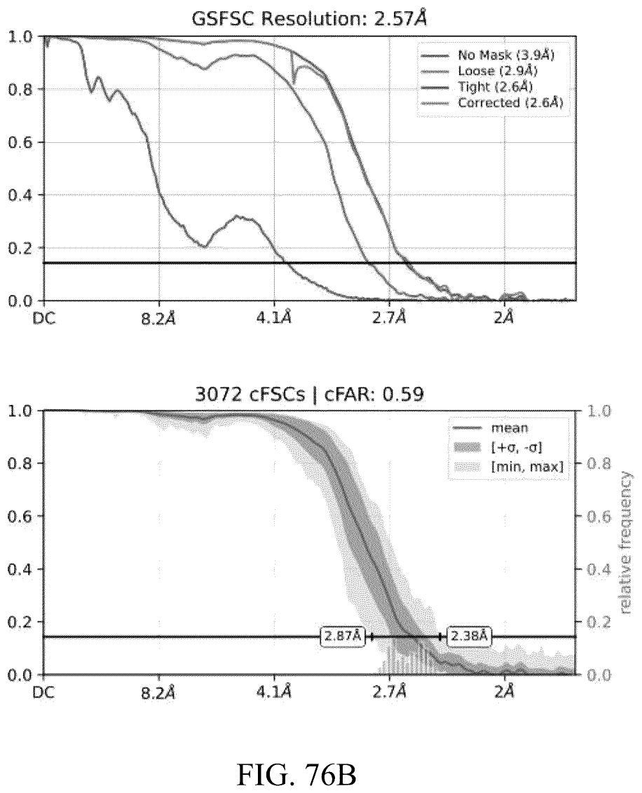

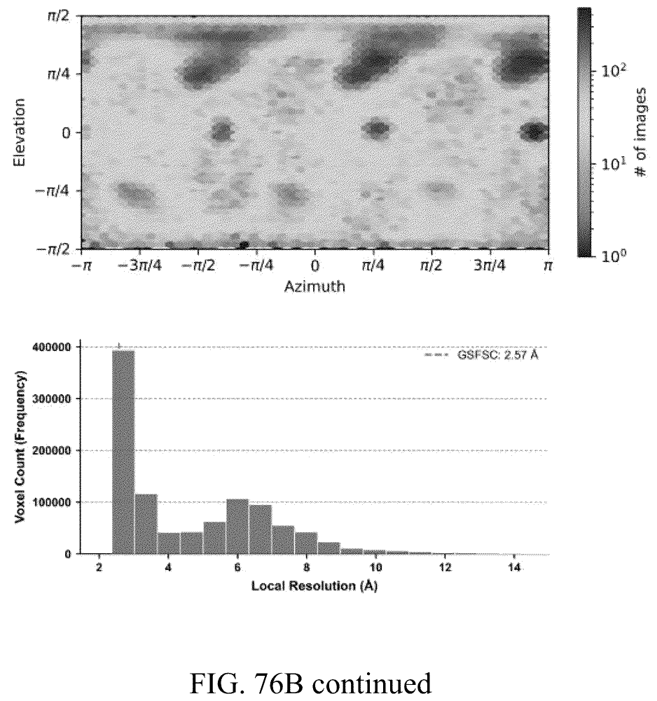

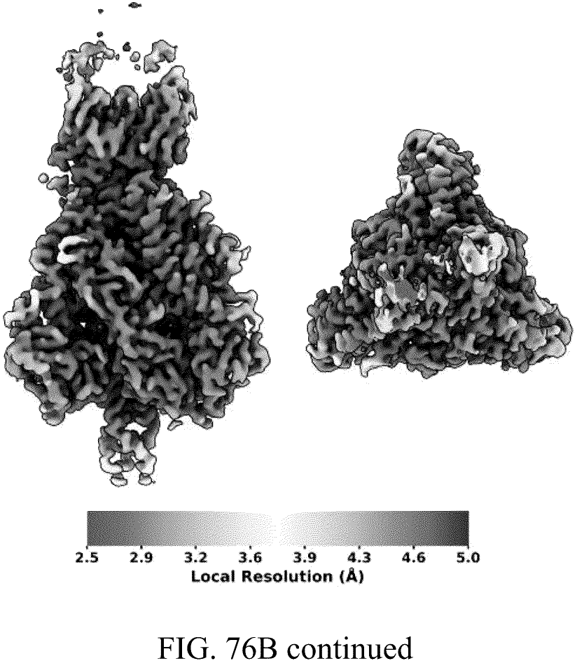

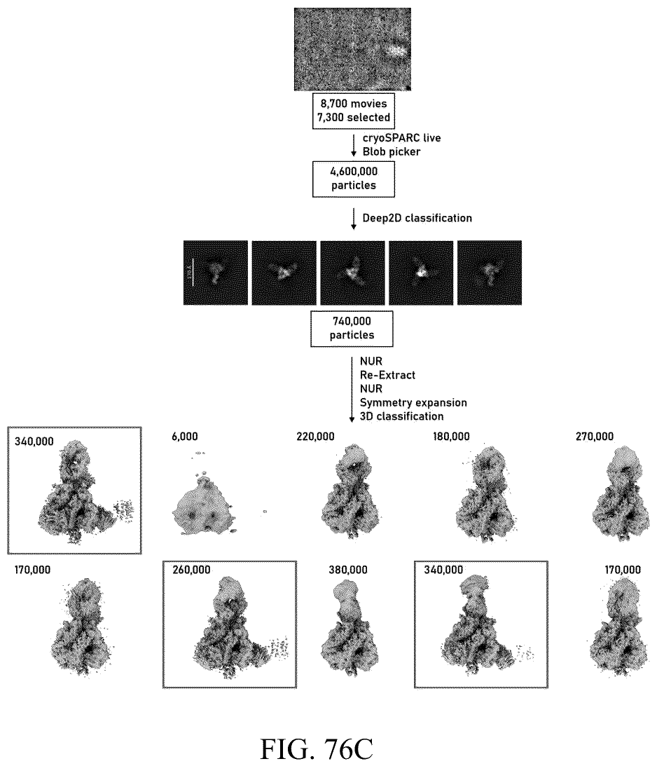

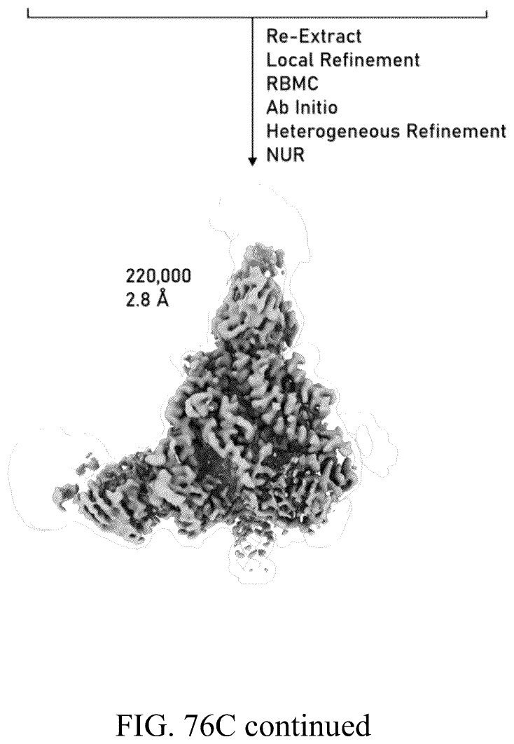

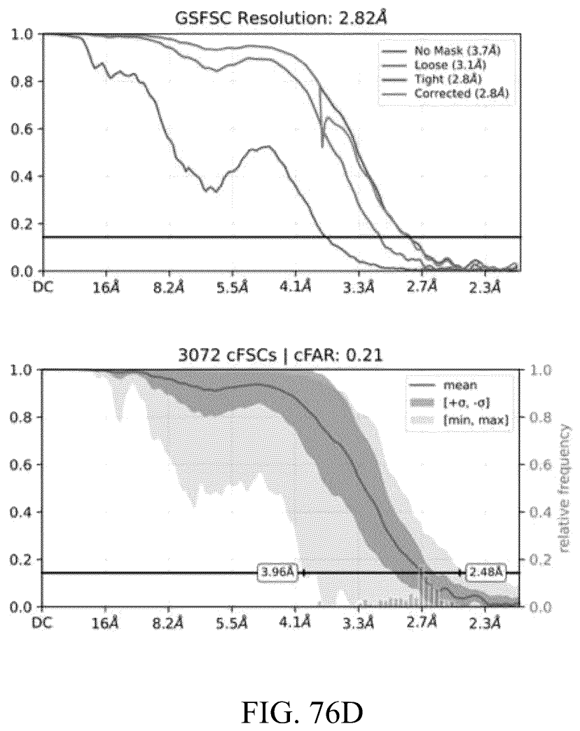

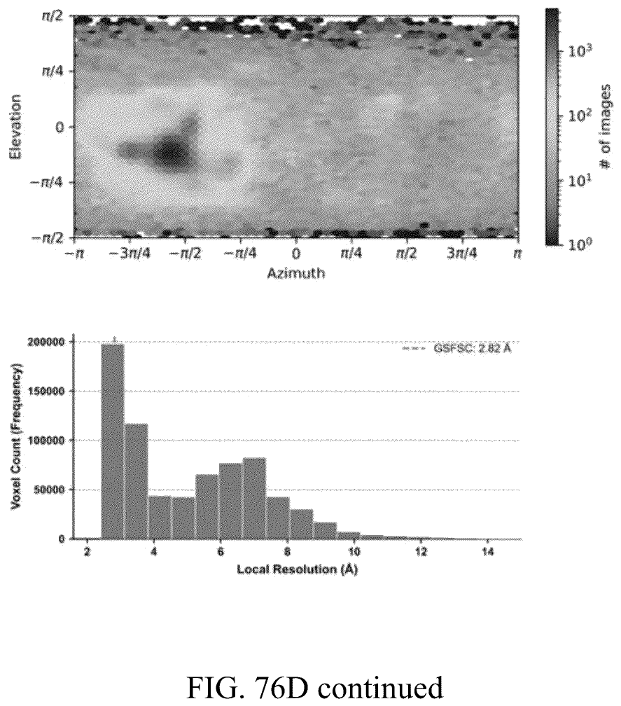

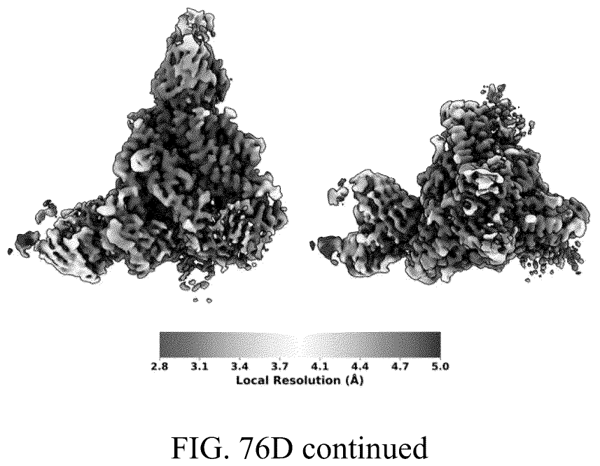

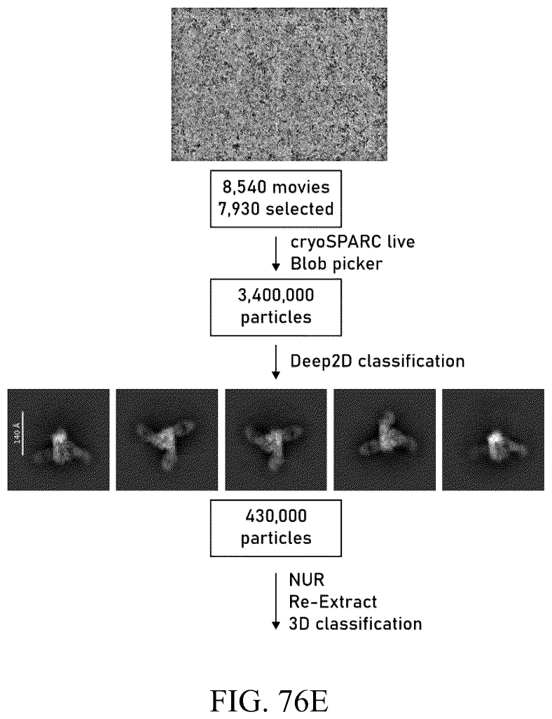

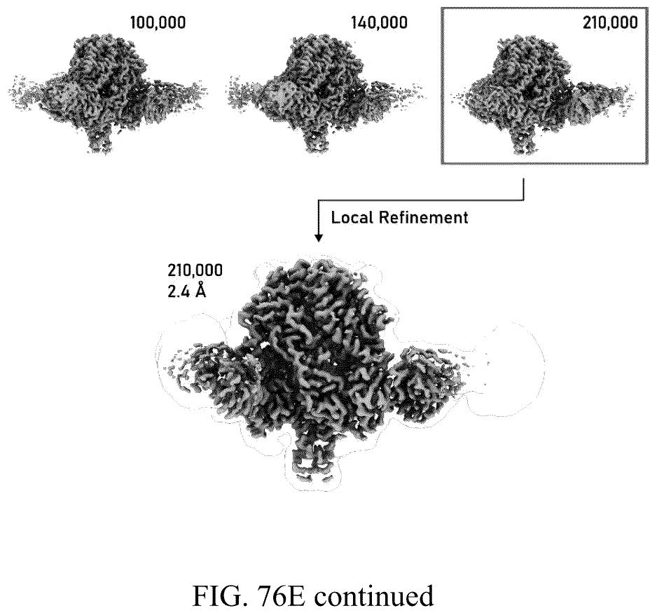

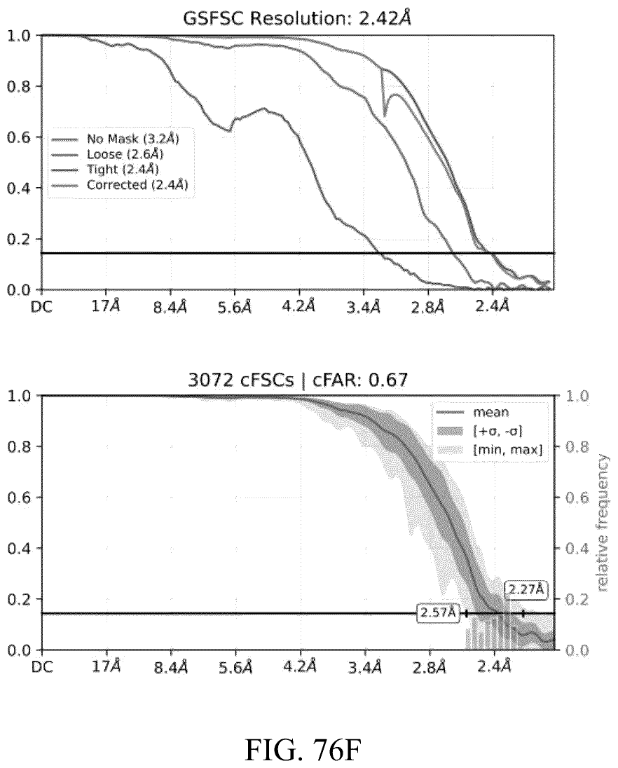

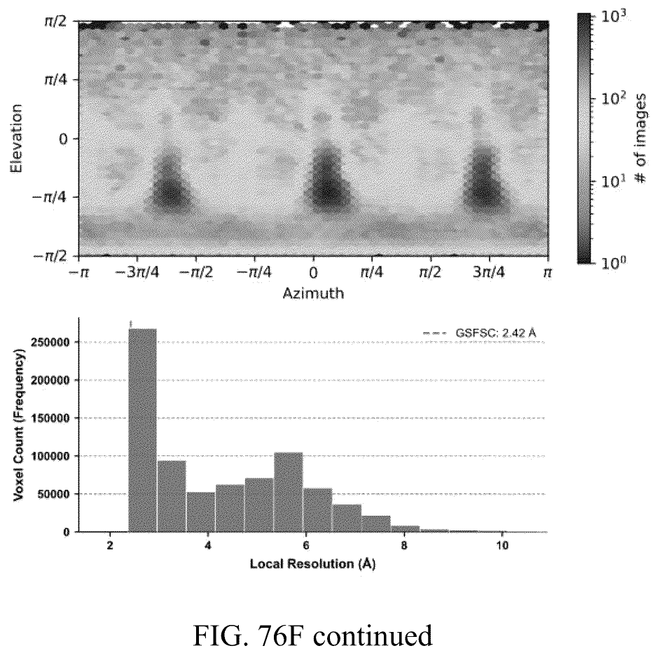

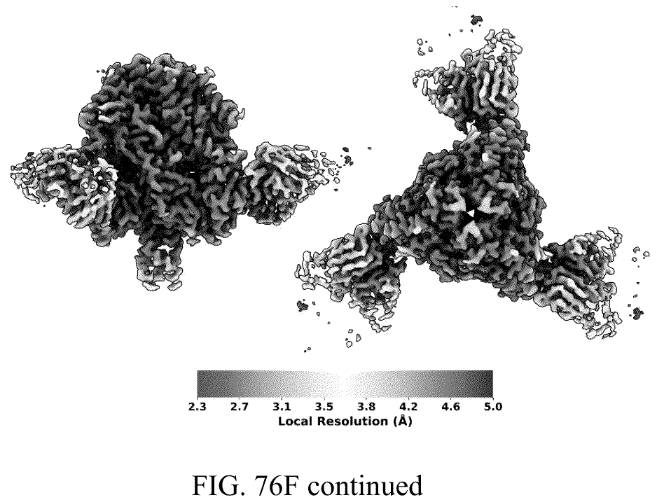

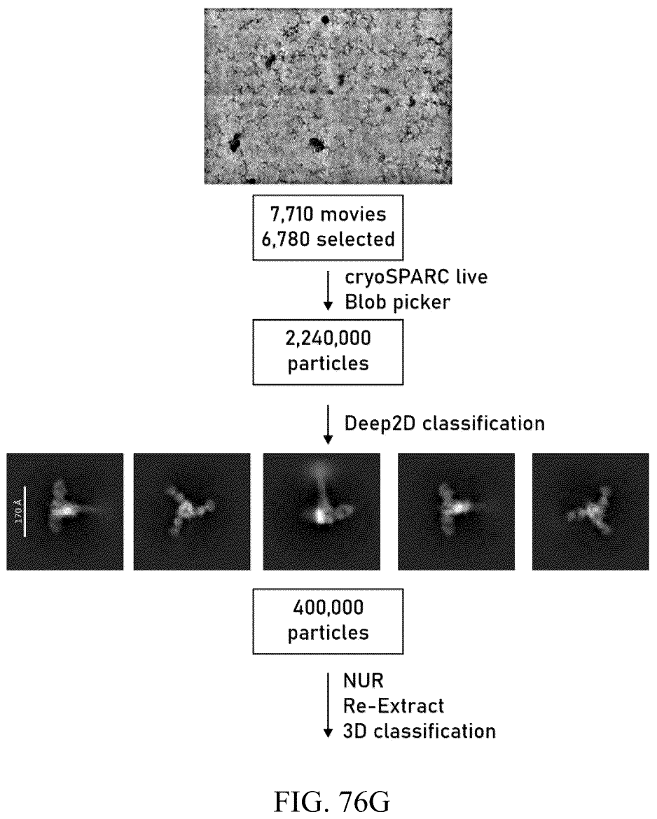

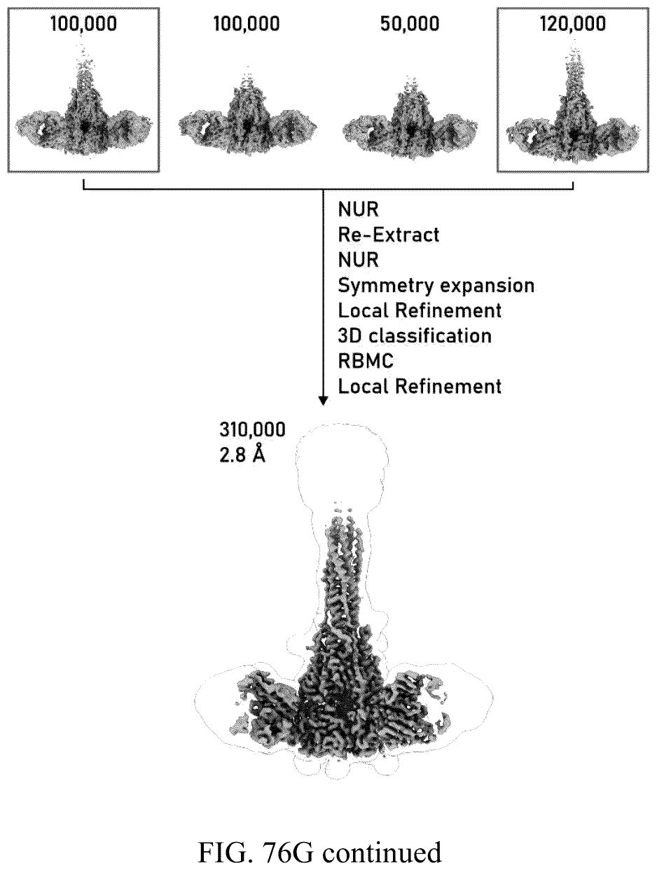

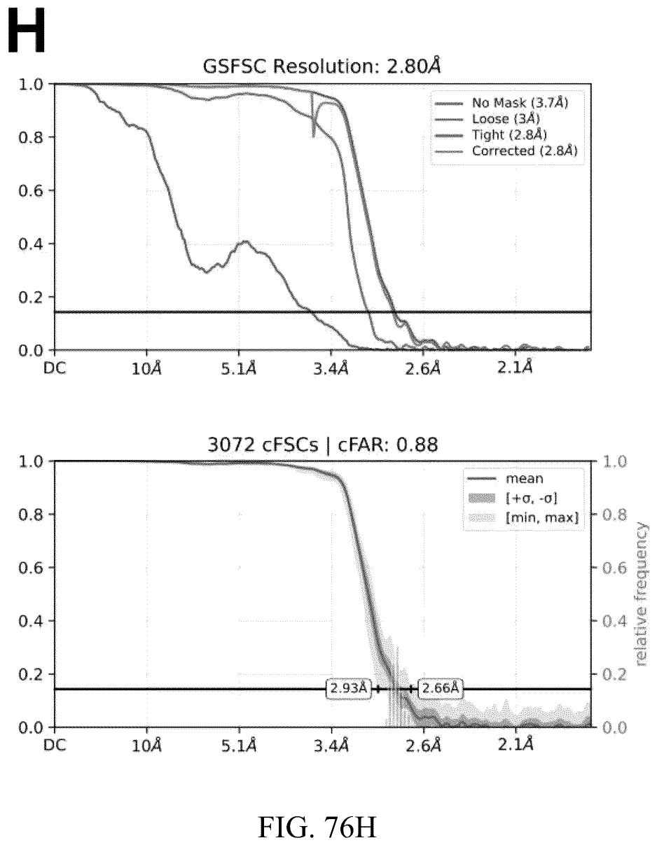

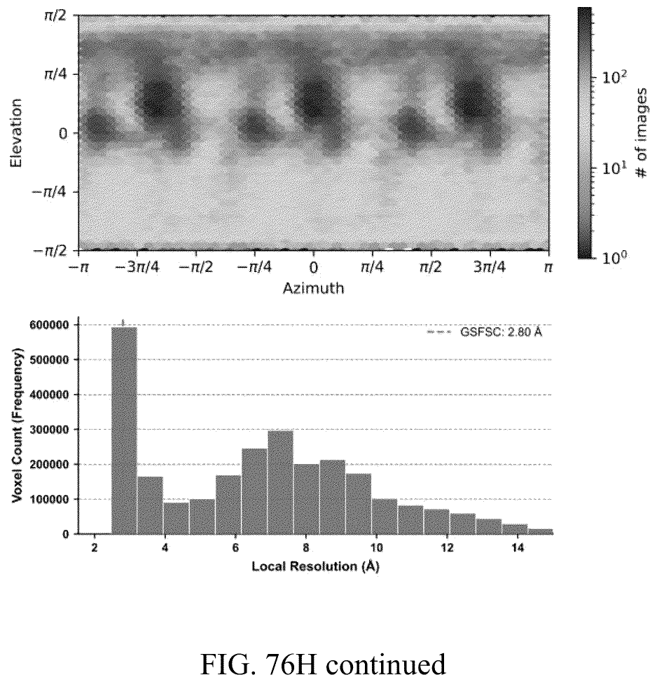

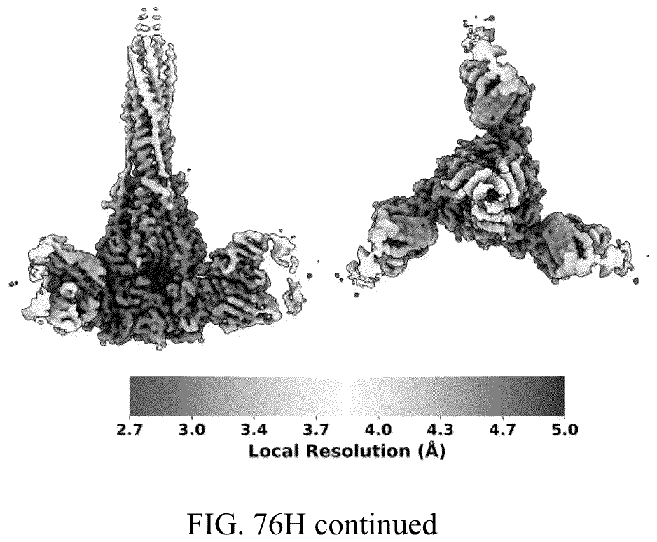

The F_{ECTO}-Y10F dataset exhibited mild preferred orientation, with many particles showing a tilted top view (FIG. 76). Despite this, Applicants reconstructed a 2.59 Å map that revealed asymmetric binding of a single Y10F Fab at the apex of F_{ECTO} (FIG. 69A). Y10F binds the region in the center of the apex, surrounded by the six glycosylation sites positioned on the three F2 subunits at positions 61 and 67 (FIG. 69A). Both heavy and light chains contribute to a dense interaction network. The heavy chain forms nine hydrogen bonds distributed across three protomers (including one F2 subunit and two F1 subunits), with residues N55 and N31 accounting for six of these nine bonds. The light chain forms five hydrogen bonds with the same two F1 chains. The buried interface area is ~870 Å², ~70% of which is contributed by the heavy chain, with a binding free energy (AG) estimated by PISA of −8.6 kcal/mol, ~85% of which arises from heavy-chain contacts (FIG. 69B).

Applicants also simultaneously determined the structure of the F_{ECTO}-bound complex with Y10F and 77.1 (FIG. 69C) to test co-occupancy and any binding-induced conformational changes resulting from simultaneous binding of these two antibodies. Although both Fabs were supplied in excess, the complex displayed partial occupancy of each Fab, with individual particles displaying one, two, or three copies of 77.1. The combination of preferred orientation, asymmetric nature of the complex, and incomplete occupancy complicated particle selection. However, through symmetry expansion and focused masking, Applicants obtained a reconstruction in which single copies of 77.1 and Y10F were fully resolved. Superposition of the Y10F-only and Y10F+77.1 models yielded a Ca RMSD of 0.79 Å, indicating no significant global rearrangement of F_{ECTO}. Local differences were restricted to the 77.1 epitope, particularly residues 433-442, which showed an RMSD of ~2 Å, consistent with previous descriptions of 77.1-induced adjustments. These data demonstrate that Y10F and 77.1 can co-bind F, supporting their use in an antibody cocktail.

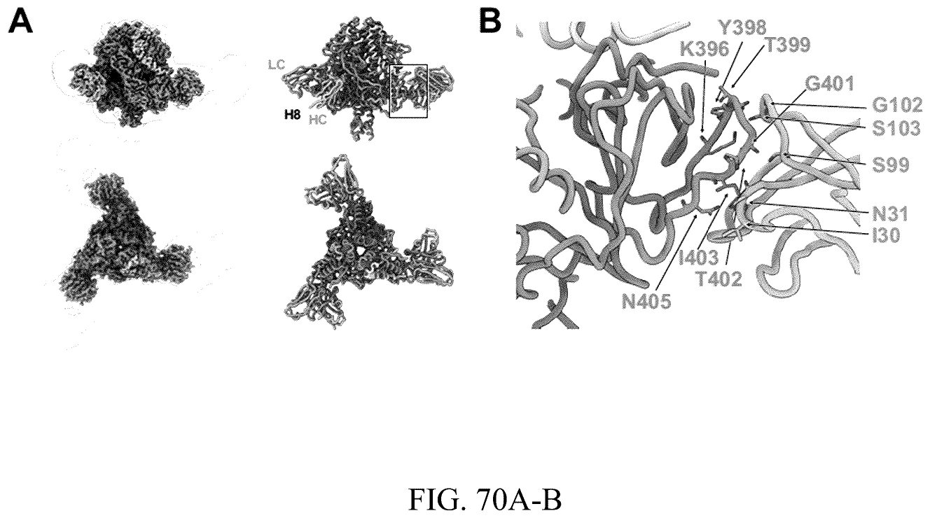

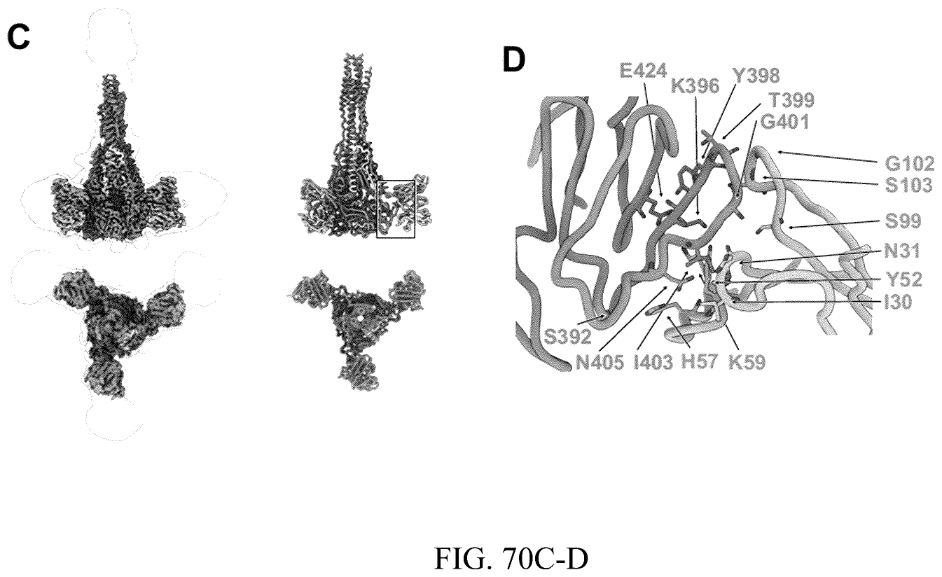

To characterize H8 binding in both conformational states of F, Applicants used two sample-preparation strategies guided by negative-stain EM (FIG. 67D) and nanoDSF (FIG. 68) results. To solve a prefusion complex (FIG. 70A), F_{ECTO} and H8 Fab were incubated for 15 min at room temperature before vitrification to promote complex formation while minimizing triggering; longer incubations increased the proportion of postfusion particles. To solve a postfusion complex (FIG. 70B), F_{ECTO} and H8 were incubated overnight at 4 °C and then applied to graphene oxide grids.

In the prefusion state, H8 bound symmetrically to all three protomers at an epitope on the outer face of domain II, opposite the F vertex contacted by 77.1, producing a triangular-like structure. The epitope centered on the single loop in domain II (residues 396-405). The binding was dominated by the heavy chain and was stabilized by ten hydrogen bonds. PISA also identified a single salt bridge attributed to the light chain in proximity to residue G115, the first residue resolved in F1, placing the binding site near the furin cleavage site. H8 binding induced a local shift of the 396-405 loop, with a maximal displacement of 2.5 Å toward the Fab

H8 recognized the same topological site on postfusion F. Because domain II rotates relative to the rest of the protein during refolding around residue G433, the Fab reoriented accordingly; whether H8 remains bound throughout this transition is unclear, as the lack of a transmembrane region in F_{ECTO} allows for greater degrees of freedom and facilitates the movement of the HRC domain. In the postfusion complex, contacts were again mediated mainly by the heavy chain, now comprising nine hydrogen bonds and two salt bridges, one per antibody chain. The interface areas in pre- and postfusion were comparable (461 vs. 486 Å²). ΔG values for both conformations were estimated to be similar (−4.2 kcal/mol for prefusion and −3.1 kcal/mol for postfusion).

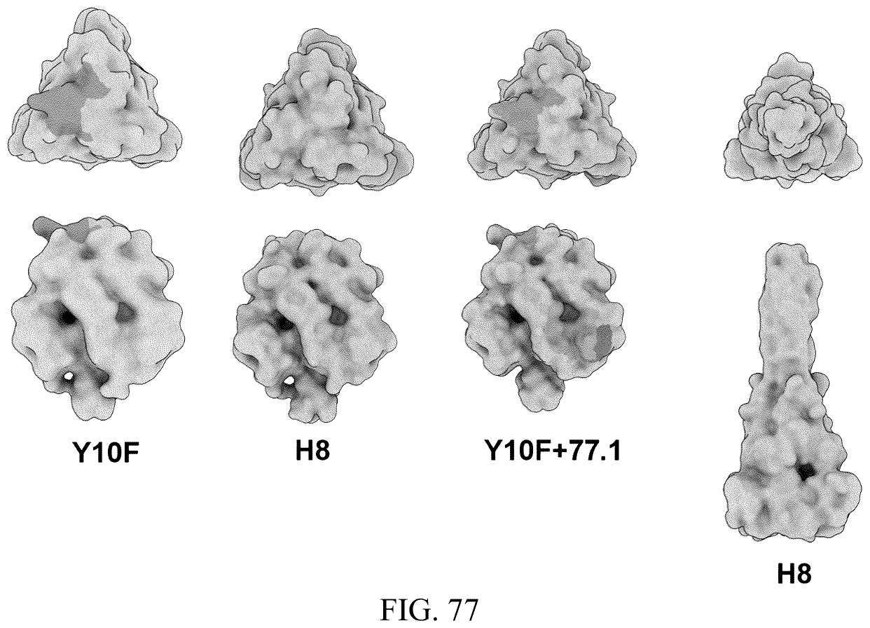

In summary, cryo-EM resolved Y10F as an apex-binding antibody that fits between glycan clusters on F2, confirmed the ability of Y10F to co-occupy F with 77.1 without major conformational changes of F, and showed that H8 targets a solvent-exposed loop in domain II that is accessible in both prefusion and postfusion conformations (see FIG. 77 for antibody footprint on the F protein).

### Ex Vivo, In Vitro and In Vivo Efficacy.

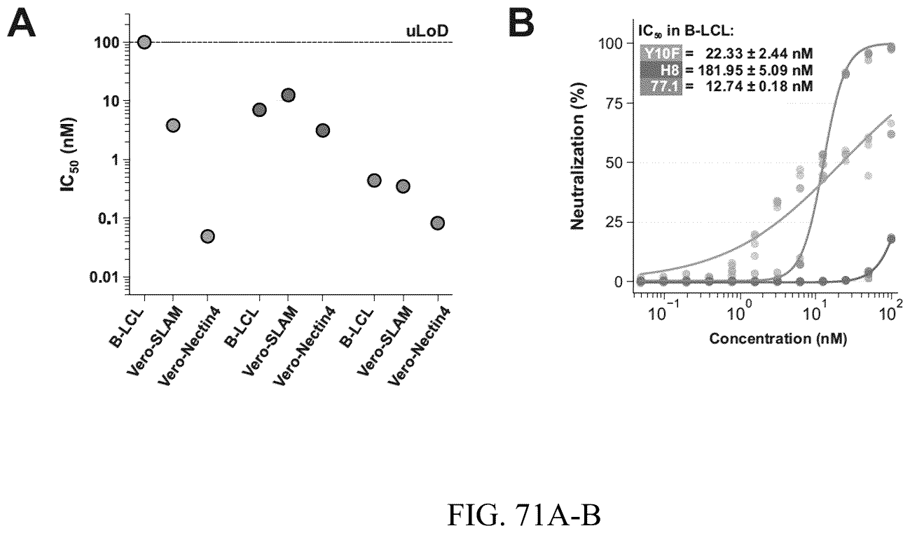

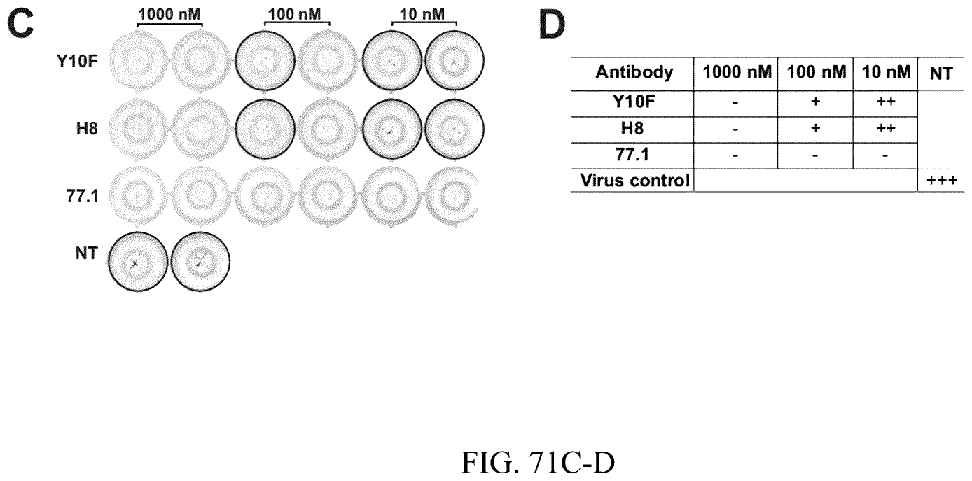

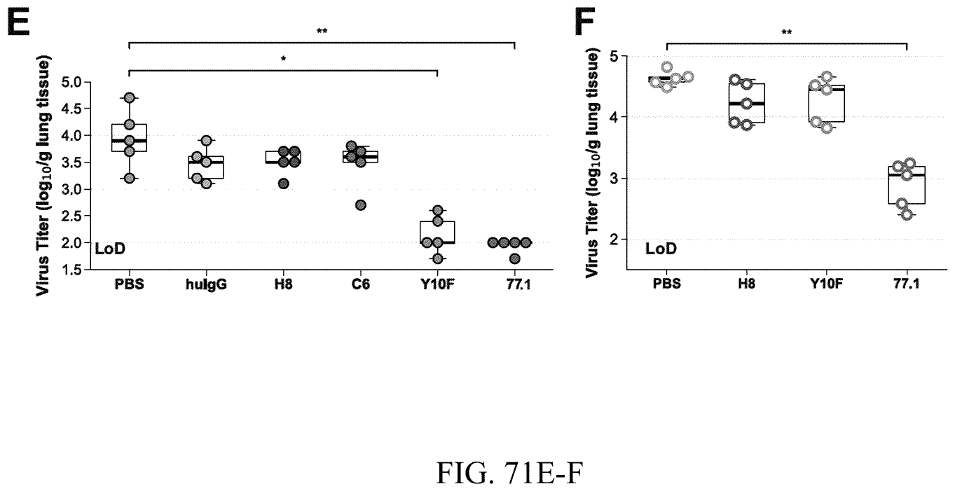

Applicants next determined whether the mAbs could inhibit MeV infection of authentic target cells (FIG. 71A-D). Vero cells stably expressing either human CD150 (Vero-hCD150) or Nectin-4 (Vero-hNectin-4), the two MeV entry receptors for wt MeV, were used initially, with parental cells serving as a negative control. As authentic immune target cells, assays were performed on human Epstein-Barr virus (EBV)-transformed B lymphoblastoid cell lines (B-LCL). 77.1 and H8 exhibited robust neutralizing activity against MeV on all receptor-positive cells; H8 with lower potency. Functionality of Y10F was variable: effective MeV neutralization on Vero-hCD150 and Vero-hNectin4 was observed (IC₅₀ of 6.25 nM and 0.34 nM, respectively), but Y10F could not prevent MeV infection of CD150-positive B-LCL (FIG. 71A). Across all conditions, 77.1 consistently outperformed Y10F and H8 in neutralizing MeV.

To further evaluate efficacy, antibodies were evaluated in a dissemination assay in human Epstein-Barr virus-transformed B-lymphoblastic cells (B-LCL). Consistent with the virus neutralization results, 77.1 inhibited MeV dissemination better than Y10F and H8, with an IC₅₀ of 12.7 nM. Interestingly, while unable to block infection in neutralization assays, Y10F could inhibit MeV dissemination in B-LCL cultures at an IC₅₀ of 22.3 nM (FIG. 71B).

To confirm functionality in a physiologically relevant epithelial cell context, bronchial ciliated human airway organoids differentiated at air-liquid interface (AO at ALI) were used. Prior to exposure to a mixture of mAbs and MeV, cultures were washed to remove mucus and mechanically disrupted to expose Nectin-4. All three mAbs inhibited MeV infection in this model (FIG. 71C). Notably, 77.1 achieved complete neutralization at all tested concentrations, whereas Y10F and H8 allowed residual infection at 10 nM and minimal infection at 100 nM (FIG. 71D).

Applicants next evaluated the prophylactic efficacy of these mAbs in vivo using a cotton rat model of MeV infection (FIG. 71E). Cotton rats were inoculated 14 hours before infection with a single dose (1 mg/kg) of the indicated mAbs. Four days after infection, lungs were collected and viral titers quantified (y-axis). A purified polyvalent human IgG (huIgG) preparation, which is used clinically for patients with primary immune deficiency and immune thrombocytopenia purpura, exhibited a neutralization titer of 640 at 60 mg/mL and served as a control. As expected, mAb 77.1 confirmed its strong in vivo efficacy previously demonstrated. The second-most potent mAb, Y10F, reduced viral titers to levels near the limit of detection. The other two mAbs (C6 and H8) performed comparably to huIgG, each reducing viral titers relative to PBS (diluent)-treated animals but without a significant difference.

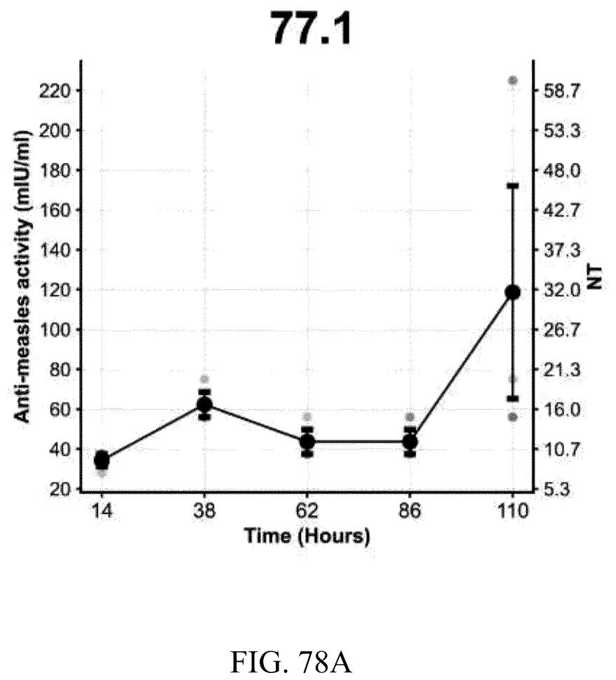

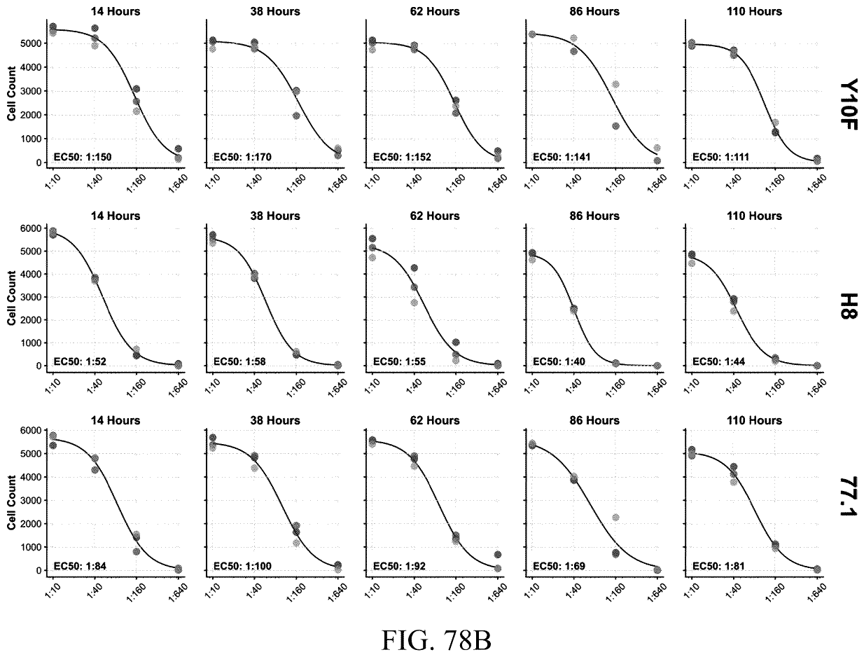

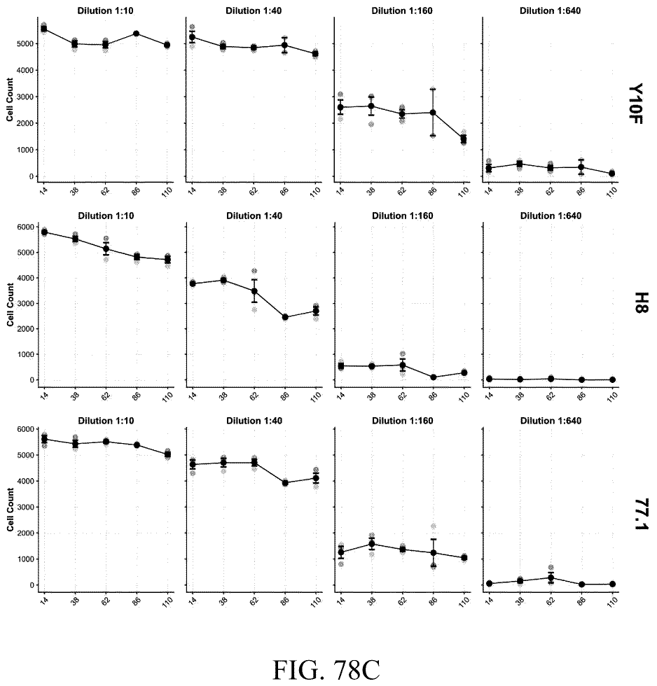

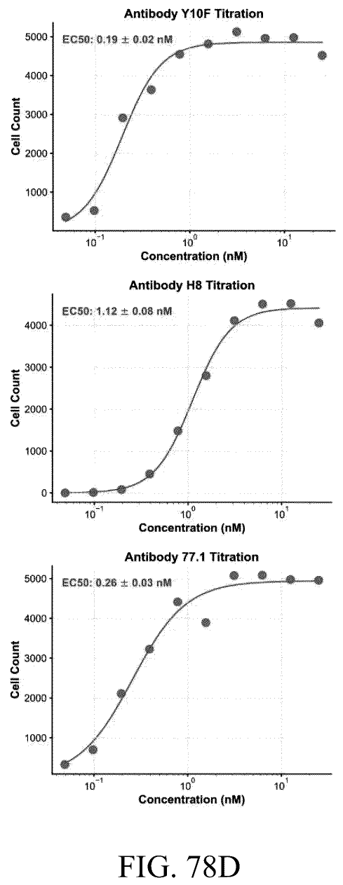

Applicants then tested mAb efficacy when administered after infection (FIG. 71F). Cotton rats were inoculated 24 hours after infection with a single dose (1 mg/kg) of the indicated mAbs. Four days post-infection, lungs were collected, and viral titers were quantified (y-axis). Under these conditions, mAb 77.1 showed significant efficacy, whereas the other mAbs did not reduce viral titers. Applicants then asked whether the lack of activity in vivo of Y10F and H8 was due to faster clearance. Applicants inoculated cotton rats intraperitoneally with 1 mg/kg of the three mAbs and the Human IgG. Blood was collected at 14, 38, 62, 86, and 110 hours post-inoculation. Applicants assessed the neutralizing activity for all and the presence of the three mAbs using a semi-quantitative cell-based ELISA at the time of infection and during the course of the challenge study (FIG. 78).

The data show that only sera from animals inoculated with mAb 77.1 exhibited neutralizing activity (FIG. 78A, expressed as mIU/mL y-axis), whereas all three mAbs remained at stable circulating levels throughout the experiment (14-110 hours post-inoculation). However, at the starting serum dilution used for the neutralization assay (1:40), the other antibodies did not block viral infection. By interpolating the serum binding data onto standard curves of purified mAbs (FIG. 78D), Applicants estimated that serum concentrations remained in the low nanomolar range for all three antibodies (Table 12). Specifically, H8 showed the highest retention, with concentrations ranging from approximately 40 to 65 nM, while Y10F and 77.1 remained stable at 17-33 nM (Table 12). These results indicate that the lack of therapeutic efficacy observed for Y10F and H8 in the post-infection setting was not due to rapid clearance or instability of the antibodies in vivo.

TABLE 12
Estimated serum concentrations and titration statistics of passively
transferred monoclonal antibodies related to FIG. 78.

| | | | | | EC₅₀ | Conc | Error |
|---|---|---|---|---|---|---|---|
| Antibody | Sample | Animal group | Unit | EC₅₀ | Error | (nM) | (nM) |
| Y10F | mAb Y10F | — | nM | 0.19 | 0.02 | | |
| | Sera 14 h | 1 | | 149.8 | 11.4 | 28.8 | 3.7 |
| | Sera 38 h | 2 | | 169.9 | 15.1 | 32.7 | 4.40 |
| | Sera 62 h | 1 | DF | 152.1 | 7.7 | 29.3 | 3.31 |
| | Sera 86 h | 2 | | 141.3 | 30.6 | 27.2 | 6.5 |
| | Sera 110 h | 1 | | 110.8 | 5.08 | 21.3 | 2.4 |
| H8 | mAb H8 | — | nM | 1.12 | 0.08 | | |
| | Sera 14 h | 3 | | 51.8 | 1.2 | 58 | 4.21 |
| | Sera 38 h | 4 | | 57.8 | 1.6 | 64.7 | 4.8 |
| | Sera 62 h | 3 | DF | 55.4 | 7.4 | 61.9 | 9.3 |
| | Sera 86 h | 4 | | 39.9 | 0.8 | 44.7 | 3.2 |
| | Sera 110 h | 3 | | 43.9 | 2.1 | 49.2 | 4.1 |
| 77.1 | mAb 77.1 | — | nM | 0.26 | 0.03 | | |
| | Sera 14 h | 5 | | 84.4 | 6.7 | 21.8 | 3.4 |
| | Sera 38 h | 6 | | 100.1 | 8.2 | 25.9 | 4.0 |
| | Sera 62 h | 5 | DF | 91.6 | 6.6 | 23.7 | 3.6 |
| | Sera 86 h | 6 | | 68.9 | 11.3 | 17.8 | 3.7 |
| | Sera 110 h | 5 | | 81.5 | 4.90 | 21.1 | 3.1 |

Summary of EC₅₀ dilution factors (DF) and calculated serum concentrations (nM) for mAbs Y10F, H8, and 77.1 at various time points post-inoculation. EC₅₀ values were derived from the logistic fits shown in FIG. 71B. Serum concentrations were estimated by interpolating these values against the standard calibration curves of purified antibodies (FIG. 71D). Data represent the calculated value ± standard error.

### Mechanism of Fusion Inhibition.

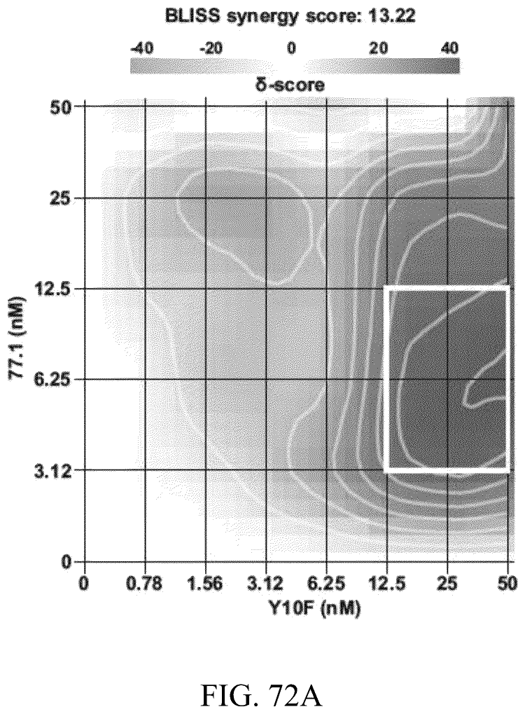

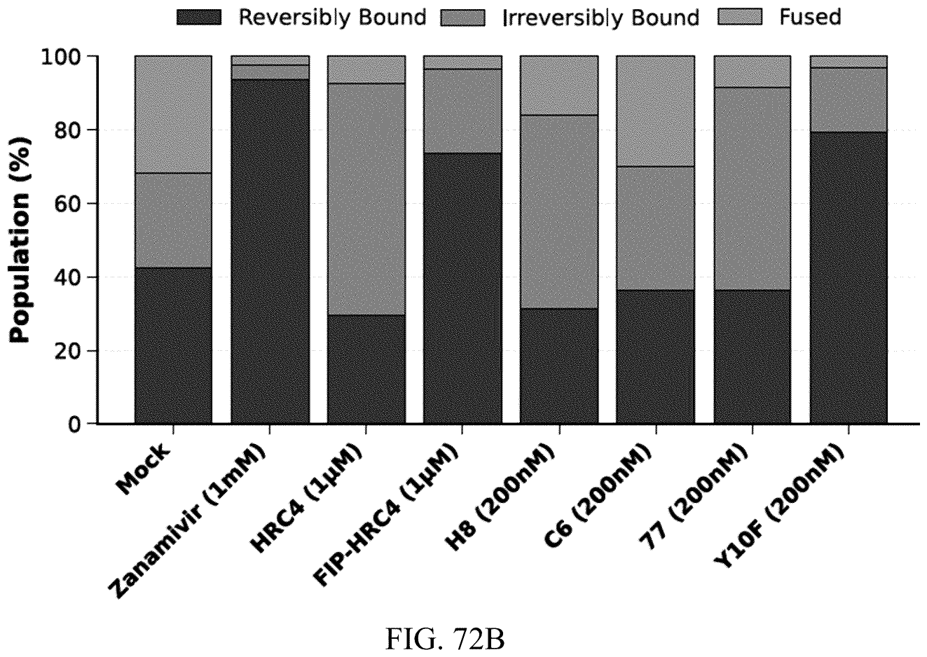

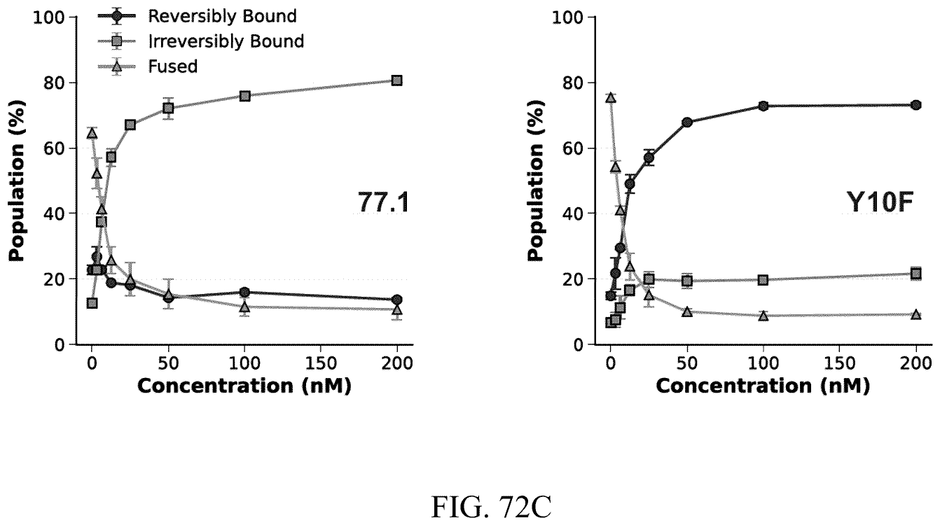

Since Y10F and 77.1 can simultaneously bind to the F protein (FIG. 69C), Applicants tested whether they exhibit synergistic inhibition of fusion. Fusion was measured using an α/ω β-galactosidase complementation assay, where HEK293T target cells expressing hCD150 and ω subunit were co-cultured with effector cells expressing MeV H/F and α subunit; and reconstituted enzyme activity was quantified by luminescence (FIG. 72A). Data were analyzed using SynergyFinder (https://synergyfinder.fimm.fi/) using the BLISS model. Applicants observed detectable moderate synergism between Y10F and 77.1 in inhibiting viral fusion.

To define the stage of the fusion process at which each mAb acts, Applicants used an assay measuring fusion of red blood cells (RBCs) with HEK293T cells expressing modified versions of MeV H and F (FIG. 72B). To distinguish F-mediated anchoring from H-mediated attachment, Applicants employed a chimeric receptor-binding protein consisting of the MeV H stalk fused to the head domain of human parainfluenza virus type 3 hemagglutinin-neuraminidase (HPIV3 HN). This H-HN chimera allows releasable attachment to sialic acid receptors (via HN) while still permitting MeV H-mediated triggering of F. As expected, RBCs bound through HN-sialic acid interactions could be released by adding Zanamivir. In untreated conditions, ~38% of RBCs fused with HEK293T cells, ~22% irreversibly bound via F (fusion initiated, but incomplete), and ~40% were reversibly bound via HN only (fusion not initiated). Zanamivir treatment, which blocks HN-mediated attachment, prevented nearly all F-mediated anchoring, confirming that receptor engagement is required for F activation. Treatment with the HRC4 peptide inhibitor yielded mostly irreversibly bound RBCs, indicating that F was activated, but fusion was blocked at a post-triggering step. By contrast, the FIP-HRC4 peptide inhibitor prevented nearly all F-mediated anchoring, consistent with inhibition of F activation. MAbs 77.1 and H8 blocked fusion in the same manner as HRC4, with most RBCs irreversibly bound, confirming that these two mAbs permit (77.1) or promote (H8) the F extension and allow fusion peptide insertion into the target cell, but block completion of the F transition. The profile observed in the presence of C6 at the tested concentration (200 nM) was statistically indistinguishable from that of mock-treated samples, consistent with their lack of efficacy. In contrast, mAb Y10F behaves similarly to FIP-HRC4, preventing the initiation of F activation. A dose-response version of this assay, with 77.1 and Y10F, confirmed that these antibodies act through distinct mechanisms: at higher concentrations, 77.1 drives cells into an irreversibly bound state with F trapped in an intermediate conformation, whereas Y10F drives cells into a reversibly bound state with F remaining untriggered (FIG. 72C).

Viral Evolution in the Presence of the mAbs 77.1 and Y10F.

Applicants first evaluated the potential for viral escape from mAb 77.1 by subjecting MeV to increasing concentrations of the antibody over five serial passages. Viral replication ceased after the fifth cycle, likely due to the accumulation of deleterious mutations. Nevertheless, viral populations from cycles four and five were harvested and subjected to deep sequencing to identify potential resistance-associated genetic changes. Remarkably, despite prolonged selective pressure from mAb 77.1, Applicants detected no significant point mutations with allele frequencies ≥10% or sequencing depths ≥20.

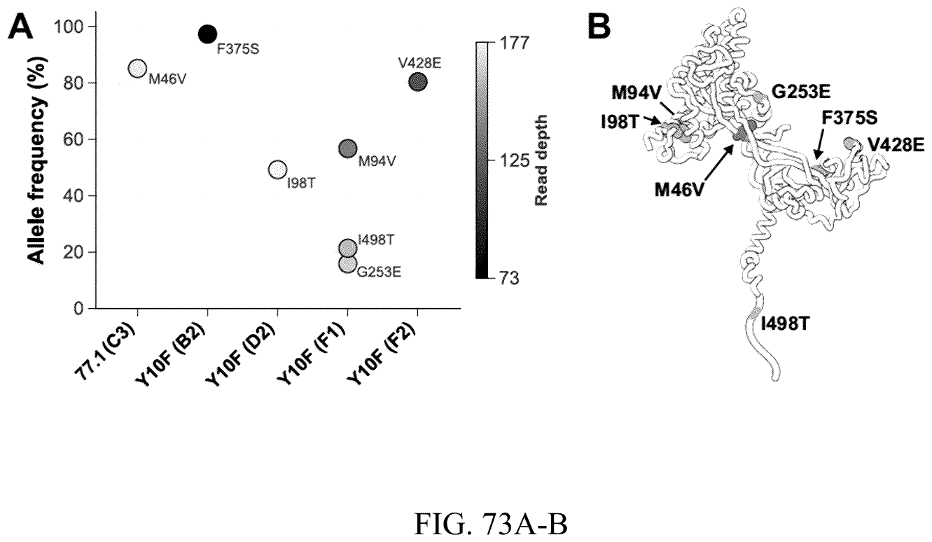

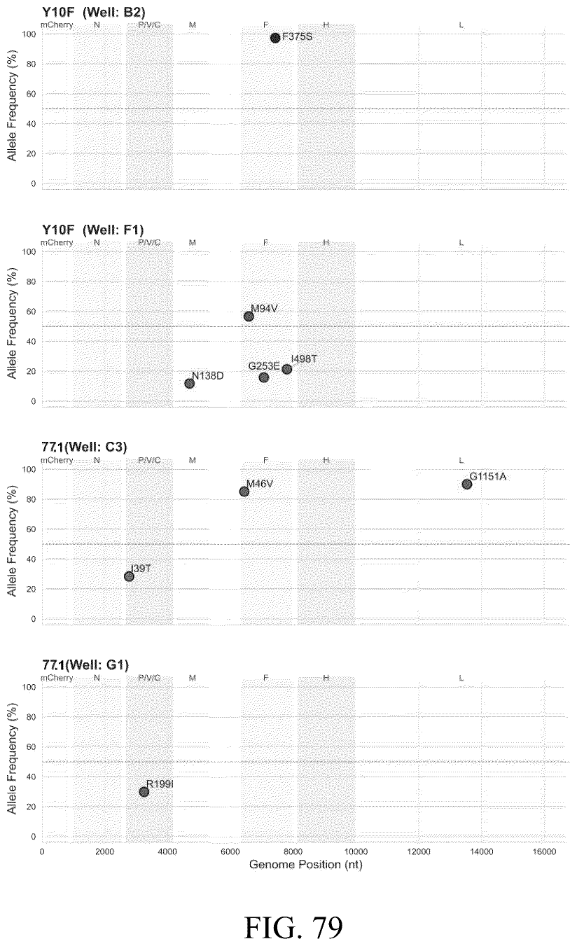

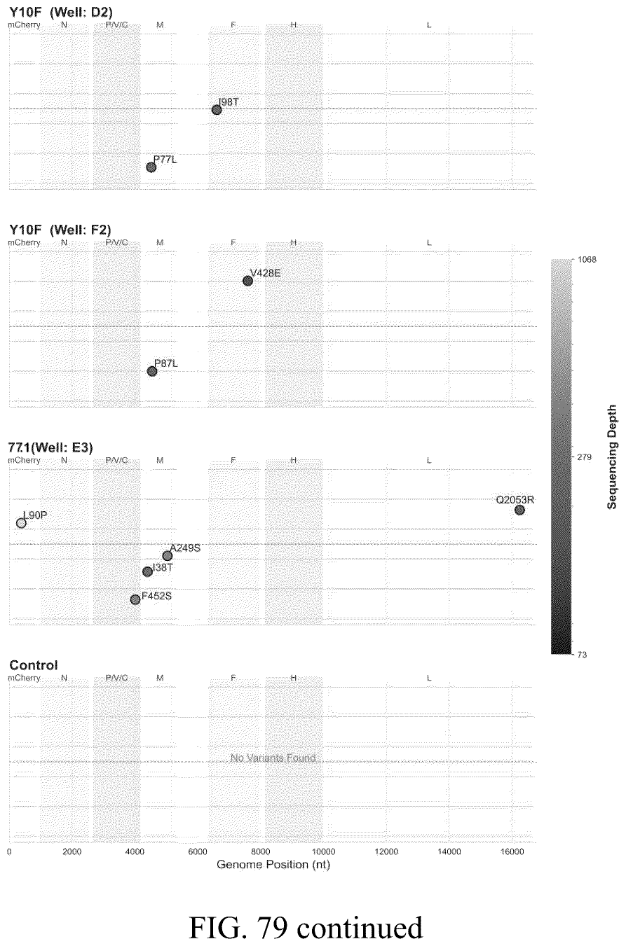

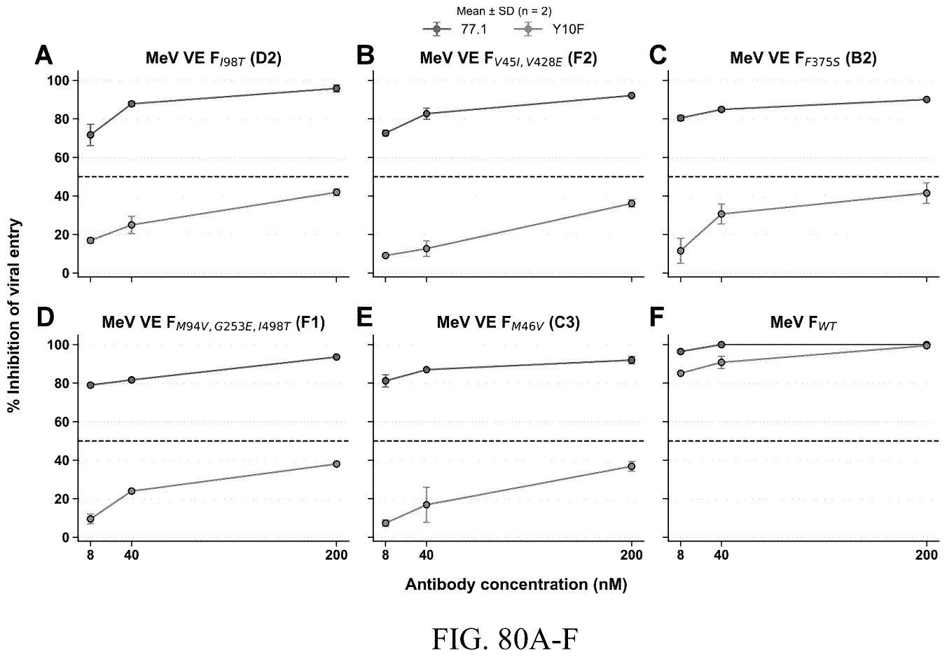

Applicants next repeated the viral evolution experiment with both 77.1 and Y10F (since these two antibodies were the most potent in vivo, as shown in FIG. 71E), using a different strategy. Instead of progressively increasing antibody concentrations, viruses were passaged for over 50 days in alternating concentrations of mAbs to permit ongoing replication (see Methods). This approach yielded eight distinct viral populations: four selected in the presence of 77.1 and four in the presence of Y10F. Three of the four populations grown with 77.1 showed no mutations in the F protein (Supplemental FIG. 79), while one population contained a single amino acid substitution (M46V) (FIG. 73A). In contrast, all four populations selected with Y10F acquired one or more amino acid substitutions in F (FIG. 73A). Among these, only the F375S variant reached complete fixation (100% allele frequency), whereas the others displayed partial penetrance. Nevertheless, all Y10F-selected populations were resistant to Y10F inhibitory activity but retained sensitivity to 77.1, albeit with reduced potency compared to the parental virus (FIG. 80). Similarly, M46V was also resistant to Y10F but retained sensitivity to 77.1, even though it was selected in the presence of 77.1. None of these mutations (FIG. 73B) mapped directly to the binding interface defined in FIGS. 69 and 70. This suggests that viral evolution under antibody pressure favors allosteric changes that functionally alter the F protein dynamics, rather than mutations within the epitope itself.

### DISCUSSION

Measles vaccines are highly effective but cannot be given to infants below the age of 9 months, or to immunocompromised or pregnant individuals, leaving them dependent on herd immunity. Population immunity to measles is fragile due to suboptimal vaccine coverage and the extreme transmissibility of MeV (R₀=12-18), driving resurgences in the U.S. and worldwide. MeV still causes 10-20 million infections and >200,000 deaths annually, with poor outcomes compounded by the post-measles “immune amnesia” that erodes immunity to other pathogens. No licensed antivirals exist, and current post-exposure prophylaxis with immune sera shows declining potency. While vaccine-induced neutralizing antibodies primarily target H, natural infection elicits potent antibodies against both the H and F proteins, highlighting F as a promising therapeutic target. Two recent preprint manuscripts from Applicants group further characterize the human antibody response to MeV. In the first, Applicants show that anti-F protein antibodies are found predominantly in older individuals, suggesting that wild-type infection elicits a stronger humoral response to the F protein than vaccination, which is consistent with prior observations in the literature. The second preprint examines the neutralizing activity of anti-F and anti-H antibodies isolated from human PBMCs. Notably, in that study, the most potent neutralizing antibodies target the F protein, supporting the conclusion that anti-F antibodies can be highly effective in vivo.

In the current study, Applicants investigated the neutralizing activity of chimeric mAbs targeting the MeV F protein. These mAbs were generated in mice, with their variable (Fab) regions grafted onto human constant (Fc) domains. One mAb, 77.1, which was previously described, binds an epitope present in both the prefusion and extended conformations of the F protein and arrests the F conformational transition at that intermediate step. Here, Applicants evaluated its potential for use in combination therapies and its barrier to viral resistance. A second mAb, Y10F, has previously been shown to possess neutralizing activity, but its sequence, epitope specificity, and mechanism of action were unknown. The other two mAbs, H8 and C6, also with unknown epitope structures, were selected for their distinct binding properties: H8 recognizes both pre- and post-triggered forms of F, whereas C6 binds only a post-triggered epitope.

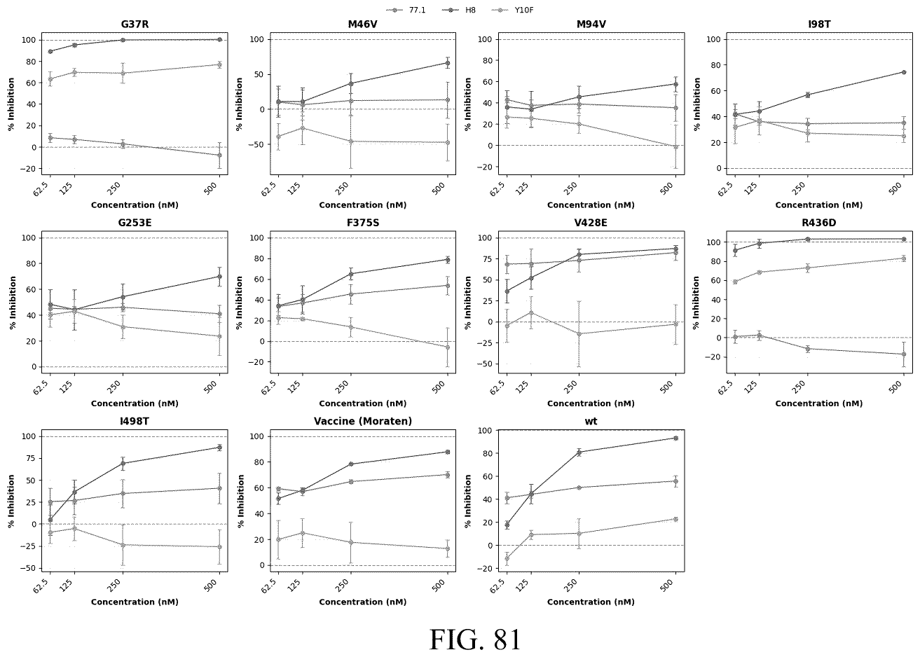

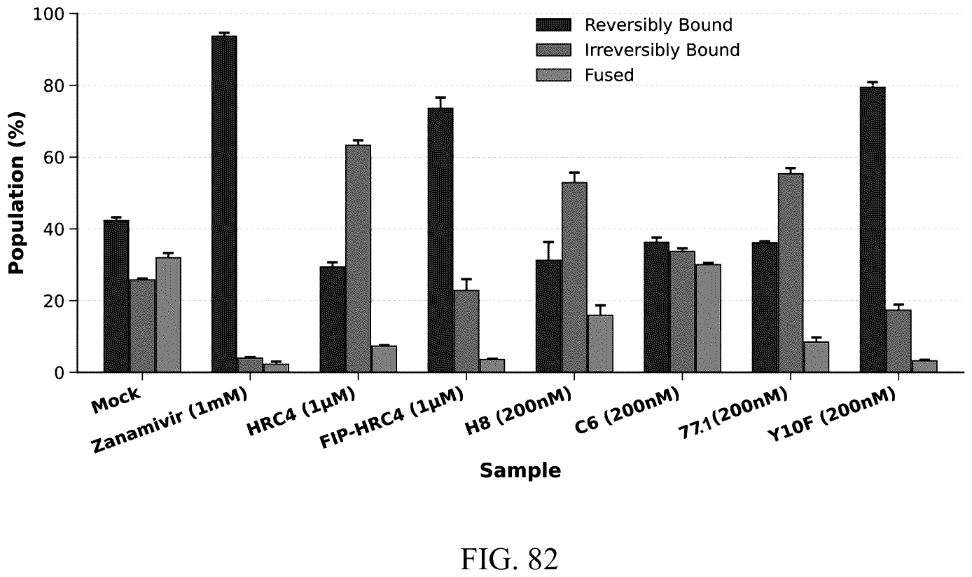

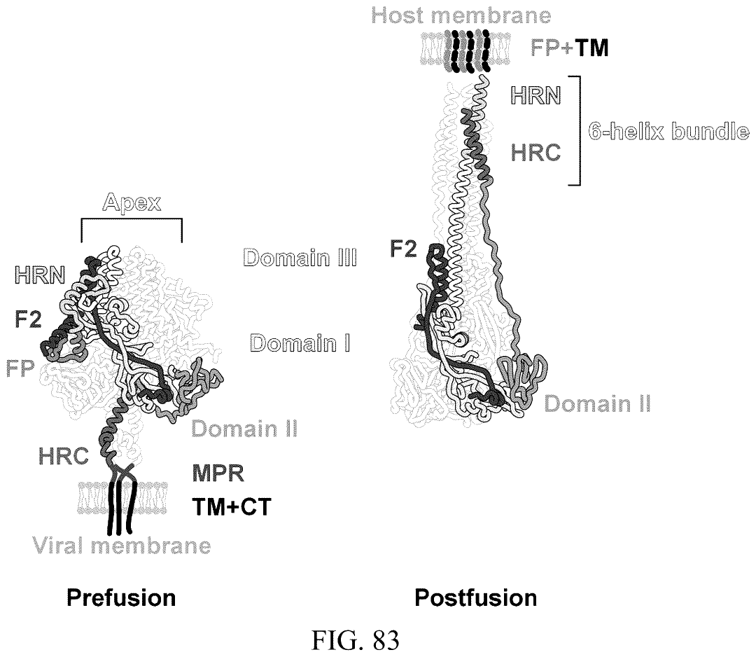

The three neutralizing mAbs (Y10F, 77.1, and H8), which target three distinct sites, collectively demonstrate that the MeV F protein contains multiple vulnerable epitopes that can be effectively targeted. Y10F binds an apical prefusion epitope, stabilizes the prefusion conformation, and neutralizes infection both in vitro and in vivo. However, for Y10F, ex vivo efficacy is reduced and viral escape can arise through allosteric mutations outside the binding interface. 77.1 recognizes F in the prefusion state but stays bound after triggering and inhibits fusion at a late stage (as Applicants have previously shown). 77.1 blocks MeV infection with high potency in authentic human tissue ex vivo, remains efficacious in vivo even when administered 24 hours post-infection, and displays a high barrier to resistance. H8 engages epitopes accessible in both the pre- and post-triggered conformations and, uniquely, promotes F activation upon binding, revealing a novel mode of antibody-mediated fusion regulation. Interestingly, H8 binds the 396-405 loop, a region critical for the H-F interaction and subsequent fusion triggering. In particular, this region includes residue Leu-394 (L394), which is notable as a known L394S mutation results in reduced fusion function and a 35-50% decrease in H-protein interaction, even though the protein itself is fully processed. While EM revealed H8-bound postfusion structures, RBC assays indicate that H8 arrests the process at a refolding step. Applicants attribute this apparent discrepancy to the soluble ectodomain's lack of transmembrane constraints, which allows it to complete refolding transitions that are energetically or sterically restricted in the membrane-embedded, full-length protein. Thus, combining these structural insights with the cell-based triggering, time-resolved SEC, and negative-stain data, Applicants hypothesize that H8 may bind this signal-receiving site, mimic the H protein, and activate F through a similar mechanism. In addition, H8 binding to F might influence the H-F interaction, thereby disrupting the glycoprotein complex on the surface. This binding profile of H8 is similar to that of the 1H1 antibody, which targets the Nipah virus F protein at an analogous epitope on the outer side of domain II. Like H8, 1H1 is also a neutralizing antibody and recognizes both the prefusion and postfusion states of the F protein. However, no data are currently available to support the 1H1 F triggering mechanism. This confirms that the premature activation induced by H8 represents a unique mechanism among this class of antibodies. In addition, H8 neutralizes a broad spectrum of MeV strains, including hyperfusogenic variants from neuropathogenic isolates and blocks fusion mediated by all the F variants obtained from the viral evolution with Y10F and 77.1 (FIG. 81). By contrast, C6, which recognizes only a post-triggered epitope in F (FIG. 74), failed to block viral entry or fusion, underscoring the importance of antibodies that target pre- and intermediate conformations.

Collectively, these findings establish the MeV F glycoprotein as a rich and underexplored source of neutralizing epitopes. A cocktail of long-acting, humanized anti-F antibodies, such as Y10F and 77.1, could provide durable protection with a high barrier to resistance, complement vaccination, and address a critical therapeutic gap for MeV-exposed seronegative health care workers, and to individuals who are unvaccinated, vaccine-ineligible (either due to early age or medical conditions), or have waning immunity.

### Methods

Ethics Statement. All studies received approval by the Institutional Animal Care and Use Committee of The Ohio State University. Inbred cotton rats (Sigmodon hispidus) were purchased from Inotiv (formerly Envigo; West Lafayette, IN) and maintained in a University Laboratory Animal Resources barrier facility at The Ohio State University. Cotton rats were housed in NexGen Rat 900 polysulfone microisolator cages (Allentown Inc., Allentown, NJ) with 12:12 hour light cycles, 22±2 °C, and 20-70% relative humidity. Age ranges were 5-7 weeks. Both sexes were used in all experimental groups.

Plasmids. The genes of MeV genotype B3, D8, or A (vaccine) H and F proteins, and the cellular receptor hCD150 were sub-cloned into the mammalian expression vector pCAGGS. The identification codes of the different F proteins are: B3-F (GS63325-1), D8-F (GS77420-3), Moraten-F (GS55588-1), L454W-F (GS55608), and E455G-F (GS74446-1). A codon-optimized F protein sequence derived from the Measles virus strain Ichinose-B95a (Taxonomy ID: 645098) was synthesized by GeneScript and subcloned into the pcDNA3.1 vector. Stabilizing point mutations (E170G and E455G) previously characterized in SSPE and MIBE Measles strains were sequentially introduced using the Q5 Site-Directed Mutagenesis kit. The ectodomain construct was generated by removing the transmembrane region and cytoplasmic tail residues (amino acids 496-550) using the Q5 Site-Directed Mutagenesis kit. To generate Drosophila S2 stable cell lines, F protein ectodomain construct E170G/E455G was subcloned from the pcDNA vector to the pMT-Puro vector, including a C-terminal enterokinase cleavage site-twin-strep-tag via NEB HiFi DNA assembly.

Antibodies. MeV F-specific murine monoclonal antibodies 77.4 and Y10F were generated as described previously. Chimeric antibody 77.1 has been functionally and structurally characterized. The remaining two antibodies, H8 and C6, were derived from murine hybridomas obtained after immunization with stabilized MeV F, cell-based ELISA was used to detect binding to pre- and postfusion state of the F protein; their Fab regions were subsequently sequenced and cloned into a human Fc backbone, as described for 77.1.

Cells. HEK293T (human kidney epithelial), Vero (African green monkey kidney), Vero-hCD150 (SLAMF1) and Vero-hNectin-4 cells were grown in Dulbecco's modified Eagle's medium (DMEM; Life Technologies; Thermo Fisher Scientific) supplemented with 10% fetal bovine serum (FBS, Life Technologies; Thermo Fisher Scientific) and antibiotics at 37 °C in 5% CO₂. The Vero-hCD150 culture media were supplemented with 1 mg/mL Geneticin (Thermo Fisher Scientific). Drosophila S2 cells were cultured in complete Schneider's Drosophila Medium (Gibco) supplemented with 10% heat-inactivated fetal bovine serum (FBS) and 1% penicillin-streptomycin (100 U/mL penicillin and 100 μg/mL streptomycin) at 25 °C without CO₂ or Lonza Insect Xpress Medium. ExpiCHO cells were maintained in ExpiCHO Expression Medium (Gibco) supplemented with 8 mM L-glutamine in a humidified incubator at 37 °C with 8% CO₂.

Viral rescue. HEK293T cells were transfected using Lipofectamine 2000 on 6-well plates coated with poly-D-lysine. The transfection mixture included purified plasmid constructs coding for viral proteins N (GS58900-1), P (GS58943), and L (GS58944), all sourced from Epoch Life Science according to Applicants design specifications. Additionally, purified plasmids for T7 polymerase (GS58929) and the genome for the MeV B3 mCherry wt virus bearing either the B3 H and F (GS69677-10), or the D8 H and F (GS77420-2), or the H and F from the Moraten vaccine strain (GS77420-1), or the B3 H and F-L454W (GS69677-1) were included. Cells were incubated overnight at 37 °C in Opti-MEM medium. The medium was then replaced with warm DMEM supplemented with 10% FBS. After 1 hour, cells were subjected to a heat shock treatment by incubating them at 42 °C in a water bath for 3 hours. Subsequently, the cells were returned to a 37 °C incubator for 48 hours. After this incubation, the HEK293T cells were detached and transferred to Vero-hCD150 cells in a 6-well plate and incubated at 32 °C for 5 days. RNA from the virus sample was then sequenced, and the virus stock was used for subsequent in vitro and in vivo experiments.

In vitro viral entry. Vero-hCD150 cells were plated in 96-well plates (1×10⁴ cells/well). The following day, cells were treated with serial dilutions of the indicated antibodies. Cells were then infected with the indicated viruses for 3 h at 37 °C (200-300 pfu/well). After 3 h, the medium was replaced with medium containing 2% of methylcellulose 2× complete medium (1:1). After 24 h, fluorescent/infected cells were counted using a Cytation 5 (BioTek) and analyzed with Gen5 3.11 software (min object size 25 μm, max object size 135 μm, threshold 3,000). The samples were tested in three separate experiments performed with technical duplicates in five-fold dilutions in 96-well plates. Data obtained from each plate was analyzed in OriginPro 2023, where all biological and technical replicates were fitted with a logistic regression function with shared IC₅₀ and Hill slope values, and A1 and A0 (highest and lowest signals) were used to normalize the data. Based on the normalized data, the percent of inhibition was calculated using the following equation: [1-X]*100, where X is the fraction of the normalized signal.

Beta-Galactosidase (Beta-Gal) complementation-based fusion assay. The Beta-Gal complementation-based fusion assay was performed as previously described. In brief, HEK293T cells transiently transfected with the hCD150 receptor and expressing the o subunit of β-galactosidase were co-cultured with cells co-expressing the MeV glycoproteins F and H together with the a subunit of β-galactosidase. Cell fusion results in α-ω complementation, which was measured in the presence or absence of inhibitory compounds. After 18 h, cells were lysed to terminate the fusion process, and fusion levels were quantified using the Tropix Galacto-Star™ chemiluminescent reporter assay system (Applied Biosystems) and read on a Tecan M1000 microplate reader. Biological replicates were recorded and globally fitted with a logistic function in OriginPro 2023. The obtained parameters were used to normalize the data per sample and plotted with standard error calculated from all data points.

Cell surface staining with conformation-specific anti-F mAbs. HEK293T cells transiently transfected with viral glycoprotein constructs were incubated overnight at 37 °C in complete medium (DMEM supplemented with 10% FBS). The following day, cells were shifted to indicated temperatures and incubation times (as shown in the figures). To detect specific MeV F conformations, cells were incubated on ice for 1 h with chimeric monoclonal antibodies (mAbs), followed by a 1 h incubation on ice with Alexa Fluor 594 or Alexa Fluor 488-conjugated anti-human secondary antibody (1:1000; Life Technologies). Cells were then fixed on ice with 4% paraformaldehyde for 10 min and counterstained with DAPI (1:1000; Thermo Fisher). Imaging was performed using a Cytation 5 system, and relative fluorescence of antibody-bound cells was quantified with BioTek Gen5 software.

Cell surface staining with conformation-specific anti-F C6 mAb: competition between H8 and Y10F mAbs. HEK293T cells were transiently transfected with viral glycoprotein constructs (either wt B3 or B3 E455G F) and incubated overnight at 37 °C in complete medium (DMEM supplemented with 10% FBS: CM). The following day, cells were brought to room temperature, and 50 μl of 2× Y10F (final concentrations: 200, 20, or 2 nM in CM) was added for 30 min. Subsequently, 50 μl of 2× H8 was added to achieve final concentrations of 100, 50, 25, 12.5, 6.25, 3.125, or 1.5625 nM in CM, resulting in diluted Y10F concentrations of 100, 10, and 1 nM, respectively. Cells were then transferred to 37 °C for 60 min. Following incubation, cells were placed at 4 °C, the supernatant was removed, and 50 μl of murine C6 mAb 1:500 dilution from an 8 mg/ml stock in CM) was added for 60 min. Cells were washed three times with CM and incubated on ice for 30 min with Alexa Fluor 488-conjugated anti-mouse secondary antibody (1:500; Life Technologies). Cells were then fixed on ice with 4% paraformaldehyde for 10 min and counterstained with DAPI (1:1000; Thermo Fisher). Imaging was performed using a Cytation 5 system, and C6-positive cells were quantified using BioTek Gen5 software.

Virus neutralization assay in cell lines. 77.1, Y10F, and H8, were serially diluted two-fold, starting at 100 nM, and incubated with 60 TCID₅₀ rMeV-KS-Venus(3) per well. Following a 1 h incubation at 37 °C with 5% CO₂, 1×10⁴ cells per well were added (Vero-hCD150, Vero-Nectin4, or Vero). Plates were incubated for 7 days and infection was monitored by Venus fluorescence. After 7 days, plates were imaged using an Amersham Typhoon scanner under predefined settings: channel Cy2 (GFP,488), pixel size 10 μm, scan speed normal, and PMT auto. The assay was performed in quadruplicate. In the Vero-hCD150 assays, wells were scored as positive or negative based on the presence or absence of Venus (endpoint titration), whereas in the Vero-hNectin4 assay the Venus positive surface area was calculated with Fiji software. Wells were scored positive if the infection covered >10%. For virus neutralization assay in B-LCLs. mAbs dilutions were prepared as described above and incubated with 10⁶ TCID₅₀ rMeV-KS-Venus (3) per well. Following a 1 h incubation at 37 °C with 5% CO₂, 1×10⁵ B-LCLs per well were added and incubated for 7 days. Plates were imaged using an Amersham Typhoon scanner under predefined settings: channel Cy2 (GFP,488), pixel size 10 μm, scan speed normal, and PMT auto. The assay was performed in quadruplicate, and wells were scored as positive or negative for infection based on the presence or absence of Venus (endpoint titration).

MeV dissemination assay in B-LCLs. A previously established MeV dissemination assay was used to assess the potential of the mAbs to prevent MeV dissemination. Briefly, B-LCLs were infected with rMeV-KS-Venus (3) for 72 hours. Infection rates in live cells were quantified by flow cytometry (BD FACS Lyric), based on Venus expression. Next, 150 MeV-infected B-LCLs were co-cultured with 6×10⁴ uninfected B-LCLs per well in the presence of a two-fold dilution series of mAbs starting at 100 nM. MeV dissemination was determined 48 hours post co-culture by measuring the percentage of Venus-positive cells by flow cytometry.

Virus neutralization assay in Airway organoids at air-liquid interface (AO at ALI). AO differentiated at ALI for >6 weeks, exhibiting ciliation and mucus production, were generated as previously described. Prior to infection, AO at ALI were rinsed with Ca²⁺/Mg²⁺-containing DPBS to remove mucus and scratched in a cross-pattern to expose Nectin-4 and facilitate viral entry. Monoclonal antibodies (1000, 100, and 10 nM) were pre-incubated with rMeV-KS-Venus (3) (MOI=0.1) for 1 hour prior to application to the apical surface. After 2 hours, the inoculum was removed to restore ALI conditions. Organoids were imaged using the Typhoon scanner at 3 days post-infection, and AO at ALI were semi-quantitatively scored for MeV infection.

Viral evolution assay. Selection of Measles Virus Escape Mutants in the Presence of Monoclonal Antibodies

### Cell Culture and Virus Infection.

### Phase 1: Cell Culture and Virus Infection.

Vero-hCD150 cells were seeded in two 96-well plates and infected with 100 plaque-forming units (PFUs) per well of recombinant Measles virus (MeV) B3 wild-type expressing mCherry. Infections were carried out in Opti-MEM (Gibco, 31985070) supplemented with 10% penicillin-streptomycin. After 2 hours, the infection medium was replaced with serially diluted concentrations of monoclonal antibodies Y10F and 77.1, starting at 10 μg/mL and diluted 1:2 down to 0.04 μg/mL.

### Phase 2: Passaging Under Antibody Pressure.

On day 10 post-infection, wells showing evidence of ongoing viral replication in the presence of highest (i.e., 10 μg/mL) antibody concentration (1-77.1: B1 and G1; 1-Y10F: F1 and E1) were selected. The contents of each well were transferred to T75 flasks containing Vero-hCD150 cells in Opti-MEM supplemented with 10% penicillin-streptomycin and 10 g/mL of the corresponding antibody.

Due to limited infection after 3 days, additional wells from the antibody selection plates were chosen and expanded: wells 2-77.1 (A3, C3, E3) and 2-Y10F (B2, D2, F2) were transferred to T75 flasks and cultured in Opti-MEM with 10% pen-strep, containing reduced antibody concentrations (1.25 μg/mL for 77.1; 2.5 μg/mL for Y10F).

On day 15, the medium from the initial T75 flasks (i.e., 1-77.1: B1 and G1; 1-Y10F: F1 and E1) was replaced with Opti-MEM+10% pen-strep without antibody. Virus supernatants were collected and frozen at −80 °C on day 16. Antibody concentrations in the second batch flasks were further reduced on day 17 due to a lack of visible infection (0.625 μg/mL for 77.1; 1.25 μg/mL for Y10F). Viral supernatants from these cultures were collected on days 23 and 28 (2-Y10F: B2, D2, F2; 2-77.1: A3, C3, E3), frozen, and subsequently used for further passages.

### Sequential Antibody Reduction and Virus Harvesting.

Phase 3: On day 28, all remaining samples were subsequently used to infect T75 flasks of Vero-SLAM cells at 10-fold lower antibody concentrations (0.125 g/mL for 2-Y10F, 0.0625 μg/mL for 2-77.1, and 0.5 μg/mL for first-round isolates). Viruses were all harvested in the following 10-12 days.

Phase 4: On day 39, these eight virus stocks were then used to infect new T75 flasks of Vero-hCD150 cells. After three hours, antibody was reintroduced at two-fold lower concentrations (0.0625 μg/mL for 2-Y10F, 0.0313 μg/mL for 2-77.1, and 0.25 μg/mL for 1-Y10F and 1-77.1).

Phase 5: On day 54, each virus was collected, titered, and sequenced.

In vivo experiments: Cotton rats. Inbred cotton rats (Sigmodon hispidus) were purchased from Envigo, Inc., Indianapolis. Both male and female cotton rats aged 5 to 7 weeks were used. For i.n. infection, 10⁵ TCID₅₀ of MeV (strain B3 wt EGFP) was inoculated intranasally to isoflurane-anesthetized cotton rats in a volume of 100 μL. Animals received either the indicated amount of the indicated antibodies, purified human IgG (Carimune, CSL Behring, measles neutralizing titer of 640 at 60 mg/mL), or vehicle 14 hours prior to infection or 24 hours post-infection. Four days after infection, the animals were euthanized by CO₂ inhalation, and their lungs were collected and weighed. Lung tissue was homogenized with a glass Dounce homogenizer. Serial 10-fold dilutions of supernatant fluids were assessed on Vero-hCD150 cells for the presence of infectious virus in 48-well plates using cytopathic effect (CPE) as the endpoint. Plates were scored for CPE microscopically after 7 days, and the tissue culture infectious dose 50 (TCID₅₀) was calculated. The results are presented as TCID₅₀/g of lung tissue.

RBC fusion assay. RBC fusion assays were performed using HEK293T cells transiently expressing the MeV H_Y17H-HPIV3_T193A chimera and MeV F (T461I). Cell monolayers were washed three times with serum-free medium and incubated at 4 °C for 30 min in CO₂-independent medium containing 1% RBCs. Cells were then exposed to varying concentrations of inhibitors and shifted to 37 °C for 60 min.

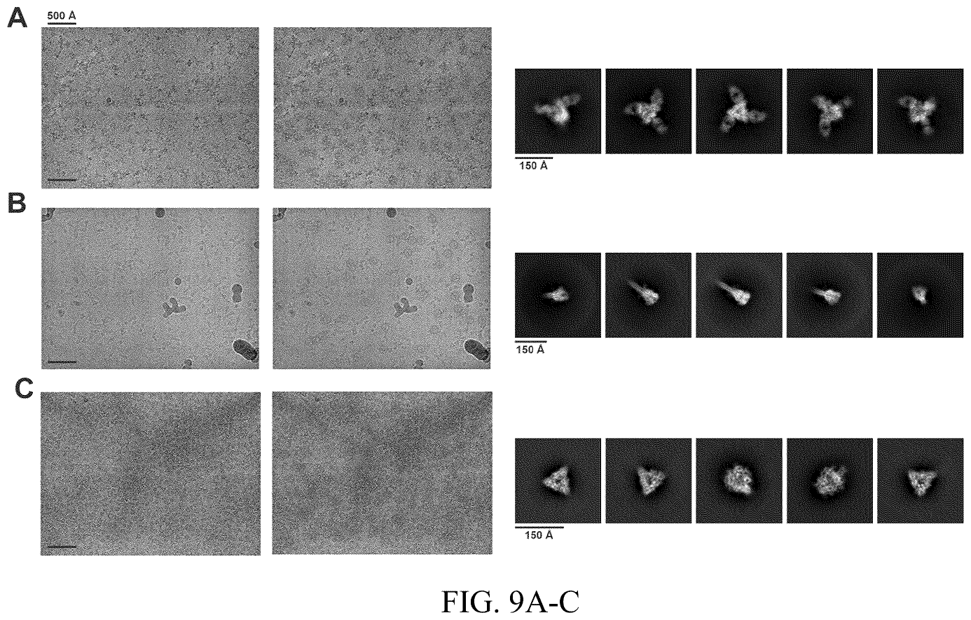

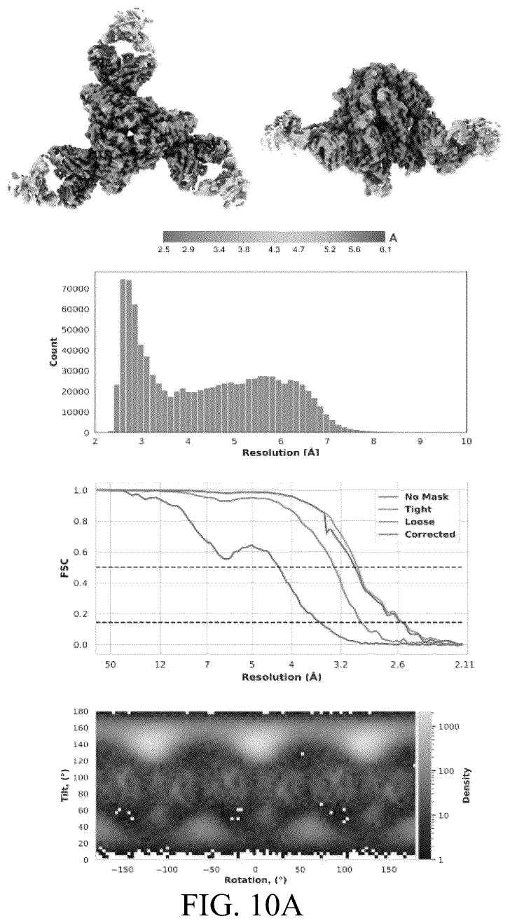

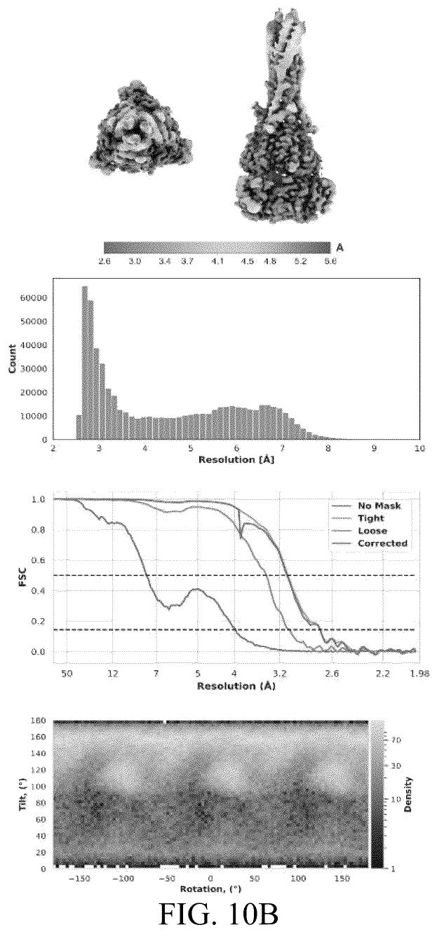

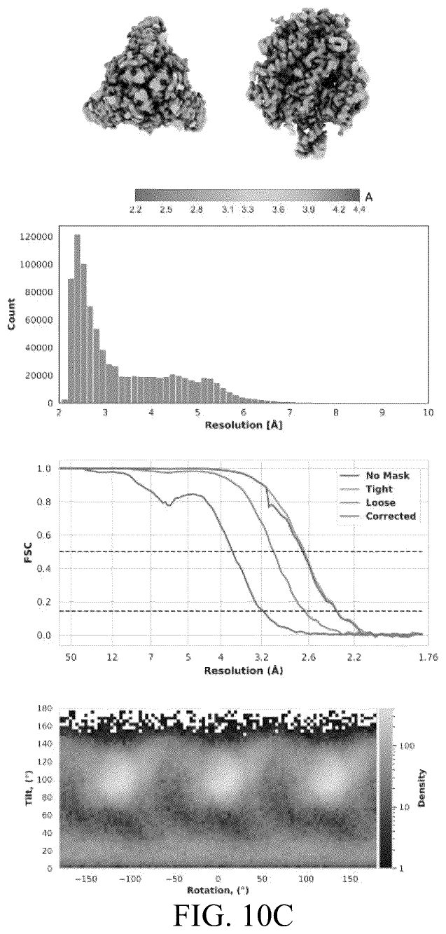

To quantify RBC binding and fusion, zanamivir was added at a final concentration of 10 mM, and the cells were incubated at 4 °C for 30 min with gentle agitation. The supernatant was collected into V-bottom 96-well plates to measure reversibly bound RBCs. The cells were subsequently incubated at 4 °C for 30 min in ACK lysing buffer, and the supernatant was collected into V-bottom 96-well plates to quantify irreversibly bound RBCs. Finally, the remaining cells were lysed in lysis buffer and transferred to flat-bottom 96-well plates to determine fused RBCs. Absorbance at 410 nm was measured using a Tecan My1000PRO microplate reader to quantify RBCs in each category. Assays were performed in CO₂-independent media at pH 7.2 for the bar graphs in FIG. 6B and Supplemental FIG. 9, and at pH 7.6 for the dose response curves in FIG. 6C.

F_{ECTO} purification. F_{ECTO} was purified as previously described from Drosophila S2 cells. Protein concentration was estimated using the molar extinction coefficient at 280 nm (48,820 M⁻¹ cm⁻¹).

Antibody expression. Monoclonal antibodies were produced in ExpiCHO cells using the manufacturer's high-titer transfection protocol with pTRIOZ-mAb plasmids encoding the antibody heavy and light chains. After seven days, cells were removed by centrifugation at 4,000×g, and the clarified medium was adjusted to pH 8.0 with 1 M HEPES (pH 8.0). Antibodies were captured by incubating the medium overnight at 4 °C with protein A agarose. The next day, beads were washed with 20 mL HBS, and bound antibody was eluted with 15 mL 0.2 M glycine (pH 2.0). The eluate was immediately neutralized by adding 10% (v/v) 1 M HEPES (pH 8.0).

For the Fab generation, purified IgG at 1 mg/mL was digested with 2% (w/v) cysteine-activated papain for 4 h at 37 °C in a water bath. The reaction was quenched by adding 0.5 M iodoacetamide to a final concentration of 50 mM. The mixture was diluted tenfold with 10 mM HEPES (pH 8.0) to reduce ionic strength and loaded onto a 1 mL Mono S column pre-equilibrated with 20 mM HEPES (pH 8.0). Fab fragments were collected in the flow-through, whereas Fc fragments were eluted in a single step with 20 mM HEPES (pH 8.0) containing 2 M NaCl. Isolated Fabs were concentrated, buffer-exchanged into HBS, and stored at 4 °C. Protein concentrations were determined from A280 using the calculated molar extinction coefficients.

Surface plasmon resonance. Binding experiments were performed on a Carterra LSA instrument. Purified monoclonal antibodies (mAbs) were covalently immobilized onto an HC30M sensor chip at a concentration of 2.5 μg mL⁻¹, with each antibody injected in duplicate. A serial dilution of the analyte (F_{ECTO}) was applied to the chip in ascending order: 3.125 nM, 6.25 nM, 12.5 nM, 25 nM, 50 nM, and 100 nM. The resulting binding responses were recorded over time. Kinetic parameters were extracted by globally fitting the sensorgrams to Equations 1-6 using OriginPro software.

$$Observed rate and equilibrium : {k}_{obs} = {k}_{a} C + {k}_{d} , {R}_{eq} = \frac{{k}_{a} C}{{k}_{a} C + {k}_{d}} {R}_{max} . \qquad Eq 1$$

$$Association : {S}_{a} ( t ) = {R}_{eq} + ( {S}_{0} - {R}_{eq} ) {e}^{- {k}_{obs} ( t - {t}_{s} )} . \qquad Eq 2$$

$$Value at the switch : S {@}_{{t}_{d}} = {R}_{eq} + ( {S}_{0} - {R}_{eq} ) {e}^{- {k}_{obs} ( {t}_{d} - {t}_{s} )} . \qquad Eq 3$$

$$Dissociation : {S}_{d} ( t ) S {=}_{{t}_{d}} {e}^{- {k}_{d} ( t - {t}_{d} )} . \qquad Eq 4$$

$$Baseline : B ( t ) = b + m ( t - {t}_{s} ) . \qquad Eq 5$$

$$Final fit : R ( t ) = \{ \begin{matrix}B ( t ) + {S}_{a} ( t ) , & t < {t}_{d} \\ B ( t ) + {S}_{d} ( t ) , & t > {t}_{d}\end{matrix} . \qquad Eq 6$$

Where: t: time(s). tₛ: association start(s). t_{d}: dissociation start(s), C: analyte concentration (M), kₐ: association rate constant (M⁻¹ s⁻¹), k_{d}: dissociation rate constant (s₋₁), K_{obs}: observed association rate (s⁻¹), Rₘₐₓ: maximum response at full occupancy (response units), R_{eq}: equilibrium response at C (response units), S₀: residual bound signal at tₛ (response units), S_{@td}: signal at the switch to buffer (response units), b: baseline offset (response units), m: linear drift (response units s⁻¹)

Time-resolved size-exclusion chromatography. F_{ECTO} was mixed with H8 Fab at a 1:1 molar ratio (monomer: Fab). The mixture was incubated at 25 °C for 15, 90, 180, or 1440 minutes. At each time point, 100 μL of the reaction was injected onto a Cytiva Superdex 200 Increase 10/300 GL column. The column was pre-equilibrated with HBS buffer containing 20 mM HEPES (pH 8.0) and 150 mM NaCl. Absorbance at 280 nm was recorded throughout the elution. Chromatograms were exported to OriginPro 2025 and baseline-corrected using the spline method. Corrected traces were used to calculate the trapezoidal area between 7 and 10 mL elution volume in Python. This region includes the column void volume and contains F_{ECTO}-H8 species associated with triggering. The resulting area values were plotted as a function of incubation time. Data were fitted with a single-exponential function in OriginPro 2025 to estimate kinetic parameters.

Time-resolved negative-stain electron microscopy. Complexes for negative-stain analysis were prepared in the same way as for size-exclusion chromatography. Aliquots were removed from the reaction after 0, 15, 60, 150, 240, and 1140 minutes. Each aliquot was immediately applied to a grid and processed for negative staining. For every time point, approximately 176 movies were collected and processed in cryoSPARC version 4.7.1. Patch motion correction and CTF estimation were performed using default settings. Particles were selected using the blob picker with a diameter range of 120-250 Å. A single round of two-dimensional classification was run with 100 classes and 500 particles per class. The protocol consisted of 30 online iterations, followed by two full iterations. Class occupancy was calculated from the number of particles assigned to each selected class. Class 5 was excluded from the final analysis because it contained heterogeneous particles such as H8 Fab bound to F fragments, Fab alone, and other mixed species. The final class occupancy values were plotted over time using Python, Matplotlib, and Seaborn.

### Grid Preparation

Negative stain. Negative-stain electron microscopy was performed using 400-mesh copper grids coated with a continuous carbon film (CF400-Cu; Electron Microscopy Sciences). Prior to sample application, the grids were rendered hydrophilic by glow-discharging them in an easyGlow Glow Discharge Cleaning System (Pelco) for 15 s at 15 mA. Protein samples were diluted to an absorbance of 0.01 at 280 nm; 3 μL of this solution was then deposited onto the grid and incubated for 1 minute. The excess sample was removed by blotting, and the grid was washed three times with 3 μL of deionized water, with blotting performed between each wash. Negative staining was performed by applying 3 μL of a 1% uranyl acetate solution to the grid, blotting twice, then adding an additional drop of stain and incubating for one minute before a final blot to remove excess stain. The grids were allowed to air-dry prior to imaging.

Cryo-EM sample preparation and data acquisition. Complex stoichiometries were guided by NS-EM. Except where noted, complexes were incubated with a twofold molar excess of Fab relative to the number of F binding sites. Accordingly, samples were prepared at trimer: Fab ratios of 1:2 for F_{ECTO}: Y10F (one F trimer to two Y10F Fabs) and 1:2:6 for F_{ECTO}: Y10F: 77.1 (twofold excess of 77.1). For H8, Fab was added at a 1:1 ratio per F binding site (1:3 trimer: H8), because this complex was not purified before imaging.

F_{ECTO}: Y10F and F_{ECTO}: Y10F: 77.1 complexes were purified by size-exclusion chromatography (SEC) before grid preparation. The F: H8 complex was applied directly to grids after a 15-minute incubation to minimize postfusion conformational transition that could occur during SEC. Data were collected on graphene oxide (GO) grids as described previously. All F_{ECTO}-Fab complexes were recorded on Quantifoil R2/1 or R2/2 grids covered with GO grids prepared as described previously. Protein with 0.25-35 absorbance values at 280 nm was placed on grids and blotted for 2-3s. Grids were loaded to the FEI Titan Krios G3 autoloader and screened for ice quality, graphene oxide layer quality, and particle distribution. Movies were recorded with an exposure rate of 70 e/Å2, with 50 frames per movie saved as non-gain normalized LWZ TIFF files. Collection statistics per dataset are reported in Table 11.

TABLE 11
Cryo-EM statistics from all recorded datasets.

| | F_{ECTO} + H8 | F_{ECTO} + H8 | F_{ECTO} + Y10F + | F_{ECTO} + |
|---|---|---|---|---|
| | (prefusion) | (postfusion) | 77 | Y10F |
| Data Collection | | | | |
| Microscope and camera | Titan Krios G3 + Gatan K3 with Bio Quantum Energy | | | |
| Voltage (kV) | 300 kV | | | |
| Magnification | 130,000 | | | |
| Raw Pixel size (Å) | 0.66 | | | |
| Electron dose (e⁻/Å²) | 70 | | | |
| Energy Filter slit (eV) | 20 | | | |
| Refinement | | | | |
| Final box size (px) | 320 | 584 | 380 | 464 |
| Final pixel size (Å) | 1.056 | 0.88 | 1.0768 | 0.88 |
| Final number of particles | 210,483 | 312,278 | 221,894 | 129,970 |
| Symmetry applied | C3 (relaxed) | C3 (relaxed) | C1 | C1 |
| Map and Model Statistics | | | | |
| 0.143 FSC Resolution (Å) | 2.42 | 2.8 | 2.82 | 2.57 |
| CC (mask) | 0.77 | 0.87 | 0.8 | 0.85 |
| 0.143 Masked Map/Model | 2.4/2.4/2.7 | 2.7/2.8/3.0 | 2.7/2.8/3.1 | 2.5/2.5/2.8 |
| FSC (Å) | | | | |
| Model Composition | | | | |
| Chains | 14 | 15 | 13 | 12 |
| Protein Residues | 2024 | 1919 | 1811 | 1585 |
| Ligands | NAG: 15, BMA: | NAG: 9 | NAG: 16 | NAG: 15, BMA: |
| | 3, MAN: 4 | | | 3, MAN: 4 |
| Water molecules | 140 | 0 | 0 | 63 |
| Total Atoms | 15835 | 14828 | 14059 | 12394 |
| Model Quality | | | | |
| MolProbity score | 1.5 | 1.33 | 1.22 | 1.36 |
| Clash score | 5.77 | 3.34 | 2.91 | 5.53 |
| Rotamer outliers (%) | 0.0 | 0.06 | 0.0 | 0.0 |
| CaBLAM outliers (%) | 1.42 | 1.66 | 1.13 | 1.29 |
| Ramachandran Plot (%) | | | | |
| Favored | 96.9 | 96.72 | 97.26 | 97.71 |
| Allowed | 3.1 | 3.28 | 2.74 | 2.29 |
| Outliers | 0.0 | 0.0 | 0.0 | 0.0 |

Cryo-EM data processing. All datasets were processed in cryoSPARC v4.5 or v4.6 using a standard workflow with dataset-specific adjustments during late refinement. Movies were corrected for beam-induced motion with Patch Motion, and defocus values were estimated with Patch CTF in cryoSPARC Live. Particles were initially picked with the blob picker using an intentionally broad particle diameter (80-120 Å) to over-pick F_{ECTO}-Fab complexes. Particles were first extracted and Fourier-cropped to 64 pixels to speed up early processing.

Particle stacks were extensively cleaned by 2D classification following the Deep2D protocol described previously, and class averages showing well-defined protein features were retained. Two independent ab initio reconstructions were run per dataset, one imposing C1 and one C3 symmetry, each with five classes; for the Y10F dataset, only C1 was used. The best ab initio models were then used for heterogeneous refinement against all classes, iterating until discrete 3D classes emerged. The best-looking class or classes were pooled and subjected to non-uniform refinement (NUR), after which particles were re-extracted with larger Fourier-cropped boxes (160-256 pixels) for further NUR and additional 3D classification to improve compositional and conformational homogeneity.

For final map generation, only particles contributing to the best-resolved class were retained and re-extracted with a Fourier crop chosen so that the 0.143 FSC resolution fell within approximately three-quarters of the box frequency (to avoid box-size-limited resolution). Final refinements included NUR, symmetry expansion where appropriate, focused/local refinement with masks, and targeted 3D classification, followed by a final round of local refinement. The final particle set was subjected to reference-based motion correction and both global and local CTF refinement, after which a final local refinement produced the maps reported.

Model building. Model building proceeded by rigid-body fitting of the previously reported F_{ECTO} structure (PDB 8UUP) into each cryo-EM map. Antibody Fab fragments were modeled with AlphaFold2 and docked into the density using UCSF ChimeraX. The combined F_{ECTO}-antibody models were then iteratively adjusted manually in Coot, followed by cycles of real-space refinement in PHENIX and additional manual corrections in Coot.

Interface interactions determination. Interface contacts, buried surface area, and estimated binding energy were calculated with PISA (CCP4 suite). Interaction counts are reported as unique contacts from all symmetry-related interactions.

Formation of F_{ECTO}-Fab Complexes for Determining Hydrodynamic Radius and Differential Scanning Fluorimetry. F_{ECTO} (F_{ECTO} E170G, E455G, residues 1-495) was mixed to a final concentration of ~1.7 μM (trimer) and incubated with Fab at room temperature for 15-30 min. Fab was added at a threefold molar excess for 77.1, H8, and C6, and at a onefold molar excess for Y10F (ratios calculated relative to the F_{ECTO} trimer). The resulting mixtures were used directly for downstream analyses.

nanoDSF and DLS measurements. Thermal unfolding of F_{ECTO} with or without Fab, and of Fab alone, was measured on a NanoTemper Prometheus Panta. F_{ECTO}-Fab complexes were assembled 30 min before acquisition and incubated at room temperature. For each experiment, three capillaries contained the F_{ECTO}-Fab complex, two capillaries contained the corresponding Fab alone, and one capillary contained HBS as a buffer control. Data were collected with a 1 °C/min temperature ramp and 100% excitation laser power. Unfolding profiles are reported as the first derivative of the intrinsic fluorescence at 330 nm. Dynamic light scattering was recorded in parallel, and the cumulant hydrodynamic radius was calculated and plotted as a function of temperature.

Next Generation Sequencing and variant calling: Viral whole-genome sequencing of MeV samples was performed using a metagenomic next-generation sequencing approach and a previously described protocol. Libraries were sequenced on NextSeq 2000 with 2x150 bp or 1x100 bp read format. Raw reads were trimmed and quality filtered with fastp (v0.23.4) with the parameters --cut_mean_quality 20 --cut_front --cut_tail --length_required 20 --low_complexity_filter --trim_poly_g --trim_poly_x. For variant calling, filtered reads were processed with the RAVA workflow (default parameters) using the MeV plasmid map as a reference (github.com/greninger-lab/RAVA_Pipeline/tree/2025-08-18_DZ_MP), (doi.org/10.1101/2019.12.17.879320). Raw sequencing data are publicly available in NCBI BioProject PRJNA1307578.

Statistics & Reproducibility. Data analysis and visualization were performed in Python using NumPy, pandas, SciPy, Matplotlib, seaborn, and statannotations. Unless stated otherwise, group comparisons used two-sided Mann-Whitney-Wilcoxon tests. When multiple pairwise comparisons were made within a figure panel, p-values were adjusted with the Holm-Bonferroni method to control the family-wise error rate. Exact p-values and test statistics (U) are reported in the figure legends. P-value annotations on plots follow this legend: ns, 5.00×10⁻² <p≤1.00×10⁰; *, 1.00×10⁻²<p≤5.00×10⁻²; **, 1.00×10⁻³<p≤1.00×10⁻²; ***, 1.00×10⁻⁴<p≤1.00×10⁻³; ****,p≤1.00×10⁻⁴.

In animal experiments, individual cotton rats represented distinct biological replicates (per group). This sample size was selected based on historical data from this infection model to achieve adequate statistical power for a non-parametric analysis (Mann-Whitney U test) without exceeding the necessary number of animals.

Chemicals and peptides. N-(3-cyanophenyl)-2-phenylacetamide (also known as 3G) was commercially acquired from ZereneX Molecular Limited (UK). The purity of 3G was tested by high-pressure liquid chromatography (HPLC) and shown to be >95% pure.

Peptides were purchased from CPC, Ltd. Bromoacetyl cholesterol and bismaleimide cholesterol derivatives were custom-made by Charnwood Molecular Ltd. Dimethylsulfoxide (DMSO), tetrahydrofuran (THF), N,N-diisopropylethylamine (DIPEA) were purchased from Sigma Aldrich, bismaleimide-PEG₁₁ from Quanta BioDesign. HPLC purification was performed on a 1100 Series Agilent HPLC system equipped with a UV diode array detector and a fraction collector using a reverse phase (RP.) Phenomenex Jupiter C4 LC column 300 Å (150×21.2 mm, particle size 5 μm). MALDI-TOF analysis was performed on a Bruker UltrafleXtreme MALDI-TOF instrument.

### Data Availability

Cryo-electron microscopy density maps and atomic coordinates have been deposited in public repositories. The Y10F-F ectodomain complex is available in the PDB (9Q5M) and EMDB (EMD-72240); the 77.1-Y10F-F ectodomain complex is available in the PDB (9Q5V) and EMDB (EMD-72242); the H8-F ectodomain complex in the prefusion state is available in the PDB (9Q7B) and EMDB (EMD-72293); and the H8-F ectodomain complex in the postfusion state is available in the PDB (9Q71) and EMDB (EMD-72283).

### REFERENCES

Acciani, M., et al. Uncovering the features of Measles-targeting human antibodies elicited by the MMR vaccine. bioRxiv (2025).
Adams, P. D. et al. PHENIX: a comprehensive Python-based system for macromolecular structure solution. Acta Crystallogr. D Biol. Crystallogr. 66, 213-221 (2010).
Ader, N. et al. Mechanism for active membrane fusion triggering by morbillivirus attachment protein. J. Virol. 87, 314-326 (2013).
Agirre, J. et al. The CCP4 suite: integrative software for macromolecular crystallography. Acta Crystallogr D Struct Biol 79, 449-461 (2023).
Alamares-Sapuay, J., et al. Mutations in the F protein of the live-attenuated respiratory syncytial virus vaccine candidate DeltaNS2/Delta1313/11314L increase the stability of infectivity and content of prefusion F protein. PLoS One 19, e0301773 (2024).
Anderer, S. Global Measles Cases Rose 20% in 1 Year as Vaccine Coverage Fell Short. JAMA 333, 279 (2025).
Anderer, S. Measles Cases in European Region Highest in More Than 25 Years. JAMA 333, 1658 (2025).
Anderer, S. Measles Resurgence Puts People With HIV at Risk. JAMA (2025).
Anderer, S. US Measles Cases Hit 25-Year High. JAMA 334, 661 (2025).
Angius, F., et al. Analysis of a Subacute Sclerosing Panencephalitis Genotype B3 Virus from the 2009-2010 South African Measles Epidemic Shows That Hyperfusogenic F Proteins Contribute to Measles Virus Infection in the Brain. J Virol 93, e01700-01718 (2019).
Anichini, G., et al. Seroprevalence to Measles Virus after Vaccination or Natural Infection in an Adult Population, in Italy. Vaccines (Basel) 8 (2020).
Apte-Sengupta, S., et al. Base of the measles virus fusion trimer head receives the signal that triggers membrane fusion. J Biol Chem 287, 33026-33035 (2012).
Audet, S., et al. Measles-virus-neutralizing antibodies in intravenous immunoglobulins. J Infect Dis 194, 781-789 (2006).
August, A. et al. A phase 1 trial of lipid-encapsulated mRNA encoding a monoclonal antibody with neutralizing activity against Chikungunya virus. Nat. Med. 27, 2224-2233 (2021).
Avanzato, V. A. et al. A structural basis for antibody-mediated neutralization of Nipah virus reveals a site of vulnerability at the fusion glycoprotein apex. Proc. Natl. Acad. Sci. U.S.A. 116, 25057-25067 (2019).
Avila, M.; et al., Molecular determinants defining the triggering range of prefusion F complexes of canine distemper virus. J Virol 2014, 88 (5), 2951-66.PMC3958098
Baker, J., et al. Equivalent immunogenicity across three RSVpreF vaccine lots in healthy adults 18-49 years of age: Results of a randomized phase 3 study. Vaccine 42, 3172-3179 (2024).
Banerjee, A.; Huang, J.; Rush, S. A.; Murray, J.; Gingerich, A. D.; Royer, F.; Hsieh, C. L.; Tripp, R. A.; Mclellan, J. S.; Mousa, J. J., Structural basis for ultrapotent antibody-mediated neutralization of human metapneumovirus. Proc Natl Acad Sci USA 2022, 119 (25), e2203326119.PMC9231621
Benton, D. J., et al. Influenza hemagglutinin membrane anchor. Proc Natl Acad Sci USA 115, 10112-10117 (2018).
Bepler, T. et al. Positive-unlabeled convolutional neural networks for particle picking in cryo-electron micrographs. Nat. Methods 16, 1153-1160 (2019).
Bertoni, M., Kiefer, F., Biasini, M., Bordoli, L. & Schwede, T. Modeling protein quaternary structure of homo- and hetero-oligomers beyond binary interactions by homology. Sci. Rep. 7, 10480 (2017).
Bhattacharyya, S. & Ferrari, M. J. Age-specific mixing generates transient outbreak risk following critical-level vaccination. Epidemiol. Infect. 145, 12-22 (2017).
Bienert, S. et al. The SWISS-MODEL Repository-new features and functionality. Nucleic Acids Res. 45, D313-D319 (2016).
Bird, G. H., et al. A stapled lipopeptide platform for preventing and treating highly pathogenic viruses of pandemic potential. Nat Commun 15, 274 (2024).
Bitnun, A. et al. Measles inclusion-body encephalitis caused by the vaccine strain of measles virus. Clin. Infect. Dis. 29, 855-861 (1999).
Bochennek, K. et al. Differential loss of humoral immunity against measles, mumps, rubella and varicella-zoster virus in children treated for cancer. Vaccine 32, 3357-3361 (2014).
Bolotin, S., et al. In Elimination Settings, Measles Antibodies Wane After Vaccination but Not After Infection: A Systematic Review and Meta-Analysis. J Infect Dis 226, 1127-1139 (2022).
Bootz, A.; et al., Protective capacity of neutralizing and non-neutralizing antibodies against glycoprotein 1 B of cytomegalovirus. PLoS Pathog 2017, 13 (8), e1006601.PMC5595347
Boukhvalova, M. S.; Prince, G. A.; Soroush, L.; Harrigan, D. C.; Vogel, S. N.; Blanco, J. C., The TLR4 agonist, monophosphoryl lipid A, attenuates the cytokine storm associated with respiratory syncytial virus vaccine-enhanced disease. Vaccine 2006, 24 (23), 5027-35
Bovier, F. T. et al. Inhibition of Measles Viral Fusion Is Enhanced by Targeting Multiple Domains of the Fusion Protein. ACS Nano 15, 12794-12803 (2021).
Brindley, S. Chaudhury, R. K. Plemper, Measles virus glycoprotein complexes preassemble intracellularly and relax during transport to the cell surface in preparation for fusion. J. Virol. 89, 1230-1241 (2015).
Byrne, P. O., et al. Structural basis for antibody recognition of vulnerable epitopes on Nipah virus F protein. Nat Commun 14, 1494 (2023).
Cabán, et al., Cross-protective antibodies against common endemic respiratory viruses. Nat. Commun. 14, 798 (2023).
Carsillo, M.; Kutala, V. K.; Puschel, K.; Blanco, J.; Kuppusamy, P.; Niewiesk, S., Nitric oxide production and nitric oxide synthase type 2 expression by cotton rat (Sigmodon hispidus) macrophages reflect the same pattern as human macrophages. Dev. Comp. Immunol. 2009, 33, 718-724
Casalino, L., et al. Beyond Shielding: The Roles of Glycans in the SARS-CoV-2 Spike Protein. ACS Cent Sci 6, 1722-1734 (2020).
Centers for Disease Control and Prevention, “Genetic analysis of measles virus” (2022); www.cdc.gov/measles/php/laboratories/genetic-analysis-of-measles-viruses.html.
Centers for Disease Control and Prevention, “Nearly 40 million children are dangerously susceptible to growing measles threat,” Press Release, (2022); www.cdc.gov/media/releases/2022/p1123-measles-threat.html.
Chang, A. & Dutch, R. E. Paramyxovirus fusion and entry: multiple paths to a common end. Viruses 4, 613-636 (2012).
Chapman, B. & Chang, J. Biopython: Python tools for computational biology. SIGBIO Newsl. 20, 15-19 (2000).
Charlier, F. et al. trevismd/statannotations: v0.5. (2022). doi:10.5281/zenodo.7213391.
Che, Y., et al. Rational design of a highly immunogenic prefusion-stabilized F glycoprotein antigen for a respiratory syncytial virus vaccine. Sci Transl Med 15, eade6422 (2023).
Chen, C. J.; Lee, P. I.; Hsieh, Y. C.; Chen, P. Y.; Ho, Y. H.; Chang, C. J.; Liu, D. P.; Chang, F. Y.; Chiu, C. H.; Huang, Y. C.; Lee, C. Y.; Lin, T. Y., Waning population immunity to measles in Taiwan. Vaccine 2012, 30 (47), 6721-7
Chen, L. et al. The structure of the fusion glycoprotein of Newcastle disease virus suggests a novel paradigm for the molecular mechanism of membrane fusion. Structure 9, 255-266 (2001).
Chiu, M. H.; Meatherall, B.; Nikolic, A.; Cannon, K.; Fonseca, K.; Joseph, J. T.; MacDonald, J.; Pabbaraju, K.; Tellier, R.; Wong, S.; Koch, M. W., Subacute sclerosing panencephalitis in pregnancy. The Lancet. Infectious diseases 2016, 16 (3), 366-75
Cleveland, N. K. et al. Many Inflammatory Bowel Disease Patients Are Not Immune to Measles or Pertussis. Dig. Dis. Sci. 61, 2972-2976 (2016).
Cock, P. J. A. et al. Biopython: freely available Python tools for computational molecular biology and bioinformatics. Bioinformatics 25, 1422-1423 (2009).
Corti, D., Purcell, L. A., Snell, G. & Veesler, D. Tackling COVID-19 with neutralizing monoclonal antibodies. Cell 184, 3086-3108 (2021).
Crank, M. C., et al. A proof of concept for structure-based vaccine design targeting RSV in humans. Science 365, 505-509 (2019).
Croll, ISOLDE: A physically realistic environment for model building into low-resolution electron-density maps. Acta Crystallogr. D Struct. Biol. 74, 519-530 (2018).
Dabbagh, A. et al. Progress Toward Regional Measles Elimination-Worldwide, 2000-2016. MMWR Morb. Mortal. Wkly. Rep. 66, 1148-1153 (2017).
Dang, H. V. et al. An antibody against the F glycoprotein inhibits Nipah and Hendra virus infections. Nat. Struct. Mol. Biol. 26, 980-987 (2019).
Dang, H. V. et al. Broadly neutralizing antibody cocktails targeting Nipah virus and Hendra virus fusion glycoproteins. Nat. Struct. Mol. Biol. 28, 426-434 (2021).
De Serres, G. et al. Higher risk of measles when the first dose of a 2-dose schedule of measles vaccine is given at 12-14 months versus 15 months of age. Clin. Infect. Dis. 55, 394-402 (2012).
de Swart, R. L., Measles studies in the macaque model. Curr Top Microbiol Immunol 2009, 330, 55-72
De Swart, R. L., Measles: what Applicants have learned from non-human primate models. Drug Discov Today Dis Mod 2017, 23 (Special Issue: Use of non-human primate disease models), 31-34
de Swart, R. L., Yüksel, S. & Osterhaus, A. D. M. E. Relative contributions of measles virus hemagglutinin- and fusion protein-specific serum antibodies to virus neutralization. J. Virol. 79, 11547-11551 (2005).
de Swart, R. L., Yüksel, S., Langerijs, C. N., Muller, C. P. & Osterhaus, A. D. M. E. Depletion of measles virus glycoprotein-specific antibodies from human sera reveals genotype-specific neutralizing antibodies. J. Gen. Virol. 90, 2982-2989 (2009).
de Swart, R. L.; Ludlow, M.; de Witte, L.; Yanagi, Y.; van Amerongen, G.; McQuaid, S.; Yuksel, S.; Geijtenbeek, T. B.; Duprex, W. P.; Osterhaus, A. D., Predominant infection of CD150+ lymphocytes and dendritic cells during measles virus infection of macaques. PLoS Pathog 2007, 3 (11), e178.PMC2077902
de Vries, R. D.; de Swart, R. L., Measles immune suppression: functional impairment or numbers game? PLoS Pathog 2014, 10 (12), e1004482.PMC4270776
de Vries, R. D.; McQuaid, S.; van Amerongen, G.; Yuksel, S.; Verburgh, R. J.; Osterhaus, A. D.; Duprex, W. P.; de Swart, R. L., Measles immune suppression: lessons from the macaque model. PLoS Pathog 2012, 8(8), e1002885.3431343
de Vries, R. D.; Mesman, A. W.; Geijtenbeek, T. B.; Duprex, W. P.; de Swart, R. L., The pathogenesis of measles. Current opinion in virology 2012, 2 (3), 248-55
de Vries, R. D., Yuksel, S., Osterhaus, A. D. & de Swart, R. L. Specific CD8(+) T-lymphocytes control dissemination of measles virus. Eur.J.Immunol. 40, 388-395 (2010).
Denisov, I. G., Grinkova, Y. V., Lazarides, A. A. & Sligar, S. G. Directed self-assembly of monodisperse phospholipid bilayer Nanodiscs with controlled size. J Am Chem Soc 126, 3477-3487 (2004).
Di Tommaso, P., et al. Nextflow enables reproducible computational workflows. Nat Biotechnol 35, 316-319 (2017).
Do, L. A. H. & Mulholland, K. Measles 2025. N Engl J Med (2025).
Du, R.; An, C.; Yao, X.; Wang, Y.; Wang, G.; Gao, F.; Bian, L.; Hu, Y.; Liu, S.; Zhao, Q.; Mao, Q.; Liang, Z., Non-neutralizing monoclonal antibody targeting VP2 EF loop of Coxsackievirus A16 can protect mice from lethal attack via Fc-dependent effector mechanism. Emerg Microbes Infect 2023, 12 (1), 2149352.PMC9788719
Duncan, C. J. A. et al. Human IFNAR2 deficiency: Lessons for antiviral immunity. Sci. Transl. Med. 7, 307ra154 (2015).
Duprex, W. P. & Dutch, R. E. Paramyxoviruses: Pathogenesis, Vaccines, Antivirals, and Prototypes for Pandemic Preparedness. J Infect Dis 228, S390-S397 (2023).
Durie, I. A.; et al., Structural characterization of protective non-neutralizing antibodies targeting Crimean-Congo hemorrhagic fever virus. Nat Commun 2022, 13 (1), 7298.PMC9701186
Durrheim, D. N. et al. A dangerous measles future looms beyond the COVID-19 pandemic. Nat. Med. 27, 360-361 (2021).
Earnest, J. T.; Holmes, A. C.; Basore, K.; Mack, M.; Fremont, D. H.; Diamond, M. S., The mechanistic basis of protection by non-neutralizing anti-alphavirus antibodies. Cell Rep 2021, 35 (1), 108962.PMC8055377
Emsley, P. & Cowtan, K. Coot: model-building tools for molecular graphics. Acta Crystallogr D Biol Crystallogr 60, 2126-2132 (2004).
Emsley, P., Lohkamp, B., Scott, W. G. & Cowtan, K. Features and development of Coot. Acta Crystallogr. D Biol. Crystallogr. 66, 486-501 (2010).
Failure to vaccinate and vaccine failure. Nat Microbiol 4, 725 (2019).
Feltes, T. F. et al. Palivizumab prophylaxis reduces hospitalization due to respiratory syncytial virus in young children with hemodynamically significant congenital heart disease. J. Pediatr. 143, 532-540 (2003).
Feng, Y.; et al., Broadly neutralizing antibodies against sarbecoviruses generated by immunization of macaques with an AS03-adjuvanted COVID-19 vaccine. Sci Transl Med 2023, 15 (695), eadg7404
Ferrari, M. J. & Moss, W. J. Rubella and measles: The beginning of the endgame. Science 388, 32-34 (2025).
Ferreira, C. S.; Frenzke, M.; Leonard, V. H.; Welstead, G. G.; Richardson, C. D.; Cattaneo, R., Measles virus infection of alveolar macrophages and dendritic cells precedes spread to lymphatic organs in transgenic mice expressing human signaling lymphocytic activation molecule (SLAM, CD150). Journal of virology 2010, 84 (6), 3033-42.2826031
Ferren, B. Horvat, C. Mathieu, Measles encephalitis: Towards new therapeutics. Viruses 11, 1017 (2019).
Fiebelkorn, A. P. et al. A comparison of postelimination measles epidemiology in the United States, 2009-2014 versus 2001-2008. J. Pediatric Infect. Dis. Soc. 6, 40-48 (2017).
Figueira, T. N. et al. In Vivo Efficacy of Measles Virus Fusion Protein-Derived Peptides Is Modulated by the Properties of Self-Assembly and Membrane Residence. J. Virol. 91, (2017).
Figueira, T. N.; Mendonca, D. A.; Gaspar, D.; Melo, M. N.; Moscona, A.; Porotto, M.; Castanho, M.; Veiga, A. S., Structure-Stability-Function Mechanistic Links in the Anti-Measles Virus Action of Tocopherol-Derivatized Peptide Nanoparticles. ACS Nano 2018, 12 (10), 9855-9865.PMC6399014
Fill, M.-M. A. et al. Notes from the Field: Measles Outbreak of Unknown Source-Shelby County, Tennessee, April-May 2016. MMWR Morb. Mortal. Wkly. Rep. 65, 1039-1040 (2016).
Forsberg, B., alisterburt & DanGonite. bforsbe/Occupy: v0.1.13. (2023). doi:10.5281/zenodo.8232297.
Forsberg, P. N. M. Shah, A. Burt, A robust normalized local filter to estimate compositional heterogeneity directly from cryo-EM maps. Nat. Commun. 14, 5802 (2023).
Fowlkes, A. L. et al. Supplemental measles vaccine antibody response among HIV-infected and -uninfected children in Malawi after 1- and 2-dose primary measles vaccination schedules. Vaccine 34, 1459-1464 (2016).
Fox, A. et al. Acute measles encephalitis in partially vaccinated adults. PLoS One 8, e71671 (2013).
Frey, G. et al. A fusion-intermediate state of HIV-1 gp41 targeted by broadly neutralizing antibodies. Proc. Natl. Acad. Sci. U.S.A 105, 3739-3744 (2008).
Fulginiti, V. A. & Kempe, C. H. Measles Exposure among Vaccine Recipients. Response to Measles Exposure and Antibody Persistence among Recipients of Measles Vaccines. Am J Dis Child 106, 450-461 (1963).
Gallone, M. S., Germinario, C., Larocca, A. & Tafuri, S. Long time immunogenicity of measles vaccine in the vaccination era: An open question. Hum. Vaccin. Immunother. 13, 117-119 (2017).
Garg, R. K.; Mahadevan, A.; Malhotra, H. S.; Rizvi, I.; Kumar, N.; Uniyal, R., Subacute sclerosing panencephalitis. Rev Med Virol 2019, e2058
Gastañaduy, et al., Measles-United States, January 1-May 23, 2014. MMWR Morb. Mortal. Wkly. Rep. 63, 496-499 (2014).
Gastañaduy, P. A. et al. Measles-United States, January 1-May 23, 2014. Am. J. Transplant 14, 1937-1940 (2014).
Genetic analysis of measles virus. www.cdc.gov/measles/lab-tools/genetic-analysis.html (2022).
Gidding, H. F., Quinn, H. E., Hueston, L., Dwyer, D. E. & McIntyre, P. B. Declining measles antibodies in the era of elimination: Australia's experience. Vaccine 36, 507-513 (2018).
Gidwani, S. V., et al. Engineered dityrosine-bonding of the RSV prefusion F protein imparts stability and potency advantages. Nat Commun 15, 2202 (2024).
Gilman, M. S. A., et al. Transient opening of trimeric prefusion RSV F proteins. Nat Commun 10, 2105 (2019).
Glasser, J. W., Feng, Z., Omer, S. B., Smith, P. J. & Rodewald, L. E. The effect of heterogeneity in uptake of the measles, mumps, and rubella vaccine on the potential for outbreaks of measles: a modelling study. Lancet Infect. Dis. 16, 599-605 (2016).
Goddard, T. D. et al. UCSF ChimeraX: Meeting modern challenges in visualization and analysis. Protein Sci. 27, 14-25 (2018).
Gomes, B.; Santos, N. C.; Porotto, M., Biophysical Properties and Antiviral Activities of Measles Fusion Protein Derived Peptide Conjugated with 25-Hydroxycholesterol. Molecules 2017, 22 (11).PMC5775476
Gonzalez, K. J., et al. A general computational design strategy for stabilizing viral class I fusion proteins. Nat Commun 15, 1335 (2024).
Gonzalez, K. J., Yim, K., Blanco, J. C., Boukhvalova, M. S. & Strauch, E. M. Systematic computer-aided disulfide design as a general strategy to stabilize prefusion class I fusion proteins. bioRxiv (2024).
Graham, B. S., Gilman, M. S. A. & Mclellan, J. S. Structure-Based Vaccine Antigen Design. Annu Rev Med 70, 91-104 (2019).
Greninger, A. L.; Rybkina, K.; Lin, M. J.; Drew-Bear, J.; Marcink, T. C.; Shean, R. C.; Makhsous, N.; Boeckh, M.; Harder, O.; Bovier, F.; Burstein, S. R.; Niewiesk, S.; Rima, B. K.; Porotto, M.; Moscona, A., Human parainfluenza virus evolution during lung infection of immunocompromised individuals promotes viral persistence. The Journal of clinical investigation 2021, 131 (23).PMC8631596
Greninger, A. L., et al. Rapid Metagenomic Next-Generation Sequencing during an Investigation of Hospital-Acquired Human Parainfluenza Virus 3 Infections. J Clin Microbiol 55, 177-182 (2017).
Griffin, D. E. Measles immunity and immunosuppression. Curr. Opin. Virol. 46, 9-14 (2021).
Griffin, D. E. Measles virus persistence and its consequences. Curr. Opin. Virol. 41, 46-51 (2020).
Griffin, D. E., Immune responses during measles virus infection. In Measles virus, Billeter, M.; ter Meulen, V., Eds. Springer: Berlin, 1995; Vol. 191, pp 117-134.
Griffin, D. E.; Lin, W. H.; Pan, C. H., Measles virus, immune control, and persistence. FEMS Microbiol Rev 2012, 36 (3), 649-62.3319515
Griffin, D. E.; Lin, W. W.; Nelson, A. N., Understanding the causes and consequences of measles virus persistence. F1000Res 2018, 7, 237.5832918
Groothuis, J. R. et al. Prophylactic administration of respiratory syncytial virus immune globulin to high-risk infants and young children. The Respiratory Syncytial Virus Immune Globulin Study Group. N. Engl. J. Med. 329, 1524-1530 (1993).
Guex, N., Peitsch, M. C. & Schwede, T. Automated comparative protein structure modeling with SWISS-MODEL and Swiss-PdbViewer: a historical perspective. Electrophoresis 30 Suppl 1, S162-73 (2009).
Gupta, A. et al. Early Treatment for Covid-19 with SARS-CoV-2 Neutralizing Antibody Sotrovimab. N. Engl. J. Med. 385, 1941-1950 (2021).
Guthmiller, J. J.; Han, J.; Utset, H. A.; Li, L.; Lan, L. Y.; Henry, C.; Stamper, C. T.; McMahon, M.; O'Dell, G.; Fernandez-Quintero, M. L.; Freyn, A. W.; Amanat, F.; Stovicek, O.; Gentles, L.; Richey, S. T.; de la Pena, A. T.; Rosado, V.; Dugan, H. L.; Zheng, N. Y.; Tepora, M. E.; Bitar, D. J.; Changrob, S.; Strohmeier, S.; Huang, M.; Garcia-Sastre, A.; Liedl, K. R.; Bloom, J. D.; Nachbagauer, R.; Palese, P.; Krammer, F.; Coughlan, L.; Ward, A. B.; Wilson, P. C., Broadly neutralizing antibodies target a haemagglutinin anchor epitope. Nature 2022, 602 (7896), 314-320.PMC8828479
Ha, M. N. et al. Mutations in the Fusion Protein of Measles Virus That Confer Resistance to the Membrane Fusion Inhibitors Carbobenzoxy-d-Phe-1-Phe-Gly and 4-Nitro-2-Phenylacetyl Amino-Benzamide. J. Virol. 91, (2017).
Hambleton, S. et al. STAT2 deficiency and susceptibility to viral illness in humans. Proc. Natl. Acad. Sci. U.S.A 110, 3053-3058 (2013).
Hamid, S., et al. Seasonality of Respiratory Syncytial Virus-United States, 2017-2023. MMWR Morb Mortal Wkly Rep 72, 355-361 (2023).
Hammitt, L. L. et al. Nirsevimab for Prevention of RSV in Healthy Late-Preterm and Term Infants. N. Engl. J. Med. 386, 837-846 (2022).
Hanft, E.; et al., Congenital Measles in a Premature 25-week Gestation Infant. Pediatr Infect Dis J 2021, 40 (8), 753-755
Haralambieva, I. H. et al. A large observational study to concurrently assess persistence of measles specific B-cell and T-cell immunity in individuals following two doses of MMR vaccine. Vaccine 29, 4485-4491 (2011).
Haralambieva, I. H., Kennedy, R. B., Ovsyannikova, I. G., Whitaker, J. A. & Poland, G. A. Variability in Humoral Immunity to Measles Vaccine: New Developments. Trends Mol. Med. 21, 789-801 (2015).
Hardie, D. R.; Albertyn, C.; Heckmann, J. M.; Smuts, H. E., Molecular characterisation of virus in the brains of patients with measles inclusion body encephalitis (MIBE). Virol J 2013, 10, 283.3847183
Harris, A. K.; Meyerson, J. R.; Matsuoka, Y.; Kuybeda, O.; Moran, A.; Bliss, D.; Das, S. R.; Yewdell, J. W.; Sapiro, G.; Subbarao, K.; Subramaniam, S., Structure and accessibility of HA trimers on intact 2009 H1N1 pandemic influenza virus to stem region-specific neutralizing antibodies. Proc Natl Acad Sci USA 2013, 110 (12), 4592-7.PMC3607006
Harris, E. Measles Outbreaks Grow Amid Declining Vaccination Rates. JAMA (2023).
Harrison, S. C. Viral membrane fusion. Nat. Struct. Mol. Biol. 15, 690-698 (2008).
Hashiguchi, T. et al. Structures of the prefusion form of measles virus fusion protein in complex with inhibitors. Proc. Natl. Acad. Sci. U.S.A 115, 2496-2501 (2018).
Hashiguchi, T., Maenaka, K. & Yanagi, Y. Measles virus hemagglutinin: structural insights into cell entry and measles vaccine. Front. Microbiol. 2, 247 (2011).
Havers, F. P., et al. Characteristics and Outcomes Among Adults Aged >/=60 Years Hospitalized with Laboratory-Confirmed Respiratory Syncytial Virus-RSV-NET, 12 States, July 2022-June 2023. MMWR Morb Mortal Wkly Rep 72, 1075-1082 (2023).
Henry Dunand, C. J.; et al., Both Neutralizing and Non-Neutralizing Human H7N9 Influenza Vaccine-Induced Monoclonal Antibodies Confer Protection. Cell Host Microbe 2016, 19 (6), 800-13.PMC4901526
Hsieh, C. L., et al. Structure-based design of prefusion-stabilized human metapneumovirus fusion proteins. Nat Commun 13, 1299 (2022).
Hughes, C. S., et al. Single-pot, solid-phase-enhanced sample preparation for proteomics experiments. Nat Protoc 14, 68-85 (2019).
Hunter, Matplotlib: A 2D Graphics Environment. Comput. Sci. Eng. 9, 90-95 (2007).
Iketani, S. et al. Viral Entry Properties Required for Fitness in Humans Are Lost through Rapid Genomic Change during Viral Isolation. MBio 9, (2018).
Iwamoto, M., et al. Measles infection in persons with secondary vaccine failure, New York City, 2018-19. Vaccine 39, 5346-5350 (2021).
Jang, B., et al. Measles Virus Neutralizing Antibody Response and Durability Two Years after One or Two Doses of Measles-Mumps-Rubella Vaccine among Young Seronegative Healthcare Workers. Vaccines (Basel) 10(2022).
Johnson, R. A.; Prince, G. A.; Suffin, S. C.; Horswood, R. L.; Chanock, R. M., Respiratory syncytial virus infection in cyclophosphamide-treated cotton rats. Infect Immun 1982, 37 (1), 369-73.347535
Jones, H. G., et al. Alternative conformations of a major antigenic site on RSV F. PLoS Pathog 15, e1007944 (2019).
Joyce, M. G., et al. Crystal Structure and Immunogenicity of the DS-Cav1-Stabilized Fusion Glycoprotein From Respiratory Syncytial Virus Subtype B. Pathog Immun 4, 294-323 (2019).
Jumper, et al., Highly accurate protein structure prediction with AlphaFold. Nature 596, 583-589 (2021).
Jurgens, E. M. et al. Measles fusion machinery is dysregulated in neuropathogenic variants. MBio 6, (2015).
Kampmann, B., et al. Bivalent Prefusion F Vaccine in Pregnancy to Prevent RSV Illness in Infants. N Engl J Med 388, 1451-1464 (2023).
Kaplon, H., Chenoweth, A., Crescioli, S. & Reichert, J. M. Antibodies to watch in 2022. MAbs 14, 2014296 (2022).
Katz, S. L. & Hinman, A. R. Summary and conclusions: measles elimination meeting, 16-17 Mar. 2000. J. Infect. Dis. 189 Suppl 1, S43-7 (2004).
Kearse, M. et al. Geneious Basic: an integrated and extendable desktop software platform for the organization and analysis of sequence data. Bioinformatics 28, 1647-1649 (2012).
Kertes, J. et al. Association between AZD7442 (tixagevimab-cilgavimab) administration and severe acute respiratory syndrome Coronavirus 2 (SARS-CoV-2) infection, hospitalization, and mortality. Clin. Infect. Dis. 76, e126-e132 (2023).
Kim, D.; Niewiesk, S., Synergistic Induction of Interferon alpha through TLR-3 and TLR-9 Agonists Identifies CD21 as Interferon alpha Receptor for the B Cell Response. PLoS Pathog 2013, 9 (3), e1003233.3597509
Kim, et al., Capture and imaging of a prehairpin fusion intermediate of the paramyxovirus PIV5. Proc. Natl. Acad. Sci. U.S.A. 108, 20992-20997 (2011).
Kim, S.; Filsinger Interrante, M. V.; Kim, P. S., Enhancing HIV-1 Neutralization by Increasing the Local Concentration of Membrane-Proximal External Region-Directed Broadly Neutralizing Antibodies. J Virol 2023, 97 (1), e0164722.PMC9888200
Kimanius, D., Dong, L., Sharov, G., Nakane, T. & Scheres, S. H. W. New tools for automated cryo-EM single-particle analysis in RELION-4.0. Biochem. J 478, 4169-4185 (2021).
Kontio, M., Jokinen, S., Paunio, M., Peltola, H. & Davidkin, I. Waning antibody levels and avidity: implications for MMR vaccine-induced protection. J. Infect. Dis. 206, 1542-1548 (2012).
Krebs, S. J., et al., Longitudinal Analysis Reveals Early Development of Three MPER-Directed Neutralizing Antibody Lineages from an HIV-1-Infected Individual. Immunity 2019, 50 (3), 677-691 e13.PMC6555550
Krissinel, E. & Henrick, K. Detection of Protein Assemblies in Crystals. in Computational Life Sciences 163-174 (Springer Berlin Heidelberg, 2005).
Kurata, T., et al. A measles outbreak from an index case with immunologically confirmed secondary vaccine failure. Vaccine 38, 1467-1475 (2020).
Lackner, C., Karbiener, M., Faltner, L., Farcet, M. R. & Kreil, T. R. Feasibility of identifying plasma donors with high measles neutralizing antibody concentrations for the use of producing a measles hyperimmune globulin for postexposure prophylaxis. Immunol Res 70, 365-370 (2022).
Laksono, B. M.; de Vries, R. D.; McQuaid, S.; Duprex, W. P.; de Swart, R. L., Measles Virus Host Invasion and Pathogenesis. Viruses 2016, 8 (8), e210.PMC4997572
Laksono, B. M.; de Vries, R. D.; Verburgh, R. J.; Visser, E. G.; de Jong, A.; Fraaij, P. L. A.; Ruijs, W. L. M.; Nieuwenhuijse, D. F.; van den Ham, H. J.; Koopmans, M. P. G.; van Zelm, M. C.; Osterhaus, A.; de Swart, R. L., Studies into the mechanism of measles-associated immune suppression during a measles outbreak in the Netherlands. Nat Commun 2018, 9 (1), 4944.6251901
Lamb, R. A., Paterson, R. G. & Jardetzky, T. S. Paramyxovirus membrane fusion: lessons from the F and HN atomic structures. Virology 344, 30-37 (2006).
Langedijk, J. P. M., et al. Universal paramyxovirus vaccine design by stabilizing regions involved in structural transformation of the fusion protein. Nat Commun 15, 4629 (2024).
Lemon, K., et al. Early target cells of measles virus after aerosol infection of non-human primates. PLoS Pathog 7, e1001263 (2011).
Levine, M. M. Monoclonal Antibody Therapy for Ebola Virus Disease. The New England journal of medicine vol. 381 2365-2366 (2019).
Liebschner, D., et al. Macromolecular structure determination using X-rays, neutrons and electrons: recent developments in Phenix. Acta Crystallogr D Struct Biol 75, 861-877 (2019).
Lin, L.; et al., Safety and Immunogenicity of an AS03 (B)-Adjuvanted Inactivated Tetravalent Dengue Virus Vaccine Administered on Varying Schedules to Healthy U.S. Adults: A Phase 1/2 Randomized Study. Am J Trop Med Hyg 2020, 103 (1), 132-141.PMC7356407
Lin, W. H.; Kouyos, R. D.; Adams, R. J.; Grenfell, B. T.; Griffin, D. E., Prolonged persistence of measles virus RNA is characteristic of primary infection dynamics. Proceedings of the National Academy of Sciences of the United States of American 2012, 109 (37), 14989-94.3443140
Loevinsohn, G., Rosman, L. & Moss, W. J. Measles Seroprevalence and Vaccine Responses in Human Immunodeficiency Virus-infected Adolescents and Adults: A Systematic Review. Clin. Infect. Dis. 69, 836-844 (2019).
Ludlow, M.; de Vries, R. D.; Lemon, K.; McQuaid, S.; Millar, E.; van Amerongen, G.; Yuksel, S.; Verburgh, R. J.; Osterhaus, A. D.; de Swart, R. L.; Duprex, W. P., Infection of lymphoid tissues in the macaque upper respiratory tract contributes to the emergence of transmissible measles virus. J.Gen.Virol. 2013, 94 (Pt 9), 1933-44
Maisner, A., et al. Recombinant measles virus requiring an exogenous protease for activation of infectivity. J.Gen. Virol. 81, 441-449 (2000).
Malvoisin, E. & Wild, F. Contribution of measles virus fusion protein in protective immunity: anti-F monoclonal antibodies neutralize virus infectivity and protect mice against challenge. J. Virol. 64, 5160-5162 (1990).
Marcink, T. C. et al. Subnanometer structure of an enveloped virus fusion complex on viral surface reveals new entry mechanisms. Sci Adv 9, eade2727 (2023).
Mathieu, C. et al. Measles Virus Bearing Measles Inclusion Body Encephalitis-Derived Fusion Protein Is Pathogenic after Infection via the Respiratory Route. J. Virol. 93, (2019).
Mathieu, C. et al. Molecular Features of the Measles Virus Viral Fusion Complex That Favor Infection and Spread in the Brain. MBio 12, e0079921 (2021).
Mathieu, C. et al. Prevention of measles virus infection by intranasal delivery of fusion inhibitor peptides. J. Virol. 89, 1143-1155 (2015).
Mathieu, C. et al. Single-chain variable fragment antibody constructs neutralize measles virus infection in vitro and in vivo. Cell. Mol. Immunol. 18, 1835-1837 (2021).
May, A. J., Pothula, K. R., Janowska, K. & Acharya, P. Structures of Langya Virus Fusion Protein Ectodomain in Pre- and Postfusion Conformation. J. Virol. 97, e0043323 (2023).
Mckinney, W. Pandas: A foundational python library for data analysis and statistics. www.dlr.de/sc/portaldata/15/resources/dokumente/pyhpc2011/submissions/pyhpc2011_subm ission_9.pdf (2011).
Mehtani, N. J., Rosman, L. & Moss, W. J. Immunogenicity and Safety of the Measles Vaccine in HIV-Infected Children: An Updated Systematic Review. Am. J. Epidemiol. 188, 2240-2251 (2019).
Mendoza, P. et al. Combination therapy with anti-HIV-1 antibodies maintains viral suppression. Nature 561, 479-484 (2018).
Meng, E. C., et al. UCSF ChimeraX: Tools for structure building and analysis. Protein Sci 32, e4792 (2023).
Mina, M. J. et al. Measles virus infection diminishes preexisting antibodies that offer protection from other pathogens. Science 366, 599-606 (2019).
Mirdita, M. et al. ColabFold: making protein folding accessible to all. Nat. Methods 19, 679-682 (2022).
Mire, C. E. et al. Human-monoclonal-antibody therapy protects nonhuman primates against advanced Lassa fever. Nat. Med. 23, 1146-1149 (2017).
Modrof, J., et al. Measles Virus Neutralizing Antibodies in Intravenous Immunoglobulins: Is an Increase by Revaccination of Plasma Donors Possible? J Infect Dis 216, 977-980 (2017).
Moscona, A.; Peluso, R. W., Relative affinity of the human parainfluenza virus type 3 hemagglutinin-neuraminidase for sialic acid correlates with virus-induced fusion activity. J Virol 1993, 67 (11), 6463-8.PMC238082
Moss, W. J. & Griffin, D. E. Measles. Lancet 379, 153-164 (2012).
Moss, W. J., Cutts, F. & Griffin, D. E. Implications of the human immunodeficiency virus epidemic for control and eradication of measles. Clin. Infect. Dis. 29, 106-112 (1999).
Mühlebach, M. D. et al. Adherens junction protein nectin-4 is the epithelial receptor for measles virus. Nature 480, 530-533 (2011).
Mukhamedova, M., et al. Vaccination with prefusion-stabilized respiratory syncytial virus fusion protein induces genetically and antigenically diverse antibody responses. Immunity 54, 769-780 e766 (2021).
Mulangu, S. et al. A randomized, controlled trial of Ebola virus disease therapeutics. N. Engl. J. Med. 381, 2293-2303 (2019).
Muñoz-Alía, M. A. et al. Measles Virus Hemagglutinin epitopes immunogenic in natural infection and vaccination are targeted by broad or genotype-specific neutralizing monoclonal antibodies. Virus Res. 236, 30-43 (2017).
Murrell, M.; Greengard, O.; Porotto, M.; Poltoratskaia, N.; Moscona, A., A single amino acid alteration in the human parainfluenza virus type 3 HN confers resistance to both binding inhibition and neuraminidase inhibition by the antiviral agent 4-GU-DANA (Zanamivir). Journal of Virology 2001, 75, 6310-6320
Navaratnarajah, C. K. et al. The measles virus hemagglutinin stalk: structures and functions of the central fusion activation and membrane-proximal segments. J. Virol. 88, 6158-6167 (2014).
Niesen, F. H., Berglund, H. & Vedadi, M. The use of differential scanning fluorimetry to detect ligand interactions that promote protein stability. Nat. Protoc. 2, 2212-2221 (2007).
Niewiesk, S. & Germann, P.-G. Development of neutralizing antibodies correlates with resolution of interstitial pneumonia after measles virus infection in cotton rats. J. Exp. Anim. Sci. 40, 201-210 (2000).
Niewiesk, S. Cotton rats (Sigmodon hispidus): an animal model to study the pathogenesis of measles virus infection. Immunol. Lett. 65, 47-50 (1999).
Niewiesk, S. Current animal models: cotton rat animal model. Curr Top Microbiol Immunol 330, 89-110 (2009).
Nilsson, A. & Chiodi, F. Measles outbreak in Africa—is there a link to the HIV-1 epidemic? PLoS Pathog. 7, e1001241 (2011).
Nomenclature for describing the genetic characteristics of wild-type measles viruses (update). Part I. Wkly. Epidemiol. Rec. 76, 242-247 (2001).
Noyce, R. S. et al. Tumor cell marker PVRL4 (nectin 4) is an epithelial cell receptor for measles virus. PLoS Pathog. 7, e1002240 (2011).
Noyce, R. S., Delpeut, S. & Richardson, C. D. Dog nectin-4 is an epithelial cell receptor for canine distemper virus that facilitates virus entry and syncytia formation. Virology 436, 210-220 (2013).
Nyathi, S. et al. The 2016 California policy to eliminate nonmedical vaccine exemptions and changes in vaccine coverage: An empirical policy analysis. PLoS Med. 16, e1002994 (2019).
Ono, N., et al. Measles viruses on throat swabs from measles patients use signaling lymphocytic activation molecule (CDw150) but not CD46 as a cellular receptor. J Virol 75, 4399-4401 (2001).
Orenstein, W. & Seib, K. Mounting a good offense against measles. N. Engl. J. Med. 371, 1661-1663 (2014).
Osman, S., et al. Population immunity to measles in Canada using Canadian Health Measures survey data-A Canadian Immunization Research Network (CIRN) study. Vaccine 40, 3228-3235 (2022).
Palermo, L. M.; Porotto, M.; Greengard, O.; Moscona, A., Fusion promotion by a paramyxovirus hemagglutinin-neuraminidase protein: pH modulation of receptor avidity of binding sites I and II. J Virol 2007, 81 (17), 9152-61.PMC1951465
Palermo, L.; Porotto, M.; Yokoyama, C.; Palmer, S.; Mungall, B.; Greengard, O.; Niewiesk, S.; Moscona, A., Human parainfluenza virus infection of the airway epithelium: the viral hemagglutinin-neuraminidase regulates fusion protein activation and modulates infectivity. J. Virol. 2009, 83 (13), 6900-6908
Palmer, S. G.; Porotto, M.; Palermo, L. M.; Cunha, L. F.; Greengard, O.; Moscona, A., Adaptation of human parainfluenza virus to airway epithelium reveals fusion properties required for growth in host tissue. mBio 2012, 3 (3), e00137-12
Pantaleo, G., Correia, B., Fenwick, C., Joo, V. S. & Perez, L. Antibodies to combat viral infections: development strategies and progress. Nat Rev Drug Discov 21, 676-696 (2022).
Papi, A., et al. Respiratory Syncytial Virus Prefusion F Protein Vaccine in Older Adults. N Engl J Med 388, 595-608 (2023).
Patel, M. K. & Orenstein, W. A. Classification of global measles cases in 2013-17 as due to policy or vaccination failure: a retrospective review of global surveillance data. The Lancet Global Health 7, e313-e320 (2019).
Patel, M. K., et al. Progress Toward Regional Measles Elimination-Worldwide, 2000-2018. MMWR Morb Mortal Wkly Rep 68, 1105-1111 (2019).
Paules, C. I., Marston, H. D. & Fauci, A. S. Measles in 2019-Going Backward. N. Engl. J. Med. 380, 2185-2187 (2019).
Pecenka, C., et al. Respiratory syncytial virus vaccination and immunoprophylaxis: realising the potential for protection of young children. Lancet 404, 1157-1170 (2024).
Pei, J. & Grishin, N. V. AL2CO: calculation of positional conservation in a protein sequence alignment. Bioinformatics 17, 700-712 (2001).
Peng, et al., Glycan shield of the ebolavirus envelope glycoprotein GP. Commun. Biol. 5, 785 (2022).
Pensieroso, S. et al. Timing of HAART defines the integrity of memory B cells and the longevity of humoral responses in HIV-1 vertically-infected children. Proc. Natl. Acad. Sci. U.S.A. 106, 7939-7944 (2009).
Petrova, V. N. et al. Incomplete genetic reconstitution of B cell pools contributes to prolonged immunosuppression after measles. Sci Immunol 4, eaay6125 (2019).
Pettersen, E. F., et al. UCSF ChimeraX: Structure visualization for researchers, educators, and developers. Protein Sci 30, 70-82 (2021).
Phadke, V. K., Bednarczyk, R. A., Salmon, D. A. & Omer, S. B. Association Between Vaccine Refusal and Vaccine-Preventable Diseases in the United States: A Review of Measles and Pertussis. JAMA 315, 1149-1158 (2016).
Phung, E., et al. Elicitation of pneumovirus-specific B cell responses by a prefusion-stabilized respiratory syncytial virus F subunit vaccine. Sci Transl Med 14, eabo5032 (2022).
Plattet, L. Alves, M. Herren, H. C. Aguilar, Measles virus fusion protein: Structure, function and inhibition. Viruses 8, 112 (2016).
Plemper, R. K., Brindley, M. A. & Iorio, R. M. Structural and mechanistic studies of measles virus illuminate paramyxovirus entry. PLoS Pathog. 7, e1002058 (2011).
Plemper, R. K.; Doyle, J.; Sun, A.; Prussia, A.; Cheng, L. T.; Rota, P. A.; Liotta, D. C.; Snyder, J. P.; Compans, R. W., Design of a small-molecule entry inhibitor with activity against primary measles virus strains. Antimicrob Agents Chemother 2005, 49 (9), 3755-61.PMC1195431
Polack, F. P., et al. Production of atypical measles in rhesus macaques: evidence for disease mediated by immune complex formation and eosinophils in the presence of fusion-inhibiting antibody. Nat Med 5, 629-634 (1999).
Poland, G. A. & Jacobson, R. M. The re-emergence of measles in developed countries: time to develop the next-generation measles vaccines? Vaccine 30, 103-104 (2012).
Poor, T. A. et al. Probing the paramyxovirus fusion (F) protein-refolding event from pre- to postfusion by oxidative footprinting. Proc. Natl. Acad. Sci. U.S.A 111, E2596-605 (2014).
Porotto, M.; Ferren, M.; Chen, Y. W.; Siu, Y.; Makhsous, N.; Rima, B.; Briese, T.; Greninger, A. L.; Snoeck, H. W.; Moscona, A., Authentic Modeling of Human Respiratory Virus Infection in Human Pluripotent Stem Cell-Derived Lung Organoids. mBio 2019, 10 (3).PMC6509192
Porotto, M.; Fornabaio, M.; Greengard, O.; Murrell, M. T.; Kellogg, G. E.; Moscona,
A., Paramyxovirus receptor-binding molecules: engagement of one site on the hemagglutinin-neuraminidase protein modulates activity at the second site. J Virol 2006, 80 (3), 1204-13.PMC1346948
Porotto, M.; Fornabaio, M.; Kellogg, G. E.; Moscona, A., A second receptor binding site on human parainfluenza virus type 3 hemagglutinin-neuraminidase contributes to activation of the fusion mechanism. J Virol 2007, 81 (7), 3216-28.PMC1866072
Porotto, M.; Yokoyama, C. C.; Orefice, G.; Kim, H. S.; Aljofan, M.; Mungall, B. A.; Moscona, A., Kinetic dependence of paramyxovirus entry inhibition. J Virol 2009, 83 (13), 6947-51.PMC2698554
Prince, G.; Hemming, V.; Horsewood, B.; Baron, P.; Chanock, R., Effectiveness of topically administered neutralizing antibodies in experimental immunotherapy of respiratory syncytial virus infection in cotton rats. J. Virol. 1987, 61, 1851-1854
Prince, G. A., Curtis, S. J., Yim, K. C. & Porter, D. D. Vaccine-enhanced respiratory syncytial virus disease in cotton rats following immunization with Lot 100 or a newly prepared reference vaccine. J.Gen. Virol. 82, 2881-2888 (2001).
Prussia, A. J.; Plemper, R. K.; Snyder, J. P., Measles virus entry inhibitors: a structural proposal for mechanism of action and the development of resistance. Biochemistry 2008, 47 (51), 13573-83
Pueschel, K.; Tietz, A.; Carsillo, M.; Steward, M.; Niewiesk, S., Measles virus-specific CD4 T-cell activity does not correlate with protection against lung infection or viral clearance. Journal of virology 2007, 81 (16), 8571-8.1951373
Punjani, A., Rubinstein, J. L., Fleet, D. J. & Brubaker, M. A. cryoSPARC: algorithms for rapid unsupervised cryo-EM structure determination. Nat. Methods 14, 290-296 (2017).
Racaniello, V., Virology. An exit strategy for measles virus. Science 2011, 334 (6063), 1650-1
Rainwater-Lovett, K., Nkamba, H. C., Mubiana-Mbewe, M., Bolton-Moore, C. & Moss, W. J. Changes in measles serostatus among HIV-infected Zambian children initiating antiretroviral therapy before and after the 2010 measles outbreak and supplemental immunization activities. J. Infect. Dis. 208, 1747-1755 (2013).
Rantalainen, K.; Berndsen, Z. T.; Antanasijevic, A.; Schiffner, T.; Zhang, X.; Lee, W. H.; Torres, J. L.; Zhang, L.; Irimia, A.; Copps, J.; Zhou, K. H.; Kwon, Y. D.; Law, W. H.; Schramm, C. A.; Verardi, R.; Krebs, S. J.; Kwong, P. D.; Doria-Rose, N. A.; Wilson, I. A.; Zwick, M. B.; Yates, J. R., 3rd; Schief, W. R.; Ward, A. B., HIV-1 Envelope and MPER Antibody Structures in Lipid Assemblies. Cell Rep 2020, 31 (4), 107583.PMC7196886
Rappazzo, C. G., et al. Potently neutralizing and protective anti-human metapneumovirus antibodies target diverse sites on the fusion glycoprotein. Immunity 55, 1710-1724 e1718 (2022).
Rayaprolu, V. et al. Structure of the Inmazeb cocktail and resistance to Ebola virus escape. Cell Host Microbe 31, 260-272.e7 (2023).
Reich, A., Erlwein, O., Niewiesk, S., ter Meulen, V. & Liebert, U. G. CD4+ T cells control measles virus infection of the central nervous system. Immunology 76, 185-191 (1992).
Rey, F. A. & Lok, S. M. Common Features of Enveloped Viruses and Implications for Immunogen Design for Next-Generation Vaccines. Cell 172, 1319-1334 (2018).
Reynard, O. et al. Nebulized fusion inhibitory peptide protects cynomolgus macaques from measles virus infection. Nat. Commun. 13, 6439 (2022).
Richardson, A. Scheid, P. W. Choppin, Specific inhibition of paramyxovirus and myxovirus replication by oligopeptides with amino acid sequences similar to those at the N-termini of the F1 or HA2 viral polypeptides. Virology 105, 205-222 (1980).
Richardson, et al., The nucleotide sequence of the mRNA encoding the fusion protein of measles virus (Edmonston strain): A comparison of fusion proteins from several different paramyxoviruses. Virology 155, 508-523 (1986).
Riddell, M. A., Rota, J. S. & Rota, P. A. Review of the temporal and geographical distribution of measles virus genotypes in the prevaccine and postvaccine eras. Virol. J. 2, 87 (2005).
Ritchie, T. K., et al. Chapter 11-Reconstitution of membrane proteins in phospholipid bilayer nanodiscs. Methods Enzymol 464, 211-231 (2009).
Rodriguez, C., et al. Fatal Measles Inclusion-Body Encephalitis in Adult with Untreated AIDS, France. Emerg Infect Dis 26, 2231-2234 (2020).
Rohou, A. & Grigorieff, N. CTFFIND4: Fast and accurate defocus estimation from electron micrographs. J. Struct. Biol. 192, 216-221 (2015).
Rossey, I., et al. A vulnerable, membrane-proximal site in human respiratory syncytial virus F revealed by a prefusion-specific single-domain antibody. J Virol 95 (2021).
Rota, P. A. & Bellini, W. J. Update on the global distribution of genotypes of wild type measles viruses. J. Infect. Dis. 187 Suppl 1, S270-6 (2003).
Rota, P. A.; Moss, W. J.; Takeda, M.; de Swart, R. L.; Thompson, K. M.; Goodson, J. L., Measles. Nat Rev Dis Primers 2016, 2 (1), 16049
Rush, S. A., et al. Characterization of prefusion-F-specific antibodies elicited by natural infection with human metapneumovirus. Cell Rep 40, 111399 (2022).
Sachs, N., et al. Long-term expanding human airway organoids for disease modeling. EMBO J 38 (2019).
Saeland, E., et al. Immunogenicity and protective efficacy of adenoviral and subunit RSV vaccines based on stabilized prefusion F protein in pre-clinical models. Vaccine 40, 934-944 (2022).
Santra, S.; et al., Human Non-neutralizing HIV-1 Envelope Monoclonal Antibodies Limit the Number of Founder Viruses during SHIV Mucosal Infection in Rhesus Macaques. PLoS Pathog 2015, 11 (8), e1005042.PMC4523205
Sapir, A., Avinoam, O., Podbilewicz, B. & Chernomordik, L. V. Viral and developmental cell fusion mechanisms: conservation and divergence. Dev. Cell 14, 11-21 (2008).
Scheepers, C.; Kgagudi, P.; Mzindle, N.; Gray, E. S.; Moyo-Gwete, T.; Lambson, B. E.; Oosthuysen, B.; Mabvakure, B.; Garrett, N. J.; Abdool Karim, S. S.; Morris, L.; Moore, P. L., Dependence on a variable residue limits the breadth of an HIV MPER neutralizing antibody, despite convergent evolution with broadly neutralizing antibodies. PLoS Pathog 2022, 18 (9), e1010450.PMC9477419
Scheres, S. H. W. RELION: implementation of a Bayesian approach to cryo-EM structure determination. J. Struct. Biol. 180, 519-530 (2012).
Schmitz, K. S., et al. Repurposing an In Vitro Measles Virus Dissemination Assay for Screening of Antiviral Compounds. Viruses 14 (2022).
Schneemann, A.; Speir, J. A.; Tan, G. S.; Khayat, R.; Ekiert, D. C.; Matsuoka, Y.; Wilson, I. A., A virus-like particle that elicits cross-reactive antibodies to the conserved stem of influenza virus hemagglutinin. J Virol 2012, 86 (21), 11686-97.PMC3486276
Schuchat, A., Fiebelkorn, A. P. & Bellini, W. Measles in the United States since the Millennium: Perils and Progress in the Postelimination Era. Microbiol Spectr 4, (2016).
Seither, et al., Coverage with selected vaccines and exemption from school vaccine requirements among children in kindergarten-United States, 2022-23 school year. MMWR Morb. Mortal. Wkly. Rep. 72, 1217-1224 (2023).
Seward, J. F. & Orenstein, W. A. Editorial commentary: A rare event: a measles outbreak in a population with high 2-dose measles vaccine coverage. Clinical infectious diseases: an official publication of the Infectious Diseases Society of America vol. 55 403-405 (2012).
Sievers, B. L., Sievers, R. E. & Sievers, E. L. Inundative, Dry-Powder, Inhaled Measles Vaccination to Prevent Deaths of Young Children in War-torn Regions. Open Forum Infect Dis 10, ofad302 (2023).
Sievers, F. & Higgins, D. G. Clustal omega. Curr. Protoc. Bioinformatics 48, 3.13.1-3.13.16 (2014).
Sneller, M. C. et al. Combination anti-HIV antibodies provide sustained virological suppression. Nature 606, 375-381 (2022).
Sowers, S. B. et al. High Concentrations of Measles Neutralizing Antibodies and High-Avidity Measles IgG Accurately Identify Measles Reinfection Cases. Clin. Vaccine Immunol. 23, 707-716 (2016).
Stewart-Jones, G. B. E. et al. Structure-based design of a quadrivalent fusion glycoprotein vaccine for human parainfluenza virus types 1-4. Proceedings of the National Academy of Sciences 115, 12265-12270 (2018).
Stokke, J. L. et al. MMR Vaccine-Associated Disseminated Measles in an Immunocompromised Adolescent. N. Engl. J. Med. 385, 1246-1248 (2021).
Stoneman, E. K. Measles. JAMA (2025).
Studer, G. et al. QMEANDisCo-distance constraints applied on model quality estimation. Bioinformatics 36, 1765-1771 (2020).
Sun, A. et al. Nonpeptide inhibitors of measles virus entry. J. Med. Chem. 49, 5080-5092 (2006).
Swanson, K. et al. Structure of the Newcastle disease virus F protein in the post-fusion conformation. Virology 402, 372-379 (2010).
Talekar, A., Moscona, A. & Porotto, M. Measles virus fusion machinery activated by sialic acid binding globular domain. J. Virol. 87, 13619-13627 (2013).
Tatsuo, H., Ono, N., Tanaka, K. & Yanagi, Y. SLAM (CDw150) is a cellular receptor for measles virus. Nature 406, 893-897 (2000).
Terwilliger, T. C., Ludtke, S. J., Read, R. J., Adams, P. D. & Afonine, P. V. Improvement of cryo-EM maps by density modification. Nat. Methods 17, 923-927 (2020).
The pandas development team, pandas-dev/pandas: Pandas [Computer software], Zenodo (2024); doi.org/10.5281/zenodo.3509134.
Toltzis, P. 50 Years Ago in The Journal of Pediatrics: Atypical Exanthem after Exposure to Natural Measles: Eleven Cases in Children Previously Inoculated with Killed Vaccine. J Pediatr 193, 84 (2018).
Tsai, Y.-X., et al. Rapid simulation of glycoprotein structures by grafting and steric exclusion of glycan conformer libraries. Cell 187, 1296-1311.e1226 (2024).
UNICEF and WHO warn of perfect storm of conditions for measles outbreaks, affecting children. www.who.int/news/item/27-04-2022-unicef-and-who-warn-of--perfect-storm--of-conditions-for-measles-outbreaks--affecting-children.
van den Hof, S., Berbers, G. A., de Melker, H. E. & Conyn-van Spaendonck, M. A. Seroepidemiology of measles antibodies in the Netherlands, a cross-sectional study in a national sample and in communities with low vaccine coverage. Vaccine 18, 931-940 (1999).
Virtanen, P. et al. SciPy 1.0: fundamental algorithms for scientific computing in Python. Nat. Methods 17, 261-272 (2020).
Vos, R. A., et al. Seroepidemiology of Measles, Mumps and Rubella on Bonaire, St. Eustatius and Saba: The First Population-Based Serosurveillance Study in Caribbean Netherlands. Vaccines (Basel) 7 (2019).
Waskom, M. seaborn: statistical data visualization. J. Open Source Softw. 6, 3021 (2021).
Waterhouse, A. et al. SWISS-MODEL: homology modelling of protein structures and complexes. Nucleic Acids Res. 46, W296-W303 (2018).
Welsch, J. C. et al. Fatal measles virus infection prevented by brain-penetrant fusion inhibitors. J. Virol. 87, 13785-13794 (2013).
Wendorf, K. A.; Winter, K.; Zipprich, J.; Schechter, R.; Hacker, J. K.; Preas, C.; Cherry, J. D.; Glaser, C.; Harriman, K., Subacute sclerosing panencephalitis: the devastating measles complication that might be more common than previously estimated. Clin Infect Dis 2017, 65 (2), 226-232
Whitaker, J. A., Shanafelt, T. D., Poland, G. A. & Kay, N. E. Room for improvement: immunizations for patients with monoclonal B-cell lymphocytosis or chronic lymphocytic leukemia. Clin. Adv. Hematol. Oncol. 12, 440-450 (2014).
White, J. M., Delos, S. E., Brecher, M. & Schornberg, K. Structures and mechanisms of viral membrane fusion proteins: multiple variations on a common theme. Crit. Rev. Biochem. Mol. Biol. 43, 189-219 (2008).
White, J. M., Ward, A. E., Odongo, L. & Tamm, L. K. Viral Membrane Fusion: A Dance Between Proteins and Lipids. Annu Rev Virol 10, 139-161 (2023).
Wiedermann, U., Garner-Spitzer, E. & Wagner, A. Primary vaccine failure to routine vaccines: Why and what to do? Hum Vaccin Immunother 12, 239-243 (2016).
Williamson, K. M., et al. A Cross-Sectional Study of Measles-Specific Antibody Levels in Australian Blood Donors-Implications for Measles Post-Elimination Countries. Vaccines (Basel) 12 (2024).
World Health Organization, “A 30-fold rise of measles cases in 2023 in the WHO European Region warrants urgent action,” 14 Dec. 2023; www.who.int/europe/news/item/14-12-2023-a-30-fold-rise-of-measles-cases-in-2023-in-the-who-european-region-warrants-urgent-action.
Wu, H. et al. Development of motavizumab, an ultra-potent antibody for the prevention of respiratory syncytial virus infection in the upper and lower respiratory tract. J. Mol. Biol. 368, 652-665 (2007).
Xiong, Y., et al. Age-related changes in serological susceptibility patterns to measles: results from a seroepidemiological study in Dongguan, China. Hum Vaccin Immunother 10, 1097-1003 (2014).
Xu, K., et al. Crystal Structure of the Pre-fusion Nipah Virus Fusion Glycoprotein Reveals a Novel Hexamer-of-Trimers Assembly. PLoS Pathog 11, e1005322 (2015).
Yanagi, Y., Ono, N., Tatsuo, H., Hashimoto, K. & Minagawa, H. Measles virus receptor SLAM (CD150). Virology 299, 155-161 (2002).
Yanagi, Y., Takeda, M., Ohno, S. & Hashiguchi, T. Measles virus receptors. Curr. Top. Microbiol. Immunol. 329, 13-30 (2009).
Yang, S.; Hiotis, G.; Wang, Y.; Chen, J.; Wang, J. H.; Kim, M.; Reinherz, E. L.; Walz, T., Dynamic HIV-1 spike motion creates vulnerability for its membrane-bound tripod to antibody attack. Nat Commun 2022, 13 (1), 6393.PMC9610346
Yin, H.-S., Paterson, R. G., Wen, X., Lamb, R. A. & Jardetzky, T. S. Structure of the uncleaved ectodomain of the paramyxovirus (hPIV3) fusion protein. Proc. Natl. Acad. Sci. U.S.A. 102, 9288-9293 (2005).
Yin, H.-S., Wen, X., Paterson, R. G., Lamb, R. A. & Jardetzky, T. S. Structure of the parainfluenza virus 5 F protein in its metastable, prefusion conformation. Nature 439, 38-44 (2006).
Zhang, L.; Irimia, A.; He, L.; Landais, E.; Rantalainen, K.; Leaman, D. P.; Vollbrecht, T.; Stano, A.; Sands, D. I.; Kim, A. S.; Investigators, I. P. G.; Poignard, P.; Burton, D. R.; Murrell, B.; Ward, A. B.; Zhu, J.; Wilson, I. A.; Zwick, M. B., An MPER antibody neutralizes HIV-1 using germline features shared among donors. Nat Commun 2019, 10 (1), 5389.PMC6879610
Zheng, S. Q. et al. MotionCor2: anisotropic correction of beam-induced motion for improved cryo-electron microscopy. Nat. Methods 14, 331-332 (2017).
Zhu, et al., A highly potent extended half-life antibody as a potential RSV vaccine surrogate for all infants. Sci. Transl. Med. 9, eaaj1928 (2017).
Zivanov, J. et al. New tools for automated high-resolution cryo-EM structure determination in RELION-3. Elife 7, (2018).
Zyla, D. dzyla/Follow_Relion_gracefully: v4. (2023). doi:10.5281/zenodo.7844516.
Zyla, D. S., et al. A neutralizing antibody prevents postfusion transition of measles virus fusion protein. Science 384, eadm8693 (2024).
Zyla, D. S., et al. Stabilized Full-Length Measles Fusion Protein Elicits Potent Immunity and Protection In Vivo. bioRxiv (2025).

Various modifications and variations of the described methods, pharmaceutical compositions, and kits of the invention can be apparent to those skilled in the art without departing from the scope and spirit of the invention. Although the invention has been described in connection with specific embodiments, it can be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed:

1. A thermostable full-length paramyxovirus fusion protein (F_{FL}), wherein the F_{FL} comprises at least one mutation that stabilizes the pre-fusion conformation, wherein the at least one mutation comprises at least one substitution mutation at a position corresponding to residue 170, residue 442, residue 455, residue 459, or residue 506 of measles virus (MeV) F protein.

2. The F_{FL} of claim 1, wherein the F_{FL} further comprises at least one mutation that removes or inactivates the furin cleavage site corresponding to the furin cleavage site of MeV F protein.

3. The F_{FL} of claim 1, wherein the F_{FL} comprises the following mutations corresponding to MeV F protein:
E455G; or
E170G/E455G; or
E170G/E455G/G506E; or
E170G/E455G/V459I/G506E; or
E170G/Y442H/E455G/G506E; or
E170G/Y442H/E455G/V459I/G506E.

4. The F_{FL} of claim 1, wherein the paramyxovirus is measles virus (MeV).

5. A paramyxovirus vaccine comprising the F_{FL} of claim 1.

6. A method of inducing protective immunity and neutralizing antibodies, the method comprising administration of a therapeutically effective dose of the paramyxovirus vaccine of claim 5 to a subject in need thereof.

7. A paramyxovirus vaccine composition comprising paramyxovirus F protein or its ectodomain, wherein the paramyxovirus F protein or its ectodomain comprises at least one mutation that stabilizes the pre-fusion conformation, wherein the at least one mutation comprises at least one substitution mutation at a position corresponding to residue 170, residue 442, residue 455, residue 459, or residue 506 of measles virus (MeV) F protein, optionally,
wherein the at least one mutation corresponds to E170G, Y442H, E455G, V459I, or G506E in MeV F protein; or
wherein the paramyxovirus F protein or its ectodomain comprises the following mutations corresponding to MeV F protein:
E455G; or
E170G/E455G; or
E170G/E455G/G506E; or
E170G/E455G/V459I/G506E; or
E170G/Y442H/E455G/G506E; or
E170G/Y442H/E455G/V459I/G506E.

8. The paramyxovirus vaccine composition of claim 7, wherein the paramyxovirus F protein or its ectodomain further comprises at least one mutation that removes or inactivates the furin cleavage site corresponding to the furin cleavage site of MeV F protein.

9. The paramyxovirus vaccine composition of claim 7, wherein the vaccine composition is a measles virus (MeV) vaccine composition.

10. A method of producing neutralizing antibodies against a paramyxovirus, the method comprising:
administering to a subject the paramyxovirus vaccine composition of claim 7;
confirming the subject produces neutralizing antibodies from the paramyxovirus vaccine composition;
isolating B cells from the subject;
culturing the B cells to form hybridomas; and
isolating the hybridoma that produces neutralizing antibodies against the paramyxovirus, optionally,
wherein the paramyxovirus is measles virus (MeV).

11. An antibody against measles virus F protein, comprising:
one or more light chain variable region complementarity determining regions (CDRs) selected from the group consisting of: a first CDR comprising the sequence RASKSISKYLA, a second CDR comprising the sequence SGSTLQS, and a third CDR comprising the sequence CQQHNEYTLT; or
one or more heavy chain variable region CDRs selected from the group consisting of: a first CDR comprising the sequence DYAWN, a second CDR comprising the sequence ISYTLTTGYNPSLKSRI, and a third CDR comprising the sequence RSGWLLPYWYFDV,
optionally, wherein the one or more light chain variable region CDRs are grafted onto a human antibody, and/or the one or more heavy chain variable region CDRs are grafted onto a human antibody.

12. The antibody of claim 11, wherein
the light chain variable region comprises a first CDR comprising the sequence RASKSISKYLA, a second CDR comprising the sequence SGSTLQS, and a third CDR comprising the sequence CQQHNEYTLT, and/or
the heavy chain variable region comprises a first CDR comprising the sequence DYAWN, a second CDR comprising the sequence ISYTLTTGYNPSLKSRI, and a third CDR comprising the sequence RSGWLLPYWYFDV.

13. The antibody of claim 11, wherein
the sequence of the light chain variable region is: DVQITQSPSYLAASPGETITINCRASKSISKYLAWYQEKPGKTNELLIYSGSTLQSGIPS RFRGSGSGTDFTLTISSLEPEDFAMYYCQQHNEYTLTFGGGTKLELKR, and/or
the sequence of the heavy chain variable region is: DVQLQESGPGLVKPSQSLSLTCTVSGYSITSDYAWNWIRQFPGNKLEWMGYISYTLT TGYNPSLKSRISITRDSSKNQFFLQLNSVTTEDTATYYCARSGWLLPYWYFDVWGAG TTVTVSS, optionally,
wherein the antibody is encoded by GS66077-1 pTRIOZ-hIgG1_HRV3C_77.1.

14. An antibody against measles virus F protein, comprising:
one or more light chain variable region complementarity determining regions (CDRs) selected from the group consisting of: a first CDR comprising the sequence RATSSVVYMH, a second CDR comprising the sequence ATSNLAS, and a third CDR comprising the sequence QQWNSNPYT; or
one or more heavy chain variable region CDRs selected from the group consisting of: a first CDR comprising the sequence NYWIE, a second CDR comprising the sequence EILPGNSYTNYNEKFRGKA, and a third CDR comprising the sequence PLSGNY,
optionally, wherein the one or more light chain variable region CDRs are grafted onto a human antibody, and/or the one or more heavy chain variable region CDRs are grafted onto a human antibody.

15. The antibody of claim 14, wherein
the light chain variable region comprises a first CDR comprising the sequence RATSSVVYMH, a second CDR comprising the sequence ATSNLAS, and a third CDR comprising the sequence QQWNSNPYT, and/or
the heavy chain variable region comprises a first CDR comprising the sequence NYWIE, a second CDR comprising the sequence EILPGNSYTNYNEKFRGKA, and a third CDR comprising the sequence PLSGNY.

16. The antibody of claim 14, wherein
the sequence of the light chain sequence is:
QIVLSQSPAILSASPGEKVTMTCRATSSVVYMHWYQQKPGSSPKPWIYAT
SNLASGVPTRFSGSGSGTSYSLTISRVEAEDAATYYCQQWNSNPYTFGGG
TKLEIKRADAAPTVSIFPPSSEQLTSGGASVVCFLNNFYPKDINVKWKID
GSERQNGVLNSWTDQDSKDSTYSMSSTLTLTKDEYERHNSYTCEATHKTS
TSPIVKSFNRNEC,
and/or
the sequence of the heavy chain sequence is:
QVQLQQSGAELMKPGASVKISCKAAGYTFSNYWIEWVKQRPGHGLEWIGE
ILPGNSYTNYNEKFRGKATITADTSSNTAYMQLSSLTSEDSAVYYCASPL
SGNYVRFAYWGQGTLVTVSTAKTTPPSVYPLAPGSAAQTNSMVTLGCLVK
GYFPEPVTVTWNSGSLSSGVHTFPAVLQSDLYTLSSSVTVPSSTWPSETV
TCNVAHPASSTKVDKKIVPRDCGCKPCICTVPEVSSVFIFPPKPKDVLTI
TLTPKVTCVVVDISKDDPEVQFSWFVDDVEVHTAQTQPREEQFNSTFRSV
SELPIMHQDWLNGKEFKCRVNSAAFPAPIEKTISKTKGRPKAPQVYTIPP
PKEQMAKDKVSLTCMITDFFPEDITVEWQWNGQPAENYKNTQPIMDTDGS
YFVYSKLNVQKSNWEAGNTFTCSVLHEGLHNHHTEKSLSHSPGK.

17. A humanized antibody comprising one or more CDRs from a single antibody selected from the group consisting of: C6, 9E1D3, H8, D9, and 10H2G3.

18. A method of neutralizing postfusion transition of measles virus (MeV) fusion protein in a subject, the method comprising administration of an effective amount of a first monoclonal antibody (mAb) selected from the group consisting of: G3, C6, F3, E11, 9E1D3, G4, H8, 10H2G3, D9, and Y10F, wherein the antibody is administered alone or in combination with mAb 77 to a subject in need thereof.

19. The method of claim 18, wherein the administration allows initial activation and refolding of the MeV F ectodomain and interrupts the refolding process of the F to prevent conformational changes required for membrane fusion and viral entry; and/or
wherein the first mAb, alone or in combination with mAb 77, forms a complex with F.

20. A method of inhibiting measles virus spread through cells or treating measles virus infection in a subject, the method comprising
administering to the cells or to the subject the humanized monoclonal antibody of claim 11, optionally,
wherein the humanized monoclonal antibody is administered onto a mucosal surface, such as by intranasal administration; or
wherein the humanized monoclonal antibody is administered within 24 hours of exposure to measles virus; or
wherein the humanized monoclonal antibody is administered no more than 12 hours before anticipated exposure to measles virus.
